(19) United States
(12) Patent Application Publication
(10) Pub. No.: US 2025/0313849 A1
(43) Pub. Date: Oct. 9, 2025

(54) OPTOGENETIC INDUCTION OF MEMBRANELESS ORGANELLES

(71) Applicant: UNIVERSITY OF PITTSBURGH-OF THE COMMONWEALTH SYSTEM OF HIGHER EDUCATION, Pittsburgh, PA (US)

(72) Inventors: Joseph Patrick CLARKE, Pittsburgh, PA (US); Christopher James DONNELLY, Pittsburgh, PA (US)

(21) Appl. No.: 19/187,075

(22) Filed: Apr. 23, 2025

Related U.S. Application Data
(62) Division of application No. 16/639,789, filed on Feb. 18, 2020, now Pat. No. 12,297,437.
(60) Provisional application No. 62/547,161, filed on Aug. 18, 2017.

(51) Int. Cl. C12N 15/63; A61N 5/06; C07K 14/415; C12Q 1/02; C12Q 1/6811

(52) U.S. Cl. CPC C12N 15/63; C07K 14/415; C12Q 1/025; C12Q 1/6811; A61N 5/062

(57) ABSTRACT

The present disclosure relates to compounds, compositions, and methods for the induction of membraneless organelles.

### CROSS REFERENCE TO RELATED APPLICATIONS

This application is a divisional application of U.S. Non-Provisional application Ser. No. 16/639,789, filed Feb. 18, 2020, which is a national stage entry of PCT International Application No. PCT/IB2018/056224, filed Aug. 17, 2018, which claims the benefit of U.S. Provisional Patent Application Ser. No. 62/547,161 filed Aug. 18, 2017, the disclosures of which are expressly incorporated herein by reference.

### REFERENCE TO SEQUENCE LISTING

A Sequence Listing conforming to the rules of WIPO Standard ST.26 is hereby incorporated by reference. Said Sequence Listing has been filed as an electronic document via PatentCenter in ASCII format encoded as XML. The electronic document, created on Apr. 17, 2025, is entitled “10504-020US2_ST26.xml”, and is 201,253 bytes in size.

### FIELD

The present disclosure relates to compounds, compositions, and methods for the induction of membraneless organelles.

### BACKGROUND

Organelles are cellular compartments that perform specific functions and are required for proper cellular homeostasis. Membraneless organelles are a class of organelles that do not contain a lipid membrane separating them from the cytoplasmic liquid. There are a number of cytoplasmic and nuclear membraneless organelles, each of which perform distinct cellular functions. The underlying biochemistry required for the formation of these organelles was long misunderstood since it was unclear how the membraneless organelles separated themselves from the cytoplasmic milieu.

Recent evidence suggests that the protein components of membraneless organelles contain low complexity domains (LCDs), or intrinsically disordered regions (IDRs). These LCDs and/or IDRs, when focally concentrated, undergo liquid-liquid phase separation (LLPS) due to self-interactions through weak multivalent attractive forces. These forces can be further stabilized by nucleic acids (RNA or DNA) and other molecules commonly found in each membraneless organelle.

There are a variety of cytoplasmic and nuclear membraneless organelles and their functions vary widely. Notably, a number of these membraneless organelles have been implicated in diseases. For example, abnormal stress granule and nucleoli dynamics are thought to contribute to the neuropathology of Amyotrophic Lateral Sclerosis, Alzheimer's Disease, Frontotemporal Dementia, and Parkinson's Disease. Mutations in components of stress granules are also found in certain cancers. Additionally, abnormal processing body (p-body) function has also been implicated in cancer pathobiology.

To date, no studies have been able to precisely control the spatial and temporal formation and/or dynamics of functional membraneless organelles, thus making their study in disease speculative. The ability to control these organelles would prove useful for manipulating specific cellular processes and would be invaluable for molecular and cellular biology. Current studies to manipulate membraneless organelle function rely on deleting key components of the structure, thus preventing their formation. This methodology, however, does not allow one to address the consequence of aberrant organelle formation, nor does it allow for researchers to control and interrogate their function. What is needed are new and improved methods for inducing membraneless organelles in mammalian cell lines and animal models.

The compounds, compositions, and methods disclosed herein address these and other needs.

### SUMMARY

Disclosed herein are compounds, compositions, and methods for inducing membraneless organelles in a cell or animal model. The inventors have developed a novel method to induce the formation of membraneless organelles using blue light stimulation. The compounds, compositions, and methods herein allow for the temporal and spatial tunability of membraneless organelle formation. These new methods enable researchers, for the first time, to stimulate the formation of these structures. These methods disclosed herein are utilized, for example, to study disease and for drug screening.

In one aspect, disclosed herein is a nucleotide sequence encoding a chimeric polypeptide, comprising: a first nucleotide sequence encoding a light-induced oligomerization domain and a second nucleotide sequence encoding a low complexity domain from a membraneless organelle target protein.

In one aspect, disclosed herein is an expression vector encoding a chimeric polypeptide, comprising: a first nucleotide sequence encoding a light-induced oligomerization domain and a second nucleotide sequence encoding a low complexity domain from a membraneless organelle target protein, wherein the first nucleotide sequence is operably linked to a promoter.

In one aspect, disclosed herein is a nucleotide sequence encoding a chimeric polypeptide, comprising: a first nucleotide sequence encoding a low complexity domain from a membraneless organelle target protein and a second nucleotide sequence encoding a light-induced oligomerization domain.

In one aspect, disclosed herein is an expression vector encoding a chimeric polypeptide, comprising: a first nucleotide sequence encoding a low complexity domain from a membraneless organelle target protein and a second nucleotide sequence encoding a light-induced oligomerization domain, wherein the first nucleotide sequence is operably linked to a promoter.

In one aspect, disclosed herein is a cell comprising a nucleotide sequence encoding a chimeric polypeptide, comprising: a first nucleotide sequence encoding a light-induced oligomerization domain and a second nucleotide sequence encoding a membraneless organelle target protein.

In one aspect, disclosed herein is a cell comprising a nucleotide sequence encoding a chimeric polypeptide, comprising: a first nucleotide sequence encoding a membraneless organelle target protein and a second nucleotide sequence encoding a light-induced oligomerization domain.

In one aspect, disclosed herein is a chimeric polypeptide comprising: a light-induced oligomerization domain; and a low complexity domain from a membraneless organelle target protein.

In one aspect, disclosed herein is a chimeric polypeptide comprising: a low complexity domain from a membraneless organelle target protein; and a light-induced oligomerization domain.

In one aspect, disclosed herein is a method of inducing a membraneless organelle in a cell, comprising the steps:
introducing into the cell an expression vector encoding a chimeric polypeptide, comprising:
   a first nucleotide sequence encoding a light-induced oligomerization domain and a second nucleotide sequence encoding a low complexity domain from a membraneless organelle target protein, wherein the first nucleotide sequence is operably linked to a promoter;
expressing the chimeric polypeptide; and
inducing oligomerization of the chimeric polypeptide by stimulation with blue light.

In another aspect, disclosed herein is a method of screening for an agent that modulates formation of a membraneless organelle, comprising the steps:
introducing into a cell an expression vector encoding a chimeric polypeptide, comprising:
   a first nucleotide sequence encoding a light-induced oligomerization domain and a second nucleotide sequence encoding a low complexity domain from a membraneless organelle target protein, wherein the first nucleotide sequence is operably linked to a promoter;
expressing the chimeric polypeptide;
introducing the agent into a culture media comprising the cell;
inducing oligomerization of the chimeric polypeptide by stimulation with blue light; and
determining modulation of the formation of the membraneless organelle by the agent.

In one aspect, disclosed herein is a method of inducing a membraneless organelle in a cell, comprising the steps:
introducing into the cell an expression vector encoding a chimeric polypeptide, comprising:
   a first nucleotide sequence encoding a low complexity domain from a membraneless organelle target protein and a second nucleotide sequence encoding a light-induced oligomerization domain, wherein the first nucleotide sequence is operably linked to a promoter;
expressing the chimeric polypeptide; and
inducing oligomerization of the chimeric polypeptide by stimulation with blue light.

In another aspect, disclosed herein is a method of screening for an agent that modulates formation of a membraneless organelle, comprising the steps:
introducing into a cell an expression vector encoding a chimeric polypeptide, comprising:
   a first nucleotide sequence encoding a low complexity domain from a membraneless organelle target protein and a second nucleotide sequence encoding a light-induced oligomerization domain, wherein the first nucleotide sequence is operably linked to a promoter;
expressing the chimeric polypeptide;
introducing the agent into a culture media comprising the cell;
inducing oligomerization of the chimeric polypeptide by stimulation with blue light; and
determining modulation of the formation of the membraneless organelle by the agent.

In one embodiment, the light-induced oligomerization domain is selected from the group consisting of CRY2 PHR, CRY2OLIG, NcVVD, NcVVDY50W, NcVVDY50W/I74V/I85V/LINKERA, NcVVDY50W/I52C/I74V/I85V/LINKERA, NcVVDY50W/C71V/I74V/I85V/LINKERA,

NcVVDY50W/I52C/C71V/I74V/I85V/LINKERA, NcVVDY50W/I74V/I85V/LINKERB, NcVVDY50W/I52C/I74V/I85V/LINKERB, NcVVDY50W/C71V/I74V/I85V/LINKERB, NcVVDY50W/I52C/C71V/I74V/I85V/LINKERB, NcLOV, and VfAU1LOV. In one embodiment, the light-induced oligomerization domain is NcVVDY50W. In one embodiment, the light-induced oligomerization domain is NcVVDY50W/I74/I85/LINKERA. In one embodiment, the light-induced oligomerization domain is NcVVDY50W/I52C/I74/I85/LINKERA. In one embodiment, the light-induced oligomerization domain is NcVVDY50W/C71V/I74/I85/LINKERA. In one embodiment, the light-induced oligomerization domain is NcVVDY50W/I52C/C71V/I74/I85/LINKERA. In one embodiment, the light-induced oligomerization domain is NcVVDY50W/I74/I85/LINKERB. In one embodiment, the light-induced oligomerization domain is NcVVDY50W/I52C/I74/I85/LINKERB. In one embodiment, the light-induced oligomerization domain is NcVVDY50W/C71V/I74/I85/LINKERB. In one embodiment, the light-induced oligomerization domain is NcVVDY50W/I52C/C71V/I74/I85/LINKERB. In one embodiment, the light-induced oligomerization domain is CRY2OLIG. In one embodiment, the light-induced oligomerization domain is CRY2 PHR. In one embodiment, the light-induced oligomerization domain comprises a LOV domain. In one embodiment, the light-induced oligomerization domain comprises a LOV domain from the VVD protein. In one embodiment, the light-induced oligomerization domain comprises a LOV domain from the LOV protein. In one embodiment, the light-induced oligomerization domain comprises a PHR domain. In one embodiment, the light-induced oligomerization domain comprises a PHR domain, from the CRY2 protein.

In one embodiment, the low complexity domain is from a membraneless organelle target protein. In one embodiment, the low complexity domain from a membraneless organelle target protein is from a cytoplasmic membraneless organelle target protein.

In one embodiment, the low complexity domain from a membraneless organelle target protein is from a stress granule target protein. In one embodiment, the low complexity domain is from a stress granule target protein selected from the group consisting of PABC1, TIAR, G3BP1, G3BP2, DDX6, TDRD3, and ATXN2. In one embodiment, the low complexity domain from a membraneless organelle target protein is G3BP1. In one embodiment, the low complexity domain from a membraneless organelle target protein is an NTF2 domain truncated G3BP1. In one embodiment, the low complexity domain from a membraneless organelle target protein is an acidic domain truncated G3BP1. In one embodiment, the low complexity domain from a membraneless organelle target protein is a PxxP domain truncated G3BP1. In one embodiment, the low complexity domain from a membraneless organelle target protein is an RRM domain truncated G3BP1. In one embodiment, the low complexity domain from a membraneless organelle target protein is an RGG domain truncated G3BP1.

In one embodiment, the low complexity domain from a membraneless organelle target protein is from a P-body target protein. In one embodiment, the low complexity domain is from a P-body target protein selected from the group consisting of DCP1A, DCP2, LSM1, TNRC6A, MEX3A, EDC4, XRN1, and DDX3X.

In one embodiment, the low complexity domain from a membraneless organelle target protein is from a ribonuclear transport granule target protein. In one embodiment, the low complexity domain is from a ribonuclear transport granule target protein selected from the group consisting of IGFBP1, STAU1, PURA, FMR1, FXR1, and FXR2.

In one embodiment, the low complexity domain from a membraneless organelle target protein is from a nuclear membraneless organelle target protein.

In one embodiment, the low complexity domain from a membraneless organelle target protein is from a nucleolus target protein. In one embodiment, the low complexity domain is from a nucleolus target protein selected from the group consisting of NCL, NPM1, and FBL.

In one embodiment, the low complexity domain from a membraneless organelle target protein is from a nuclear speckle target protein. In one embodiment, the low complexity domain is from a nuclear speckle target protein selected from the group consisting of SRSF2, PNN, and SRSF1.

In one embodiment, the low complexity domain from a membraneless organelle target protein is from a nuclear stress body target protein. In one embodiment, the low complexity domain is from nuclear stress body target protein SAFB.

In one embodiment, the low complexity domain from a membraneless organelle target protein is from a nuclear paraspeckle target protein. In one embodiment, the low complexity domain is from a nuclear paraspeckle target protein selected from the group consisting of SFPQ, NONO, and PSPC1.

In one embodiment, the low complexity domain from a membraneless organelle target protein is from a cajal body target protein. In one embodiment, the low complexity domain is from cajal body target protein COIL.

In one embodiment, the low complexity domain from a membraneless organelle target protein is from a PML body target protein. In one embodiment, the low complexity domain is from PML body target protein PML.

In one embodiment, the low complexity domain from a membraneless organelle target protein is from a chromatoid body/germ granule target protein. In one embodiment, the low complexity domain is from chromatoid body/germ granule target protein DDX4.

In one embodiment, the cell is a mammalian cell. In one embodiment, the cell is a human cell.

In one embodiment, the blue light has a wavelength between 405 nm and 499 nm. In one embodiment, the blue light has a wavelength of about 465 nm.

### BRIEF DESCRIPTION OF THE DRAWINGS

The accompanying figures, which are incorporated in and constitute a part of this specification, illustrate several aspects described below.

FIG. 1. Schematic of a dimerizing photoreceptor upon light stimulation. When fused to proteins that make up the core of membraneless organelles and often contain LCDs/IDRs, and employing a specific light stimulation paradigm, this photoreceptor is used for oligomerization. The oligomerization of core proteins of membraneless organelles initiate their formation. These novel light stimulation paradigms are used induce oligomerization of fusions proteins with LCDs.

FIGS. 2A-2B. Blue light induced stress granules using a NcVVDY50W-G3BP1 protein arrangement. FIG. 2A) The stress granules exhibit a composition that matches the heat shock induced stress granules and contain the stress granule component, Ataxin-2 protein. FIG. 2B) The stress granules exhibit a composition that matches the heat shock induced stress granules and contain the stress granule component, TIAR protein. A DNA arrangement that generates the NcVVDY50W-G3BP1 was developed and expressed in HEK293 cells. Under normal conditions, the NcVVDY50W-G3BP1 remains diffuse in the cytoplasm. When endogenous stress granule formation is induced with heat shock, the NcVVDY50W-G3BP1 forms stress granules that stain positive for key markers including Ataxin-2 and TIAR. To test if blue light exposure can induce stress granule formation, a light treatment paradigm was developed and the resulting light-induced stress granules are similarly stained positive for Ataxin-2 and TIAR.

FIG. 3. Blue light induced stress granule using G3BP1-NcVVDY50W protein arrangement exhibit a composition that matches sodium arsenite (NaAsO₂) induced stress granules and contain the stress granule components, Ataxin-2 and eIF4G. Under normal conditions, G3BP1-NcVVDY50W remains diffuse in the cytoplasm. When endogenous stress granule formation is induced with NaAsO₂ (0.5 mM, 30 min treatment), the G3BP1-NcVVDY50W forms stress granules that stain positive for the key translation factor eIF4G, and the stress granule marker Ataxin-2. Blue light induced stress granules were also found to colocalize with the key translation factor eIF4G, and the stress granule marker Ataxin-2.

FIG. 4. Blue light induced stress granules using G3BP1-NcVVDY50W protein arrangement exhibit a composition that matches sodium arsenite (NaAsO₂) induced stress granules and contain the stress granule components, TIAR and G3BP. Under normal conditions, G3BP1-NcVVDY50W remains diffuse in the cytoplasm. When endogenous stress granule formation is induced with NaAsO₂ (0.5 mM, 30 min treatment), the G3BP1-NcVVDY50W forms stress granules that stain positive for key markers TIAR and G3BP. Blue light induced stress granules were also found to colocalize with the key stress granule markers TIAR and G3BP. Under normal conditions, the G3BP1-NcVVDY50W remains diffuse in the cytoplasm. When endogenous stress granule formation were induced with sodium arsenite, the G3BP1-NcVVDY50W forms stress granules that probe positive for key markers, including TIAR and G3BP. To test if blue light exposure can induce stress granule formation, a light treatment paradigm was developed to produce light-induced stress granules that also probe positive for TIAR and G3BP. Inserts are 3D images of stress granules indicated by arrows.

FIG. 5. Blue light induced stress granules using G3BP1-NcVVDY50W protein arrangement exhibit a composition that matches sodium arsenite induced (NaAsO₂) stress granules and contain the stress granule components, TDP-43 and eIF4G. Under normal conditions, G3BP1-NcVVDY50W remains diffuse in the cytoplasm. When endogenous stress granule formation is induced with NaAsO₂ (0.5 mM, 30 min treatment), the G3BP1-NcVVDY50W forms stress granules that stain positive for key markers TDP-43 and eIF4G. Blue light induced stress granules were also found to colocalize with the key stress granule markers TIAR and G3BP. As an example of this technology, DNA arrangements were developed that generated G3BP1-NcVVDY50W, and expressed in HEK293 cells. Under normal conditions, the G3BP1-NcVVDY50W remains diffuse in the cytoplasm. When endogenous stress granule formation is induced with sodium arsenite, the G3BP1-NcVVDY50W forms stress granules that probe positive for key markers, including TDP-43 and eIF4G. To test if blue light exposure can induce stress granule formation, a light treatment paradigm was developed to produce light-induced stress granules that also probe positive for TDP-43 and eIF4G. Inserts are 3D images of stress granules indicated by arrows.

FIGS. 6A-6B. Blue light induced stress granules using G3BP1-NcVVDY50W protein arrangement exhibit a composition that matches sodium arsenite (NaAsO₂) induced stress granules and contain the stress granule components, RPS6 and eIF4G (FIG. 6A), as well as Ataxin-2 and RPS3 (FIG. 6B). Under normal conditions, G3BP1-NcVVDY50W remains diffuse in the cytoplasm. When endogenous stress granule formation is induced with NaAsO₂ (0.5 mM, 30 min treatment), the G3BP1-NcVVDY50W forms stress granules that stain positive for key markers RPS6 and eIF4G (FIG. 6A), as well as Ataxin-2 and RPS3 (FIG. 6B). Blue light induced stress granules were also found to colocalize with the key stress granule markers RPS6 and eIF4G (FIG. 6A), as well as Ataxin-2 and RPS3 (FIG. 6B).

FIGS. 7A-7B. Blue light induced stress granules using G3BP1-NcVVDY50W protein arrangement exhibit a composition that matches sodium arsenite (NaAsO₂) induced stress granules and contain the stress granule components, PABPC1 and G3BP (FIG. 7A), as well as eIF3H and eIF4G (FIG. 7B). Under normal conditions, G3BP1-NcVVDY50W remains diffuse in the cytoplasm. When endogenous stress granule formation is induced with NaAsO₂ (0.5 mM, 30 min treatment), the G3BP1-NcVVDY50W forms stress granules that stain positive for key markers PABPC1 and G3BP (FIG. 7A), as well as eIF3H and eIF4G (FIG. 7B). Blue light induced stress granules were also found to colocalize with the key stress granule markers PABPC1 and G3BP (FIG. 7A), as well as eIF3H and eIF4G (FIG. 7B).

FIG. 8. Blue light induced stress granules using NcVVDY50W-G3BP1 protein arrangement exhibit a composition that matches heat shock induced stress granules and contain the stress granule component, eIF3H protein. Under normal conditions, NcVVDY50W-G3BP1 remains diffuse in the cytoplasm. When endogenous stress granule formation is induced with heat shock, the NcVVDY50W-G3BP1 forms stress granules that stain positive for the key translation factor eIF3H. Blue light induced stress granules were also found to colocalize with the key stress granule marker eIF3H.

FIG. 9. Blue light induced stress granules using G3BP1-NcVVDY50W protein arrangement exhibit a composition that matches sodium arsenite induced (NaAsO₂) stress granules and contain the stress granule components, FUS and eIF4G. Under normal conditions, G3BP1-NcVVDY50W remains diffuse in the cytoplasm. When endogenous stress granule formation is induced with NaAsO₂ (0.5 mM, 30 min treatment), the G3BP1-NcVVDY50W forms stress granules that stain positive for key markers FUS and eIF4G. Blue light induced stress granules were also found to colocalize with the key stress granule markers FUS and G3BP.

FIG. 10. Blue light induced stress granules using G3BP1-NcVVDY50W protein arrangement exhibit a composition that matches sodium arsenite induced (NaAsO₂) stress granules and contain the stress granule component Ataxin-2, but not RPL36a. RPL36a is a component of the 60S ribosome and is not recruited to endogenous stress granules. Under normal conditions, G3BP1-NcVVDY50W remains diffuse in the cytoplasm. When endogenous stress granule formation is induced with NaAsO₂ (0.5 mM, 30 min treatment), the G3BP1-NcVVDY50W forms stress granules that stain positive for the key marker Ataxin-2, but not RPL36a. Blue light induced stress granules were also found to colocalize with the key stress granule marker Ataxin-2, but not RPL36a. This indicates that seeding light induced stress granule core through G3BP1 oligomerization forms granules that mimic endogenous stress granule structure.

FIGS. 11A-11D. Blue light induced stress granules using G3BP1-NcVVDY50W protein arrangement exhibit a cellular occurrence and structural characteristics that matches sodium arsenite induced (NaAsO₂) stress granules and contain the stress granule component Ataxin-2. Under normal conditions, G3BP1-NcVVDY50W remains diffuse in the cytoplasm. When endogenous stress granule formation is induced with NaAsO₂ (0.5 mM, 30 min treatment), the G3BP1-NcVVDY50W forms stress granules that stain positive for the key marker Ataxin-2, and occur in the cell 11 times on average (FIG. 11A). They also have a defined shape that gives them a defined area (FIG. 11B), diameter (FIG. 11C) and circularity (FIG. 11D). Blue light induced stress granules were also found to colocalize with the key stress granule marker Ataxin-2, and also have similar characteristics as endogenously formed stress granules (FIG. 11A-11D).

FIG. 12. Blue light induced stress granules using NcVVDY50W-G3BP1 protein arrangement sequester mRNA, indicating they are functional. Under normal conditions, NcVVDY50W-G3BP1 remains diffuse in the cytoplasm. When endogenous stress granule formation was induced with heat shock, the NcVVDY50W-G3BP1 formed stress granules that sequester mRNA, the primary function of stress granules. Blue light induced stress granules also sequester cytoplasmic mRNA indicating they are functional.

FIG. 13. Blue light induced stress granules using G3BP1-NcVVDY50W protein arrangement sequester mRNA, indicating they are functional. Under normal conditions, G3BP1-NcVVDY50W remains diffuse in the cytoplasm. When endogenous stress granule formation was induced with sodium arsenite (NaAsO₂), the G3BP1-NcVVDY50W formed stress granules that sequester mRNA, the primary function of stress granules, and colocalized with the key stress granule marker TIAR. Blue light induced stress granules also sequester cytoplasmic mRNA indicating they are functional, and colocalized with the key stress granule marker TIAR.

FIGS. 14A-14B. Optogenetically induced stress granules function as endogenous stress granules and stall protein synthesis. The major function of endogenous stress granules is to bind mRNAs and halt protein synthesis until the cellular stress is removed. To test this, cells expressing G3BP1-NcVVDY50W were either exposed to blue light stimulation or not for 30 minutes and protein translation was assessed. Prior to the translation assay, cells were treated with cyclohexamide for one hour to inhibit protein synthesis and then the cyclohexamide was removed and cells were provided L-Homopropargylglycine (HPG) for one hour. HPG acts as a methionine amino acid analog and is incorporated into newly synthesized proteins. Click-iT™ chemistry was then performed to detect HPG and cells were imaged. Green signal indicates newly synthesized proteins during the time at which HPG is present in the media. Cells with light-induced stress granules show little to no HPG signal indicating protein synthesis was stalled during the HPG incubation due to blue-light induced stress granules (FIG. 14A). Analysis indicates that there was a 40% reduction in protein synthesis with light-induced stress granules (FIG. 14B). These results indicate that the light-induced stress granules perform the same key function as endogenous stress granules and inhibit protein synthesis throughout the time of their formation.

FIG. 15. Optogenetically induced stress granules are tunable. NcVVDY50W-G3BP1 was expressed in HEK293 cells. Stress granules can be controlled with light stimulation and repeatedly formed. NcVVDY50W-G3BP1 expressing HEK293 cells were longitudinally imaged every 30 seconds and exposed to blue light in between each image for 30 minutes. The light was then removed and cells were imaged every 30 seconds without light in between each image to study the rate of formation and dissociation of the light induced stress granules.

FIGS. 16A-16B. Quantification of optogenetically induced stress granules dynamics. FIG. 16A shows a representative image of time lapsed image during blue light on/off treatment at different time points from FIG. 15. FIG. 16B shows quantification of puncta formation and dissociation rate over time from FIG. 15. Light induced stress granules are dynamic and can be repeatedly stimulated to control their function in the absence of toxic cellular stressors.

FIGS. 17A-17B. Optogenetically induced stress granules are tunable with different voltages of blue light stimulation. FIG. 17A) NcVVDY50W-G3BP1 was expressed in HEK293 cells. Stress granule formation can be temporally controlled with light stimulation at different voltages. FIGS. 17B) NcVVDY50W-G3BP1 expressing HEK293 cells were longitudinally imaged every 30 seconds and exposed to different voltages (1.0V, or 5.0V, or 10.0V) of blue light in between each image for 30 minutes, over a time-course of 6 hours. The light was then removed and cells were imaged every 30 seconds without light in between each image to study the rate of formation of the light induced stress granules.

FIGS. 18A-18E. Optogenetically induced stress granules dynamics can be chronically formed for hours in the absence of cell stressors. NcVVDY50W-G3BP1 was expressed in HEK293 cells. FIG. 18A shows stress granules can be chronically formed for hours with light stimulation in the absence of toxic cell stressors. Light paradigms were developed to chronically form stress granules with light for up to 18 hours. The light induced stress granules are positive for both key markers such as PABPC1 and sequester mRNA throughout the time course, indicating they are functional. FIG. 18B shows the number of puncta per cell over time after stimulation with blue light. FIG. 18C shows the puncta volume over time after stimulation with blue light. FIG. 18D shows the puncta area over time after stimulation with blue light. FIG. 18E shows the colocalization of puncta and TIAR over time after stimulation with blue light. The stress granules decrease in number and increase in size over the time course indicating their fluid nature, a characteristic of stress granules. No previous study has been able to induce stress granule formation for more than 2-3 hours as the cells die from the treatment to induce stress granules. Thus, these are the first studies to create chronic stress granules for over 2-3 hours.

FIG. 19. Optogenetically induced stress granules can be formed with a Cry2Olig-TIA1 protein arrangement and blue light stimulation in rodent cortical neurons. In addition to G3BP, TIA1 is another core component of stress granules and required for their formation in cells. To show that optogenetic induction of membraneless organelles can be performed with a variety of core protein components that 1) contain low complexity domains and 2) the organelle cannot form under endogenous conditions when that specific core component is removed, a Cry2Olig-TIA1 DNA construct was generated and expressed it in rat cortical neuron cultures. The cells were then exposed to 30 minutes of blue light stimulation, or not, and counterstained for markers of stress granules. It was observed that TIA1 clusters form with blue light stimulation (bottom) and these colocalize with G3BP, a marker of stress granules. This data shows that the methodology is valid for a variety of membraneless organelles using their core LCD-containing protein components and photoreceptor arrangement along with defined blue light stimulation paradigms.

FIG. 20. Blue light induced stress granules using a G3BP1-NcVVDY50W protein arrangement in ReNCell differentiated cortical neuron-like cells. A DNA arrangement that generates the G3BP1-NcVVDY50W was developed and expressed in ReNCell differentiated cortical neuron-like cells. To confirm differentiation of ReN cells to cortical neuron-like cells, cultures were probed for MAP2, a protein that is only expressed in neurons. Under normal conditions, the NcVVDY50W-G3BP1 remains diffuse in the cytoplasm. Blue light induced stress granules were found to colocalize with the key stress granule marker Ataxin-2.

FIGS. 21A-21B. Optogenetically induced stress granules are specifically formed by blue light and do not use a cell stress. Total cellular protein from blue light induced stress granules using G3BP1-NcVVDY50W protein arrangement was collected. Subsequent Western blotting of collected total protein, probing for phospho-eIF2a (FIG. 21A) and ATF4 (FIG. 21B), both of which are components of the integrated stress response (ISR) pathway, indicates that optogenetically induced stress granules do not induce a cell stress to form stress granules using the G3BP1-NcVVDY50W protein arrangement.

FIGS. 22A-22B. Optogenetically induced stress granule formation is not affected by cell stress pathway inhibitor GSK2606414 (PERK inhibitor; PERKi). When endogenous stress granule formation is induced with NaAsO₂ (0.5 mM, 30 min treatment), the G3BP1-NcVVDY50W forms stress granules that stain positive for key markers TIAR and eIF4G. When GSK2606414, a PERK kinase inhibitor, is added 1 hour before NaAsO₂ treatment, endogenous stress granule formation is attenuated (FIG. 22A). When GSK2606414 is added 1 hour before blue light stimulation, there is no change in stress granule formation (FIG. 22B). These results further indicate that optogenetically induced stress granules do not use a cell stress to form.

FIGS. 23A-23B. Translational inhibition with cycloheximide inhibits optogenetically induced stress granule formation. When endogenous stress granule formation is induced with NaAsO₂ (0.5 mM, 30 min treatment), the G3BP1-NcVVDY50W forms stress granules that stain positive for the key marker eIF4G. When cycloheximide, a translational inhibitor, is added 1 and a half hours before NaAsO₂ treatment, endogenous stress granule formation is attenuated (FIG. 23A). When cycloheximide is added 1 and a half hours before blue light stimulation, optogenetically induced stress granules fail to form (FIG. 23B). These results indicate that like endogenous stress granules, optogenetically induced stress granules require cellular translational machinery to form.

FIGS. 24A-24E. Replacing the G3BP1 NTF2 domain, which is required for G3BP1 self-oligomerization (optoSG), with oligomerizing photoreceptors, is sufficient to initiate the formation of optogenetic stress granules. FIG. 24A) Schematics of an additional approach to seed optoStress Granules (optoSGs) employing truncated G3BP1 protein and replacing the NTF2 oligomerizing domain with a photoreceptor with the capacity to oligomerize (in this instance Cry2-PHR, but also works with chronic stimulation of dimerizing photoreceptor). FIG. 24B) HEK cells expressing Cry2 PHR-□NTF-G3BP1 were exposed to blue light LED stimulation for 30 minutes to induce the formation of optoSGs. SGs were monitored for 165 seconds for fission and fusion and exhibit the liquid-like droplet properties of endogenously formed stress granules. FIG. 24C) Cry2 PHR-□NTF-G3BP1 optoSGs colocalize with endogenous stress granule markers including PABPC1 and the 40S ribosomal component RPS3, FIG. 24D) but not the 60S ribosomal component RPL36a. FIG. 24E) De novo protein synthesis was analyzed as described above using Click-iT biochemistry and show that light induced Cry2 PHR-INTF-G3BP1 optoSGs inhibit cellular protein synthesis to a similar degree as sodium arsenite induced stress granules.

FIGS. 25A-25C. OptoStress Granules (optoSGs) (Cry2-delta NTF-G3BP1) display liquid-like properties of membraneless organelles and colocalize with translation initiation factors and mRNA. FIG. 25A) Longitudinal live imaging of HEKs expressing Cry2 PHR-NTF-G3BP1 exposed to blue light LED stimulation show that optoSGs are form after 15 minutes and exhibit a residence time of approximately 2-3 minutes before dissipating. FIG. 25B) Cry2 PHR-□NTF-G3BP1 optoSGs colocalize with mature stress granule markers Ataxin2 and eIF4G translational factors as well as FIG. 25C) mRNA and TDP-43. Together these data indicate that optoSGs using the Cry2 PHR-□NTF-G3BP1 approach as well as the full length G3BP1 protein seed core of stress granule like structures that function as endogenous stress granules to inhibit protein synthesis in the absence of the intrinsic stress response.

### DETAILED DESCRIPTION

Disclosed herein are compounds, compositions, and methods for inducing membraneless organelles in a cell or animal. The inventors have developed a novel method to induce the formation of membraneless organelles using blue light stimulation. The compounds, compositions, and methods herein allow for the temporal and spatial tunability of membraneless organelle formation. These new methods enable researchers, for the first time, to stimulate the formation of these structures. These methods are used for disease studies and for drug screening.

Reference will now be made in detail to the embodiments of the invention, examples of which are illustrated in the drawings and the examples. This invention may, however, be embodied in many different forms and should not be construed as limited to the embodiments set forth herein.

Unless defined otherwise, all technical and scientific terms used herein have the same meaning as commonly understood to one of ordinary skill in the art to which this invention belongs. The following definitions are provided for the full understanding of terms used in this specification.

### Terminology

As used herein, the article “a,” “an,” and “the” means “at least one,” unless the context in which the article is used clearly indicates otherwise.

The term “nucleic acid” as used herein means a polymer composed of nucleotides, e.g. deoxyribonucleotides or ribonucleotides.

The terms “ribonucleic acid” and “RNA” as used herein mean a polymer composed of ribonucleotides.

The terms “deoxyribonucleic acid” and “DNA” as used herein mean a polymer composed of deoxyribonucleotides.

The term “oligonucleotide” denotes single- or double-stranded nucleotide multimers of from about 2 to up to about 100 nucleotides in length. Suitable oligonucleotides may be prepared by the phosphoramidite method described by Beaucage and Carruthers, Tetrahedron Lett., 22:1859-1862 (1981), or by the triester method according to Matteucci, et al., J. Am. Chem. Soc., 103:3185 (1981), both incorporated herein by reference, or by other chemical methods using either a commercial automated oligonucleotide synthesizer or VLSIPS™ technology. When oligonucleotides are referred to as “double-stranded,” it is understood by those of skill in the art that a pair of oligonucleotides exist in a hydrogen-bonded, helical array typically associated with, for example, DNA. In addition to the 100% complementary form of double-stranded oligonucleotides, the term “double-stranded,” as used herein is also meant to refer to those forms which include such structural features as bulges and loops, described more fully in such biochemistry texts as Stryer, Biochemistry, Third Ed., (1988), incorporated herein by reference for all purposes.

The term “polynucleotide”, “nucleotide sequence”, or “nucleic acid sequence” refers to a single or double stranded polymer composed of nucleotide monomers.

The term “polypeptide” refers to a compound made up of a single chain of D- or L-amino acids or a mixture of D- and L-amino acids joined by peptide bonds.

The term “complementary” refers to the topological compatibility or matching together of interacting surfaces of a probe molecule and its target. Thus, the target and its probe can be described as complementary, and furthermore, the contact surface characteristics are complementary to each other.

The term “hybridization” refers to a process of establishing a non-covalent, sequence-specific interaction between two or more complementary strands of nucleic acids into a single hybrid, which in the case of two strands is referred to as a duplex.

The term “anneal” refers to the process by which a single-stranded nucleic acid sequence pairs by hydrogen bonds to a complementary sequence, forming a double-stranded nucleic acid sequence, including the reformation (renaturation) of complementary strands that were separated by heat (thermally denatured).

The term “melting” refers to the denaturation of a double-stranded nucleic acid sequence due to high temperatures, resulting in the separation of the double strand into two single strands by breaking the hydrogen bonds between the strands.

The term “target” refers to a molecule that has an affinity for a given probe. Targets may be naturally-occurring or man-made molecules. Also, they can be employed in their unaltered state or as aggregates with other species.

The term “promoter” or “regulatory element” refers to a region or sequence determinants located upstream or downstream from the start of transcription and which are involved in recognition and binding of RNA polymerase and other proteins to initiate transcription. Promoters need not be of bacterial origin, for example, promoters derived from viruses or from other organisms can be used in the compositions, systems, or methods described herein. The term “regulatory element” is intended to include promoters, enhancers, internal ribosomal entry sites (IRES), and other expression control elements (e.g. transcription termination signals, such as polyadenylation signals and poly-U sequences). Such regulatory elements are described, for example, in Goeddel, Gene Expression Technology: Methods in Enzymology 185, Academic Press, San Diego, Calif. (1990). Regulatory elements include those that direct constitutive expression of a nucleotide sequence in many types of host cell and those that direct expression of the nucleotide sequence only in certain host cells (e.g., tissue-specific regulatory sequences). A tissue-specific promoter may direct expression primarily in a desired tissue of interest, such as muscle, neuron, bone, skin, blood, specific organs (e.g. liver, pancreas), or particular cell types (e.g. lymphocytes). Regulatory elements may also direct expression in a temporal-dependent manner, such as in a cell-cycle dependent or developmental stage-dependent manner, which may or may not also be tissue or cell-type specific. In some embodiments, a vector comprises one or more pol III promoter (e.g. 1, 2, 3, 4, 5, or more pol I promoters), one or more pol II promoters (e.g. 1, 2, 3, 4, 5, or more pol II promoters), one or more pol I promoters (e.g. 1, 2, 3, 4, 5, or more pol I promoters), or combinations thereof. Examples of pol III promoters include, but are not limited to, U6 and H1 promoters. Examples of pol II promoters include, but are not limited to, the retroviral Rous sarcoma virus (RSV) LTR promoter (optionally with the RSV enhancer), the cytomegalovirus (CMV) promoter (optionally with the CMV enhancer) [see, e.g., Boshart et al, Cell, 41:521-530 (1985)], the SV40 promoter, the dihydrofolate reductase promoter, the β-actin promoter, the phosphoglycerol kinase (PGK) promoter, and the EFla promoter. Also encompassed by the term “regulatory element” are enhancer elements, such as WPRE; CMV enhancers; the R-U5′ segment in LTR of HTLV-I (Mol. Cell. Biol., Vol. 8(1), p. 466-472, 1988); SV40 enhancer; and the intron sequence between exons 2 and 3 of rabbit β-globin (Proc. Natl. Acad. Sci. USA., Vol. 78(3), p. 1527-31, 1981). It will be appreciated by those skilled in the art that the design of the expression vector can depend on such factors as the choice of the host cell to be transformed, the level of expression desired, etc.

The term “recombinant” refers to a human manipulated nucleic acid (e.g. polynucleotide) or a copy or complement of a human manipulated nucleic acid (e.g. polynucleotide), or if in reference to a protein (i.e, a “recombinant protein”), a protein encoded by a recombinant nucleic acid (e.g. polynucleotide). In embodiments, a recombinant expression cassette comprising a promoter operably linked to a second nucleic acid (e.g. polynucleotide) may include a promoter that is heterologous to the second nucleic acid (e.g. polynucleotide) as the result of human manipulation (e.g., by methods described in Sambrook et al., Molecular Cloning—A Laboratory Manual, Cold Spring Harbor Laboratory, Cold Spring Harbor, N.Y., (1989) or Current Protocols in Molecular Biology Volumes 1-3, John Wiley & Sons, Inc. (1994-1998)). In another example, a recombinant expression cassette may comprise nucleic acids (e.g. polynucleotides) combined in such a way that the nucleic acids (e.g. polynucleotides) are extremely unlikely to be found in nature. For instance, human manipulated restriction sites or plasmid vector sequences may flank or separate the promoter from the second nucleic acid (e.g. polynucleotide). One of skill will recognize that nucleic acids (e.g. polynucleotides) can be manipulated in many ways and are not limited to the examples above.

The term “expression cassette” or “expression vector” refers to a nucleic acid construct, which when introduced into a host cell, results in transcription and/or translation of a RNA or polypeptide, respectively. In embodiments, an expression cassette comprising a promoter operably linked to a second nucleic acid (e.g. polynucleotide) may include a promoter that is heterologous to the second nucleic acid (e.g. polynucleotide) as the result of human manipulation (e.g., by methods described in Sambrook et al., Molecular Cloning—A Laboratory Manual, Cold Spring Harbor Laboratory, Cold Spring Harbor, N.Y., (1989) or Current Protocols in Molecular Biology Volumes 1-3, John Wiley & Sons, Inc. (1994-1998)).

In some embodiments, an expression cassette comprising a terminator (or termination sequence) operably linked to a second nucleic acid (e.g. polynucleotide) may include a terminator that is heterologous to the second nucleic acid (e.g. polynucleotide) as the result of human manipulation. In some embodiments, the expression cassette comprises a promoter operably linked to a second nucleic acid (e.g. polynucleotide) and a terminator operably linked to the second nucleic acid (e.g. polynucleotide) as the result of human manipulation. In some embodiments, the expression cassette comprises an endogenous promoter. In some embodiments, the expression cassette comprises an endogenous terminator. In some embodiments, the expression cassette comprises a synthetic (or non-natural) promoter. In some embodiments, the expression cassette comprises a synthetic (or non-natural) terminator.

The terms “identical” or percent “identity,” in the context of two or more nucleic acids or polypeptide sequences, refer to two or more sequences or subsequences that are the same or have a specified percentage of amino acid residues or nucleotides that are the same (i.e., about 60% identity, preferably 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or higher identity over a specified region when compared and aligned for maximum correspondence over a comparison window or designated region) as measured using a BLAST or BLAST 2.0 sequence comparison algorithms with default parameters described below, or by manual alignment and visual inspection (see, e.g., NCBI web site or the like). Such sequences are then said to be “substantially identical.” This definition also refers to, or may be applied to, the compliment of a test sequence. The definition also includes sequences that have deletions and/or additions, as well as those that have substitutions. As described below, the preferred algorithms can account for gaps and the like. Preferably, identity exists over a region that is at least about 10 amino acids or 20 nucleotides in length, or more preferably over a region that is 10-50 amino acids or 20-50 nucleotides in length. As used herein, percent (%) amino acid sequence identity is defined as the percentage of amino acids in a candidate sequence that are identical to the amino acids in a reference sequence, after aligning the sequences and introducing gaps, if necessary, to achieve the maximum percent sequence identity. Alignment for purposes of determining percent sequence identity can be achieved in various ways that are within the skill in the art, for instance, using publicly available computer software such as BLAST, BLAST-2, ALIGN, ALIGN-2 or Megalign (DNASTAR) software. Appropriate parameters for measuring alignment, including any algorithms needed to achieve maximal alignment over the full-length of the sequences being compared can be determined by known methods.

For sequence comparisons, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Preferably, default program parameters can be used, or alternative parameters can be designated. The sequence comparison algorithm then calculates the percent sequence identities for the test sequences relative to the reference sequence, based on the program parameters.

One example of an algorithm that is suitable for determining percent sequence identity and sequence similarity are the BLAST and BLAST 2.0 algorithms, which are described in Altschul et al. (1977) Nuc. Acids Res. 25:3389-3402, and Altschul et al. (1990) J. Mol. Biol. 215:403-410, respectively. Software for performing BLAST analyses is publicly available through the National Center for Biotechnology Information (http://www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al. (1990) J. Mol. Biol. 215:403-410). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) or 10, M=5, N=−4 and a comparison of both strands. For amino acid sequences, the BLASTP program uses as defaults a wordlength of 3, and expectation (E) of 10, and the BLOSUM62 scoring matrix (see Henikoff and Henikoff (1989) Proc. Natl. Acad. Sci. USA 89:10915) alignments (B) of 50, expectation (E) of 10, M=5, N=−4, and a comparison of both strands.

The BLAST algorithm also performs a statistical analysis of the similarity between two sequences (see, e.g., Karlin and Altschul (1993) Proc. Natl. Acad. Sci. USA 90:5873-5787). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01.

The phrase “codon optimized” as it refers to genes or coding regions of nucleic acid molecules for the transformation of various hosts, refers to the alteration of codons in the gene or coding regions of polynucleic acid molecules to reflect the typical codon usage of a selected organism without altering the polypeptide encoded by the DNA. Such optimization includes replacing at least one, or more than one, or a significant number, of codons with one or more codons that are more frequently used in the genes of that selected organism.

Nucleic acid is “operably linked” when it is placed into a functional relationship with another nucleic acid sequence. For example, DNA for a presequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are near each other, and, in the case of a secretory leader, contiguous and in reading phase. However, operably linked nucleic acids (e.g. enhancers and coding sequences) do not have to be contiguous. Linking is accomplished by ligation at convenient restriction sites. If such sites do not exist, the synthetic oligonucleotide adaptors or linkers are used in accordance with conventional practice. In embodiments, a promoter is operably linked with a coding sequence when it is capable of affecting (e.g. modulating relative to the absence of the promoter) the expression of a protein from that coding sequence (i.e., the coding sequence is under the transcriptional control of the promoter).

The term “membraneless organelle” as used herein refers to a class of organelles that do not contain a lipid membrane separating them from the nuclear or cytoplasmic liquid. Membraneless organelles contain proteins with low complexity domains or intrinsically disordered regions (LCDs, IDRs) which, when focally concentrated, undergo liquid-liquid phase separation (LLPS) due to attractive forces that force them to self-interact through weak multivalent attractive forces. These forces can be further stabilized by nucleic acids (RNA or DNA) and other molecules commonly found in each membraneless organelle. Nonlimiting examples of membraneless organelles include stress granules, P-bodies, transport RNP, nucleolus, nuclear speckles, nuclear stress bodies, PML bodies, Cajal bodies, and nuclear paraspeckles. The term “membraneless organelle target protein” as used herein refers to any protein containing: a low complexity domain or intrinsically disordered region (LCD, IDR) which, when focally concentrated, undergoes liquid-liquid phase separation (LLPS) due to attractive forces that force them to self-interact through weak multivalent attractive forces; and forming part of a membraneless organelle.

The term “variant” or “derivative” as used herein refers to an amino acid sequence derived from the amino acid sequence of the parent protein having one or more amino acid substitutions, insertions, and/or deletions.

### Chimeric Constructs

In one aspect, disclosed herein is a nucleotide sequence encoding a chimeric polypeptide, comprising: a first nucleotide sequence encoding a light-induced oligomerization domain and a second nucleotide sequence encoding a low complexity domain from a membraneless organelle target protein.

In one aspect, disclosed herein is a nucleotide sequence encoding a chimeric polypeptide, comprising: a first nucleotide sequence encoding a low complexity domain from a membraneless organelle target protein and a second nucleotide sequence encoding a light-induced oligomerization domain.

In some embodiments, the low complexity domain from a membraneless organelle target protein has been previously shown to be required for the formation of a membraneless organelle. In some embodiments, the target LCD-containing protein has been shown to prevent specific organelle formation when removed from the cell.

In one aspect, disclosed herein is an expression vector encoding a chimeric polypeptide, comprising: a first nucleotide sequence encoding a light-induced oligomerization domain and a second nucleotide sequence encoding a low complexity domain from a membraneless organelle target protein, wherein the first nucleotide sequence is operably linked to a promoter.

In some embodiments, the expression vector encoding a chimeric polypeptide is comprised in a plasmid or in a virus or viral vector. A plasmid or a viral vector can be capable of extrachromosomal replication or, optionally, can integrate into the host genome. As used herein, the term “integrated” used in reference to an expression vector (e.g., a plasmid or viral vector) means the expression vector, or a portion thereof, is incorporated (physically inserted or ligated) into the chromosomal DNA of a host cell. As used herein, a “viral vector” refers to a virus-like particle containing genetic material which can be introduced into a eukaryotic cell without causing substantial pathogenic effects to the eukaryotic cell. A wide range of viruses or viral vectors can be used for transduction, but should be compatible with the cell type the virus or viral vector are transduced into (e.g., low toxicity, capability to enter cells). Suitable viruses and viral vectors include adenovirus, lentivirus, retrovirus, among others. In some embodiments, the expression vector encoding a chimeric polypeptide is a naked DNA or is comprised in a nanoparticle (e.g., liposomal vesicle, porous silicon nanoparticle, gold-DNA conjugate particle, polyethylenimine polymer particle, cationic peptides, etc.).

The fusion constructs disclosed herein can be fused with the light-induced oligomerization domain at either the 5′ end or the 3′ end of the nucleic acid sequence or protein sequence. Thus, for every chimeric construct disclosed herein, the fusion of the chimeric polypeptide with the light-induced oligomerization and the membraneless organelle target protein reversed (in a 5′ to 3′ direction) is also disclosed. In one aspect, disclosed herein is an expression vector encoding a chimeric polypeptide, comprising: a first nucleotide sequence encoding a low complexity domain from a membraneless organelle target protein and a second nucleotide sequence encoding a light-induced oligomerization domain, wherein the first nucleotide sequence is operably linked to a promoter.

In some embodiments, the first nucleotide sequence is positioned upstream of the second nucleotide sequence. In some embodiments, the first nucleotide sequence is positioned downstream of the second nucleotide sequence.

In some embodiments, the nucleotide sequence (nucleic acid sequence) is an isolated or purified nucleotide sequence (nucleic acid sequence). In some embodiments, the chimeric polypeptide is an isolated or purified chimeric polypeptide.

In some embodiments, where the sequences disclosed herein contain a methionine at the start of the protein, the protein without the methionine is also disclosed. In some embodiments, where the sequences disclosed herein do not contain a methionine at the start of the protein, the protein with the methionine at the start of the protein is also disclosed.

In one aspect, disclosed herein is a cell comprising a nucleotide sequence encoding a chimeric polypeptide, comprising: a first nucleotide sequence encoding a light-induced oligomerization domain and a second nucleotide sequence encoding a membraneless organelle target protein.

In one aspect, disclosed herein is a cell comprising a nucleotide sequence encoding a chimeric polypeptide, comprising: a first nucleotide sequence encoding a membraneless organelle target protein and a second nucleotide sequence encoding a light-induced oligomerization domain.

In one aspect, disclosed herein is a chimeric polypeptide comprising: a light-induced oligomerization domain; and a low complexity domain from a membraneless organelle target protein.

In one aspect, disclosed herein is a chimeric polypeptide comprising: a low complexity domain from a membraneless organelle target protein; and a light-induced oligomerization domain.

In one embodiment, the light-induced oligomerization domain is selected from the group consisting of CRY2 PHR, CRY2OLIG, NcVVD, NcVVDY50W, NcVVDY50W/I74V/I85V/LINKERA, NcVVDY50W/I52C/I74V/I85V/LINKERA, NcVVDY50W/C71V/I74V/I85V/LINKERA, NcVVDY50W/I52C/C71V/I74V/I85V/LINKERA, NcVVDY50W/I74V/I85V/LINKERB, NcVVDY50W/I52C/I74V/I85V/LINKERB, NcVVDY50W/C71V/I74V/I85V/LINKERB, NcVVDY50W/I52C/C71V/I74V/I85V/LINKERB, NcLOV, and VfAU1LOV. In one embodiment, the light-induced oligomerization domain is selected from the group CRY2 PHR, CRY2OLIG, NcVVD, NcVVDY50W, NcVVDY50W/I74V/I85V/LINKERA, NcVVDY50W/I52C/I74V/I85V/LINKERA, NcVVDY50W/C71V/I74V/I85V/LINKERA, NcVVDY50W/I52C/C71V/I74V/I85V/LINKERA, NcVVDY50W/I74V/I85V/LINKERB, NcVVDY50W/I52C/I74V/I85V/LINKERB, NcVVDY50W/C71V/I74V/I85V/LINKERB, NcVVDY50W/I52C/C71V/I74V/I85V/LINKERB, NcLOV, VfAU1LOV, YtvA, EL222, RsLOV, AsLOV2, a variant thereof, or a fragment thereof.

In one embodiment, the light-induced oligomerization domain is NcVVDY50W. In one embodiment, the light-induced oligomerization domain is NcVVDY50W/I74/I85/LINKERA. In one embodiment, the light-induced oligomerization domain is NcVVDY50W/I52C/I74/I85/LINKERA. In one embodiment, the light-induced oligomerization domain is NcVVDY50W/C71V/I74/I85/LINKERA. In one embodiment, the light-induced oligomerization domain is NcVVDY50W/I52C/C71V/I74/I85/LINKERA. In one embodiment, the light-induced oligomerization domain is NcVVDY50W/I74/I85/LINKERB. In one embodiment, the light-induced oligomerization domain is NcVVDY50W/I52C/I74/I85/LINKERB. In one embodiment, the light-induced oligomerization domain is NcVVDY50W/C71V/I74/I85/LINKERB. In one embodiment, the light-induced oligomerization domain is NcVVDY50W/I52C/C71V/I74/I85/LINKERB. In one embodiment, the light-induced oligomerization domain is CRY2OLIG. In one embodiment, the light-induced oligomerization domain is CRY2 PHR. In one embodiment, the light-induced oligomerization domain is VfAU1LOV. In one embodiment, the light-induced oligomerization domain is YtvA. In one embodiment, the light-induced oligomerization domain is EL222. In one embodiment, the light-induced oligomerization domain is RsLOV. In one embodiment, the light-induced oligomerization domain is AsLOV2.

In one embodiment, the light-induced oligomerization domain comprises a LOV domain. In one embodiment, the light-induced oligomerization domain comprises a LOV domain from the VVD protein. In one embodiment, the light-induced oligomerization domain comprises a LOV domain from the LOV protein. In one embodiment, the light-induced oligomerization domain comprises a PHR domain. In one embodiment, the light-induced oligomerization domain comprises a PHR domain, from the CRY2 protein.

In one embodiment, the light-induced oligomerization domain is at least 90% identical to CRY2 PHR. In one embodiment, the light-induced oligomerization domain is at least 90% identical to NcVVD. In one embodiment, the light-induced oligomerization domain is at least 90% identical to NcVVDY50W. In one embodiment, the light-induced oligomerization domain is at least 90% identical to NcLOV. In one embodiment, the light-induced oligomerization domain is CRY2OLIG. In one embodiment, the light-induced oligomerization domain is at least 90% identical to CRY2 PHR. In one embodiment, the light-induced oligomerization domain is at least 90% identical to VfAU1LOV. In one embodiment, the light-induced oligomerization domain is at least 60% (for example, at least 60%, at least 70%, at least 80%, at least 90%, at least 95%) identical to CRY2 PHR, CRY2OLIG, NcVVD, NcVVDY50W, NcLOV, and VfAU1LOV. In one embodiment, the light-induced oligomerization domain is at least 60% (for example, at least 60%, at least 70%, at least 80%, at least 90%, at least 95%) identical to the PHR or LOV domain from the following proteins: CRY2 PHR, CRY2OLIG, NcVVD, NcVVDY50W, NcLOV, and VfAU1LOV.

In one embodiment, the light-induced oligomerization domain is at least 60% (for example, at least 60%, at least 70%, at least 80%, at least 90%, at least 95%) identical to a sequence selected from the group consisting of SEQ ID NO:1, SEQ ID NO:2, SEQ ID NO:3, SEQ ID NO:4, SEQ ID NO:5, SEQ ID NO:6, SEQ ID NO:7, SEQ ID NO:107, SEQ ID NO: 108, SEQ ID NO:109, and SEQ ID NO:110. In some embodiments, a protein fragment of SEQ ID NO:1, SEQ ID NO:2, SEQ ID NO:3, SEQ ID NO:4, SEQ ID NO:5, SEQ ID NO:6, SEQ ID NO:7, SEQ ID NO:107, SEQ ID NO: 108, SEQ ID NO:109, or SEQ ID NO:110 is used as the light-induced oligomerization domain. In some embodiments, a protein fragment of SEQ ID NO:1, SEQ ID NO:2, SEQ ID NO:3, SEQ ID NO:4, SEQ ID NO:5, SEQ ID NO:6, SEQ ID NO:7, SEQ ID NO:107, SEQ ID NO:108, SEQ ID NO:109, or SEQ ID NO:110 is used as the light-induced oligomerization domain, wherein the fragment is sufficient for light-induced oligomerization.

In one embodiment, the light-induced oligomerization domain is a LOV domain. In one embodiment, the light-induced oligomerization domain is a PHR domain. In one embodiment, the light-induced oligomerization domain is a LOV domain selected from NcVVD, NcVVDY50W, and NcLOV. In one embodiment, the light-induced oligomerization domain is a PHR domain selected from CRY2 PHR and CRY2OLIG.

In one embodiment, the low complexity domain is from a membraneless organelle target protein. In one embodiment, the low complexity domain from a membraneless organelle target protein is from a cytoplasmic membraneless organelle target protein.

In one embodiment, the low complexity domain from a membraneless organelle target protein is from a stress granule target protein. In one embodiment, the low complexity domain is from a stress granule target protein selected from the group consisting of PABC1, TIAR, G3BP1, G3BP2, DDX6, TDRD3, ATXN2, variants thereof, and fragments thereof. In one embodiment, the low complexity domain is from a stress granule target protein selected from the group consisting of an amino acid sequence with at least 60% (for example, at least 60%, at least 70%, at least 80%, at least 90%, at least 95%) identity to PABC1, TIAR, G3BP1, G3BP2, DDX6, TDRD3, ATXN2, variants thereof, and fragments thereof.

In one embodiment, the low complexity domain from a membraneless organelle target protein is G3BP1. In one embodiment, the low complexity domain from a membraneless organelle target protein is the G3BP1 protein lacking an NTF2 domain. In one embodiment, the low complexity domain from a membraneless organelle target protein is the G3BP1 protein lacking an NTF2 domain and replaced with a oligomerizing or dimerizing photoreceptor. In one embodiment, the low complexity domain from a membraneless organelle target protein is an acidic domain truncated G3BP1. In one embodiment, the low complexity domain from a membraneless organelle target protein is a PxxP domain truncated G3BP1. In one embodiment, the low complexity domain from a membraneless organelle target protein is an RRM domain truncated G3BP1. In one embodiment, the low complexity domain from a membraneless organelle target protein is an RGG domain truncated G3BP1.

In one embodiment, the low complexity domain is from a stress granule target protein selected from the group consisting of PABC1, Gle1, TIA1, TIAR, G3BP1, mTOR, DYRK3, Staufen, eIF3H, eIF4A, eIF4E, eIF4G, TTP, HuR, FMRP, FXR1/2, ZBP1, RSK2, NXF1, Mcm2, Mcm4, DDX6 (Dhh1), DPYSL3, DCTN1, USP10, CAPRIN1, variants thereof, and fragments thereof. In one embodiment, the low complexity domain is from a stress granule target protein selected from the group consisting of an amino acid sequence with at least 60% (for example, at least 60%, at least 70%, at least 80%, at least 90%, at least 95%) identity to PABC1, Gle1, TIA1, TIAR, G3BP1, mTOR, DYRK3, Staufen, eIF3H, eIF4A, eIF4E, eIF4G, TTP, HuR, FMRP, FXR1/2, ZBP1, RSK2, NXF1, Mcm2, Mcm4, DDX6 (Dhh1), DPYSL3, DCTN1, USP10, CAPRIN1, variants thereof, and fragments thereof.

In one embodiment, the low complexity domain from a membraneless organelle target protein is from a P-body target protein. In one embodiment, the low complexity domain is from a P-body target protein selected from the group consisting of DCP1A, DCP2, LSM1, TNRC6A, MEX3A, EDC4, XRN1, DDX3X, variants thereof, and fragments thereof. In one embodiment, the low complexity domain is from a P-body target protein selected from the group consisting of Dcp1, Dcp2, Lsm1, Hedis, GW182, Pop2, Pan2, Mex3A, Pat1, Edc4, Xrn1, DDX3, DDX6, variants thereof, and fragments thereof. In one embodiment, the low complexity domain is from a P-body target protein selected from the group consisting of an amino acid sequence with at least 60% (for example, at least 60%, at least 70%, at least 80%, at least 90%, at least 95%) identity to Dcp1, Dcp2, Lsm1, Hedis, GW182, Pop2, Pan2, Mex3A, Pat1, Edc4, Xrn1, DDX3, DDX6, variants thereof, and fragments thereof.

In one embodiment, the low complexity domain from a membraneless organelle target protein is from a ribonuclear transport granule target protein. In one embodiment, the low complexity domain is from a ribonuclear transport granule target protein selected from the group consisting of IGFBP1, STAU1, PURA, FMR1, FXR1, FXR2, variants thereof, and fragments thereof. In one embodiment, the low complexity domain is from a ribonuclear transport granule target protein selected from the group consisting of an amino acid sequence with at least 60% (for example, at least 60%, at least 70%, at least 80%, at least 90%, at least 95%) identity to IGFBP1, STAU1, PURA, FMR1, FXR1, FXR2, variants thereof, and fragments thereof.

In one embodiment, the low complexity domain from a membraneless organelle target protein is from a nuclear membraneless organelle target protein.

In one embodiment, the low complexity domain from a membraneless organelle target protein is from a nucleolus target protein. In one embodiment, the low complexity domain is from a nucleolus target protein selected from the group consisting of NCL, NPM1, FBL, variants thereof, and fragments thereof. In one embodiment, the low complexity domain is from a nucleolus target protein selected from the group consisting of NCL, NPM1, FBL, HNRNPA3, HspA8, RPS19, HMGB2, RCC2, KI-67, variants thereof, and fragments thereof. In one embodiment, the low complexity domain is from a nucleolus target protein selected from the group consisting of an amino acid sequence with at least 60% (for example, at least 60%, at least 70%, at least 80%, at least 90%, at least 95%) identity to NCL, NPM1, FBL, HNRNPA3, HspA8, RPS19, HMGB2, RCC2, KI-67, variants thereof, and fragments thereof.

In one embodiment, the low complexity domain from a membraneless organelle target protein is from a nuclear speckle target protein. In one embodiment, the low complexity domain is from a nuclear speckle target protein selected from the group consisting of SRSF2, PNN, SRSF1, variants thereof, and fragments thereof. In one embodiment, the low complexity domain is from a nuclear speckle target protein selected from the group consisting of eIF4E, eIF4AIII, SC35, Pinin, SRSF1, variants thereof, and fragments thereof. In one embodiment, the low complexity domain is from a nuclear speckle target protein selected from the group consisting of an amino acid sequence with at least 60% (for example, at least 60%, at least 70%, at least 80%, at least 90%, at least 95%) identity to eIF4E, eIF4AIII, SC35, Pinin, SRSF1, variants thereof, and fragments thereof.

In one embodiment, the low complexity domain from a membraneless organelle target protein is from a nuclear stress body target protein. In one embodiment, the low complexity domain is from a nuclear stress body target protein selected from the group consisting of SAFB, variants thereof, and fragments thereof. In one embodiment, the low complexity domain is from a nuclear stress body target protein selected from the group consisting of an amino acid sequence with at least 60% (for example, at least 60%, at least 70%, at least 80%, at least 90%, at least 95%) identity to SAFB, variants thereof, and fragments thereof.

In one embodiment, the low complexity domain from a membraneless organelle target protein is from a nuclear paraspeckle target protein. In one embodiment, the low complexity domain is from a nuclear paraspeckle target protein selected from the group consisting of SFPQ, NONO, PSPC1, variants thereof, and fragments thereof. In one embodiment, the low complexity domain is from a nuclear paraspeckle target protein selected from the group consisting of PSF/SFPQ, P54NRB/NONO, PSPC1, variants thereof, and fragments thereof. In one embodiment, the low complexity domain is from a nuclear paraspeckle target protein selected from the group consisting of an amino acid sequence with at least 60% (for example, at least 60%, at least 70%, at least 80%, at least 90%, at least 95%) identity to PSF/SFPQ, P54NRB/NONO, PSPC1, variants thereof, and fragments thereof.

In one embodiment, the low complexity domain from a membraneless organelle target protein is from a cajal body target protein. In one embodiment, the low complexity domain is from a cajal body target protein selected from the group consisting of COIL. In one embodiment, the low complexity domain is from a cajal body target protein selected from the group consisting of Coilin, SMN1, SMN2, FLASH (CASP8AP2), variants thereof, and fragments thereof. In one embodiment, the low complexity domain is from a cajal body target protein selected from the group consisting of an amino acid sequence with at least 60% (for example, at least 60%, at least 70%, at least 80%, at least 90%, at least 95%) identity to Coilin, SMN1, SMN2, FLASH (CASP8AP2), variants thereof, and fragments thereof.

In one embodiment, the low complexity domain from a membraneless organelle target protein is from a PML body target protein. In one embodiment, the low complexity domain is from a PML body target protein selected from the group consisting of PML, variants thereof, and fragments thereof. In one embodiment, the low complexity domain is from a PML body target protein selected from the group consisting of an amino acid sequence with at least 60% (for example, at least 60%, at least 70%, at least 80%, at least 90%, at least 95%) identity to PML, variants thereof, and fragments thereof.

In one embodiment, the low complexity domain from a membraneless organelle target protein is from a chromatoid body/germ granule target protein. In one embodiment, the low complexity domain is from a chromatoid body/germ granule target protein selected from the group consisting of DDX4, variants thereof, and fragments thereof. In one embodiment, the low complexity domain is from a chromatoid body/germ granule target protein selected from the group consisting of an amino acid sequence with at least 60% (for example, at least 60%, at least 70%, at least 80%, at least 90%, at least 95%) identity to DDX4, variants thereof, and fragments thereof.

In one embodiment, the cell is a mammalian cell. In one embodiment, the cell is a human cell. In one embodiment, the cell is an isolated cell.

In one embodiment, the blue light has a wavelength between 405 nm and 499 nm. In one embodiment, the blue light has a wavelength of about 465 nm.

In one embodiment, the low complexity domain from a membraneless organelle target protein is selected from Tables 2-10. In one embodiment, the low complexity domain is from any membraneless organelle target protein. In one embodiment, the low complexity domain is from any membraneless organelle target protein that, when focally concentrated, undergoes liquid-liquid phase separation (LLPS) due to attractive forces that force them to self-interact through weak multivalent attractive forces.

In one embodiment, the low complexity domain from a membraneless organelle target protein is selected from the group consisting of an amino acid sequence with at least 90% identity to a membraneless organelle target protein is selected from Tables 2-10. In one embodiment, the low complexity domain from a membraneless organelle target protein is selected from the group consisting of an amino acid sequence with at least 90% identity to a low complexity domain from a membraneless organelle target protein is selected from Table 2. In one embodiment, the low complexity domain from a membraneless organelle target protein is selected from the group consisting of an amino acid sequence with at least 90% identity to a low complexity domain from a membraneless organelle target protein is selected from Table 3. In one embodiment, the low complexity domain from a membraneless organelle target protein is selected from the group consisting of an amino acid sequence with at least 90% identity to a low complexity domain from a membraneless organelle target protein is selected from Table 4. In one embodiment, the low complexity domain from a membraneless organelle target protein is selected from the group consisting of an amino acid sequence with at least 90% identity to a low complexity domain from a membraneless organelle target protein is selected from Table 5. In one embodiment, the low complexity domain from a membraneless organelle target protein is selected from the group consisting of an amino acid sequence with at least 90% identity to a low complexity domain from a membraneless organelle target protein is selected from Table 6. In one embodiment, the low complexity domain from a membraneless organelle target protein is selected from the group consisting of an amino acid sequence with at least 90% identity to a low complexity domain from a membraneless organelle target protein is selected from Table 7. In one embodiment, the low complexity domain from a membraneless organelle target protein is selected from the group consisting of an amino acid sequence with at least 90% identity to a low complexity domain from a membraneless organelle target protein is selected from Table 8. In one embodiment, the low complexity domain from a membraneless organelle target protein is selected from the group consisting of an amino acid sequence with at least 90% identity to a low complexity domain from a membraneless organelle target protein is selected from Table 9. In one embodiment, the low complexity domain from a membraneless organelle target protein is selected from the group consisting of an amino acid sequence with at least 90% identity to a low complexity domain from a membraneless organelle target protein is selected from Table 10. In one embodiment, the low complexity domain from a membraneless organelle target protein is selected from an orthologue of the group consisting of a membraneless organelle target protein selected from Tables 2-10.

In one embodiment, the low complexity domain from a membraneless organelle target protein is selected from the group consisting of an amino acid sequence with at least 60% (for example, at least 60%, at least 70%, at least 80%, at least 90%, at least 95%) identity to a membraneless organelle target protein is selected from Tables 2-10. In one embodiment, the low complexity domain is a variant or a fragment of a membraneless organelle target protein, wherein the membraneless organelle target protein is selected from Tables 2-10.

In one embodiment, the low complexity domain from a membraneless organelle target protein is G3BP1. In one embodiment, the low complexity domain from a membraneless organelle target protein is selected from the group consisting of an amino acid sequence with at least 60% (for example, at least 60%, at least 70%, at least 80%, at least 90%, at least 95%) identity to G3BP1.

In one embodiment, a VVD light-induced oligomerization domain is fused to a low complexity domain of G3BP1. In one embodiment, a VVD light-induced oligomerization is fused to full length G3BP1 (comprising a low complexity domain). In one embodiment, a NcVVDY50W light-induced oligomerization domain is fused to a low complexity domain of G3BP1. In one embodiment, a NcVVDY50W light-induced oligomerization is fused to full length G3BP1 (comprising a low complexity domain).

In one embodiment, the nucleotide sequence encoding the chimeric polypeptide may further comprise a nucleotide sequence encoding a fluorescent protein (to allow visualization of the membraneless organelles by fluorescence). In one embodiment, the fluorescent protein is mCherry (mCh). In some embodiments, the fluorescent protein is GFP or YFP.

In some embodiments, the chimeric polypeptide comprises an amino acid sequence selected from SEQ ID NO:9, SEQ ID NO:10, SEQ ID NO: 11, SEQ ID NO:12, SEQ ID NO:13, SEQ ID NO:14, SEQ ID NO:15, SEQ ID NO: 16, SEQ ID NO:17, SEQ ID NO:18, SEQ ID NO: 19, SEQ ID NO:20 SEQ ID NO:21, SEQ ID NO:22, SEQ ID NO:23, SEQ ID NO:24, SEQ ID NO: 25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO: 36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO: 47, SEQ ID NO:48, SEQ ID NO:49, SEQ ID NO:50, SEQ ID NO:51, SEQ ID NO:52, SEQ ID NO:53, SEQ ID NO:54, SEQ ID NO:55, SEQ ID NO:56, SEQ ID NO:57, SEQ ID NO: 58, SEQ ID NO:59, SEQ ID NO:60, SEQ ID NO:61, SEQ ID NO:62, SEQ ID NO:63, SEQ ID NO:64, SEQ ID NO:65, SEQ ID NO:66, SEQ ID NO:67, SEQ ID NO:68, SEQ ID NO: 69, SEQ ID NO:70, SEQ ID NO:71, SEQ ID NO:72, SEQ ID NO:73, SEQ ID NO:74, SEQ ID NO:75, SEQ ID NO:76, SEQ ID NO:77, SEQ ID NO:78, SEQ ID NO:79, SEQ ID NO: 80, SEQ ID NO:81, SEQ ID NO:82, SEQ ID NO:83, SEQ ID NO:84, SEQ ID NO:85, SEQ ID NO:86, SEQ ID NO:87, SEQ ID NO:88, SEQ ID NO:89, SEQ ID NO:90, SEQ ID NO: 91, SEQ ID NO:92, SEQ ID NO:93, SEQ ID NO:94, SEQ ID NO:95, SEQ ID NO:96, SEQ ID NO:97, SEQ ID NO:98, SEQ ID NO:99, SEQ ID NO: 100, SEQ ID NO:101, SEQ ID NO: 102, SEQ ID NO: 103, SEQ ID NO: 104, SEQ ID NO: 105, or SEQ ID NO:106.

In some embodiments, the chimeric polypeptide comprises an amino acid sequence that is at least 60% (for example, at least 60%, at least 70%, at least 80%, at least 90%, at least 95%) identical to a sequence selected from one of SEQ ID NO:9 to 106. In some embodiments, a protein fragment of one of SEQ ID NO:9 to 106 is used. In some embodiments, a protein fragment of one of SEQ ID NO:9 to 106 is used, wherein the fragment is sufficient for light-induced oligomerization.

### Methods

In one aspect, disclosed herein is a method of inducing a membraneless organelle in a cell, comprising the steps:
introducing into the cell an expression vector encoding a chimeric polypeptide, comprising:
   a first nucleotide sequence encoding a light-induced oligomerization domain and a second nucleotide sequence encoding a low complexity domain from a membraneless organelle target protein, wherein the first nucleotide sequence is operably linked to a promoter;
expressing the chimeric polypeptide; and
inducing oligomerization of the chimeric polypeptide by stimulation with blue light.

In another aspect, disclosed herein is a method of screening for an agent that modulates formation of a membraneless organelle, comprising the steps:
introducing into a cell an expression vector encoding a chimeric polypeptide, comprising:
   a first nucleotide sequence encoding a light-induced oligomerization domain and a second nucleotide sequence encoding a low complexity domain from a membraneless organelle target protein, wherein the first nucleotide sequence is operably linked to a promoter;
expressing the chimeric polypeptide;
introducing the agent into a culture media comprising the cell;
inducing oligomerization of the chimeric polypeptide by stimulation with blue light; and
determining modulation of the formation of the membraneless organelle by the agent.

In one aspect, disclosed herein is a method of inducing a membraneless organelle in a cell, comprising the steps:
introducing into the cell an expression vector encoding a chimeric polypeptide, comprising:
   a first nucleotide sequence encoding a low complexity domain from a membraneless organelle target protein and a second nucleotide sequence encoding a light-induced oligomerization domain, wherein the first nucleotide sequence is operably linked to a promoter;
expressing the chimeric polypeptide; and
inducing oligomerization of the chimeric polypeptide by stimulation with blue light.

In another aspect, disclosed herein is a method of screening for an agent that modulates formation of a membraneless organelle, comprising the steps:
introducing into a cell an expression vector encoding a chimeric polypeptide, comprising:
   a first nucleotide sequence encoding a low complexity domain from a membraneless organelle target protein and a second nucleotide sequence encoding a light-induced oligomerization domain, wherein the first nucleotide sequence is operably linked to a promoter;
expressing the chimeric polypeptide;
introducing the agent into a culture media comprising the cell;
inducing oligomerization of the chimeric polypeptide by stimulation with blue light; and
determining modulation of the formation of the membraneless organelle by the agent.

In some embodiments, the modulation includes the increase in formation of membraneless organelles. In some embodiments, the modulation includes the decrease in formation of membraneless organelles.

In one embodiment, the light-induced oligomerization domain is selected from the group consisting of CRY2 PHR, CRY2OLIG, NcVVD, NcVVDY50W, NcVVDY50W/I74V/I85V/LINKERA, NcVVDY50W/I52C/I74V/I85V/LINKERA, NcVVDY50W/C71V/I74V/I85V/LINKERA, NcVVDY50W/I52C/C71V/I74V/I85V/LINKERA, NcVVDY50W/I74V/I85V/LINKERB, NcVVDY50W/I52C/I74V/I85V/LINKERB, NcVVDY50W/C71V/I74V/I85V/LINKERB, NcVVDY50W/I52C/C71V/I74V/I85V/LINKERB, NcLOV, and VfAU1LOV. In one embodiment, the light-induced oligomerization domain is selected from the group CRY2 PHR, CRY2OLIG, NcVVD, NcVVDY50W, NcVVDY50W/I74V/I85V/LINKERA, NcVVDY50W/C71V/I74V/I85V/LINKERA, NcVVDY50W/I52C/I74V/I85V/LINKERA, NcVVDY50W/I52C/C71V/I74V/I85V/LINKERA, NcVVDY50W/I74V/I85V/LINKERB, NcVVDY50W/I52C/I74V/I85V/LINKERB, NcVVDY50W/C71V/I74V/I85V/LINKERB, NcVVDY50W/I52C/C71V/I74V/I85V/LINKERB, NcLOV, VfAU1LOV, YtvA, EL222, RsLOV, AsLOV2, a variant thereof, or a fragment thereof. In one embodiment, the light-induced oligomerization domain is NcVVDY50W. In one embodiment, the light-induced oligomerization domain is CRY2OLIG. In one embodiment the light-induced oligomerization domain in CRY2 PHR.

In one embodiment, the low complexity domain from a membraneless organelle target protein is selected from the group consisting of an amino acid sequence of a membraneless organelle target protein listed in Tables 2-10. In one embodiment, the low complexity domain from a membraneless organelle target protein is selected from the group consisting of an amino acid sequence with at least 90% identity to a membraneless organelle target protein listed in Tables 2-10. In one embodiment, the low complexity domain from a membraneless organelle target protein is selected from the group consisting of an amino acid sequence with at least 60% (for example, at least 60%, at least 70%, at least 80%, at least 90%, at least 95%) identity to a membraneless organelle target protein is selected from Tables 2-10. In one embodiment, the low complexity domain from a membraneless organelle target protein is G3BP1. In one embodiment, the low complexity domain from a membraneless organelle target protein is an NTF2 domain truncated G3BP1. In one embodiment, the low complexity domain from a membraneless organelle target protein is an acidic domain truncated G3BP1. In one embodiment, the low complexity domain from a membraneless organelle target protein is a PxxP domain truncated G3BP1. In one embodiment, the low complexity domain from a membraneless organelle target protein is an RRM domain truncated G3BP1. In one embodiment, the low complexity domain from a membraneless organelle target protein is an RGG domain truncated G3BP1.

In one embodiment, the cell is a mammalian cell. In one embodiment, the cell is a human cell. In one embodiment, the cell is selected from the group consisting of yeast, insect, avian, fish, worm, amphibian, xenopus, bacteria, algae and mammalian cells. In one embodiment, disclosed herein is a non-human transgenic organism, wherein the organism is an insect, fish, bird, worm, amphibian, xenopus, or non-human mammal. In one embodiment, the cell can be a glial cell or a neuronal cell.

As used herein, the term “introducing,” “introduce,” and grammatical variations thereof, as it relates to introducing an expression vector into a cell, refers to any method suitable for transferring the expression vector into the cell. The term includes as examples, but is not limited to, conjugation, transformation/transfection (e.g., divalent cation exposure, heat shock, electroporation), nuclear microinjection, incubation with calcium phosphate polynucleotide precipitate, high velocity bombardment with polynucleotide-coated microprojectiles (e.g., via gene gun), lipofection, cationic polymer complexation (e.g., DEAE-dextran, polyethylenimine), dendrimer complexation, mechanical deformation of cell membranes (e.g., cell-squeezing), sonoporation, optical transfection, impalefection, hydrodynamic polynucleotide delivery, Agrobacterium-mediated transformation, transduction (e.g., transduction with a virus or viral vector), natural or artificial competence, protoplast fusion, magnetofection, nucleofection, or combinations thereof. An introduced expression vector, or a polynucleotide therefrom, can be genetically integrated or exist extrachromosomally.

A range of blue light wavelengths can be used in the disclosed methods. In one embodiment, the blue light has a wavelength from about 400 nm to about 500 nm. In one embodiment, the blue light has a wavelength from about 405 nm to about 499 nm. In one embodiment, the blue light has a wavelength from about 420 nm to about 490 nm. In one embodiment, the blue light has a wavelength from about 450 nm to about 490 nm. In one embodiment, the blue light has a wavelength from about 460 nm to about 495 nm. In one embodiment, the blue light has a wavelength of about 488 nm. In one embodiment, the blue light has a wavelength of about 475 nm. In one embodiment, the blue light has a wavelength of about 465 nm. In one embodiment, the blue light has a wavelength between 405 nm and 499 nm.

In one embodiment, the blue light has a wavelength of about 405 nm, about 410 nm, about 415 nm, about 420 nm, about 425 nm, about 430 nm, about 435 nm, about 440 nm, about 445 nm, about 450 nm, about 455 nm, about 460 nm, about 465 nm, about 470 nm, about 475 nm, about 480 nm, about 485 nm, about 490 nm, about 495 nm, or about 500 nm.

The methods can include various degrees of blue light stimulation. In some embodiments, the stimulation is acute or, optionally, chronic. Acute stimulation refers stimulation with pulses of blue light from about 0.2 to about 60 seconds, wherein the wavelength of the blue light can be any herein disclosed blue light wavelength. In some embodiments, the acute stimulation includes pulses of blue light from about 0.5 seconds to about 30 seconds, from about 1 second to about 20 seconds, or about 5 seconds. The blue light can be provided by a blue light source or a broad-spectrum light source filtered for the disclosed wavelengths.

In some embodiments, acute stimulation can result in temporary aggregation of a light-induced oligomerization domain (e.g., cytoplasmic prion-like domains/LCD/IDD protein fragments). Temporary aggregation, in some embodiments, includes protein aggregation observable by the herein disclosed methods for less than about twenty minutes or, optionally, less than about fifteen minutes, less than about ten minutes, or about five minutes or less. In some embodiments, acute stimulation does not result in aggregation of cytoplasmic prion-like domains/LCD/IDD protein fragments for twenty minutes or more.

Chronic stimulation is defined by exposure to blue light having a wavelength from about 400 nm to about 500 nm for a duration of about 1 minute or longer (for example, at least 1 minute, at least 5 minutes, at least 10 minutes, at least 30 minutes, at least 60 minutes, at least 2 hours, at least 4 hours, at least 8 hours, at least 12 hours, at least 24 hours, at least 36 hours, or more) from about 0.1 mW/cm2 to 8 mW/cm2 (within 400 nm-500 nm wavelength).

### EXAMPLES

The following examples are set forth below to illustrate the compounds, compositions, methods, and results according to the disclosed subject matter. These examples are not intended to be inclusive of all aspects of the subject matter disclosed herein, but rather to illustrate representative methods and results. These examples are not intended to exclude equivalents and variations of the present invention which are apparent to one skilled in the art.

### Example 1. Optogenetic Induction of Membraneless Organelles

A series of DNA arrangements have been developed consisting of the CRY2 (OLIG or PHR) or LOV photoreceptor domain which cluster or homodimerize in response to blue light exposure and require intracellular FAD (Table 1). These DNA arrangements comprise the DNA sequence of genes that encode for proteins that contain low complexity domains (LCDs) and comprise a membraneless organelle target protein (Tables 2-10).

Table 1 indicates the protein domains that exhibit photoreceptor activity in response to blue light stimulation. These domains are used to generate fusion proteins with the core proteins of membraneless organelles that seed these structures when exposed to specific light paradigms. Some of these photoreceptors self-oligomerize and some can dimerize. A novel method was employed using LEDs to chronically treat cells with light to force dimerizing proteins to oligomerize and seed organelle structures when fused to the core protein. Photoreceptor protein sequences were modified and the generated synthetic sequences were used to investigate the light-responsiveness of the LOV domain to enhance or mitigate dimerizing formation and dissociation time.

Tables 2-10 includes a list of core components of membraneless organelles (stress granules, P-bodies, transport RNP, nucleolus, nuclear speckles, nuclear stress bodies, PML bodies, Cajal bodies, nuclear paraspeckles) that also contain low complexity domains/intrinsically disordered regions (LCD/IDR). Arrangements of the photoreceptors listed in Table 1 are fused to the genes encoding the key components of membraneless organelles listed in Tables 2-10. When expressed in cells and exposed to specific light treatments, the photoreceptor enhances the focal concentration of the membraneless organelle core protein and this promotes LCD/IDR self-interaction which initiates the formation of membraneless organelles through the recruitment of adaptor proteins and other molecules. Together, this allows for the formation of light-induced membraneless organelles.

TABLE 1
List of photoreceptors/light-induced oligomerization domains to optogenetically induce membraneless
organelles in cells with blue light exposure.

| | | | Light | | |
|---|---|---|---|---|---|
| | | | Stimuli | | |
| Nomenclature | Protein Domain | Organism | (nm) | Light Response | Features |
| CRYPHR | Photolyase | Arabidopsis | 405-499 | homo- | Endogenous protein |
| | homology region | | | oligomerization | domain |
| | (PHR) | | | | |
| CRY2OLIG | Photolyase | Arabidopsis | 405-499 | homo- | E490G Mutation to |
| | homology region | | | oligomerization | enhance clustering |
| | (PHR) with | | | | |
| NcVVD | Light-oxygen- | Neurospora | 405-499 | homo- | LOV domain of VVD |
| | voltage-sensing | | | dimerization | gene |
| | (LOV) domain | | | | |
| NcVVDY50W | Light-oxygen- | Neurospora | 405-499 | homo- | VVD Y50G Mutation |
| | voltage-sensing | | | dimerization | to enhance |
| | (LOV) domain | | | | clustering |
| NcLOV | Light-oxygen- | Neurospora | 405-499 | homo- | Clustering LOV |
| | voltage-sensing | | | dimerization | domain from NcVVD |
| | (LOV) domain | | | | with no linker |
| Vfau1 | Light-oxygen- | Vucheria | 405-499 | homo- | |
| | voltage-sensing | frigida | | dimerization | |
| | (LOV) domain | | | | |
| YtvA | Light-oxygen- | Bacillus | 405-499 | homo- | — |
| | voltage-sensing | subtillis | | dimerization | |
| | (LOV) domain | | | | |
| EL222 | Light-oxygen- | Erythrobacter | 405-499 | homo- | |
| | voltage-sensing | litoralis | | dimerization | |
| | (LOV) domain | | | | |
| RsLOV | Light-oxygen- | Rhodobacter | 405-499 | homo- | |
| | voltage-sensing | sphaeroides | | dimerization | |
| | (LOV) domain | | | | |
| AsLOV2 | Light-oxygen- | Avena sativa | 405-499 | Intramolecular | |
| | voltage-sensing | | | conformational | |
| | (LOV) domain | | | change | |

Modified
Photoreceptors

| | | | | | |
|---|---|---|---|---|---|
| NcVVDY50W, | Light-oxygen- | Neurospora | 405-499 | homo- | LOV domain of VVD |
| I52C, LINKER A | voltage-sensing | | | dimerization | gene with altered |
| | (LOV) domain | | | | light responsive |
| | | | | | properties and |
| | | | | | linker domains |
| NcVVDY50W, | Light-oxygen- | Neurospora | 405-499 | homo- | LOV domain of VVD |
| LINKER A | voltage-sensing | | dimerization | gene with altered | light responsive |
| | (LOV) domain | | | | properties and |
| | | | | | linker domains |
| NcVVDY50W, | Light-oxygen- | Neurospora | 405-499 | homo- | LOV domain of VVD |
| I52C, LINKER A | voltage-sensing | | | dimerization | gene with altered |
| | (LOV) domain | | | | light responsive |
| | | | | | properties and |
| | | | | | linker domains |
| NcVVDY50W, | Light-oxygen- | Neurospora | 405-499 | homo- | LOV domain of VVD |
| I52C, LINKER A | voltage-sensing | | | dimerization | gene with altered |
| | (LOV) domain | | | | light responsive |
| | | | | | properties and |
| | | | | | linker domains |
| NcVVDY50W, | Light-oxygen- | Neurospora | 405-499 | homo- | LOV domain of VVD |
| LINKER B | voltage-sensing | | | dimerization | gene with altered |
| | (LOV) domain | | | | light responsive |
| | | | | | properties and |
| | | | | | linker domains |
| NcVVDY50W, | Light-oxygen- | Neurospora | 405-499 | homo- | LOV domain of VVD |
| I52C, LINKER B | voltage-sensing | | | dimerization | gene with altered |
| | (LOV) domain | | | | light responsive |
| | | | | | properties and |
| | | | | | linker domains |
| NcVVDY50W, | Light-oxygen- | Neurospora | 405-499 | homo- | LOV domain of VVD |
| I52C, LINKER B | voltage-sensing | | | dimerization | gene with altered |
| | (LOV) domain | | | | light responsive |
| | | | | | properties and |
| | | | | | linker domains |
| NcVVDY50W, | Light-oxygen- | Neurospora | 405-499 | homo- | LOV domain of VVD |
| I52C, LINKER B | voltage-sensing | | | dimerization | gene with altered |
| | (LOV) domain | | | | light responsive |
| | | | | | properties and |
| | | | | | linker domains |

TABLE 2
Stress Granule Genes and Proteins
Stress Granules

| Gene | | |
|---|---|---|
| Name | Human Gene | Human Protein |
| PABC1 | NC_000005.10 | XP_016864445.1, |
| | | XP_005248287.1 |
| TIA1 | NG_029967.1 | NP_001338437.1, |
| | | NP_001338438.1, |
| | | NP_001338439.1, |
| | | NP_001338440.1, |
| | | NP_001338441.1, |
| | | NP_001338442.1, |
| | | NP_001338443.1, |
| | | NP_001338444.1, |
| | | NP_001338445.1, |
| | | NP_001338446.1, |
| | | NP_001338447.1, |
| | | NP_001338448.1, |
| | | NP_001338449.1, |
| | | NP_001338450.1, |
| | | NP_001338451.1, |
| | | NP_001338452.1, |
| | | NP_001338453.1, |
| | | NP_001338454.1 |
| TIAR | NC_027300.1 | XP_014053672.1 |
| G3BP1 | NC_000005.10 | XP_006714812.1, |
| | | XP_006714813.1, |
| | | XP_016864411.1, |
| | | XP_016864412.1 |
| G3BP2 | NC_000004.12 | NP_036429.2 |
| | | NP_987101.1, |
| | | NP_987100.1 |
| DDX6 | NC_000011.10 | NP_001244120 |
| TDRD3 | NC_000013.11 | NP_001139542.1, |
| | | NP_001139543.1 |
| ATXN2 | NC_027903.1 | XP_015008288.1, |
| | | XP_015008298.1, |
| | | XP_015008295.1, |
| | | XP_015008297.1, |
| | | XP_015008300.1, |
| | | XP_002808037.2 |

TABLE 3
P-Body Genes and Proteins
P-Bodies

| Gene | | |
|---|---|---|
| Name | Human Gene | Human Protein |
| DCP1A | NC_000003.12 | NP_001277133.1, |
| | | NP_001277134.1, |
| | | NP_001277135, |
| | | NP_001277136.1, |
| | | NP_060873.4 |
| DCP2 | NC_000005.10 | XP_016864652.1 |
| LSM1 | NC_000008.11 | NP_055277.1 |
| TNRC6A | NC_000016.10 | XP_005255314.1, |
| | | XP_005255311.1, |
| | | XP_016878643.1, |
| | | XP_016878635.1, |
| | | XP_016878640.1, |
| | | XP_016878637.1, |
| | | XP_016878638.1, |
| | | XP_016878639.1, |
| | | XP_016878641.1, |
| | | XP_016878633.1, |
| | | XP_016878632.1, |
| | | XP_016878634.1, |
| | | XP_016878636.1 |
| MEX3A | NC_000001.11 | NP_001087194.1 |
| EDC4 | NC_000016.10 | NP_055144.3 |
| XRN1 | NC_000003.12 | NP_001269786.1, |
| | | NP_001269788.1, |
| | | NP_061874.3 |
| DDX3X | NC_000023.11 | NP_001180345.1, |
| | | NP_001180346.1, |
| | | NP_001347.3 |

TABLE 4
Ribonuclear Transport Granule Genes and
Proteins
Ribonuclear Transport Granules

| Gene | | |
|---|---|---|
| Name | Human Gene | Human Protein |
| IGFBP1 | NC_000017.11 | NP_006537.3, |
| | | NP_001153895.1 |
| STAU1 | NC_000020.11 | NP_001032405.1, |
| | | NP_001306063.1, |
| | | NP_001306064.1, |
| | | NP_001309856.1, |
| | | NP_001309857.1, |
| | | NP_001309858.1, |
| | | NP_001309859.1, |
| | | NP_001309860.1, |
| | | NP_001309861.1, |
| | | NP_004593.2, |
| | | NP_059347.2, |
| | | NP_059348.2 |
| PURA | NC_000005.10 | NP_005850.1 |
| FMR1 | NC_000023.11 | NP_001172004.1, |
| | | NP_001172005.1, |
| | | NP_001172010.1, |
| | | NP_001172011.1, |
| | | NP_002015.1 |
| FXR1 | NC_000003.12 | NP_001013456.1, |
| | | NP_001013457.1, |
| | | NP_005078.2 |
| FXR2 | NC_000017.11 | NP_004851.2 |

TABLE 5
Nucleolus Genes and Proteins
Nucleolus

| Gene | | |
|---|---|---|
| Name | Human Gene | Human Protein |
| NCL | NC_000002.12 | NP_005372.2 |
| NPM1 | NC_000005.10 | NP_954654.1, |
| | | NP_002511.1, |
| | | NP_001032827.1 |
| FBL | NC_000019.10 | XP_005258708.1, |
| | | XP_011524925.1 |

TABLE 6
Nuclear Speckles - Genes and Proteins
Nuclear Speckles

| Gene | | |
|---|---|---|
| Name | Human Gene | Human Protein |
| SRSF2 | NC_000017.11 | NP_003007.2, |
| | | NP_001182356.1 |
| PNN | NC_000014.9 | NP_002678.2 |
| SRSF1 | NC_000017.11 | NP-001071634.1, |
| | | NP_008855.1 |

TABLE 7
Nuclear Stress Bodies - Genes and Proteins
Nuclear Stress Bodies

| Gene | | |
|---|---|---|
| Name | Human Gene | Human Protein |
| SAFB | NC_000019.10 | NP_002958.2, |
| | | NP_001307501.1, |
| | | NP_001307500.1, |
| | | NP_001188269.1, |
| | | NP_001188268.1, |
| | | NP_001188267.1 |

TABLE 8
PML Bodies - Genes and Proteins
PML Bodies

| Gene | | |
|---|---|---|
| Name | Human Gene | Human Protein |
| PML | NC_000015.10 | NP_002666.1, |
| | | NP_150241.2, |
| | | NP_150242.1, |
| | | NP_150243.2, |
| | | NP_150247.2, |
| | | NP_150249.1, |
| | | NP_150250.2, |
| | | NP_150252.1, |
| | | NP_150253.2 |

TABLE 9
Cajal Bodies - Genes and Proteins
Cajal Bodies

| Gene | | |
|---|---|---|
| Name | Human Gene | Human Protein |
| COIL | NC_000017.11 | NP_004636.1 |

TABLE 10
Nuclear Paraspeckles - Genes and Proteins
Nuclear Paraspeckles

| Gene | | |
|---|---|---|
| Name | Human Gene | Human Protein |
| SFPQ | NC_000001.11 | NP_005057.1 |
| NONO | NC_000023.11 | NP_031389.3, |
| (PNN) | | NP_001138882.1, |
| | | NP_001138881.1, |
| | | NP_001138880.1 |
| PSPC1 | NC_000013.11 | NP_001035879.1 |

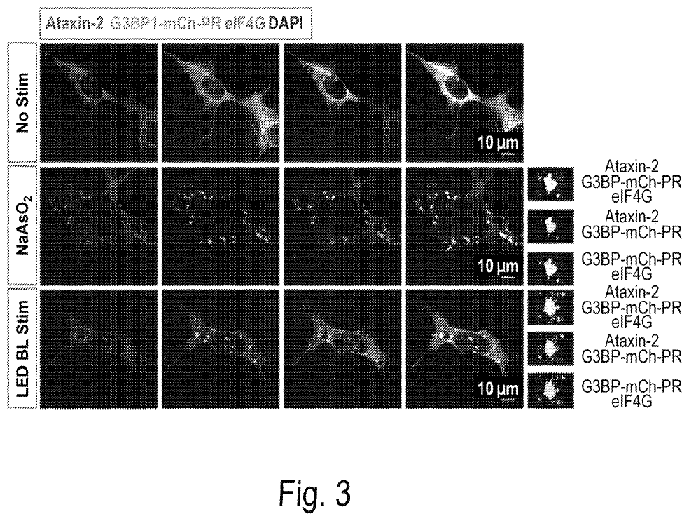

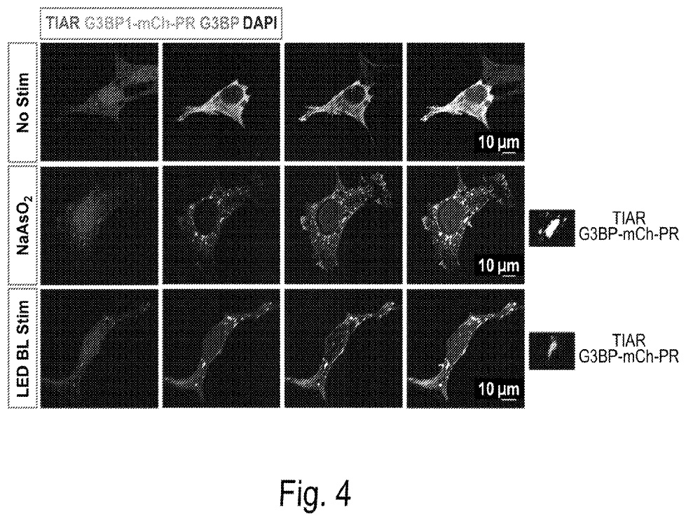

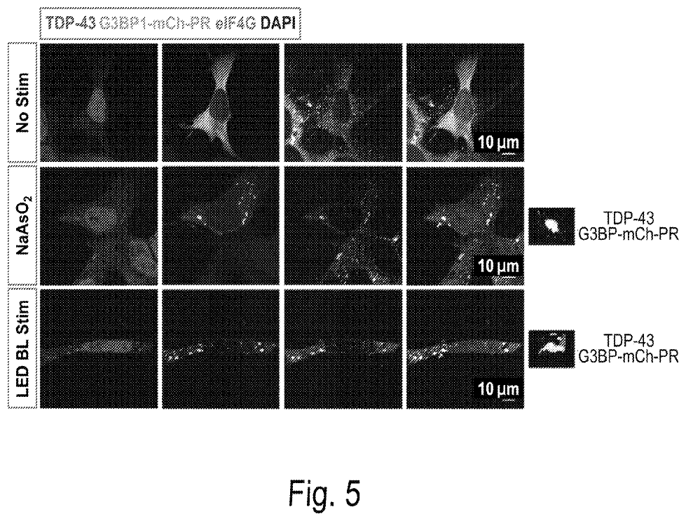

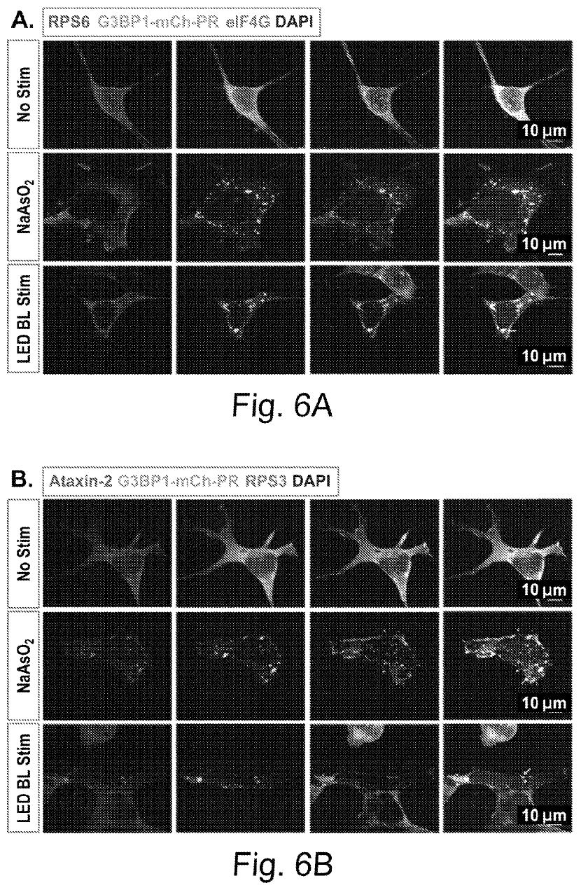

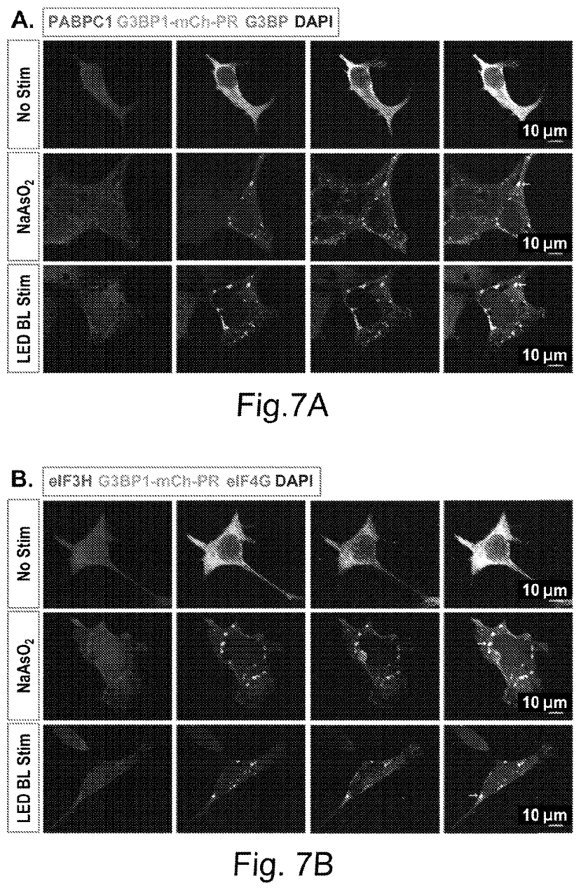

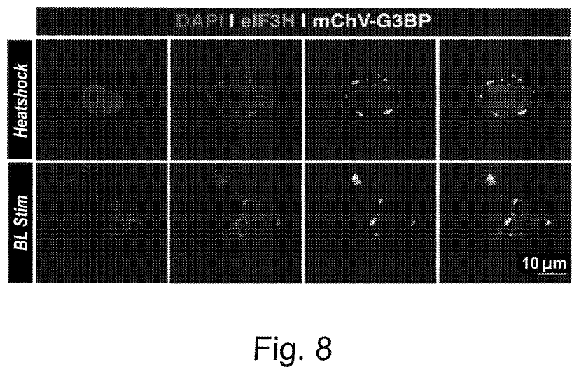

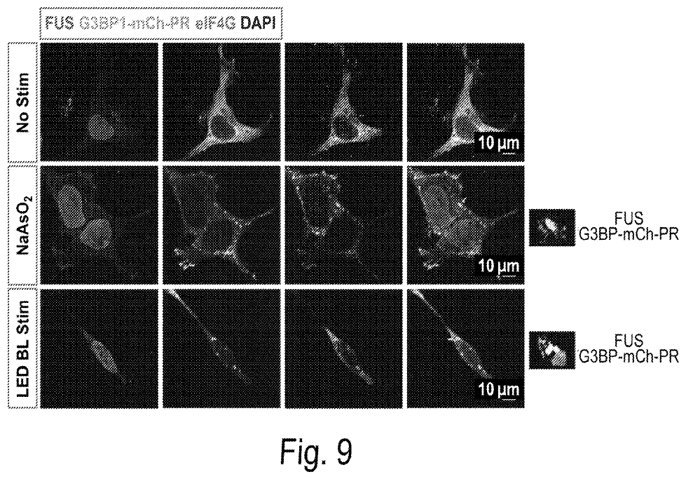

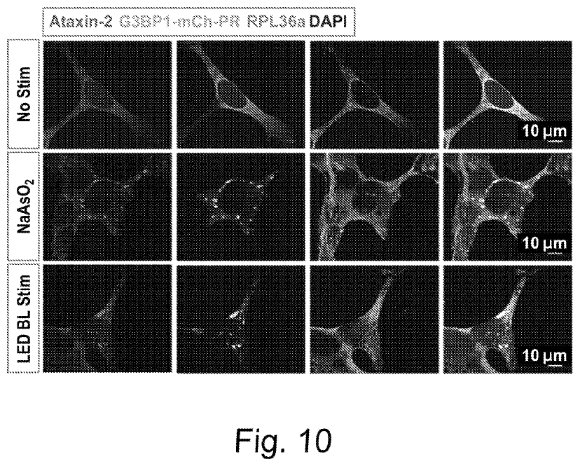

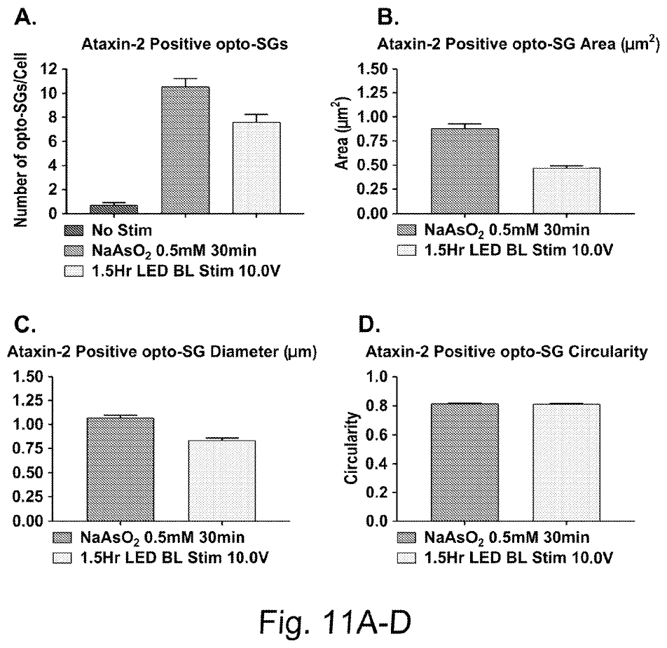

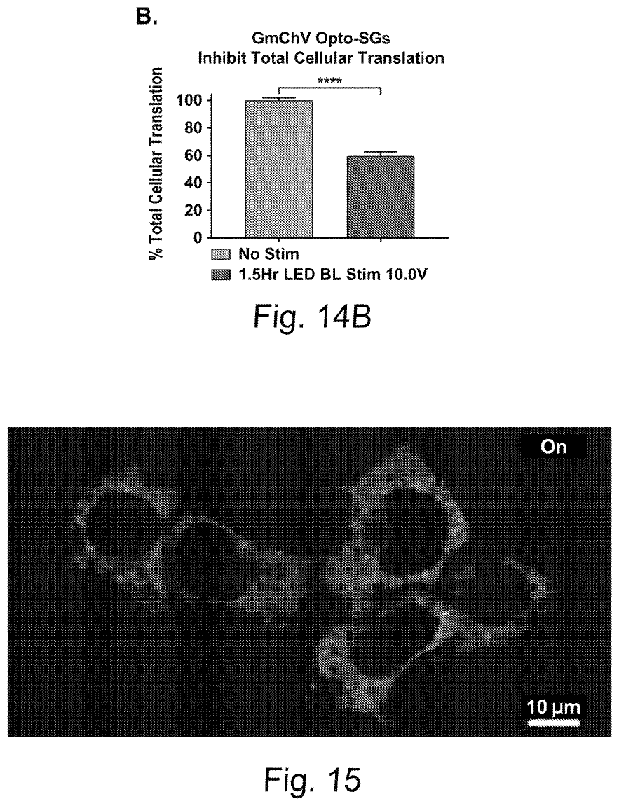

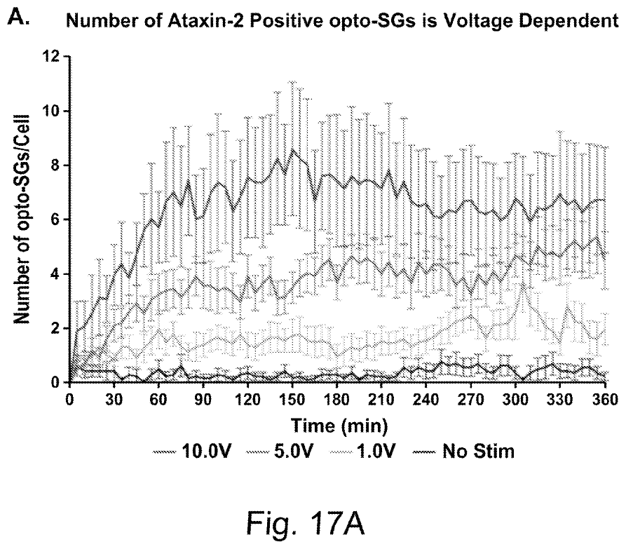

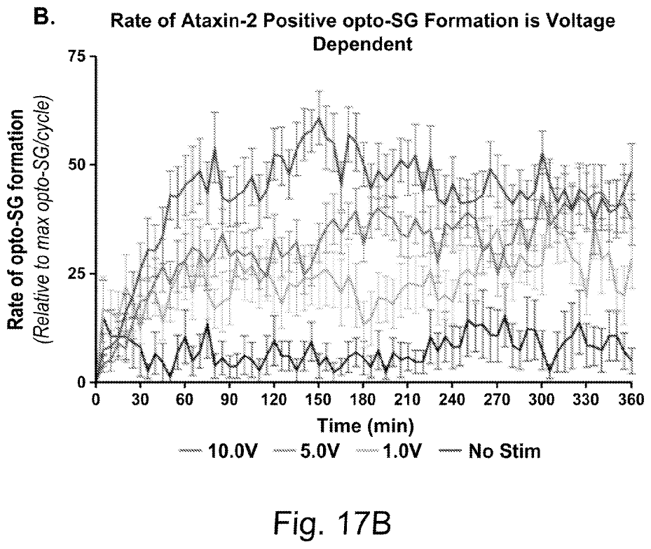

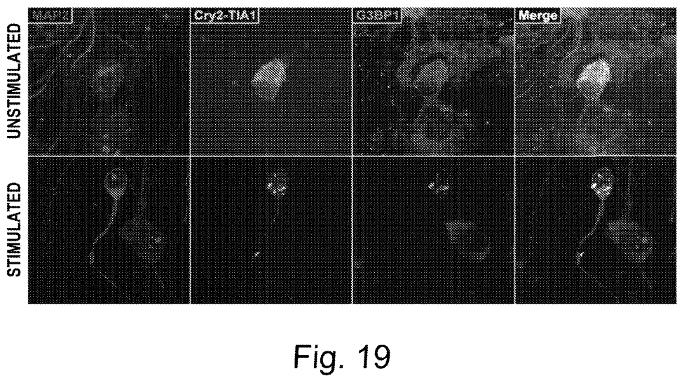

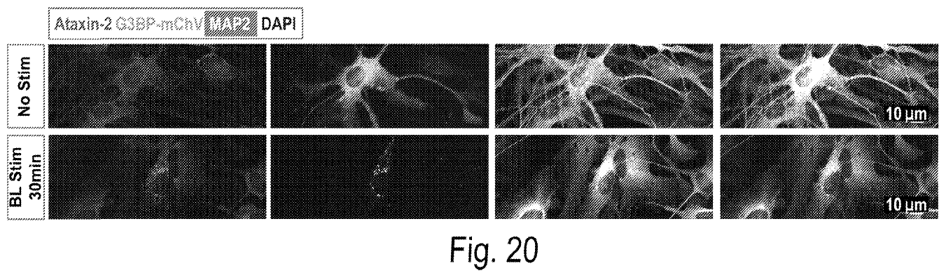

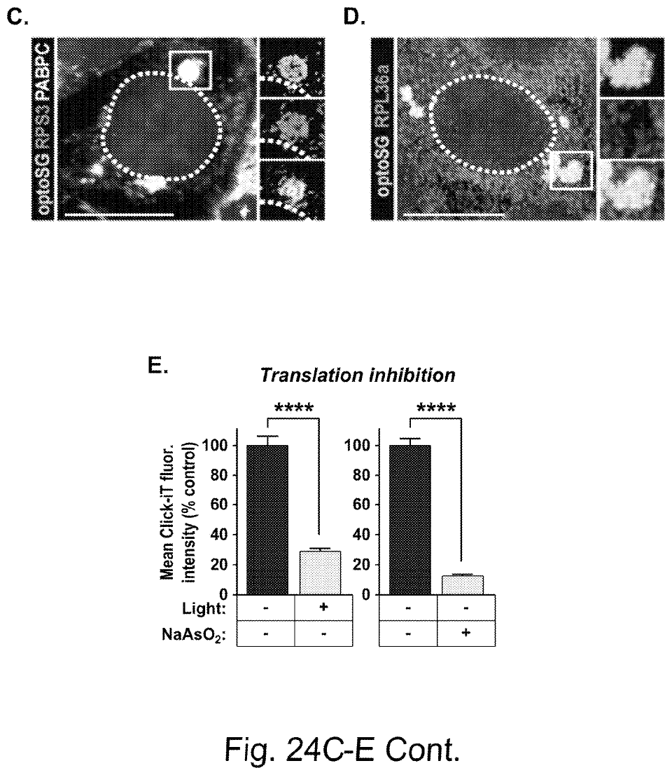

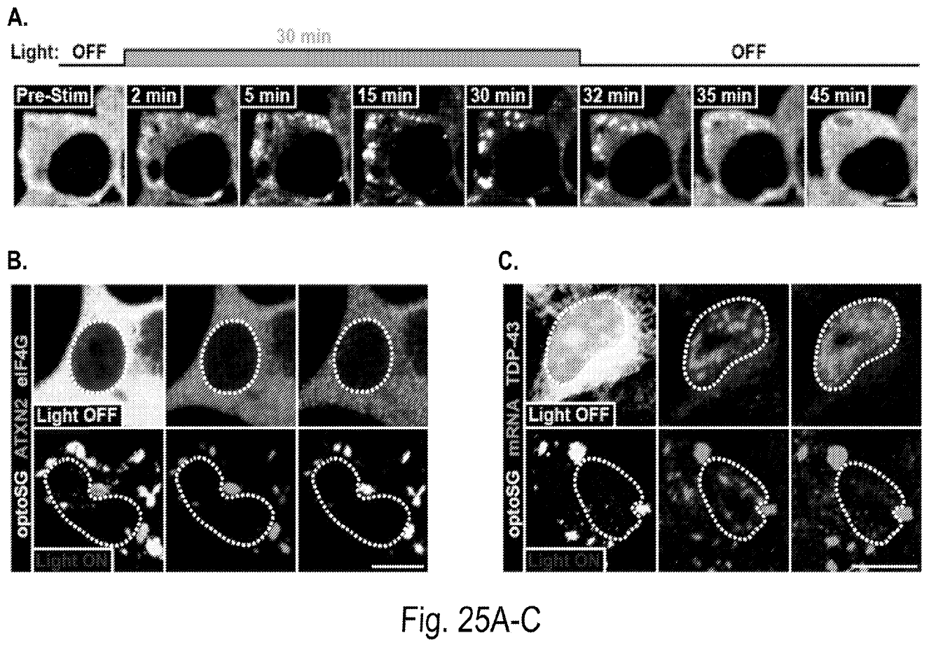

When these unique DNA arrangements are expressed in cells, they generate fusion proteins. Light exposure forces the fusion protein into close proximity and by employing acute, chronic, or repeated light stimulation, intracellular phase separation of the core proteins that comprise the membraneless organelle is driven by the LCD/IDR domain of the respective proteins. This seeding of the core structure allows for the recruitment of additional proteins and factors for the maturation of the organelle (FIGS. 3-25). This temporal and spatial control of organelle formation is novel and is used in both basic biological studies regarding the role of these organelles in cell function and in diseases, including for example, neurodegeneration and cancer.

In this example, cytoplasmic stress granules are formed using the methods disclosed herein. Cytoplasmic stress granules are membraneless organelles that form in the presence of extracellular stressors such as oxidative stress, osmotic stress, and heat shock. Their primary function is to inhibit the translation or mRNAs until the stressor is removed. Stress granules have been implicated in the initiation of many neurodegenerative disease pathologies, such as Amyotrophic Lateral Sclerosis, Alzheimer's Disease, Frontotemporal Dementia, and Parkinson's Disease, since the key proteins of these are found in stress granules. Stress granule dysfunction is also implicated in some cancers since certain cancer-causing mutations (e.g. DDX3X) are key components of these organelles.

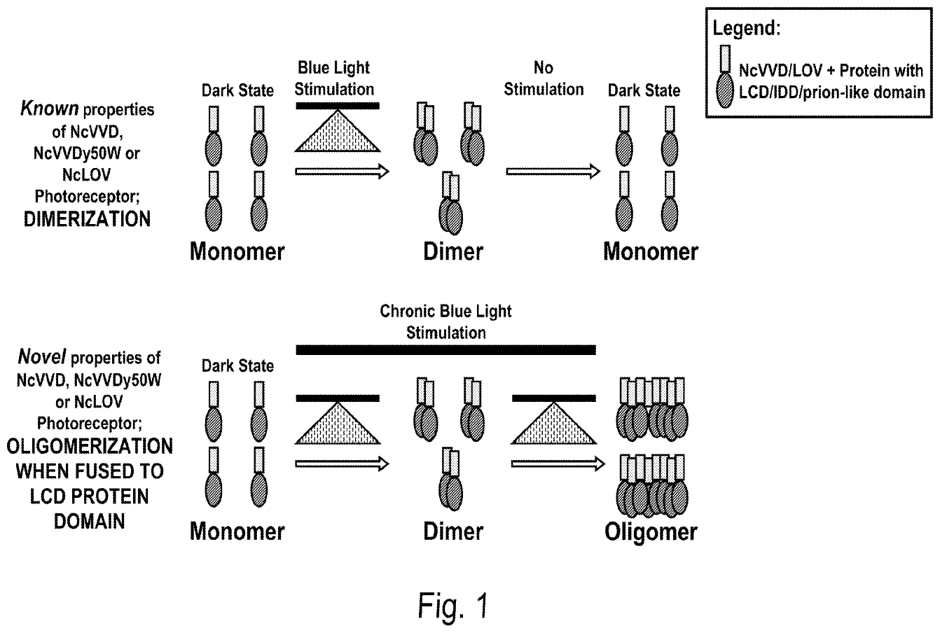

While stress granule dynamics are shown to be altered in these diseases, the true contributions of stress granules to disease pathobiology is unknown since the only current method to form these organelles is to introduce an extracellular stressor (e.g. heat stress at 43° C., exposure to sodium arsenite) which is toxic to cells and ultimately kills the cells being studied. The longest studies of stress granules to date are only on the order of a few hours and so, the importance of these organelles in the initiation of neurodegeneration or cancer cannot be assessed. Therefore, a method to control stress granule formation was developed using light, without the need for any extracellular stressors. To achieve this, the DNA encoding specific photoreceptor protein domains that self-bind with blue light exposure were linked to the DNA of a key protein required for the formation of stress granules, G3BP1 or TIA1. These arrangements were then expressed in HEK293 cells and were tested for their ability to form functional stress granules with various blue light treatment paradigms. It was shown that the LOV photoreceptor domain, from the Vivid protein of N. crassa, which is known to dimerize with blue light exposure, when fused to G3BP1 and exposed to 15-1080 min of 465 nm light at 0.1-1.0 mW/cm2 initiates the formation of a core stress granule structure consisting of the photoreceptors fused to G3BP1 that recruits additional G3BP1 and accessory proteins that comprise stress granules and allow for their maturation. This likely involved the liquid-liquid phase separation (LLPS) and dimerization and oligomerization of G3BP1 (concept represented in FIG. 1).

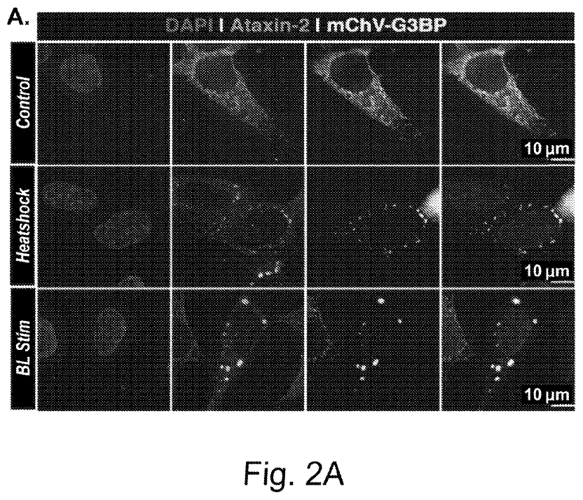

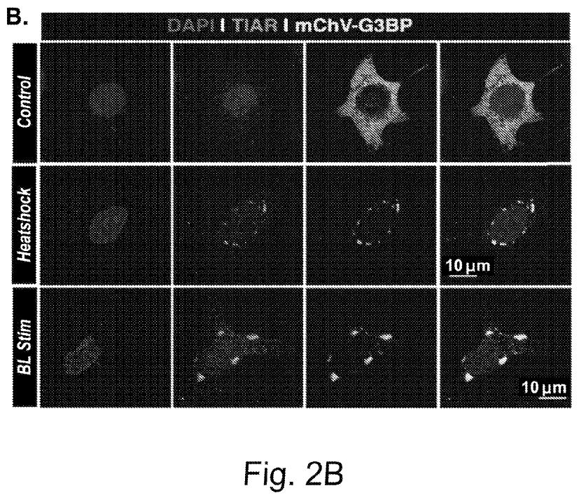

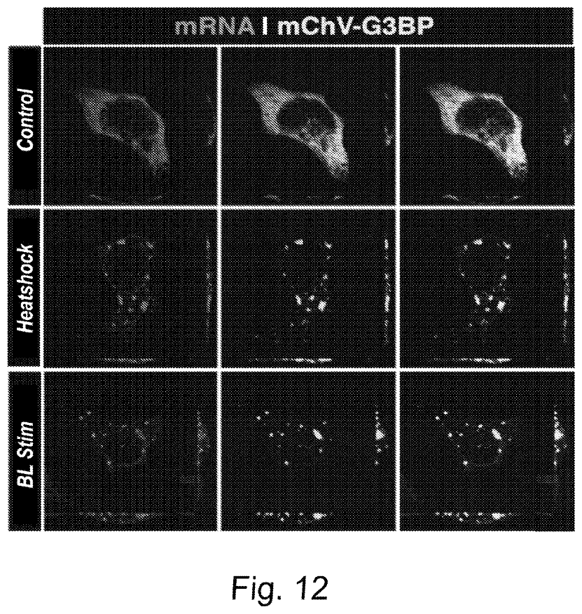

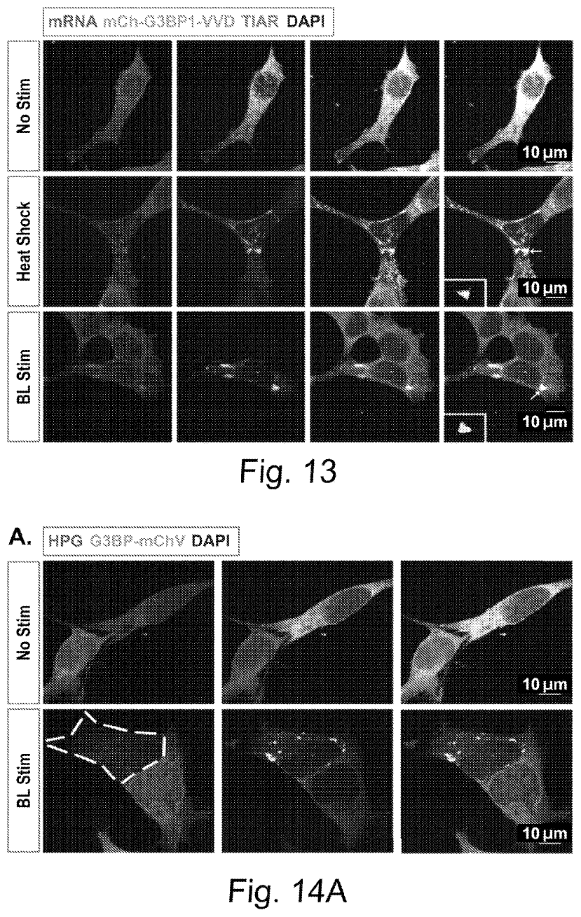

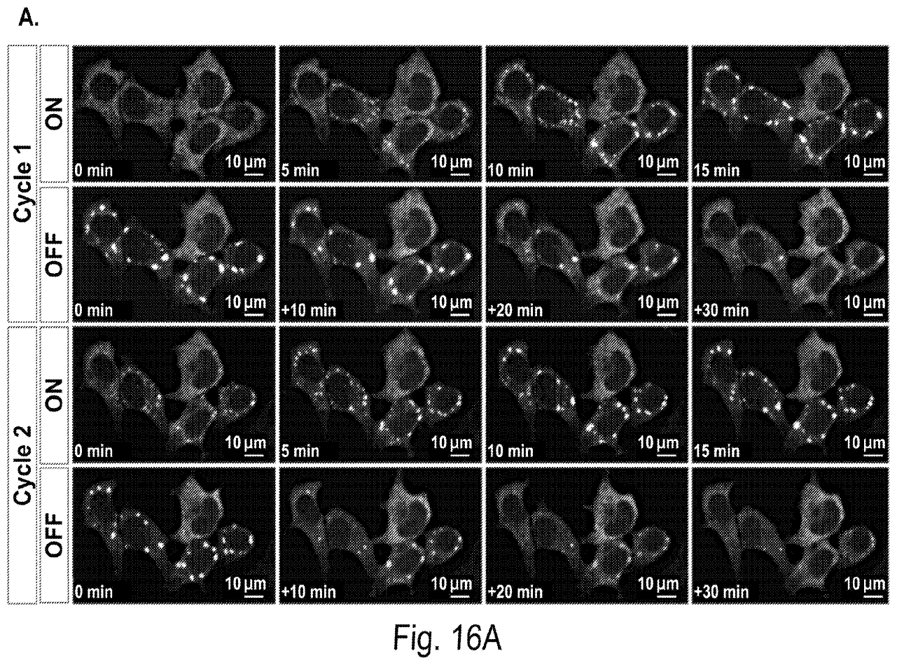

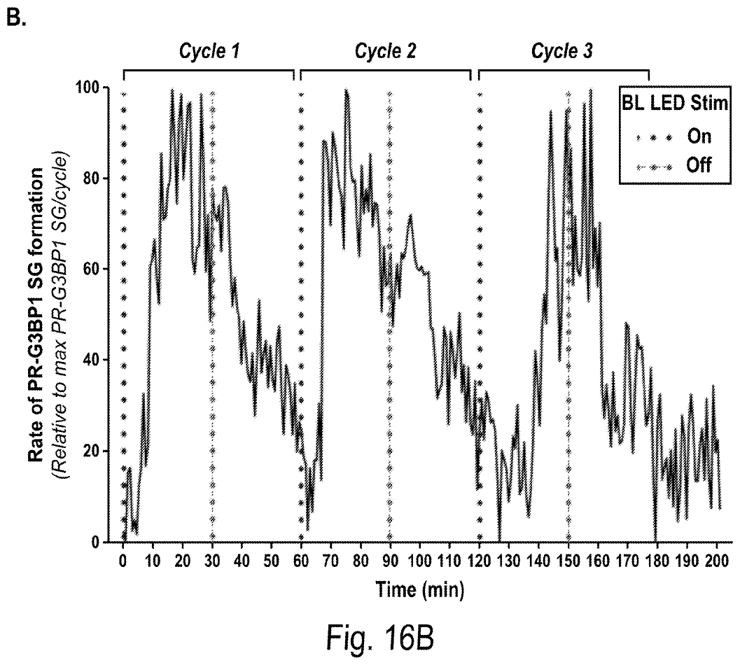

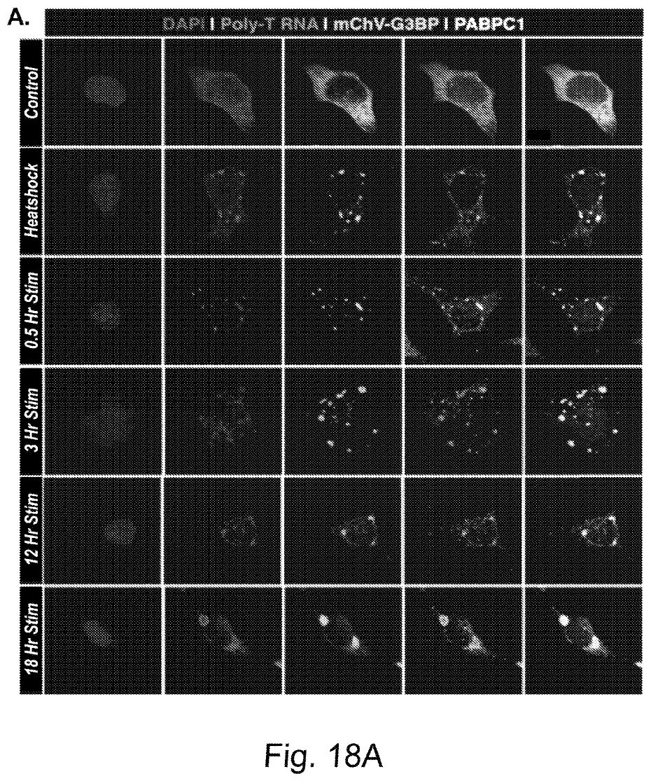

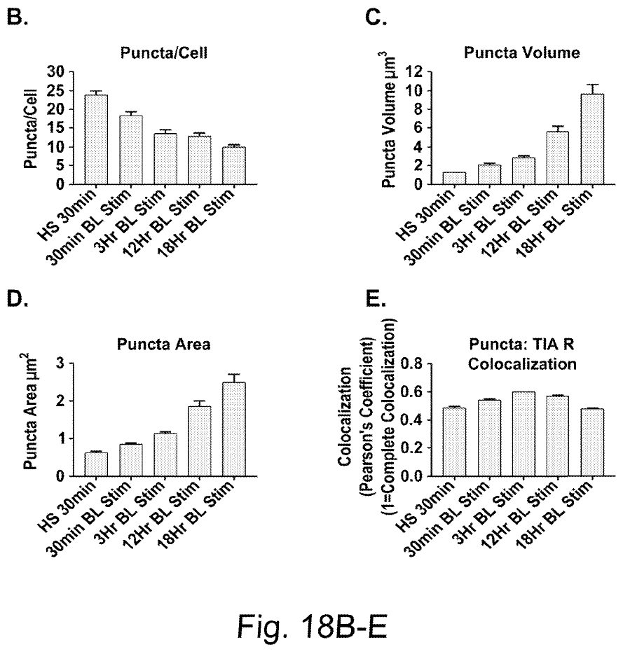

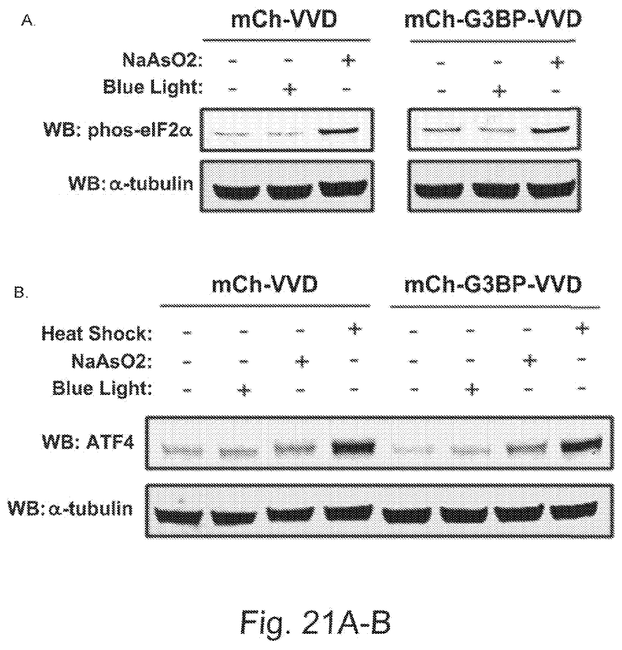

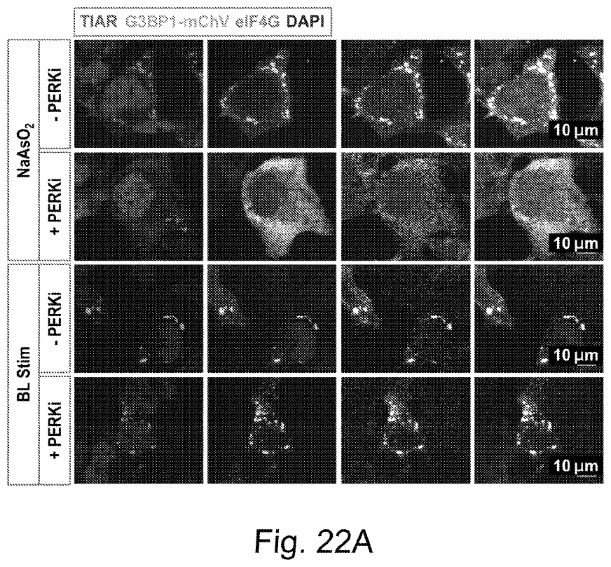

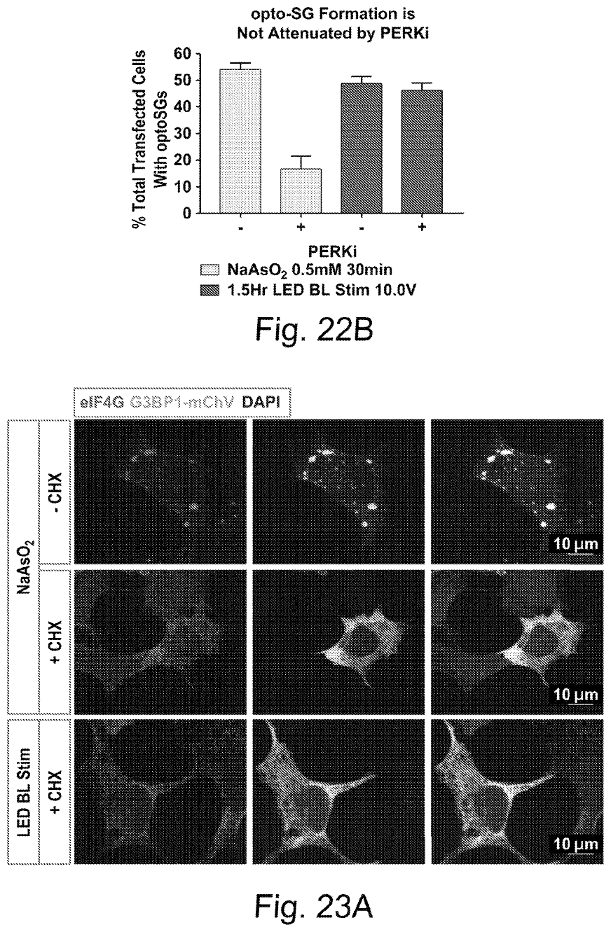

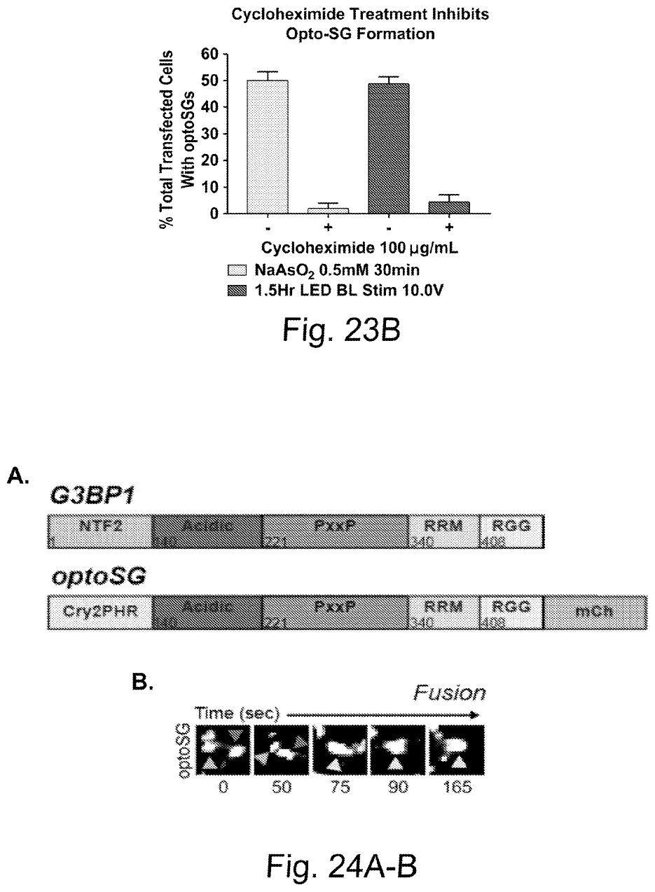

These light induced stress granules colocalize with key stress granule components, including Ataxin-2, TIAR, G3BP, eIF4G, eIF3H, RPS3, RPS6, TDP-43, FUS mRNA, and PABPC1 (FIGS. 2-9, 12, 13, 18, 20, 22, 23, 24, 25). Furthermore, the colocalization with mRNA (FIGS. 12 and 13) indicates the light induced stress granules are likely inhibiting mRNA translation. Note that the light induced stress granules appear identical to naturally occurring stress granules induced by heat shock treatment for 30 minutes at 42° C. (FIG. 2, 8, 12, 13, 18), or 0.5 mM sodium arsenite treatment for 30 minutes (FIGS. 3-7, 9, 10, 20-25). The light induced stress granules are tunable and can be initiated over multiple rounds with light as shown in FIG. 16 (and represented at different time points in FIG. 16), enabling an unprecedented level of control over the light induced stress granule that has not been previously achieved. Finally, light induced stress granules can be generated for multiple hours with chronic light treatment (FIG. 18) and colocalize with key stress granule markers throughout the entire time course. The stress granules are dynamic as they increase in size and decrease in number over time with chronic stimulation, suggesting fusion events (FIGS. 15 and 16). This is a hallmark characteristic of membraneless organelles. Light induced optoStress Granules (optoSGs) exhibit similar physiological properties as endogenously induced stress granules but in the absence of extracellular cell stress. Namely, western blot analysis shows that light induced stress granules do not activate the intrinsic stress response (FIG. 21). Consistent with this, optoSGs are not inhibited by PERK inhibitors (FIG. 22) which prevent eIF2 phosphorylation. However, they are inhibited in response to the translational inhibitor, cycloheximide (FIG. 23), suggesting that while the stress response is not necessary for their formation, the recruitment of ribosomal subunits is required. Together, these data show that the light induced stress granules mimic stress granules formed using extracellular stressors but in the absence of the intrinsic stress response (ISR).

The data shown here focuses on the formation of light-induced stress granules. In addition, this technology is also applied to other cytoplasmic or nuclear organelles. These methods only require that the photoreceptor has access to the FAD cofactor, which is present in both the cytoplasm and nucleus, and that the target membraneless organelle core protein contains a LCDs/IDRs to initiate liquid-liquid phase separation (LLPS). In some embodiments, the arrangement of any combination of DNA/protein fusion sequences listed in Tables 1 and 2-10 are included within the scope of the present disclosure. In some embodiments, the light treatments can consist of acute (<1 min) or chronic stimulation (>1 min) paradigms of blue light (405-495 nm) at 0.1-10 mW/cm2.

The DNA arrangement disclosed herein can encode for the CRY2 (PHR domain) or VVD/LOV (LOV domain) photoreceptor proteins (or for example, proteins within 90% similarity) (Table 1) fused to core proteins that contain low complexity domains (LCDs) and are required for the formation of membraneless organelles listed in Table 2 and the methodology of employing blue light exposure treatment paradigms times to induce the formation of functional membraneless organelles.

Finally, the compounds and compositions disclosed herein can be used in this methodology, and the resulting protein aggregates and cell viability are determined as a readout for drug screening that targets alterations in the function of these organelles.

The methods herein are used for the generation of novel model systems in concert with light treatment. These photoreceptor sequences are inserted into the genome of various in vitro and in vivo systems which act as a new model to study these organelles and to study the relationship of these organelles in disease. Nonlimiting examples include: a) In vitro: human and rodent cell lines, induced pluripotent stem cells (iPSCs), yeast, and/or b) In vivo invertebrates: Drosophila melanogaster (fruit fly), Caenorhabditis elegans (round worm), Danio rerio (zebrafish), and/or c) In vivo vertebrates: mouse, rat, non-human primate

The described models, (such as iPSC) with edited genomes are used in basic science studies and for high throughput drug screening systems. To achieve this, the formation, or disruption, of these organelles is initiated under temporal and spatial control by stimulating cells with light. Cell viability is then monitored and the formation and residence time of the membraneless organelle or other relevant pathologies are measured in the presence of a compound (or compound libraries). Additional assays include employing survival and neuropathology of in vivo models following induction with light.

To date, no one has successfully created functional membraneless organelles. In this example, novel DNA/protein arrangements are disclosed comprising fusions of 1) photoreceptors and 2) target proteins that are core components of membraneless organelles along with specific light treatment paradigms to create these functional structures. The photoreceptor protein domains have previously been identified and known to dimerize (self-bind) with blue light as it is their natural function in their host organism. The core components of the various membraneless organelles are identified from a number of studies over the past three decades. A common feature of these core proteins is that they contain intrinsically disordered regions/low complexity domains that, when in a high concentration in a liquid solution such as the cytosol in the cell, undergo liquid-liquid phase separation (LLPS) due to weak, attractive multivalent forces conferred by the intrinsically disordered regions/low complexity domains. Disclosed herein are arrangements of photoreceptors that self-bind and the core components of membraneless organelles that contain intrinsically disordered regions/low complexity domains to initiate LLPS and subsequently generate a functional membraneless organelle. For these results, the example of a cytoplasmic stress granule was analyzed. The methods disclosed herein are also applied to any other cytoplasmic or nuclear membraneless organelles. Also disclosed herein are novel photoreceptor and membraneless organelle DNA/protein arrangements that initiate LLPS and recruit additional factors necessary to create a functional membraneless organelle in the cell cytoplasm or the nucleus. Light stimulation paradigms to treat the arrangements are also disclosed herein. The combinations in Tables 1 and 2-10 can be used in cell or animal models to create functional membraneless organelles.

Disclosed herein are DNA/protein arrangements using the Cry2-target protein arrangement. Also disclosed are studies that employ the LOV domain of the Vivid protein from N. crassa. This photoreceptor is smaller than Cry2. However, the LOV domain has not been shown to oligomerize, and has only been published to dimerize. Herein, protein arrangements of the Vivid LOV domain and the stress granule core protein, G3BP1 (as well as other core proteins of membraneless organelles) have been developed, along with specific blue light treatments to force this dimerizing protein to cluster, and thus creating functional G3BP1 stress granule cores which then recruit other stress granule factors to generate a light-induced membraneless organelle. These findings are further unexpected since the Vivid LOV domain has not previously been shown to oligomerize with light. With the treatments and DNA arrangement disclosed herein, the Vivid LOV domain is used to force the oligomerization of proteins that contain low complexity domains/intrinsically disordered regions.

These tools and methods disclosed herein are used in the field of neurodegeneration since stress granules, nucleoli, p-bodies and other membraneless organelles have been linked to a number of diseases including Alzheimer's Disease, Frontotemporal Dementia, Parkinson's Disease, Lewy body Dementia, and Traumatic Brain Injury to name a few. Beyond neurodegeneration, aberrant function of many listed membraneless organelles such as stress granules (Table 2), are observed in cancers. This technology allows development of these structures in a tunable manner, thus providing a platform to investigate their relevance in a variety of disorders and can be used to screen for drugs or therapeutics as modifiers of the induced organelles.

In addition to use in drug discovery using the DNA/protein arrangements, cell and animal models for each membraneless organelle are of great interest to the scientific research community. This technology allows researchers, for the first time, to control the initiation of these functional organelles to study their role in basic biological processes that span a variety of fields including neuroscience, molecular and cellular biology, chemistry and biochemistry, cancer biology, pharmacology, and bioengineering.

### SEQUENCES

Sequences of Photoreceptor Tools:
Amino Acid Sequences of the Photolyase-Homologous Region (PHR) domain of the Arabidopsis
Cryptochrome 2 protein:

Cryptochrome 2 PHR Domain; Cry2PHR:
MKMDKKTIVWFRRDLRIEDNPALAAAAHEGSVFPVFIWCPEEEGQFYPGRASRWWMKQSLAHLSQSLKALGSDL
TLIKTHNTISAILDCIRVTGATKVVFNHLYDPVSLVRDHTVKEKLVERGISVQSYNGDLLYEPWEIYCEKGKPFTSFNSY
WKKCLDMSIESVMLPPPWRLMPITAAAEAIWACSIEELGLENEAEKPSNALLTRAWSPGWSNADKLLNEFIEKQLI
DYAKNSKKVVGNSTSLLSPYLHFGEISVRHVFQCARMKQIIWARDKNSEGEESADLFLRGIGLREYSRYICFNFPFTH
EQSLLSHLRFFPWDADVDKFKAWRQGRTGYPLVDAGMRELWATGWMHNRIRVIVSSFAVKFLLLPWKWGMKY
FWDTLLDADLECDILGWQYISGSIPDGHELDRLDNPALQGAKYDPEGEYIRQWLPELARLPTEWIHHPWDAPLTVL
KASGVELGTNYAKPIVDIDTARELLAKAISRTREAQIMIGAA (SEQ ID NO: 1)
Cryptochrome 2 PHR Domain with E490G substitution; Cry2Olig:
MKMDKKTIVWFRRDLRIEDNPALAAAAHEGSVFPVFIWCPEEEGQFYPGRASRWWMKQSLAHLSQSLKALGSDL
TLIKTHNTISAILDCIRVTGATKVVFNHLYDPVSLVRDHTVKEKLVERGISVQSYNGDLLYEPWEIYCEKGKPFTSFNSY
WKKCLDMSIESVMLPPPWRLMPITAAAEAIWACSIEELGLENEAEKPSNALLTRAWSPGWSNADKLLNEFIEKQLI
DYAKNSKKVVGNSTSLLSPYLHFGEISVRHVFQCARMKQIIWARDKNSEGEESADLFLRGIGLREYSRYICFNFPFTH
EQSLLSHLRFFPWDADVDKFKAWRQGRTGYPLVDAGMRELWATGWMHNRIRVIVSSFAVKFLLLPWKWGMKY
FWDTLLDADLECDILGWQYISGSIPDGHELDRLDNPALQGAKYDPEGEYIRQWLPELARLPTEWIHHPWDAPLTVL
KASGVELGTNYAKPIVDIDTARELLAKAISRTRGAQIMIGAA (SEQ ID NO: 2)
Amino Acid Sequences containing the Light-Oxygen-Voltage-Sensing Domain (LOV) from
Neurospora crassa Vivid protein:
VVD LOV Domain only:
MTSCALILCDLKQKDTPIVYASEAFLYMTGYSNAEVLGRNCRFLQSPDGMVKPKSTRKYVDSNTINTMRKAIDRNA
EVQVEVVNFKKNGQRFVNFLTMIPVRDETGEYRYSMGFQCETE (SEQ ID NO: 3)
NcVivid (NcVVD):
MHTLYAPGGYDIMGYLIQIMNRPNPQVELGPVDTSCALILCDLKQKDTPIVYASEAFLYMTGYSNAEVLGRNCRFL
QSPDGMVKPKSTRKYVDSNTINTMRKAIDRNAEVQVEVVNFKKNGQRFVNFLTMIPVRDETGEYRYSMGFQCET
E (SEQ ID NO: 4)
NcVivid Y50W substitution; NcVVDY50W:
MHTLYAPGGYDIMGWLIQIMNRPNPQVELGPVDTSCALILCDLKQKDTPIVYASEAFLYMTGYSNAEVLGRNCRFL
QSPDGMVKPKSTRKYVDSNTINTMRKAIDRNAEVQVEVVNFKKNGQRFVNFLTMIPVRDETGEYRYSMGFQCET
E (SEQ ID NO: 5)
Amino Acid Sequences of the Vaucheria frigida (Yellow-green alga) (Conferva frigida)
Aureochrome1 protein (Gene is AUREO1):
VfAU1 (A8QW55):
MNGLTPPLMFCSRSDDPSSTSNINLDDVFADVFFNSNGELLDIDEIDDFGDNTCPKSSMSVDDDASSQVFQGHLF
GNALSSIALSDSGDLSTGIYESQGNASRGKSLRTKSSGSISSELTEAQKVERRERNREHAKRSRVRKKFLLESLQQSVN
ELNHENNCLKESIREHLGPRGDSLIAQCSPEADTLLTDNPSKANRILEDPDYSLVKALQMAQQNFVITDASLPDNPIV
YASRGFLTLTGYSLDQILGRNCRFLQGPETDPRAVDKIRNAITKGVDTSVCLLNYRQDGTTFWNLFFVAGLRDSKGN
IVNYVGVQSKVSEDYAKLLVNEQNIEYKGVRTSNMLRRK (SEQ ID NO: 6)
VfAU1-LOV domain:
PDYSLVKALQMAQQNFVITDASLPDNPIVYASRGFLTLTGYSLDQILGRNCRFLQGPETDPRAVDKIRNAITKGVDT
SVCLLNYRQDGTTFWNLFFVAGLRDSKGNIVNYVGVQSKVSEDYAKLLVNEQNIEYKGVRTSNMLRRK (SEQ ID
NO: 7)
VfAU-DNA sequence-VfAU1-LOV domain (No Start codon):
Cctgactacagtctcgtgaaggctctgcaaatggcacaacagaattttgtcattacagacgcctccctcccagacaaccctatcgtctacgccagt
agagggtttctgacactgacaggctattctctcgaccagatcctgggcaggaactgcaggtttctgcaagggccagaaacagacccaagagctg
tggataagatcaggaatgccatcaccaaaggcgttgataccagtgtctgtctgctgaattatagacaggatggcacaaccttctggaatctcttct
tcgtggctggactcagagattctaagggcaatattgtcaactacgtcggagtgcagtcaaaggtgagcgaagattatgccaagctgctggtcaa
cgagcagaacattgagtacaaaggtgtgcgcaccagtaacatgctgcgcagaaag (SEQ ID NO: 8)
NcVivid Y50W with G3BP1 Full Length Protein; NcVVDY50W-G3BP1
MHTLYAPGGYDIMGWLIQIMNRPNPQVELGPVDTSCALILCDLKQKDTPIVYASEAFLYMTGYSNAEVLGRNCRFL
QSPDGMVKPKSTRKYVDSNTINTMRKAIDRNAEVQVEVVNFKKNGQRFVNFLTMIPVRDETGEYRYSMGFQCET
EMVMEKPSPLLVGREFVRQYYTLLNQAPDMLHRFYGKNSSYVHGGLDSNGKPADAVYGQKEIHRKVMSQNFTN
CHTKIRHVDAHATLNDGVVVQVMGLLSNNNQALRRFMQTFVLAPEGSVANKFYVHNDIFRYQDEVFGGFVTEP
QEESEEEVEEPEERQQTPEVVPDDSGTFYDQAVVSNDMEEHLEEPVAEPEPDPEPEPEQEPVSEIQEEKPEPVLEET
APEDAQKSSSPAPADIAQTVQEDLRTFSWASVTSKNLPPSGAVPVTGIPPHVVKVPASQPRPESKPESQIPPQRPQ
RDQRVREQRINIPPQRGPRPIREAGEQGDIEPRRMVRHPDSHQLFIGNLPHEVDKSELKDFFQSYGNVVELRINSG
GKLPNFGFVVFDDSEPVQKVLSNRPIMFRGEVRLNVEEKKTRAAREGDRRDNRLRGPGGPRGGLGGGMRGPPR
GGMVQKPGFGVGRGLAPRQKLIPMARRAWLEHGHSCFLCEIVIRSQFHTTYEPEA (SEQ ID NO: 9)
NcVivid Y50W with G3BP1 NTF2 Domain Deletion Mutation; NcVVDY50W-G3BP1dNTF2
MHTLYAPGGYDIMGWLIQIMNRPNPQVELGPVDTSCALILCDLKQKDTPIVYASEAFLYMTGYSNAEVLGRNCRFL
QSPDGMVKPKSTRKYVDSNTINTMRKAIDRNAEVQVEVVNFKKNGQRFVNFLTMIPVRDETGEYRYSMGFQCET
EMGFVTEPQEESEEEVEEPEERQQTPEVVPDDSGTFYDQAVVSNDMEEHLEEPVAEPEPDPEPEPEQEPVSEIQEE
KPEPVLEETAPEDAQKSSSPAPADIAQTVQEDLRTFSWASVTSKNLPPSGAVPVTGIPPHVVKVPASQPRPESKPES
QIPPQRPQRDQRVREQRINIPPQRGPRPIREAGEQGDIEPRRMVRHPDSHQLFIGNLPHEVDKSELKDFFQSYGNV
VELRINSGGKLPNFGFVVFDDSEPVQKVLSNRPIMFRGEVRLNVEEKKTRAAREGDRRDNRLRGPGGPRGGLGGG
MRGPPRGGMVQKPGFGVGRGLAPRQKLIPMARRAWLEHGHSCFLCEIVIRSQFHTTYEPEA (SEQ ID NO: 10)
NcVivid Y50W with G3BP1 Acidic Domain Deletion Mutation; NcVVDY50W-G3BP1dAcidic
MHTLYAPGGYDIMGWLIQIMNRPNPQVELGPVDTSCALILCDLKQKDTPIVYASEAFLYMTGYSNAEVLGRNCRFL
QSPDGMVKPKSTRKYVDSNTINTMRKAIDRNAEVQVEVVNFKKNGQRFVNFLTMIPVRDETGEYRYSMGFQCET
EMVMEKPSPLLVGREFVRQYYTLLNQAPDMLHRFYGKNSSYVHGGLDSNGKPADAVYGQKEIHRKVMSQNFTN
CHTKIRHVDAHATLNDGVVVQVMGLLSNNNQALRRFMQTFVLAPEGSVANKFYVHNDIFRYQDEVFGTAPEDA
QKSSSPAPADIAQTVQEDLRTFSWASVTSKNLPPSGAVPVTGIPPHVVKVPASQPRPESKPESQIPPQRPQRDQRV
REQRINIPPQRGPRPIREAGEQGDIEPRRMVRHPDSHQLFIGNLPHEVDKSELKDFFQSYGNVVELRINSGGKLPNF
GFVVFDDSEPVQKVLSNRPIMFRGEVRLNVEEKKTRAAREGDRRDNRLRGPGGPRGGLGGGMRGPPRGGMVQ
KPGFGVGRGLAPRQKLIPMARRAWLEHGHSCFLCEIVIRSQFHTTYEPEA (SEQ ID NO: 11)
NcVivid Y50W with G3BP1 PxxP Domain Deletion Mutation; NcVVDY50W-G3BP1dPxxP
MHTLYAPGGYDIMGWLIQIMNRPNPQVELGPVDTSCALILCDLKQKDTPIVYASEAFLYMTGYSNAEVLGRNCRFL
QSPDGMVKPKSTRKYVDSNTINTMRKAIDRNAEVQVEVVNFKKNGQRFVNFLTMIPVRDETGEYRYSMGFQCET
EMVMEKPSPLLVGREFVRQYYTLLNQAPDMLHRFYGKNSSYVHGGLDSNGKPADAVYGQKEIHRKVMSQNFTN
CHTKIRHVDAHATLNDGVVVQVMGLLSNNNQALRRFMQTFVLAPEGSVANKFYVHNDIFRYQDEVFGGFVTEP
QEESEEEVEEPEERQQTPEVVPDDSGTFYDQAVVSNDMEEHLEEPVAEPEPDPEPEPEQEPVSEIQEEKPEPVLEE
QLFIGNLPHEVDKSELKDFFQSYGNVVELRINSGGKLPNFGFVVFDDSEPVQKVLSNRPIMFRGEVRLNVEEKKTRA
AREGDRRDNRLRGPGGPRGGLGGGMRGPPRGGMVQKPGFGVGRGLAPRQKLIPMARRAWLEHGHSCFLCEIVI
RSQFHTTYEPEA (SEQ ID NO: 12)
NcVivid Y50W with G3BP1 RRM Domain Deletion Mutation; NcVVDY50W-G3BP1dRRM
MHTLYAPGGYDIMGWLIQIMNRPNPQVELGPVDTSCALILCDLKQKDTPIVYASEAFLYMTGYSNAEVLGRNCRFL
QSPDGMVKPKSTRKYVDSNTINTMRKAIDRNAEVQVEVVNFKKNGQRFVNFLTMIPVRDETGEYRYSMGFQCET
EMVMEKPSPLLVGREFVRQYYTLLNQAPDMLHRFYGKNSSYVHGGLDSNGKPADAVYGQKEIHRKVMSQNFTN
CHTKIRHVDAHATLNDGVVVQVMGLLSNNNQALRRFMQTFVLAPEGSVANKFYVHNDIFRYQDEVFGGFVTEP
QEESEEEVEEPEERQQTPEVVPDDSGTFYDQAVVSNDMEEHLEEPVAEPEPDPEPEPEQEPVSEIQEEKPEPVLEET
APEDAQKSSSPAPADIAQTVQEDLRTFSWASVTSKNLPPSGAVPVTGIPPHVVKVPASQPRPESKPESQIPPQRPQ
RDQRVREQRINIPPQRGPRPIREAGEQGDIEPRRMVRHPDSHEKKTRAAREGDRRDNRLRGPGGPRGGLGGGM
RGPPRGGMVQKPGFGVGRGLAPRQKLIPMARRAWLEHGHSCFLCEIVIRSQFHTTYEPEA (SEQ ID NO: 13)
NcVivid Y50W with G3BP1 RGG Domain Deletion Mutation; NcVVDY50W-G3BP1dRGG
MHTLYAPGGYDIMGWLIQIMNRPNPQVELGPVDTSCALILCDLKQKDTPIVYASEAFLYMTGYSNAEVLGRNCRFL
QSPDGMVKPKSTRKYVDSNTINTMRKAIDRNAEVQVEVVNFKKNGQRFVNFLTMIPVRDETGEYRYSMGFQCET
EMVMEKPSPLLVGREFVRQYYTLLNQAPDMLHRFYGKNSSYVHGGLDSNGKPADAVYGQKEIHRKVMSQNFTN
CHTKIRHVDAHATLNDGVVVQVMGLLSNNNQALRRFMQTFVLAPEGSVANKFYVHNDIFRYQDEVFGGFVTEP
QEESEEEVEEPEERQQTPEVVPDDSGTFYDQAVVSNDMEEHLEEPVAEPEPDPEPEPEQEPVSEIQEEKPEPVLEET
APEDAQKSSSPAPADIAQTVQEDLRTFSWASVTSKNLPPSGAVPVTGIPPHVVKVPASQPRPESKPESQIPPQRPQ
RDQRVREQRINIPPQRGPRPIREAGEQGDIEPRRMVRHPDSHQLFIGNLPHEVDKSELKDFFQSYGNVVELRINSG
GKLPNFGFVVFDDSEPVQKVLSNRPIMFRGEVRLNVE (SEQ ID NO: 14)
NcVivid Y50W with G3BP1 RRMRGG Domain Deletion Mutation; NcVVDY50W-G3BP1dRRMRGG
MHTLYAPGGYDIMGWLIQIMNRPNPQVELGPVDTSCALILCDLKQKDTPIVYASEAFLYMTGYSNAEVLGRNCRFL
QSPDGMVKPKSTRKYVDSNTINTMRKAIDRNAEVQVEVVNFKKNGQRFVNFLTMIPVRDETGEYRYSMGFQCET
EMVMEKPSPLLVGREFVRQYYTLLNQAPDMLHRFYGKNSSYVHGGLDSNGKPADAVYGQKEIHRKVMSQNFTN
CHTKIRHVDAHATLNDGVVVQVMGLLSNNNQALRRFMQTFVLAPEGSVANKFYVHNDIFRYQDEVFGGFVTEP
QEESEEEVEEPEERQQTPEVVPDDSGTFYDQAVVSNDMEEHLEEPVAEPEPDPEPEPEQEPVSEIQEEKPEPVLEET
APEDAQKSSSPAPADIAQTVQEDLRTFSWASVTSKNLPPSGAVPVTGIPPHVVKVPASQPRPESKPESQIPPQRPQ
RDQRVREQRINIPPQRGPRPIREAGEQGDIEPRRMVRHPDSH (SEQ ID NO: 15)
G3BP1 Full Length Protein with NcVivid Y50W; G3BP1-NcVVDY50W
MVMEKPSPLLVGREFVRQYYTLLNQAPDMLHRFYGKNSSYVHGGLDSNGKPADAVYGQKEIHRKVMSQNFTNC
HTKIRHVDAHATLNDGVVVQVMGLLSNNNQALRRFMQTFVLAPEGSVANKFYVHNDIFRYQDEVFGGFVTEPQE
ESEEEVEEPEERQQTPEVVPDDSGTFYDQAVVSNDMEEHLEEPVAEPEPDPEPEPEQEPVSEIQEEKPEPVLEETAP
EDAQKSSSPAPADIAQTVQEDLRTFSWASVTSKNLPPSGAVPVTGIPPHVVKVPASQPRPESKPESQIPPQRPQRD
QRVREQRINIPPQRGPRPIREAGEQGDIEPRRMVRHPDSHQLFIGNLPHEVDKSELKDFFQSYGNVVELRINSGGKL
PNFGFVVFDDSEPVQKVLSNRPIMFRGEVRLNVEEKKTRAAREGDRRDNRLRGPGGPRGGLGGGMRGPPRGGM
VQKPGFGVGRGLAPRQKLIPMARRAWLEHGHSCFLCEIVIRSQFHTTYEPEAMHTLYAPGGYDIMGWLIQIMNRP
NPQVELGPVDTSCALILCDLKQKDTPIVYASEAFLYMTGYSNAEVLGRNCRFLQSPDGMVKPKSTRKYVDSNTINT
MRKAIDRNAEVQVEVVNFKKNGQRFVNFLTMIPVRDETGEYRYSMGFQCETE (SEQ ID NO: 16)
G3BP1 NTF2 Domain Deletion Mutation with NcVivid Y50W; G3BP1dNTF2-NcVivid Y50W
MGFVTEPQEESEEEVEEPEERQQTPEVVPDDSGTFYDQAVVSNDMEEHLEEPVAEPEPDPEPEPEQEPVSEIQEEK
PEPVLEETAPEDAQKSSSPAPADIAQTVQEDLRTFSWASVTSKNLPPSGAVPVTGIPPHVVKVPASQPRPESKPESQ
IPPQRPQRDQRVREQRINIPPQRGPRPIREAGEQGDIEPRRMVRHPDSHQLFIGNLPHEVDKSELKDFFQSYGNVV
ELRINSGGKLPNFGFVVFDDSEPVQKVLSNRPIMFRGEVRLNVEEKKTRAAREGDRRDNRLRGPGGPRGGLGGG
MRGPPRGGMVQKPGFGVGRGLAPRQKLIPMARRAWLEHGHSCFLCEIVIRSQFHTTYEPEAMHTLYAPGGYDIM
GWLIQIMNRPNPQVELGPVDTSCALILCDLKQKDTPIVYASEAFLYMTGYSNAEVLGRNCRFLQSPDGMVKPKSTR
KYVDSNTINTMRKAIDRNAEVQVEVVNFKKNGQRFVNFLTMIPVRDETGEYRYSMGFQCETE (SEQ ID NO: 17)
G3BP1 Acidic Domain Deletion Mutation with NcVivid Y50W; G3BP1dAcidic-NcVivid Y50W
MVMEKPSPLLVGREFVRQYYTLLNQAPDMLHRFYGKNSSYVHGGLDSNGKPADAVYGQKEIHRKVMSQNFTNC
HTKIRHVDAHATLNDGVVVQVMGLLSNNNQALRRFMQTFVLAPEGSVANKFYVHNDIFRYQDEVFGTAPEDAQ
KSSSPAPADIAQTVQEDLRTFSWASVTSKNLPPSGAVPVTGIPPHVVKVPASQPRPESKPESQIPPQRPQRDQRVR
EQRINIPPQRGPRPIREAGEQGDIEPRRMVRHPDSHQLFIGNLPHEVDKSELKDFFQSYGNVVELRINSGGKLPNFG
FVVFDDSEPVQKVLSNRPIMFRGEVRLNVEEKKTRAAREGDRRDNRLRGPGGPRGGLGGGMRGPPRGGMVQKP
GFGVGRGLAPRQKLIPMARRAWLEHGHSCFLCEIVIRSQFHTTYEPEAMHTLYAPGGYDIMGWLIQIMNRPNPQ
VELGPVDTSCALILCDLKQKDTPIVYASEAFLYMTGYSNAEVLGRNCRFLQSPDGMVKPKSTRKYVDSNTINTMRK
AIDRNAEVQVEVVNFKKNGQRFVNFLTMIPVRDETGEYRYSMGFQCETE (SEQ ID NO: 18)
G3BP1 PxxP Domain Deletion Mutation with NcVivid Y50W; G3BP1dPxxP-NcVivid Y50W
MVMEKPSPLLVGREFVRQYYTLLNQAPDMLHRFYGKNSSYVHGGLDSNGKPADAVYGQKEIHRKVMSQNFTNC
HTKIRHVDAHATLNDGVVVQVMGLLSNNNQALRRFMQTFVLAPEGSVANKFYVHNDIFRYQDEVFGGFVTEPQE
ESEEEVEEPEERQQTPEVVPDDSGTFYDQAVVSNDMEEHLEEPVAEPEPDPEPEPEQEPVSEIQEEKPEPVLEEQLFI
GNLPHEVDKSELKDFFQSYGNVVELRINSGGKLPNFGFVVFDDSEPVQKVLSNRPIMFRGEVRLNVEEKKTRAARE
GDRRDNRLRGPGGPRGGLGGGMRGPPRGGMVQKPGFGVGRGLAPRQKLIPMARRAWLEHGHSCFLCEIVIRS
QFHTTYEPEAMHTLYAPGGYDIMGWLIQIMNRPNPQVELGPVDTSCALILCDLKQKDTPIVYASEAFLYMTGYSN
AEVLGRNCRFLQSPDGMVKPKSTRKYVDSNTINTMRKAIDRNAEVQVEVVNFKKNGQRFVNFLTMIPVRDETGEY
RYSMGFQCETE (SEQ ID NO: 19)
G3BP1 RRM Domain Deletion Mutation with NcVivid Y50W; G3BP1dRRM-NcVivid Y50W
MVMEKPSPLLVGREFVRQYYTLLNQAPDMLHRFYGKNSSYVHGGLDSNGKPADAVYGQKEIHRKVMSQNFTNC
HTKIRHVDAHATLNDGVVVQVMGLLSNNNQALRRFMQTFVLAPEGSVANKFYVHNDIFRYQDEVFGGFVTEPQE
ESEEEVEEPEERQQTPEVVPDDSGTFYDQAVVSNDMEEHLEEPVAEPEPDPEPEPEQEPVSEIQEEKPEPVLEETAP
EDAQKSSSPAPADIAQTVQEDLRTFSWASVTSKNLPPSGAVPVTGIPPHVVKVPASQPRPESKPESQIPPQRPQRD
QRVREQRINIPPQRGPRPIREAGEQGDIEPRRMVRHPDSHEKKTRAAREGDRRDNRLRGPGGPRGGLGGGMRG
PPRGGMVQKPGFGVGRGLAPRQKLIPMARRAWLEHGHSCFLCEIVIRSQFHTTYEPEAMHTLYAPGGYDIMGWL
IQIMNRPNPQVELGPVDTSCALILCDLKQKDTPIVYASEAFLYMTGYSNAEVLGRNCRFLQSPDGMVKPKSTRKYV
DSNTINTMRKAIDRNAEVQVEVVNFKKNGQRFVNFLTMIPVRDETGEYRYSMGFQCETE (SEQ ID NO: 20)
G3BP1 RGG Domain Deletion Mutation with NcVivid Y50W; G3BP1dRGG-NcVivid Y50W
MVMEKPSPLLVGREFVRQYYTLLNQAPDMLHRFYGKNSSYVHGGLDSNGKPADAVYGQKEIHRKVMSQNFTNC
HTKIRHVDAHATLNDGVVVQVMGLLSNNNQALRRFMQTFVLAPEGSVANKFYVHNDIFRYQDEVFGGFVTEPQE
ESEEEVEEPEERQQTPEVVPDDSGTFYDQAVVSNDMEEHLEEPVAEPEPDPEPEPEQEPVSEIQEEKPEPVLEETAP
EDAQKSSSPAPADIAQTVQEDLRTFSWASVTSKNLPPSGAVPVTGIPPHVVKVPASQPRPESKPESQIPPQRPQRD
QRVREQRINIPPQRGPRPIREAGEQGDIEPRRMVRHPDSHQLFIGNLPHEVDKSELKDFFQSYGNVVELRINSGGKL
PNFGFVVFDDSEPVQKVLSNRPIMFRGEVRLNVEMHTLYAPGGYDIMGWLIQIMNRPNPQVELGPVDTSCALILC
DLKQKDTPIVYASEAFLYMTGYSNAEVLGRNCRFLQSPDGMVKPKSTRKYVDSNTINTMRKAIDRNAEVQVEVVN
FKKNGQRFVNFLTMIPVRDETGEYRYSMGFQCETE (SEQ ID NO: 21)
G3BP1 RRMRGG Domain Deletion Mutation with NcVivid Y50W; G3BP1dRRMRGG-NcVivid Y50W
MVMEKPSPLLVGREFVRQYYTLLNQAPDMLHRFYGKNSSYVHGGLDSNGKPADAVYGQKEIHRKVMSQNFTNC
HTKIRHVDAHATLNDGVVVQVMGLLSNNNQALRRFMQTFVLAPEGSVANKFYVHNDIFRYQDEVFGGFVTEPQE
ESEEEVEEPEERQQTPEVVPDDSGTFYDQAVVSNDMEEHLEEPVAEPEPDPEPEPEQEPVSEIQEEKPEPVLEETAP
EDAQKSSSPAPADIAQWQEDLRTFSWASVTSKNLPPSGAVPVTGIPPHVVKVPASQPRPESKPESQIPPQRPQRD
QRVREQRINIPPQRGPRPIREAGEQGDIEPRRMVRHPDSHMHTLYAPGGYDIMGWLIQIMNRPNPQVELGPVDT
SCALILCDLKQKDTPIVYASEAFLYMTGYSNAEVLGRNCRFLQSPDGMVKPKSTRKYVDSNTINTMRKAIDRNAEV
QVEVVNFKKNGQRFVNFLTMIPVRDETGEYRYSMGFQCETE (SEQ ID NO: 22)
NcVivid Y50W, I74V and I85V substitutions with a short 3′ GSG repeat linker sequence, and G3BP1
Full Length Protein; NcVVDY50W/I74V/I85V-LINKERA-G3BP1:
MHTLYAPGGYDIMGWLIQIMNRPNPQVELGPVDTSCALVLCDLKQKDTPVVYASEAFLYMTGYSNAEVLGRNCR
FLQSPDGMVKPKSTRKYVDSNTINTMRKAIDRNAEVQVEVVNFKKNGQRFVNFLTMIPVRDETGEYRYSMGFQC
ETEGSGGSGGSGGSGMVMEKPSPLLVGREFVRQYYTLLNQAPDMLHRFYGKNSSYVHGGLDSNGKPADAVYGQ
KEIHRKVMSQNFTNCHTKIRHVDAHATLNDGVVVQVMGLLSNNNQALRRFMQTFVLAPEGSVANKFYVHNDIF
RYQDEVFGGFVTEPQEESEEEVEEPEERQQTPEVVPDDSGTFYDQAVVSNDMEEHLEEPVAEPEPDPEPEPEQEP
VSEIQEEKPEPVLEETAPEDAQKSSSPAPADIAQTVQEDLRTFSWASVTSKNLPPSGAVPVTGIPPHVVKVPASQPR
PESKPESQIPPQRPQRDQRVREQRINIPPQRGPRPIREAGEQGDIEPRRMVRHPDSHQLFIGNLPHEVDKSELKDFF
QSYGNVVELRINSGGKLPNFGFVVFDDSEPVQKVLSNRPIMFRGEVRLNVEEKKTRAAREGDRRDNRLRGPGGPR
GGLGGGMRGPPRGGMVQKPGFGVGRGLAPRQKLIPMARRAWLEHGHSCFLCEIVIRSQFHTTYEPEA (SEQ ID
NO: 23)
NcVivid Y50W, I74V and I85V substitutions with a short 3′ GSG repeat linker sequence, and G3BP1
NTF2 Domain Deletion Mutation; NcVVDY50W/I74V/I85V-LINKERA-G3BP1dNTF2:
MHTLYAPGGYDIMGWLIQIMNRPNPQVELGPVDTSCALVLCDLKQKDTPVVYASEAFLYMTGYSNAEVLGRNCR
FLQSPDGMVKPKSTRKYVDSNTINTMRKAIDRNAEVQVEVVNFKKNGQRFVNFLTMIPVRDETGEYRYSMGFQC
ETEGSGGSGGSGGSGMGFVTEPQEESEEEVEEPEERQQTPEVVPDDSGTFYDQAVVSNDMEEHLEEPVAEPEPD
PEPEPEQEPVSEIQEEKPEPVLEETAPEDAQKSSSPAPADIAQTVQEDLRTFSWASVTSKNLPPSGAVPVTGIPPHVV
KVPASQPRPESKPESQIPPQRPQRDQRVREQRINIPPQRGPRPIREAGEQGDIEPRRMVRHPDSHQLFIGNLPHEV
DKSELKDFFQSYGNVVELRINSGGKLPNFGFVVFDDSEPVQKVLSNRPIMFRGEVRLNVEEKKTRAAREGDRRDNR
LRGPGGPRGGLGGGMRGPPRGGMVQKPGFGVGRGLAPRQKLIPMARRAWLEHGHSCFLCEIVIRSQFHTTYEPE
A (SEQ ID NO: 24)
NcVivid Y50W, I74V and I85V substitutions with a short 3′ GSG repeat linker sequence, and G3BP1
Acidic Domain Deletion Mutation; NcVVDY50W/I74V/I85V-LINKERA-G3BP1dAcidic:
MHTLYAPGGYDIMGWLIQIMNRPNPQVELGPVDTSCALVLCDLKQKDTPVVYASEAFLYMTGYSNAEVLGRNCR
FLQSPDGMVKPKSTRKYVDSNTINTMRKAIDRNAEVQVEVVNFKKNGQRFVNFLTMIPVRDETGEYRYSMGFQC
ETEGSGGSGGSGGSGMVMEKPSPLLVGREFVRQYYTLLNQAPDMLHRFYGKNSSYVHGGLDSNGKPADAVYGQ
KEIHRKVMSQNFTNCHTKIRHVDAHATLNDGVVVQVMGLLSNNNQALRRFMQTFVLAPEGSVANKFYVHNDIF
RYQDEVFGTAPEDAQKSSSPAPADIAQTVQEDLRTFSWASVTSKNLPPSGAVPVTGIPPHVVKVPASQPRPESKPE
SQIPPQRPQRDQRVREQRINIPPQRGPRPIREAGEQGDIEPRRMVRHPDSHQLFIGNLPHEVDKSELKDFFQSYGN
VVELRINSGGKLPNFGFVVFDDSEPVQKVLSNRPIMFRGEVRLNVEEKKTRAAREGDRRDNRLRGPGGPRGGLGG
GMRGPPRGGMVQKPGFGVGRGLAPRQKLIPMARRAWLEHGHSCFLCEIVIRSQFHTTYEPEA (SEQ ID NO: 25)
NcVivid Y50W, I74V and I85V substitutions with a short 3′ GSG repeat linker sequence, and G3BP1
PxxP Domain Deletion Mutation; NcVVDY50W/I74V/I85V-LINKERA-G3BP1dPxxP:
MHTLYAPGGYDIMGWLIQIMNRPNPQVELGPVDTSCALVLCDLKQKDTPVVYASEAFLYMTGYSNAEVLGRNCR
FLQSPDGMVKPKSTRKYVDSNTINTMRKAIDRNAEVQVEVVNFKKNGQRFVNFLTMIPVRDETGEYRYSMGFQC
ETEGSGGSGGSGGSGMVMEKPSPLLVGREFVRQYYTLLNQAPDMLHRFYGKNSSYVHGGLDSNGKPADAVYGQ
KEIHRKVMSQNFTNCHTKIRHVDAHATLNDGVVVQVMGLLSNNNQALRRFMQTFVLAPEGSVANKFYVHNDIF
RYQDEVFGGFVTEPQEESEEEVEEPEERQQTPEVVPDDSGTFYDQAVVSNDMEEHLEEPVAEPEPDPEPEPEQEP
VSEIQEEKPEPVLEEQLFIGNLPHEVDKSELKDFFQSYGNVVELRINSGGKLPNFGFVVFDDSEPVQKVLSNRPIMFR
GEVRLNVEEKKTRAAREGDRRDNRLRGPGGPRGGLGGGMRGPPRGGMVQKPGFGVGRGLAPRQKLIPMARRA
WLEHGHSCFLCEIVIRSQFHTTYEPEA (SEQ ID NO: 26)
NcVivid Y50W, I74V and I85V substitutions with a short 3′ GSG repeat linker sequence, and G3BP1
RRM Domain Deletion Mutation; NcVVDY50W/I74V/I85V-LINKERA-G3BP1dRRM:
MHTLYAPGGYDIMGWLIQIMNRPNPQVELGPVDTSCALVLCDLKQKDTPVVYASEAFLYMTGYSNAEVLGRNCR
FLQSPDGMVKPKSTRKYVDSNTINTMRKAIDRNAEVQVEVVNFKKNGQRFVNFLTMIPVRDETGEYRYSMGFQC
ETEGSGGSGGSGGSGMVMEKPSPLLVGREFVRQYYTLLNQAPDMLHRFYGKNSSYVHGGLDSNGKPADAVYGQ
KEIHRKVMSQNFTNCHTKIRHVDAHATLNDGVVVQVMGLLSNNNQALRRFMQTFVLAPEGSVANKFYVHNDIF
RYQDEVFGGFVTEPQEESEEEVEEPEERQQTPEVVPDDSGTFYDQAVVSNDMEEHLEEPVAEPEPDPEPEPEQEP
VSEIQEEKPEPVLEETAPEDAQKSSSPAPADIAQTVQEDLRTFSWASVTSKNLPPSGAVPVTGIPPHVVKVPASQPR
PESKPESQIPPQRPQRDQRVREQRINIPPQRGPRPIREAGEQGDIEPRRMVRHPDSHEKKTRAAREGDRRDNRLR
GPGGPRGGLGGGMRGPPRGGMVQKPGFGVGRGLAPRQKLIPMARRAWLEHGHSCFLCEIVIRSQFHTTYEPEA
(SEQ ID NO: 27)
NcVivid Y50W, I74V and I85V substitutions with a short 3′ GSG repeat linker sequence, and G3BP1
RGG Domain Deletion Mutation; NcVVDY50W/I74V/I85V-LINKERA-G3BP1dRGG:
MHTLYAPGGYDIMGWLIQIMNRPNPQVELGPVDTSCALVLCDLKQKDTPVVYASEAFLYMTGYSNAEVLGRNCR
FLQSPDGMVKPKSTRKYVDSNTINTMRKAIDRNAEVQVEVVNFKKNGQRFVNFLTMIPVRDETGEYRYSMGFQC
ETEGSGGSGGSGGSGMVMEKPSPLLVGREFVRQYYTLLNQAPDMLHRFYGKNSSYVHGGLDSNGKPADAVYGQ
KEIHRKVMSQNFTNCHTKIRHVDAHATLNDGVVVQVMGLLSNNNQALRRFMQTFVLAPEGSVANKFYVHNDIF
RYQDEVFGGFVTEPQEESEEEVEEPEERQQTPEVVPDDSGTFYDQAVVSNDMEEHLEEPVAEPEPDPEPEPEQEP
VSEIQEEKPEPVLEETAPEDAQKSSSPAPADIAQTVQEDLRTFSWASVTSKNLPPSGAVPVTGIPPHVVKVPASQPR
PESKPESQIPPQRPQRDQRVREQRINIPPQRGPRPIREAGEQGDIEPRRMVRHPDSHQLFIGNLPHEVDKSELKDFF
QSYGNVVELRINSGGKLPNFGFVVFDDSEPVQKVLSNRPIMFRGEVRLNVE (SEQ ID NO: 28)
NcVivid Y50W, I74V and I85V substitutions with a short 3′ GSG repeat linker sequence, and G3BP1
RRMRGG Domain Deletion Mutation; NcVVDY50W/I74V/I85V-LINKERA-G3BP1dRRMRGG:
MHTLYAPGGYDIMGWLIQIMNRPNPQVELGPVDTSCALVLCDLKQKDTPVVYASEAFLYMTGYSNAEVLGRNCR
FLQSPDGMVKPKSTRKYVDSNTINTMRKAIDRNAEVQVEVVNFKKNGQRFVNFLTMIPVRDETGEYRYSMGFQC
ETEGSGGSGGSGGSGMVMEKPSPLLVGREFVRQYYTLLNQAPDMLHRFYGKNSSYVHGGLDSNGKPADAVYGQ
KEIHRKVMSQNFTNCHTKIRHVDAHATLNDGVVVQVMGLLSNNNQALRRFMQTFVLAPEGSVANKFYVHNDIF
RYQDEVFGGFVTEPQEESEEEVEEPEERQQTPEVVPDDSGTFYDQAVVSNDMEEHLEEPVAEPEPDPEPEPEQEP
VSEIQEEKPEPVLEETAPEDAQKSSSPAPADIAQTVQEDLRTFSWASVTSKNLPPSGAVPVTGIPPHVVKVPASQPR
PESKPESQIPPQRPQRDQRVREQRINIPPQRGPRPIREAGEQGDIEPRRMVRHPDSH (SEQ ID NO: 29)
NcVivid Y50W, I52C, I74V and I85V substitutions with a short 3′ GSG repeat linker sequence, and
G3BP1 Full Length Protein; NcVVDY50W/I74V/I85V-LINKERA-G3BP1:
MHTLYAPGGYDIMGWLCQIMNRPNPQVELGPVDTSCALVLCDLKQKDTPVVYASEAFLYMTGYSNAEVLGRNC
RFLQSPDGMVKPKSTRKYVDSNTINTMRKAIDRNAEVQVEVVNFKKNGQRFVNFLTMIPVRDETGEYRYSMGFQ
CETEGSGGSGGSGGSGMVMEKPSPLLVGREFVRQYYTLLNQAPDMLHRFYGKNSSYVHGGLDSNGKPADAVYG
QKEIHRKVMSQNFTNCHTKIRHVDAHATLNDGVVVQVMGLLSNNNQALRRFMQTFVLAPEGSVANKFYVHNDI
FRYQDEVFGGFVTEPQEESEEEVEEPEERQQTPEVVPDDSGTFYDQAVVSNDMEEHLEEPVAEPEPDPEPEPEQEP
VSEIQEEKPEPVLEETAPEDAQKSSSPAPADIAQTVQEDLRTFSWASVTSKNLPPSGAVPVTGIPPHVVKVPASQPR
PESKPESQIPPQRPQRDQRVREQRINIPPQRGPRPIREAGEQGDIEPRRMVRHPDSHQLFIGNLPHEVDKSELKDFF
QSYGNVVELRINSGGKLPNFGFVVFDDSEPVQKVLSNRPIMFRGEVRLNVEEKKTRAAREGDRRDNRLRGPGGPR
GGLGGGMRGPPRGGMVQKPGFGVGRGLAPRQKLIPMARRAWLEHGHSCFLCEIVIRSQFHTTYEPEA (SEQ ID
NO: 30)
NcVivid Y50W, I52C, I74V and I85V substitutions with a short 3′ GSG repeat linker sequence, and
G3BP1 NTF2 Domain Deletion Mutation; NcVVDY50W/I74V/I85V-LINKERA-G3BP1dNTF2:
MHTLYAPGGYDIMGWLCQIMNRPNPQVELGPVDTSCALVLCDLKQKDTPVVYASEAFLYMTGYSNAEVLGRNC
RFLQSPDGMVKPKSTRKYVDSNTINTMRKAIDRNAEVQVEVVNFKKNGQRFVNFLTMIPVRDETGEYRYSMGFQ
CETEGSGGSGGSGGSGMGFVTEPQEESEEEVEEPEERQQTPEVVPDDSGTFYDQAVVSNDMEEHLEEPVAEPEP
DPEPEPEQEPVSEIQEEKPEPVLEETAPEDAQKSSSPAPADIAQTVQEDLRTFSWASVTSKNLPPSGAVPVTGIPPH
VVKVPASQPRPESKPESQIPPQRPQRDQRVREQRINIPPQRGPRPIREAGEQGDIEPRRMVRHPDSHQLFIGNLPH
EVDKSELKDFFQSYGNVVELRINSGGKLPNFGFVVFDDSEPVQKVLSNRPIMFRGEVRLNVEEKKTRAAREGDRRD
NRLRGPGGPRGGLGGGMRGPPRGGMVQKPGFGVGRGLAPRQKLIPMARRAWLEHGHSCFLCEIVIRSQFHTTY
EPEA (SEQ ID NO: 31)
NcVivid Y50W, I52C, I74V and I85V substitutions with a short 3′ GSG repeat linker sequence, and
G3BP1 Acidic Domain Deletion Mutation; NcVVDY50W/I74V/I85V-LINKERA-G3BP1dAcidic:
MHTLYAPGGYDIMGWLCQIMNRPNPQVELGPVDTSCALVLCDLKQKDTPVVYASEAFLYMTGYSNAEVLGRNC
RFLQSPDGMVKPKSTRKYVDSNTINTMRKAIDRNAEVQVEVVNFKKNGQRFVNFLTMIPVRDETGEYRYSMGFQ
CETEGSGGSGGSGGSGMVMEKPSPLLVGREFVRQYYTLLNQAPDMLHRFYGKNSSYVHGGLDSNGKPADAVYG
QKEIHRKVMSQNFTNCHTKIRHVDAHATLNDGVVVQVMGLLSNNNQALRRFMQTFVLAPEGSVANKFYVHNDI
FRYQDEVFGTAPEDAQKSSSPAPADIAQTVQEDLRTFSWASVTSKNLPPSGAVPVTGIPPHVVKVPASQPRPESKP
ESQIPPQRPQRDQRVREQRINIPPQRGPRPIREAGEQGDIEPRRMVRHPDSHQLFIGNLPHEVDKSELKDFFQSYG
NVVELRINSGGKLPNFGFVVFDDSEPVQKVLSNRPIMFRGEVRLNVEEKKTRAAREGDRRDNRLRGPGGPRGGLG
GGMRGPPRGGMVQKPGFGVGRGLAPRQKLIPMARRAWLEHGHSCFLCEIVIRSQFHTTYEPEA (SEQ ID
NO: 32)
NcVivid Y50W, I52C, I74V and I85V substitutions with a short 3′ GSG repeat linker sequence, and
G3BP1 PxxP Domain Deletion Mutation; NcVVDY50W/I74V/I85V-LINKERA-G3BP1dPxxP:
MHTLYAPGGYDIMGWLCQIMNRPNPQVELGPVDTSCALVLCDLKQKDTPVVYASEAFLYMTGYSNAEVLGRNC
RFLQSPDGMVKPKSTRKYVDSNTINTMRKAIDRNAEVQVEVVNFKKNGQRFVNFLTMIPVRDETGEYRYSMGFQ
CETEGSGGSGGSGGSGMVMEKPSPLLVGREFVRQYYTLLNQAPDMLHRFYGKNSSYVHGGLDSNGKPADAVYG
QKEIHRKVMSQNFTNCHTKIRHVDAHATLNDGVVVQVMGLLSNNNQALRRFMQTFVLAPEGSVANKFYVHNDI
FRYQDEVFGGFVTEPQEESEEEVEEPEERQQTPEVVPDDSGTFYDQAVVSNDMEEHLEEPVAEPEPDPEPEPEQEP
VSEIQEEKPEPVLEEQLFIGNLPHEVDKSELKDFFQSYGNVVELRINSGGKLPNFGFVVFDDSEPVQKVLSNRPIMFR
GEVRLNVEEKKTRAAREGDRRDNRLRGPGGPRGGLGGGMRGPPRGGMVQKPGFGVGRGLAPRQKLIPMARRA
WLEHGHSCFLCEIVIRSQFHTTYEPEA (SEQ ID NO: 33)
NcVivid Y50W, I52C, I74V and I85V substitutions with a short 3′ GSG repeat linker sequence, and
G3BP1 RRM Domain Deletion Mutation; NcVVDY50W/I74V/I85V-LINKERA-G3BP1dRRM:
MHTLYAPGGYDIMGWLCQIMNRPNPQVELGPVDTSCALVLCDLKQKDTPVVYASEAFLYMTGYSNAEVLGRNC
RFLQSPDGMVKPKSTRKYVDSNTINTMRKAIDRNAEVQVEVVNFKKNGQRFVNFLTMIPVRDETGEYRYSMGFQ
CETEGSGGSGGSGGSGMVMEKPSPLLVGREFVRQYYTLLNQAPDMLHRFYGKNSSYVHGGLDSNGKPADAVYG
QKEIHRKVMSQNFTNCHTKIRHVDAHATLNDGVVVQVMGLLSNNNQALRRFMQTFVLAPEGSVANKFYVHNDI
FRYQDEVFGGFVTEPQEESEEEVEEPEERQQTPEVVPDDSGTFYDQAVVSNDMEEHLEEPVAEPEPDPEPEPEQEP
VSEIQEEKPEPVLEETAPEDAQKSSSPAPADIAQTVQEDLRTFSWASVTSKNLPPSGAVPVTGIPPHVVKVPASQPR
PESKPESQIPPQRPQRDQRVREQRINIPPQRGPRPIREAGEQGDIEPRRMVRHPDSHEKKTRAAREGDRRDNRLR
GPGGPRGGLGGGMRGPPRGGMVQKPGFGVGRGLAPRQKLIPMARRAWLEHGHSCFLCEIVIRSQFHTTYEPEA
(SEQ ID NO: 34)
NcVivid Y50W, I52C, I74V and I85V substitutions with a short 3′ GSG repeat linker sequence, and
G3BP1 RGG Domain Deletion Mutation; NcVVDY50W/I74V/I85V-LINKERA-G3BP1dRGG:
MHTLYAPGGYDIMGWLCQIMNRPNPQVELGPVDTSCALVLCDLKQKDTPVVYASEAFLYMTGYSNAEVLGRNC
RFLQSPDGMVKPKSTRKYVDSNTINTMRKAIDRNAEVQVEVVNFKKNGQRFVNFLTMIPVRDETGEYRYSMGFQ
CETEGSGGSGGSGGSGMVMEKPSPLLVGREFVRQYYTLLNQAPDMLHRFYGKNSSYVHGGLDSNGKPADAVYG
QKEIHRKVMSQNFTNCHTKIRHVDAHATLNDGVVVQVMGLLSNNNQALRRFMQTFVLAPEGSVANKFYVHNDI
FRYQDEVFGGFVTEPQEESEEEVEEPEERQQTPEVVPDDSGTFYDQAVVSNDMEEHLEEPVAEPEPDPEPEPEQEP
VSEIQEEKPEPVLEETAPEDAQKSSSPAPADIAQTVQEDLRTFSWASVTSKNLPPSGAVPVTGIPPHVVKVPASQPR
PESKPESQIPPQRPQRDQRVREQRINIPPQRGPRPIREAGEQGDIEPRRMVRHPDSHQLFIGNLPHEVDKSELKDFF
QSYGNVVELRINSGGKLPNFGFVVFDDSEPVQKVLSNRPIMFRGEVRLNVE (SEQ ID NO: 35)
NcVivid Y50W, I52C, I74V and I85V substitutions with a short 3′ GSG repeat linker sequence, and
G3BP1 RRMRGG Domain Deletion Mutation; NcVVDY50W/I74V/I85V-LINKERA-G3BP1dRRMRGG:
MHTLYAPGGYDIMGWLCQIMNRPNPQVELGPVDTSCALVLCDLKQKDTPVVYASEAFLYMTGYSNAEVLGRNC
RFLQSPDGMVKPKSTRKYVDSNTINTMRKAIDRNAEVQVEVVNFKKNGQRFVNFLTMIPVRDETGEYRYSMGFQ
CETEGSGGSGGSGGSGMVMEKPSPLLVGREFVRQYYTLLNQAPDMLHRFYGKNSSYVHGGLDSNGKPADAVYG
QKEIHRKVMSQNFTNCHTKIRHVDAHATLNDGVVVQVMGLLSNNNQALRRFMQTFVLAPEGSVANKFYVHNDI
FRYQDEVFGGFVTEPQEESEEEVEEPEERQQTPEVVPDDSGTFYDQAVVSNDMEEHLEEPVAEPEPDPEPEPEQEP
VSEIQEEKPEPVLEETAPEDAQKSSSPAPADIAQTVQEDLRTFSWASVTSKNLPPSGAVPVTGIPPHVVKVPASQPR
PESKPESQIPPQRPQRDQRVREQRINIPPQRGPRPIREAGEQGDIEPRRMVRHPDSH (SEQ ID NO: 36)
NcVivid Y50W, C71V, I74V and I85V substitutions with a short 3′ GSG repeat linker sequence, and
G3BP1 Full Length Protein; NcVVDY50W/I74V/I85V-LINKERA-G3BP1:
MHTLYAPGGYDIMGWLIQIMNRPNPQVELGPVDTSVALVLCDLKQKDTPVVYASEAFLYMTGYSNAEVLGRNCR
FLQSPDGMVKPKSTRKYVDSNTINTMRKAIDRNAEVQVEVVNFKKNGQRFVNFLTMIPVRDETGEYRYSMGFQC
ETEGSGGSGGSGGSGMVMEKPSPLLVGREFVRQYYTLLNQAPDMLHRFYGKNSSYVHGGLDSNGKPADAVYGQ
KEIHRKVMSQNFTNCHTKIRHVDAHATLNDGVVVQVMGLLSNNNQALRRFMQTFVLAPEGSVANKFYVHNDIF
RYQDEVFGGFVTEPQEESEEEVEEPEERQQTPEVVPDDSGTFYDQAVVSNDMEEHLEEPVAEPEPDPEPEPEQEP
VSEIQEEKPEPVLEETAPEDAQKSSSPAPADIAQTVQEDLRTFSWASVTSKNLPPSGAVPVTGIPPHVVKVPASQPR
PESKPESQIPPQRPQRDQRVREQRINIPPQRGPRPIREAGEQGDIEPRRMVRHPDSHQLFIGNLPHEVDKSELKDFF
QSYGNVVELRINSGGKLPNFGFVVFDDSEPVQKVLSNRPIMFRGEVRLNVEEKKTRAAREGDRRDNRLRGPGGPR
GGLGGGMRGPPRGGMVQKPGFGVGRGLAPRQKLIPMARRAWLEHGHSCFLCEIVIRSQFHTTYEPEA (SEQ ID
NO: 37)
NcVivid Y50W, C71V, I74V and I85V substitutions with a short 3′ GSG repeat linker sequence, and
G3BP1 NTF2 Domain Deletion Mutation; NcVVDY50W/I74V/I85V-LINKERA-G3BP1dNTF2:
MHTLYAPGGYDIMGWLIQIMNRPNPQVELGPVDTSVALVLCDLKQKDTPVVYASEAFLYMTGYSNAEVLGRNCR
FLQSPDGMVKPKSTRKYVDSNTINTMRKAIDRNAEVQVEVVNFKKNGQRFVNFLTMIPVRDETGEYRYSMGFQC
ETEGSGGSGGSGGSGMGFVTEPQEESEEEVEEPEERQQTPEVVPDDSGTFYDQAVVSNDMEEHLEEPVAEPEPD
PEPEPEQEPVSEIQEEKPEPVLEETAPEDAQKSSSPAPADIAQTVQEDLRTFSWASVTSKNLPPSGAVPVTGIPPHVV
KVPASQPRPESKPESQIPPQRPQRDQRVREQRINIPPQRGPRPIREAGEQGDIEPRRMVRHPDSHQLFIGNLPHEV
DKSELKDFFQSYGNVVELRINSGGKLPNFGFVVFDDSEPVQKVLSNRPIMFRGEVRLNVEEKKTRAAREGDRRDNR
LRGPGGPRGGLGGGMRGPPRGGMVQKPGFGVGRGLAPRQKLIPMARRAWLEHGHSCFLCEIVIRSQFHTTYEPE
A (SEQ ID NO: 38)
NcVivid Y50W, C71V, I74V and I85V substitutions with a short 3′ GSG repeat linker sequence, and
G3BP1 Acidic Domain Deletion Mutation; NcVVDY50W/I74V/I85V-LINKERA-G3BP1dAcidic:
MHTLYAPGGYDIMGWLIQIMNRPNPQVELGPVDTSVALVLCDLKQKDTPVVYASEAFLYMTGYSNAEVLGRNCR
FLQSPDGMVKPKSTRKYVDSNTINTMRKAIDRNAEVQVEVVNFKKNGQRFVNFLTMIPVRDETGEYRYSMGFQC
ETEGSGGSGGSGGSGMVMEKPSPLLVGREFVRQYYTLLNQAPDMLHRFYGKNSSYVHGGLDSNGKPADAVYGQ
KEIHRKVMSQNFTNCHTKIRHVDAHATLNDGVVVQVMGLLSNNNQALRRFMQTFVLAPEGSVANKFYVHNDIF
RYQDEVFGTAPEDAQKSSSPAPADIAQTVQEDLRTFSWASVTSKNLPPSGAVPVTGIPPHVVKVPASQPRPESKPE
SQIPPQRPQRDQRVREQRINIPPQRGPRPIREAGEQGDIEPRRMVRHPDSHQLFIGNLPHEVDKSELKDFFQSYGN
VVELRINSGGKLPNFGFVVFDDSEPVQKVLSNRPIMFRGEVRLNVEEKKTRAAREGDRRDNRLRGPGGPRGGLGG
GMRGPPRGGMVQKPGFGVGRGLAPRQKLIPMARRAWLEHGHSCFLCEIVIRSQFHTTYEPEA (SEQ ID NO: 39)
NcVivid Y50W, C71V, I74V and I85V substitutions with a short 3′ GSG repeat linker sequence, and
G3BP1 PxxP Domain Deletion Mutation; NcVVDY50W/I74V/I85V-LINKERA-G3BP1dPxxP:
MHTLYAPGGYDIMGWLIQIMNRPNPQVELGPVDTSVALVLCDLKQKDTPVVYASEAFLYMTGYSNAEVLGRNCR
FLQSPDGMVKPKSTRKYVDSNTINTMRKAIDRNAEVQVEVVNFKKNGQRFVNFLTMIPVRDETGEYRYSMGFQC
ETEGSGGSGGSGGSGMVMEKPSPLLVGREFVRQYYTLLNQAPDMLHRFYGKNSSYVHGGLDSNGKPADAVYGQ
KEIHRKVMSQNFTNCHTKIRHVDAHATLNDGVVVQVMGLLSNNNQALRRFMQTFVLAPEGSVANKFYVHNDIF
RYQDEVFGGFVTEPQEESEEEVEEPEERQQTPEVVPDDSGTFYDQAVVSNDMEEHLEEPVAEPEPDPEPEPEQEP
VSEIQEEKPEPVLEEQLFIGNLPHEVDKSELKDFFQSYGNVVELRINSGGKLPNFGFVVFDDSEPVQKVLSNRPIMFR
GEVRLNVEEKKTRAAREGDRRDNRLRGPGGPRGGLGGGMRGPPRGGMVQKPGFGVGRGLAPRQKLIPMARRA
WLEHGHSCFLCEIVIRSQFHTTYEPEA (SEQ ID NO: 40)
NcVivid Y50W, C71V, I74V and I85V substitutions with a short 3′ GSG repeat linker sequence, and
G3BP1 RRM Domain Deletion Mutation; NcVVDY50W/I74V/I85V-LINKERA-G3BP1dRRM:
MHTLYAPGGYDIMGWLIQIMNRPNPQVELGPVDTSVALVLCDLKQKDTPVVYASEAFLYMTGYSNAEVLGRNCR
FLQSPDGMVKPKSTRKYVDSNTINTMRKAIDRNAEVQVEVVNFKKNGQRFVNFLTMIPVRDETGEYRYSMGFQC
ETEGSGGSGGSGGSGMVMEKPSPLLVGREFVRQYYTLLNQAPDMLHRFYGKNSSYVHGGLDSNGKPADAVYGQ
KEIHRKVMSQNFTNCHTKIRHVDAHATLNDGVVVQVMGLLSNNNQALRRFMQTFVLAPEGSVANKFYVHNDIF
RYQDEVFGGFVTEPQEESEEEVEEPEERQQTPEVVPDDSGTFYDQAVVSNDMEEHLEEPVAEPEPDPEPEPEQEP
VSEIQEEKPEPVLEETAPEDAQKSSSPAPADIAQTVQEDLRTFSWASVTSKNLPPSGAVPVTGIPPHVVKVPASQPR
PESKPESQIPPQRPQRDQRVREQRINIPPQRGPRPIREAGEQGDIEPRRMVRHPDSHEKKTRAAREGDRRDNRLR
GPGGPRGGLGGGMRGPPRGGMVQKPGFGVGRGLAPRQKLIPMARRAWLEHGHSCFLCEIVIRSQFHTTYEPEA
(SEQ ID NO: 41)
NcVivid Y50W, C71V, I74V and I85V substitutions with a short 3′ GSG repeat linker sequence, and
G3BP1 RGG Domain Deletion Mutation; NcVVDY50W/I74V/I85V-LINKERA-G3BP1dRGG:
MHTLYAPGGYDIMGWLIQIMNRPNPQVELGPVDTSVALVLCDLKQKDTPVVYASEAFLYMTGYSNAEVLGRNCR
FLQSPDGMVKPKSTRKYVDSNTINTMRKAIDRNAEVQVEVVNFKKNGQRFVNFLTMIPVRDETGEYRYSMGFQC
ETEGSGGSGGSGGSGMVMEKPSPLLVGREFVRQYYTLLNQAPDMLHRFYGKNSSYVHGGLDSNGKPADAVYGQ
KEIHRKVMSQNFTNCHTKIRHVDAHATLNDGVVVQVMGLLSNNNQALRRFMQTFVLAPEGSVANKFYVHNDIF
RYQDEVFGGFVTEPQEESEEEVEEPEERQQTPEVVPDDSGTFYDQAVVSNDMEEHLEEPVAEPEPDPEPEPEQEP
VSEIQEEKPEPVLEETAPEDAQKSSSPAPADIAQTVQEDLRTFSWASVTSKNLPPSGAVPVTGIPPHVVKVPASQPR
PESKPESQIPPQRPQRDQRVREQRINIPPQRGPRPIREAGEQGDIEPRRMVRHPDSHQLFIGNLPHEVDKSELKDFF
QSYGNVVELRINSGGKLPNFGFVVFDDSEPVQKVLSNRPIMFRGEVRLNVE (SEQ ID NO: 42)
NcVivid Y50W, C71V, I74V and I85V substitutions with a short 3′ GSG repeat linker sequence, and
G3BP1 RRMRGG Domain Deletion Mutation; NcVVDY50W/I74V/I85V-LINKERA-G3BP1dRRMRGG:
MHTLYAPGGYDIMGWLIQIMNRPNPQVELGPVDTSVALVLCDLKQKDTPVVYASEAFLYMTGYSNAEVLGRNCR
FLQSPDGMVKPKSTRKYVDSNTINTMRKAIDRNAEVQVEVVNFKKNGQRFVNFLTMIPVRDETGEYRYSMGFQC
ETEGSGGSGGSGGSGMVMEKPSPLLVGREFVRQYYTLLNQAPDMLHRFYGKNSSYVHGGLDSNGKPADAVYGQ
KEIHRKVMSQNFTNCHTKIRHVDAHATLNDGVVVQVMGLLSNNNQALRRFMQTFVLAPEGSVANKFYVHNDIF
RYQDEVFGGFVTEPQEESEEEVEEPEERQQTPEVVPDDSGTFYDQAVVSNDMEEHLEEPVAEPEPDPEPEPEQEP
VSEIQEEKPEPVLEETAPEDAQKSSSPAPADIAQTVQEDLRTFSWASVTSKNLPPSGAVPVTGIPPHVVKVPASQPR
PESKPESQIPPQRPQRDQRVREQRINIPPQRGPRPIREAGEQGDIEPRRMVRHPDSH (SEQ ID NO: 43)
NcVivid Y50W, I52C, C71V, I74V and I85V substitutions with a short 3′ GSG repeat linker sequence,
and G3BP1 Full Length Protein; NcVVDY50W/I74V/I85V-LINKERA-G3BP1:
MHTLYAPGGYDIMGWLCQIMNRPNPQVELGPVDTSVALVLCDLKQKDTPVVYASEAFLYMTGYSNAEVLGRNC
RFLQSPDGMVKPKSTRKYVDSNTINTMRKAIDRNAEVQVEVVNFKKNGQRFVNFLTMIPVRDETGEYRYSMGFQ
CETEGSGGSGGSGGSGMVMEKPSPLLVGREFVRQYYTLLNQAPDMLHRFYGKNSSYVHGGLDSNGKPADAVYG
QKEIHRKVMSQNFTNCHTKIRHVDAHATLNDGVVVQVMGLLSNNNQALRRFMQTFVLAPEGSVANKFYVHNDI
FRYQDEVFGGFVTEPQEESEEEVEEPEERQQTPEVVPDDSGTFYDQAVVSNDMEEHLEEPVAEPEPDPEPEPEQEP
VSEIQEEKPEPVLEETAPEDAQKSSSPAPADIAQTVQEDLRTFSWASVTSKNLPPSGAVPVTGIPPHVVKVPASQPR
PESKPESQIPPQRPQRDQRVREQRINIPPQRGPRPIREAGEQGDIEPRRMVRHPDSHQLFIGNLPHEVDKSELKDFF
QSYGNVVELRINSGGKLPNFGFVVFDDSEPVQKVLSNRPIMFRGEVRLNVEEKKTRAAREGDRRDNRLRGPGGPR
GGLGGGMRGPPRGGMVQKPGFGVGRGLAPRQKLIPMARRAWLEHGHSCFLCEIVIRSQFHTTYEPEA (SEQ ID
NO: 44)
NcVivid Y50W, I52C, C71V, I74V and I85V substitutions with a short 3′ GSG repeat linker sequence,
and G3BP1 NTF2 Domain Deletion Mutation; NcVVDY50W/I74V/I85V-LINKERA-G3BP1dNTF2:
MHTLYAPGGYDIMGWLCQIMNRPNPQVELGPVDTSVALVLCDLKQKDTPVVYASEAFLYMTGYSNAEVLGRNC
RFLQSPDGMVKPKSTRKYVDSNTINTMRKAIDRNAEVQVEVVNFKKNGQRFVNFLTMIPVRDETGEYRYSMGFQ
CETEGSGGSGGSGGSGMGFVTEPQEESEEEVEEPEERQQTPEVVPDDSGTFYDQAVVSNDMEEHLEEPVAEPEP
DPEPEPEQEPVSEIQEEKPEPVLEETAPEDAQKSSSPAPADIAQTVQEDLRTFSWASVTSKNLPPSGAVPVTGIPPH
VVKVPASQPRPESKPESQIPPQRPQRDQRVREQRINIPPQRGPRPIREAGEQGDIEPRRMVRHPDSHQLFIGNLPH
EVDKSELKDFFQSYGNVVELRINSGGKLPNFGFVVFDDSEPVQKVLSNRPIMFRGEVRLNVEEKKTRAAREGDRRD
NRLRGPGGPRGGLGGGMRGPPRGGMVQKPGFGVGRGLAPRQKLIPMARRAWLEHGHSCFLCEIVIRSQFHTTY
EPEA (SEQ ID NO: 45)
NcVivid Y50W, I52C, C71V, I74V and I85V substitutions with a short 3′ GSG repeat linker sequence,
and G3BP1 Acidic Domain Deletion Mutation; NcVVDY50W/I74V/I85V-LINKERA-G3BP1dAcidic:
MHTLYAPGGYDIMGWLCQIMNRPNPQVELGPVDTSVALVLCDLKQKDTPVVYASEAFLYMTGYSNAEVLGRNC
RFLQSPDGMVKPKSTRKYVDSNTINTMRKAIDRNAEVQVEVVNFKKNGQRFVNFLTMIPVRDETGEYRYSMGFQ
CETEGSGGSGGSGGSGMVMEKPSPLLVGREFVRQYYTLLNQAPDMLHRFYGKNSSYVHGGLDSNGKPADAVYG
QKEIHRKVMSQNFTNCHTKIRHVDAHATLNDGVVVQVMGLLSNNNQALRRFMQTFVLAPEGSVANKFYVHNDI
FRYQDEVFGTAPEDAQKSSSPAPADIAQTVQEDLRTFSWASVTSKNLPPSGAVPVTGIPPHVVKVPASQPRPESKP
ESQIPPQRPQRDQRVREQRINIPPQRGPRPIREAGEQGDIEPRRMVRHPDSHQLFIGNLPHEVDKSELKDFFQSYG
NVVELRINSGGKLPNFGFVVFDDSEPVQKVLSNRPIMFRGEVRLNVEEKKTRAAREGDRRDNRLRGPGGPRGGLG
GGMRGPPRGGMVQKPGFGVGRGLAPRQKLIPMARRAWLEHGHSCFLCEIVIRSQFHTTYEPEA (SEQ ID
NO: 46)
NcVivid Y50W, I52C, C71V, I74V and I85V substitutions with a short 3′ GSG repeat linker sequence,
and G3BP1 PxxP Domain Deletion Mutation; NcVVDY50W/I74V/I85V-LINKERA-G3BP1dPxxP:
MHTLYAPGGYDIMGWLCQIMNRPNPQVELGPVDTSVALVLCDLKQKDTPVVYASEAFLYMTGYSNAEVLGRNC
RFLQSPDGMVKPKSTRKYVDSNTINTMRKAIDRNAEVQVEVVNFKKNGQRFVNFLTMIPVRDETGEYRYSMGFQ
CETEGSGGSGGSGGSGMVMEKPSPLLVGREFVRQYYTLLNQAPDMLHRFYGKNSSYVHGGLDSNGKPADAVYG
QKEIHRKVMSQNFTNCHTKIRHVDAHATLNDGVVVQVMGLLSNNNQALRRFMQTFVLAPEGSVANKFYVHNDI
FRYQDEVFGGFVTEPQEESEEEVEEPEERQQTPEVVPDDSGTFYDQAVVSNDMEEHLEEPVAEPEPDPEPEPEQEP
VSEIQEEKPEPVLEEQLFIGNLPHEVDKSELKDFFQSYGNVVELRINSGGKLPNFGFVVFDDSEPVQKVLSNRPIMFR
GEVRLNVEEKKTRAAREGDRRDNRLRGPGGPRGGLGGGMRGPPRGGMVQKPGFGVGRGLAPRQKLIPMARRA
WLEHGHSCFLCEIVIRSQFHTTYEPEA (SEQ ID NO: 47)
NcVivid Y50W, I52C, C71V, I74V and I85V substitutions with a short 3′ GSG repeat linker sequence,
and G3BP1 RRM Domain Deletion Mutation; NcVVDY50W/I74V/I85V-LINKERA-G3BP1dRRM:
MHTLYAPGGYDIMGWLCQIMNRPNPQVELGPVDTSVALVLCDLKQKDTPVVYASEAFLYMTGYSNAEVLGRNC
RFLQSPDGMVKPKSTRKYVDSNTINTMRKAIDRNAEVQVEVVNFKKNGQRFVNFLTMIPVRDETGEYRYSMGFQ
CETEGSGGSGGSGGSGMVMEKPSPLLVGREFVRQYYTLLNQAPDMLHRFYGKNSSYVHGGLDSNGKPADAVYG
QKEIHRKVMSQNFTNCHTKIRHVDAHATLNDGVVVQVMGLLSNNNQALRRFMQTFVLAPEGSVANKFYVHNDI
FRYQDEVFGGFVTEPQEESEEEVEEPEERQQTPEVVPDDSGTFYDQAVVSNDMEEHLEEPVAEPEPDPEPEPEQEP
VSEIQEEKPEPVLEETAPEDAQKSSSPAPADIAQTVQEDLRTFSWASVTSKNLPPSGAVPVTGIPPHVVKVPASQPR
PESKPESQIPPQRPQRDQRVREQRINIPPQRGPRPIREAGEQGDIEPRRMVRHPDSHEKKTRAAREGDRRDNRLR
GPGGPRGGLGGGMRGPPRGGMVQKPGFGVGRGLAPRQKLIPMARRAWLEHGHSCFLCEIVIRSQFHTTYEPEA
(SEQ ID NO: 48)
NcVivid Y50W, I52C, C71V, I74V and I85V substitutions with a short 3′ GSG repeat linker sequence,
and G3BP1 RGG Domain Deletion Mutation; NcVVDY50W/I74V/I85V-LINKERA-G3BP1dRGG:
MHTLYAPGGYDIMGWLCQIMNRPNPQVELGPVDTSVALVLCDLKQKDTPVVYASEAFLYMTGYSNAEVLGRNC
RFLQSPDGMVKPKSTRKYVDSNTINTMRKAIDRNAEVQVEVVNFKKNGQRFVNFLTMIPVRDETGEYRYSMGFQ
CETEGSGGSGGSGGSGMVMEKPSPLLVGREFVRQYYTLLNQAPDMLHRFYGKNSSYVHGGLDSNGKPADAVYG
QKEIHRKVMSQNFTNCHTKIRHVDAHATLNDGVVVQVMGLLSNNNQALRRFMQTFVLAPEGSVANKFYVHNDI
FRYQDEVFGGFVTEPQEESEEEVEEPEERQQTPEVVPDDSGTFYDQAVVSNDMEEHLEEPVAEPEPDPEPEPEQEP
VSEIQEEKPEPVLEETAPEDAQKSSSPAPADIAQTVQEDLRTFSWASVTSKNLPPSGAVPVTGIPPHVVKVPASQPR
PESKPESQIPPQRPQRDQRVREQRINIPPQRGPRPIREAGEQGDIEPRRMVRHPDSHQLFIGNLPHEVDKSELKDFF
QSYGNVVELRINSGGKLPNFGFVVFDDSEPVQKVLSNRPIMFRGEVRLNVE (SEQ ID NO: 49)
NcVivid Y50W, I52C, C71V, I74V and I85V substitutions with a short 3′ GSG repeat linker sequence,
and G3BP1 RRMRGG Domain Deletion Mutation; NcVVDY50W/I74V/I85V-LINKERA-G3BP1dRRMRGG:
MHTLYAPGGYDIMGWLCQIMNRPNPQVELGPVDTSVALVLCDLKQKDTPVVYASEAFLYMTGYSNAEVLGRNC
RFLQSPDGMVKPKSTRKYVDSNTINTMRKAIDRNAEVQVEVVNFKKNGQRFVNFLTMIPVRDETGEYRYSMGFQ
CETEGSGGSGGSGGSGMVMEKPSPLLVGREFVRQYYTLLNQAPDMLHRFYGKNSSYVHGGLDSNGKPADAVYG
QKEIHRKVMSQNFTNCHTKIRHVDAHATLNDGVVVQVMGLLSNNNQALRRFMQTFVLAPEGSVANKFYVHNDI
FRYQDEVFGGFVTEPQEESEEEVEEPEERQQTPEVVPDDSGTFYDQAVVSNDMEEHLEEPVAEPEPDPEPEPEQEP
VSEIQEEKPEPVLEETAPEDAQKSSSPAPADIAQTVQEDLRTFSWASVTSKNLPPSGAVPVTGIPPHVVKVPASQPR
PESKPESQIPPQRPQRDQRVREQRINIPPQRGPRPIREAGEQGDIEPRRMVRHPDSH (SEQ ID NO: 50)
NcVivid Y50W, I74V and I85V substitutions with a long 3′ GSG repeat linker sequence, and G3BP1
Full Length Protein; NcVVDY50W/I74V/I85V-LINKERB-G3BP1:
MHTLYAPGGYDIMGWLIQIMNRPNPQVELGPVDTSCALVLCDLKQKDTPVVYASEAFLYMTGYSNAEVLGRNCR
FLQSPDGMVKPKSTRKYVDSNTINTMRKAIDRNAEVQVEVVNFKKNGQRFVNFLTMIPVRDETGEYRYSMGFQC
ETEGSGGSGGSGGSGGTGGSGGSGMVMEKPSPLLVGREFVRQYYTLLNQAPDMLHRFYGKNSSYVHGGLDSNG
KPADAVYGQKEIHRKVMSQNFTNCHTKIRHVDAHATLNDGVVVQVMGLLSNNNQALRRFMQTFVLAPEGSVA
NKFYVHNDIFRYQDEVFGGFVTEPQEESEEEVEEPEERQQTPEVVPDDSGTFYDQAVVSNDMEEHLEEPVAEPEP
DPEPEPEQEPVSEIQEEKPEPVLEETAPEDAQKSSSPAPADIAQTVQEDLRTFSWASVTSKNLPPSGAVPVTGIPPH
VVKVPASQPRPESKPESQIPPQRPQRDQRVREQRINIPPQRGPRPIREAGEQGDIEPRRMVRHPDSHQLFIGNLPH
EVDKSELKDFFQSYGNVVELRINSGGKLPNFGFVVFDDSEPVQKVLSNRPIMFRGEVRLNVEEKKTRAAREGDRRD
NRLRGPGGPRGGLGGGMRGPPRGGMVQKPGFGVGRGLAPRQKLIPMARRAWLEHGHSCFLCEIVIRSQFHTTY
EPEA (SEQ ID NO: 51)
NcVivid Y50W, I74V and I85V substitutions with a long 3′ GSG repeat linker sequence, and G3BP1
NTF2 Domain Deletion Mutation; NcVVDY50W/I74V/I85V-LINKERB-G3BP1dNTF2:
MHTLYAPGGYDIMGWLIQIMNRPNPQVELGPVDTSCALVLCDLKQKDTPVVYASEAFLYMTGYSNAEVLGRNCR
FLQSPDGMVKPKSTRKYVDSNTINTMRKAIDRNAEVQVEVVNFKKNGQRFVNFLTMIPVRDETGEYRYSMGFQC
ETEGSGGSGGSGGSGGTGGSGGSGMGFVTEPQEESEEEVEEPEERQQTPEVVPDDSGTFYDQAVVSNDMEEHL
EEPVAEPEPDPEPEPEQEPVSEIQEEKPEPVLEETAPEDAQKSSSPAPADIAQTVQEDLRTFSWASVTSKNLPPSGAV
PVTGIPPHVVKVPASQPRPESKPESQIPPQRPQRDQRVREQRINIPPQRGPRPIREAGEQGDIEPRRMVRHPDSHQ
LFIGNLPHEVDKSELKDFFQSYGNVVELRINSGGKLPNFGFVVFDDSEPVQKVLSNRPIMFRGEVRLNVEEKKTRAA
REGDRRDNRLRGPGGPRGGLGGGMRGPPRGGMVQKPGFGVGRGLAPRQKLIPMARRAWLEHGHSCFLCEIVIR
SQFHTTYEPEA (SEQ ID NO: 52)
NcVivid Y50W, I74V and I85V substitutions with a long 3′ GSG repeat linker sequence, and G3BP1
Acidic Domain Deletion Mutation; NcVVDY50W/I74V/I85V-LINKERB-G3BP1dAcidic:
MHTLYAPGGYDIMGWLIQIMNRPNPQVELGPVDTSCALVLCDLKQKDTPVVYASEAFLYMTGYSNAEVLGRNCR
FLQSPDGMVKPKSTRKYVDSNTINTMRKAIDRNAEVQVEVVNFKKNGQRFVNFLTMIPVRDETGEYRYSMGFQC
ETEGSGGSGGSGGSGGTGGSGGSGMVMEKPSPLLVGREFVRQYYTLLNQAPDMLHRFYGKNSSYVHGGLDSNG
KPADAVYGQKEIHRKVMSQNFTNCHTKIRHVDAHATLNDGVVVQVMGLLSNNNQALRRFMQTFVLAPEGSVA
NKFYVHNDIFRYQDEVFGTAPEDAQKSSSPAPADIAQTVQEDLRTFSWASVTSKNLPPSGAVPVTGIPPHVVKVPA
SQPRPESKPESQIPPQRPQRDQRVREQRINIPPQRGPRPIREAGEQGDIEPRRMVRHPDSHQLFIGNLPHEVDKSE
LKDFFQSYGNVVELRINSGGKLPNFGFVVFDDSEPVQKVLSNRPIMFRGEVRLNVEEKKTRAAREGDRRDNRLRGP
GGPRGGLGGGMRGPPRGGMVQKPGFGVGRGLAPRQKLIPMARRAWLEHGHSCFLCEIVIRSQFHTTYEPEA
(SEQ ID NO: 53)
NcVivid Y50W, I74V and I85V substitutions with a long 3′ GSG repeat linker sequence, and G3BP1
PxxP Domain Deletion Mutation; NcVVDY50W/I74V/I85V-LINKERB-G3BP1dPxxP:
MHTLYAPGGYDIMGWLIQIMNRPNPQVELGPVDTSCALVLCDLKQKDTPVVYASEAFLYMTGYSNAEVLGRNCR
FLQSPDGMVKPKSTRKYVDSNTINTMRKAIDRNAEVQVEVVNFKKNGQRFVNFLTMIPVRDETGEYRYSMGFQC
ETEGSGGSGGSGGSGGTGGSGGSGMVMEKPSPLLVGREFVRQYYTLLNQAPDMLHRFYGKNSSYVHGGLDSNG
KPADAVYGQKEIHRKVMSQNFTNCHTKIRHVDAHATLNDGVVVQVMGLLSNNNQALRRFMQTFVLAPEGSVA
NKFYVHNDIFRYQDEVFGGFVTEPQEESEEEVEEPEERQQTPEVVPDDSGTFYDQAVVSNDMEEHLEEPVAEPEP
DPEPEPEQEPVSEIQEEKPEPVLEEQLFIGNLPHEVDKSELKDFFQSYGNVVELRINSGGKLPNFGFVVFDDSEPVQK
VLSNRPIMFRGEVRLNVEEKKTRAAREGDRRDNRLRGPGGPRGGLGGGMRGPPRGGMVQKPGFGVGRGLAPR
QKLIPMARRAWLEHGHSCFLCEIVIRSQFHTTYEPEA (SEQ ID NO: 54)
NcVivid Y50W, I74V and I85V substitutions with a long 3′ GSG repeat linker sequence, and G3BP1
RRM Domain Deletion Mutation; NcVVDY50W/I74V/I85V-LINKERB-G3BP1dRRM:
MHTLYAPGGYDIMGWLIQIMNRPNPQVELGPVDTSCALVLCDLKQKDTPVVYASEAFLYMTGYSNAEVLGRNCR
FLQSPDGMVKPKSTRKYVDSNTINTMRKAIDRNAEVQVEVVNFKKNGQRFVNFLTMIPVRDETGEYRYSMGFQC
ETEGSGGSGGSGGSGGTGGSGGSGMVMEKPSPLLVGREFVRQYYTLLNQAPDMLHRFYGKNSSYVHGGLDSNG
KPADAVYGQKEIHRKVMSQNFTNCHTKIRHVDAHATLNDGVVVQVMGLLSNNNQALRRFMQTFVLAPEGSVA
NKFYVHNDIFRYQDEVFGGFVTEPQEESEEEVEEPEERQQTPEVVPDDSGTFYDQAVVSNDMEEHLEEPVAEPEP
DPEPEPEQEPVSEIQEEKPEPVLEETAPEDAQKSSSPAPADIAQTVQEDLRTFSWASVTSKNLPPSGAVPVTGIPPH
VVKVPASQPRPESKPESQIPPQRPQRDQRVREQRINIPPQRGPRPIREAGEQGDIEPRRMVRHPDSHEKKTRAARE
GDRRDNRLRGPGGPRGGLGGGMRGPPRGGMVQKPGFGVGRGLAPRQKLIPMARRAWLEHGHSCFLCEIVIRS
QFHTTYEPEA (SEQ ID NO: 55)
NcVivid Y50W, I74V and I85V substitutions with a long 3′ GSG repeat linker sequence, and G3BP1
RGG Domain Deletion Mutation; NcVVDY50W/I74V/I85V-LINKERB-G3BP1dRGG:
MHTLYAPGGYDIMGWLIQIMNRPNPQVELGPVDTSCALVLCDLKQKDTPVVYASEAFLYMTGYSNAEVLGRNCR
FLQSPDGMVKPKSTRKYVDSNTINTMRKAIDRNAEVQVEVVNFKKNGQRFVNFLTMIPVRDETGEYRYSMGFQC
ETEGSGGSGGSGGSGGTGGSGGSGMVMEKPSPLLVGREFVRQYYTLLNQAPDMLHRFYGKNSSYVHGGLDSNG
KPADAVYGQKEIHRKVMSQNFTNCHTKIRHVDAHATLNDGVVVQVMGLLSNNNQALRRFMQTFVLAPEGSVA
NKFYVHNDIFRYQDEVFGGFVTEPQEESEEEVEEPEERQQTPEVVPDDSGTFYDQAVVSNDMEEHLEEPVAEPEP
DPEPEPEQEPVSEIQEEKPEPVLEETAPEDAQKSSSPAPADIAQTVQEDLRTFSWASVTSKNLPPSGAVPVTGIPPH
VVKVPASQPRPESKPESQIPPQRPQRDQRVREQRINIPPQRGPRPIREAGEQGDIEPRRMVRHPDSHQLFIGNLPH
EVDKSELKDFFQSYGNVVELRINSGGKLPNFGFVVFDDSEPVQKVLSNRPIMFRGEVRLNVE (SEQ ID NO: 56)
NcVivid Y50W, I74V and I85V substitutions with a long 3′ GSG repeat linker sequence, and G3BP1
RRMRGG Domain Deletion Mutation; NcVVDY50W/I74V/I85V-LINKERB-G3BP1dRRMRGG:
MHTLYAPGGYDIMGWLIQIMNRPNPQVELGPVDTSCALVLCDLKQKDTPVVYASEAFLYMTGYSNAEVLGRNCR
FLQSPDGMVKPKSTRKYVDSNTINTMRKAIDRNAEVQVEVVNFKKNGQRFVNFLTMIPVRDETGEYRYSMGFQC
ETEGSGGSGGSGGSGGTGGSGGSGMVMEKPSPLLVGREFVRQYYTLLNQAPDMLHRFYGKNSSYVHGGLDSNG
KPADAVYGQKEIHRKVMSQNFTNCHTKIRHVDAHATLNDGVVVQVMGLLSNNNQALRRFMQTFVLAPEGSVA
NKFYVHNDIFRYQDEVFGGFVTEPQEESEEEVEEPEERQQTPEVVPDDSGTFYDQAVVSNDMEEHLEEPVAEPEP
DPEPEPEQEPVSEIQEEKPEPVLEETAPEDAQKSSSPAPADIAQTVQEDLRTFSWASVTSKNLPPSGAVPVTGIPPH
VVKVPASQPRPESKPESQIPPQRPQRDQRVREQRINIPPQRGPRPIREAGEQGDIEPRRMVRHPDSH (SEQ ID
NO: 57)
NcVivid Y50W, I52C, I74V and I85V substitutions with a long 3′ GSG repeat linker sequence, and
G3BP1 Full Length Protein; NcVVDY50W/I74V/I85V-LINKERB-G3BP1:
MHTLYAPGGYDIMGWLCQIMNRPNPQVELGPVDTSCALVLCDLKQKDTPVVYASEAFLYMTGYSNAEVLGRNC
RFLQSPDGMVKPKSTRKYVDSNTINTMRKAIDRNAEVQVEVVNFKKNGQRFVNFLTMIPVRDETGEYRYSMGFQ
CETEGSGGSGGSGGSGGTGGSGGSGMVMEKPSPLLVGREFVRQYYTLLNQAPDMLHRFYGKNSSYVHGGLDSN
GKPADAVYGQKEIHRKVMSQNFTNCHTKIRHVDAHATLNDGVVVQVMGLLSNNNQALRRFMQTFVLAPEGSV
ANKFYVHNDIFRYQDEVFGGFVTEPQEESEEEVEEPEERQQTPEVVPDDSGTFYDQAVVSNDMEEHLEEPVAEPE
PDPEPEPEQEPVSEIQEEKPEPVLEETAPEDAQKSSSPAPADIAQTVQEDLRTFSWASVTSKNLPPSGAVPVTGIPPH
VVKVPASQPRPESKPESQIPPQRPQRDQRVREQRINIPPQRGPRPIREAGEQGDIEPRRMVRHPDSHQLFIGNLPH
EVDKSELKDFFQSYGNVVELRINSGGKLPNFGFVVFDDSEPVQKVLSNRPIMFRGEVRLNVEEKKTRAAREGDRRD
NRLRGPGGPRGGLGGGMRGPPRGGMVQKPGFGVGRGLAPRQKLIPMARRAWLEHGHSCFLCEIVIRSQFHTTY
EPEA (SEQ ID NO: 58)
NcVivid Y50W, I52C, I74V and I85V substitutions with a long 3′ GSG repeat linker sequence, and
G3BP1 NTF2 Domain Deletion Mutation; NcVVDY50W/I74V/I85V-LINKERB-G3BP1dNTF2:
MHTLYAPGGYDIMGWLCQIMNRPNPQVELGPVDTSCALVLCDLKQKDTPVVYASEAFLYMTGYSNAEVLGRNC
RFLQSPDGMVKPKSTRKYVDSNTINTMRKAIDRNAEVQVEVVNFKKNGQRFVNFLTMIPVRDETGEYRYSMGFQ
CETEGSGGSGGSGGSGGTGGSGGSGMGFVTEPQEESEEEVEEPEERQQTPEVVPDDSGTFYDQAVVSNDMEEH
LEEPVAEPEPDPEPEPEQEPVSEIQEEKPEPVLEETAPEDAQKSSSPAPADIAQTVQEDLRTFSWASVTSKNLPPSGA
VPVTGIPPHVVKVPASQPRPESKPESQIPPQRPQRDQRVREQRINIPPQRGPRPIREAGEQGDIEPRRMVRHPDSH
QLFIGNLPHEVDKSELKDFFQSYGNVVELRINSGGKLPNFGFVVFDDSEPVQKVLSNRPIMFRGEVRLNVEEKKTRA
AREGDRRDNRLRGPGGPRGGLGGGMRGPPRGGMVQKPGFGVGRGLAPRQKLIPMARRAWLEHGHSCFLCEIVI
RSQFHTTYEPEA (SEQ ID NO: 59)
NcVivid Y50W, I52C, I74V and I85V substitutions with a long 3′ GSG repeat linker sequence, and
G3BP1 Acidic Domain Deletion Mutation; NcVVDY50W/I74V/I85V-LINKERB-G3BP1dAcidic:
MHTLYAPGGYDIMGWLCQIMNRPNPQVELGPVDTSCALVLCDLKQKDTPVVYASEAFLYMTGYSNAEVLGRNC
RFLQSPDGMVKPKSTRKYVDSNTINTMRKAIDRNAEVQVEVVNFKKNGQRFVNFLTMIPVRDETGEYRYSMGFQ
CETEGSGGSGGSGGSGGTGGSGGSGMVMEKPSPLLVGREFVRQYYTLLNQAPDMLHRFYGKNSSYVHGGLDSN
GKPADAVYGQKEIHRKVMSQNFTNCHTKIRHVDAHATLNDGVVVQVMGLLSNNNQALRRFMQTFVLAPEGSV
ANKFYVHNDIFRYQDEVFGTAPEDAQKSSSPAPADIAQTVQEDLRTFSWASVISKNLPPSGAVPVTGIPPHVVKVP
ASQPRPESKPESQIPPQRPQRDQRVREQRINIPPQRGPRPIREAGEQGDIEPRRMVRHPDSHQLFIGNLPHEVDKS
ELKDFFQSYGNVVELRINSGGKLPNFGFVVFDDSEPVQKVLSNRPIMFRGEVRLNVEEKKTRAAREGDRRDNRLRG
PGGPRGGLGGGMRGPPRGGMVQKPGFGVGRGLAPRQKLIPMARRAWLEHGHSCFLCEIVIRSQFHTTYEPEA
(SEQ ID NO: 60)
NcVivid Y50W, I52C, I74V and I85V substitutions with a long 3′ GSG repeat linker sequence, and
G3BP1 PxxP Domain Deletion Mutation; NcVVDY50W/I74V/I85V-LINKERB-G3BP1dPxxP:
MHTLYAPGGYDIMGWLCQIMNRPNPQVELGPVDTSCALVLCDLKQKDTPVVYASEAFLYMTGYSNAEVLGRNC
RFLQSPDGMVKPKSTRKYVDSNTINTMRKAIDRNAEVQVEVVNFKKNGQRFVNFLTMIPVRDETGEYRYSMGFQ
CETEGSGGSGGSGGSGGTGGSGGSGMVMEKPSPLLVGREFVRQYYTLLNQAPDMLHRFYGKNSSYVHGGLDSN
GKPADAVYGQKEIHRKVMSQNFTNCHTKIRHVDAHATLNDGVVVQVMGLLSNNNQALRRFMQTFVLAPEGSV
ANKFYVHNDIFRYQDEVFGGFVTEPQEESEEEVEEPEERQQTPEVVPDDSGTFYDQAVVSNDMEEHLEEPVAEPE
PDPEPEPEQEPVSEIQEEKPEPVLEEQLFIGNLPHEVDKSELKDFFQSYGNVVELRINSGGKLPNFGFVVFDDSEPVQ
KVLSNRPIMFRGEVRLNVEEKKTRAAREGDRRDNRLRGPGGPRGGLGGGMRGPPRGGMVQKPGFGVGRGLAP
RQKLIPMARRAWLEHGHSCFLCEIVIRSQFHTTYEPEA (SEQ ID NO: 61)
NcVivid Y50W, I52C, I74V and I85V substitutions with a long 3′ GSG repeat linker sequence, and
G3BP1 RRM Domain Deletion Mutation; NcVVDY50W/I74V/I85V-LINKERB-G3BP1dRRM:
MHTLYAPGGYDIMGWLCQIMNRPNPQVELGPVDTSCALVLCDLKQKDTPVVYASEAFLYMTGYSNAEVLGRNC
RFLQSPDGMVKPKSTRKYVDSNTINTMRKAIDRNAEVQVEVVNFKKNGQRFVNFLTMIPVRDETGEYRYSMGFQ
CETEGSGGSGGSGGSGGTGGSGGSGMVMEKPSPLLVGREFVRQYYTLLNQAPDMLHRFYGKNSSYVHGGLDSN
GKPADAVYGQKEIHRKVMSQNFTNCHTKIRHVDAHATLNDGVVVQVMGLLSNNNQALRRFMQTFVLAPEGSV
ANKFYVHNDIFRYQDEVFGGFVTEPQEESEEEVEEPEERQQTPEVVPDDSGTFYDQAVVSNDMEEHLEEPVAEPE
PDPEPEPEQEPVSEIQEEKPEPVLEETAPEDAQKSSSPAPADIAQTVQEDLRTFSWASVTSKNLPPSGAVPVTGIPPH
VVKVPASQPRPESKPESQIPPQRPQRDQRVREQRINIPPQRGPRPIREAGEQGDIEPRRMVRHPDSHEKKTRAARE
GDRRDNRLRGPGGPRGGLGGGMRGPPRGGMVQKPGFGVGRGLAPRQKLIPMARRAWLEHGHSCFLCEIVIRS
QFHTTYEPEA (SEQ ID NO: 62)
NcVivid Y50W, I52C, I74V and I85V substitutions with a long 3′ GSG repeat linker sequence, and
G3BP1 RGG Domain Deletion Mutation; NcVVDY50W/I74V/I85V-LINKERB-G3BP1dRGG:
MHTLYAPGGYDIMGWLCQIMNRPNPQVELGPVDTSCALVLCDLKQKDTPVVYASEAFLYMTGYSNAEVLGRNC
RFLQSPDGMVKPKSTRKYVDSNTINTMRKAIDRNAEVQVEVVNFKKNGQRFVNFLTMIPVRDETGEYRYSMGFQ
CETEGSGGSGGSGGSGGTGGSGGSGMVMEKPSPLLVGREFVRQYYTLLNQAPDMLHRFYGKNSSYVHGGLDSN
GKPADAVYGQKEIHRKVMSQNFTNCHTKIRHVDAHATLNDGVVVQVMGLLSNNNQALRRFMQTFVLAPEGSV
ANKFYVHNDIFRYQDEVFGGFVTEPQEESEEEVEEPEERQQTPEVVPDDSGTFYDQAVVSNDMEEHLEEPVAEPE
PDPEPEPEQEPVSEIQEEKPEPVLEETAPEDAQKSSSPAPADIAQTVQEDLRTFSWASVTSKNLPPSGAVPVTGIPPH
VVKVPASQPRPESKPESQIPPQRPQRDQRVREQRINIPPQRGPRPIREAGEQGDIEPRRMVRHPDSHQLFIGNLPH
EVDKSELKDFFQSYGNVVELRINSGGKLPNFGFVVFDDSEPVQKVLSNRPIMFRGEVRLNVE (SEQ ID NO: 63)
NcVivid Y50W, I52C, I74V and I85V substitutions with a long 3′ GSG repeat linker sequence, and
G3BP1 RRMRGG Domain Deletion Mutation; NcVVDY50W/I74V/I85V-LINKERB-G3BP1dRRMRGG:
MHTLYAPGGYDIMGWLCQIMNRPNPQVELGPVDTSCALVLCDLKQKDTPVVYASEAFLYMTGYSNAEVLGRNC
RFLQSPDGMVKPKSTRKYVDSNTINTMRKAIDRNAEVQVEVVNFKKNGQRFVNFLTMIPVRDETGEYRYSMGFQ
CETEGSGGSGGSGGSGGTGGSGGSGMVMEKPSPLLVGREFVRQYYTLLNQAPDMLHRFYGKNSSYVHGGLDSN
GKPADAVYGQKEIHRKVMSQNFTNCHTKIRHVDAHATLNDGVVVQVMGLLSNNNQALRRFMQTFVLAPEGSV
ANKFYVHNDIFRYQDEVFGGFVTEPQEESEEEVEEPEERQQTPEVVPDDSGTFYDQAVVSNDMEEHLEEPVAEPE
PDPEPEPEQEPVSEIQEEKPEPVLEETAPEDAQKSSSPAPADIAQTVQEDLRTFSWASVTSKNLPPSGAVPVTGIPPH
VVKVPASQPRPESKPESQIPPQRPQRDQRVREQRINIPPQRGPRPIREAGEQGDIEPRRMVRHPDSH (SEQ ID
NO: 64)
NcVivid Y50W, C71V, I74V and I85V substitutions with a long 3′ GSG repeat linker sequence, and
G3BP1 Full Length Protein; NcVVDY50W/I74V/I85V-LINKERB-G3BP1:
MHTLYAPGGYDIMGWLIQIMNRPNPQVELGPVDTSVALVLCDLKQKDTPVVYASEAFLYMTGYSNAEVLGRNCR
FLQSPDGMVKPKSTRKYVDSNTINTMRKAIDRNAEVQVEVVNFKKNGQRFVNFLTMIPVRDETGEYRYSMGFQC
ETEGSGGSGGSGGSGGTGGSGGSGMVMEKPSPLLVGREFVRQYYTLLNQAPDMLHRFYGKNSSYVHGGLDSNG
KPADAVYGQKEIHRKVMSQNFTNCHTKIRHVDAHATLNDGVVVQVMGLLSNNNQALRRFMQTFVLAPEGSVA
NKFYVHNDIFRYQDEVFGGFVTEPQEESEEEVEEPEERQQTPEVVPDDSGTFYDQAVVSNDMEEHLEEPVAEPEP
DPEPEPEQEPVSEIQEEKPEPVLEETAPEDAQKSSSPAPADIAQTVQEDLRTFSWASVTSKNLPPSGAVPVTGIPPH
VVKVPASQPRPESKPESQIPPQRPQRDQRVREQRINIPPQRGPRPIREAGEQGDIEPRRMVRHPDSHQLFIGNLPH
EVDKSELKDFFQSYGNVVELRINSGGKLPNFGFVVFDDSEPVQKVLSNRPIMFRGEVRLNVEEKKTRAAREGDRRD
NRLRGPGGPRGGLGGGMRGPPRGGMVQKPGFGVGRGLAPRQKLIPMARRAWLEHGHSCFLCEIVIRSQFHTTY
EPEA (SEQ ID NO: 65)
NcVivid Y50W, C71V, I74V and I85V substitutions with a long 3′ GSG repeat linker sequence, and
G3BP1 NTF2 Domain Deletion Mutation; NcVVDY50W/I74V/I85V-LINKERB-G3BP1dNTF2:
MHTLYAPGGYDIMGWLIQIMNRPNPQVELGPVDTSVALVLCDLKQKDTPVVYASEAFLYMTGYSNAEVLGRNCR
FLQSPDGMVKPKSTRKYVDSNTINTMRKAIDRNAEVQVEVVNFKKNGQRFVNFLTMIPVRDETGEYRYSMGFQC
ETEGSGGSGGSGGSGGTGGSGGSGMGFVTEPQEESEEEVEEPEERQQTPEVVPDDSGTFYDQAVVSNDMEEHL
EEPVAEPEPDPEPEPEQEPVSEIQEEKPEPVLEETAPEDAQKSSSPAPADIAQTVQEDLRTFSWASVTSKNLPPSGAV
PVTGIPPHVVKVPASQPRPESKPESQIPPQRPQRDQRVREQRINIPPQRGPRPIREAGEQGDIEPRRMVRHPDSHQ
LFIGNLPHEVDKSELKDFFQSYGNVVELRINSGGKLPNFGFVVFDDSEPVQKVLSNRPIMFRGEVRLNVEEKKTRAA
REGDRRDNRLRGPGGPRGGLGGGMRGPPRGGMVQKPGFGVGRGLAPRQKLIPMARRAWLEHGHSCFLCEIVIR
SQFHTTYEPEA (SEQ ID NO: 66)
NcVivid Y50W, C71V, I74V and I85V substitutions with a long 3′ GSG repeat linker sequence, and
G3BP1 Acidic Domain Deletion Mutation; NcVVDY50W/I74V/I85V-LINKERB-G3BP1dAcidic:
MHTLYAPGGYDIMGWLIQIMNRPNPQVELGPVDTSVALVLCDLKQKDTPVVYASEAFLYMTGYSNAEVLGRNCR
FLQSPDGMVKPKSTRKYVDSNTINTMRKAIDRNAEVQVEVVNFKKNGQRFVNFLTMIPVRDETGEYRYSMGFQC
ETEGSGGSGGSGGSGGTGGSGGSGMVMEKPSPLLVGREFVRQYYTLLNQAPDMLHRFYGKNSSYVHGGLDSNG
KPADAVYGQKEIHRKVMSQNFTNCHTKIRHVDAHATLNDGVVVQVMGLLSNNNQALRRFMQTFVLAPEGSVA
NKFYVHNDIFRYQDEVFGTAPEDAQKSSSPAPADIAQTVQEDLRTFSWASVTSKNLPPSGAVPVTGIPPHVVKVPA
SQPRPESKPESQIPPQRPQRDQRVREQRINIPPQRGPRPIREAGEQGDIEPRRMVRHPDSHQLFIGNLPHEVDKSE
LKDFFQSYGNVVELRINSGGKLPNFGFVVFDDSEPVQKVLSNRPIMFRGEVRLNVEEKKTRAAREGDRRDNRLRGP
GGPRGGLGGGMRGPPRGGMVQKPGFGVGRGLAPRQKLIPMARRAWLEHGHSCFLCEIVIRSQFHTTYEPEA
(SEQ ID NO: 67)
NcVivid Y50W, C71V, I74V and I85V substitutions with a long 3′ GSG repeat linker sequence, and
G3BP1 PxxP Domain Deletion Mutation; NcVVDY50W/I74V/I85V-LINKERB-G3BP1dPxxP:
MHTLYAPGGYDIMGWLIQIMNRPNPQVELGPVDTSVALVLCDLKQKDTPVVYASEAFLYMTGYSNAEVLGRNCR
FLQSPDGMVKPKSTRKYVDSNTINTMRKAIDRNAEVQVEVVNFKKNGQRFVNFLTMIPVRDETGEYRYSMGFQC
ETEGSGGSGGSGGSGGTGGSGGSGMVMEKPSPLLVGREFVRQYYTLLNQAPDMLHRFYGKNSSYVHGGLDSNG
KPADAVYGQKEIHRKVMSQNFTNCHTKIRHVDAHATLNDGVVVQVMGLLSNNNQALRRFMQTFVLAPEGSVA
NKFYVHNDIFRYQDEVFGGFVTEPQEESEEEVEEPEERQQTPEVVPDDSGTFYDQAVVSNDMEEHLEEPVAEPEP
DPEPEPEQEPVSEIQEEKPEPVLEEQLFIGNLPHEVDKSELKDFFQSYGNVVELRINSGGKLPNFGFVVFDDSEPVQK
VLSNRPIMFRGEVRLNVEEKKTRAAREGDRRDNRLRGPGGPRGGLGGGMRGPPRGGMVQKPGFGVGRGLAPR
QKLIPMARRAWLEHGHSCFLCEIVIRSQFHTTYEPEA (SEQ ID NO: 68)
NcVivid Y50W, C71V, I74V and I85V substitutions with a long 3′ GSG repeat linker sequence, and
G3BP1 RRM Domain Deletion Mutation; NcVVDY50W/I74V/I85V-LINKERB-G3BP1dRRM:
MHTLYAPGGYDIMGWLIQIMNRPNPQVELGPVDTSVALVLCDLKQKDTPVVYASEAFLYMTGYSNAEVLGRNCR
FLQSPDGMVKPKSTRKYVDSNTINTMRKAIDRNAEVQVEVVNFKKNGQRFVNFLTMIPVRDETGEYRYSMGFQC
ETEGSGGSGGSGGSGGTGGSGGSGMVMEKPSPLLVGREFVRQYYTLLNQAPDMLHRFYGKNSSYVHGGLDSNG
KPADAVYGQKEIHRKVMSQNFTNCHTKIRHVDAHATLNDGVVVQVMGLLSNNNQALRRFMQTFVLAPEGSVA
NKFYVHND1FRYQDEVFGGFVTEPQEESEEEVEEPEERQQTPEVVPDDSGTFYDQAVVSNDMEEHLEEPVAEPEP
DPEPEPEQEPVSEIQEEKPEPVLEETAPEDAQKSSSPAPADIAQTVQEDLRTFSWASVTSKNLPPSGAVPVTGIPPH
VVKVPASQPRPESKPESQIPPQRPQRDQRVREQRINIPPQRGPRPIREAGEQGDIEPRRMVRHPDSHEKKTRAARE
GDRRDNRLRGPGGPRGGLGGGMRGPPRGGMVQKPGFGVGRGLAPRQKLIPMARRAWLEHGHSCFLCEIVIRS
QFHTTYEPEA (SEQ ID NO: 69)
NcVivid Y50W, C71V, I74V and I85V substitutions with a long 3′ GSG repeat linker sequence, and
G3BP1 RGG Domain Deletion Mutation; NcVVDY50W/I74V/I85V-LINKERB-G3BP1dRGG:
MHTLYAPGGYDIMGWLIQIMNRPNPQVELGPVDTSVALVLCDLKQKDTPVVYASEAFLYMTGYSNAEVLGRNCR
FLQSPDGMVKPKSTRKYVDSNTINTMRKAIDRNAEVQVEVVNFKKNGQRFVNFLTMIPVRDETGEYRYSMGFQC
ETEGSGGSGGSGGSGGTGGSGGSGMVMEKPSPLLVGREFVRQYYTLLNQAPDMLHRFYGKNSSYVHGGLDSNG
KPADAVYGQKEIHRKVMSQNFTNCHTKIRHVDAHATLNDGVVVQVMGLLSNNNQALRRFMQTFVLAPEGSVA
NKFYVHNDIFRYQDEVFGGFVTEPQEESEEEVEEPEERQQTPEVVPDDSGTFYDQAVVSNDMEEHLEEPVAEPEP
DPEPEPEQEPVSEIQEEKPEPVLEETAPEDAQKSSSPAPADIAQTVQEDLRTFSWASVTSKNLPPSGAVPVTGIPPH
VVKVPASQPRPESKPESQIPPQRPQRDQRVREQRINIPPQRGPRPIREAGEQGDIEPRRMVRHPDSHQLFIGNLPH
EVDKSELKDFFQSYGNVVELRINSGGKLPNFGFVVFDDSEPVQKVLSNRPIMFRGEVRLNVE (SEQ ID NO: 70)
NcVivid Y50W, C71V, I74V and I85V substitutions with a long 3′ GSG repeat linker sequence, and
G3BP1 RRMRGG Domain Deletion Mutation; NcVVDY50W/I74V/I85V-LINKERB-G3BP1dRRMRGG:
MHTLYAPGGYDIMGWLIQIMNRPNPQVELGPVDTSVALVLCDLKQKDTPVVYASEAFLYMTGYSNAEVLGRNCR
FLQSPDGMVKPKSTRKYVDSNTINTMRKAIDRNAEVQVEVVNFKKNGQRFVNFLTMIPVRDETGEYRYSMGFQC
ETEGSGGSGGSGGSGGTGGSGGSGMVMEKPSPLLVGREFVRQYYTLLNQAPDMLHRFYGKNSSYVHGGLDSNG
KPADAVYGQKEIHRKVMSQNFTNCHTKIRHVDAHATLNDGVVVQVMGLLSNNNQALRRFMQTFVLAPEGSVA
NKFYVHNDIFRYQDEVFGGFVTEPQEESEEEVEEPEERQQTPEVVPDDSGTFYDQAVVSNDMEEHLEEPVAEPEP
DPEPEPEQEPVSEIQEEKPEPVLEETAPEDAQKSSSPAPADIAQTVQEDLRTFSWASVTSKNLPPSGAVPVTGIPPH
VVKVPASQPRPESKPESQIPPQRPQRDQRVREQRINIPPQRGPRPIREAGEQGDIEPRRMVRHPDSH (SEQ ID
NO: 71)
NcVivid Y50W, I52C, C71V, I74V and I85V substitutions with a long 3′ GSG repeat linker sequence,
and G3BP1 Full Length Protein; NcVVDY50W/I74V/I85V-LINKERB-G3BP1:
MHTLYAPGGYDIMGWLCQIMNRPNPQVELGPVDTSVALVLCDLKQKDTPVVYASEAFLYMTGYSNAEVLGRNC
RFLQSPDGMVKPKSTRKYVDSNTINTMRKAIDRNAEVQVEVVNFKKNGQRFVNFLTMIPVRDETGEYRYSMGFQ
CETEGSGGSGGSGGSGGTGGSGGSGMVMEKPSPLLVGREFVRQYYTLLNQAPDMLHRFYGKNSSYVHGGLDSN
GKPADAVYGQKEIHRKVMSQNFTNCHTKIRHVDAHATLNDGVVVQVMGLLSNNNQALRRFMQTFVLAPEGSV
ANKFYVHNDIFRYQDEVFGGFVTEPQEESEEEVEEPEERQQTPEVVPDDSGTFYDQAVVSNDMEEHLEEPVAEPE
PDPEPEPEQEPVSEIQEEKPEPVLEETAPEDAQKSSSPAPADIAQTVQEDLRTFSWASVTSKNLPPSGAVPVTGIPPH
VVKVPASQPRPESKPESQIPPQRPQRDQRVREQRINIPPQRGPRPIREAGEQGDIEPRRMVRHPDSHQLFIGNLPH
EVDKSELKDFFQSYGNVVELRINSGGKLPNFGFVVFDDSEPVQKVLSNRPIMFRGEVRLNVEEKKTRAAREGDRRD
NRLRGPGGPRGGLGGGMRGPPRGGMVQKPGFGVGRGLAPRQKLIPMARRAWLEHGHSCFLCEIVIRSQFHTTY
EPEA (SEQ ID NO: 72)
NcVivid Y50W, I52C, C71V, I74V and I85V substitutions with a long 3′ GSG repeat linker sequence,
and G3BP1 NTF2 Domain Deletion Mutation; NcVVDY50W/I74V/I85V-LINKERB-G3BP1dNTF2:
MHTLYAPGGYDIMGWLCQIMNRPNPQVELGPVDTSVALVLCDLKQKDTPVVYASEAFLYMTGYSNAEVLGRNC
RFLQSPDGMVKPKSTRKYVDSNTINTMRKAIDRNAEVQVEVVNFKKNGQRFVNFLTMIPVRDETGEYRYSMGFQ
CETEGSGGSGGSGGSGGTGGSGGSGMGFVTEPQEESEEEVEEPEERQQTPEVVPDDSGTFYDQAVVSNDMEEH
LEEPVAEPEPDPEPEPEQEPVSEIQEEKPEPVLEETAPEDAQKSSSPAPADIAQTVQEDLRTFSWASVTSKNLPPSGA
VPVTGIPPHVVKVPASQPRPESKPESQIPPQRPQRDQRVREQRINIPPQRGPRPIREAGEQGDIEPRRMVRHPDSH
QLFIGNLPHEVDKSELKDFFQSYGNVVELRINSGGKLPNFGFVVFDDSEPVQKVLSNRPIMFRGEVRLNVEEKKTRA
AREGDRRDNRLRGPGGPRGGLGGGMRGPPRGGMVQKPGFGVGRGLAPRQKLIPMARRAWLEHGHSCFLCEIVI
RSQFHTTYEPEA (SEQ ID NO: 73)
NcVivid Y50W, I52C, C71V, I74V and I85V substitutions with a long 3′ GSG repeat linker sequence,
and G3BP1 Acidic Domain Deletion Mutation; NcVVDY50W/I74V/I85V-LINKERB-G3BP1dAcidic:
MHTLYAPGGYDIMGWLCQIMNRPNPQVELGPVDTSVALVLCDLKQKDTPVVYASEAFLYMTGYSNAEVLGRNC
RFLQSPDGMVKPKSTRKYVDSNTINTMRKAIDRNAEVQVEVVNFKKNGQRFVNFLTMIPVRDETGEYRYSMGFQ
CETEGSGGSGGSGGSGGTGGSGGSGMVMEKPSPLLVGREFVRQYYTLLNQAPDMLHRFYGKNSSYVHGGLDSN
GKPADAVYGQKEIHRKVMSQNFTNCHTKIRHVDAHATLNDGVVVQVMGLLSNNNQALRRFMQTFVLAPEGSV
ANKFYVHNDIFRYQDEVFGTAPEDAQKSSSPAPADIAQTVQEDLRTFSWASVISKNLPPSGAVPVTGIPPHVVKVP
ASQPRPESKPESQIPPQRPQRDQRVREQRINIPPQRGPRPIREAGEQGDIEPRRMVRHPDSHQLFIGNLPHEVDKS
ELKDFFQSYGNVVELRINSGGKLPNFGFVVFDDSEPVQKVLSNRPIMFRGEVRLNVEEKKTRAAREGDRRDNRLRG
PGGPRGGLGGGMRGPPRGGMVQKPGFGVGRGLAPRQKLIPMARRAWLEHGHSCFLCEIVIRSQFHTTYEPEA
(SEQ ID NO: 74)
NcVivid Y50W, I52C, C71V, I74V and I85V substitutions with a long 3′ GSG repeat linker sequence,
and G3BP1 PxxP Domain Deletion Mutation; NcVVDY50W/I74V/I85V-LINKERB-G3BP1dPxxP:
MHTLYAPGGYDIMGWLCQIMNRPNPQVELGPVDTSVALVLCDLKQKDTPVVYASEAFLYMTGYSNAEVLGRNC
RFLQSPDGMVKPKSTRKYVDSNTINTMRKAIDRNAEVQVEVVNFKKNGQRFVNFLTMIPVRDETGEYRYSMGFQ
CETEGSGGSGGSGGSGGTGGSGGSGMVMEKPSPLLVGREFVRQYYTLLNQAPDMLHRFYGKNSSYVHGGLDSN
GKPADAVYGQKEIHRKVMSQNFTNCHTKIRHVDAHATLNDGVVVQVMGLLSNNNQALRRFMQTFVLAPEGSV
ANKFYVHNDIFRYQDEVFGGFVTEPQEESEEEVEEPEERQQTPEVVPDDSGTFYDQAVVSNDMEEHLEEPVAEPE
PDPEPEPEQEPVSEIQEEKPEPVLEEQLFIGNLPHEVDKSELKDFFQSYGNVVELRINSGGKLPNFGFVVFDDSEPVQ
KVLSNRPIMFRGEVRLNVEEKKTRAAREGDRRDNRLRGPGGPRGGLGGGMRGPPRGGMVQKPGFGVGRGLAP
RQKLIPMARRAWLEHGHSCFLCEIVIRSQFHTTYEPEA (SEQ ID NO: 75)
NcVivid Y50W, I52C, C71V, I74V and I85V substitutions with a long 3′ GSG repeat linker sequence,
and G3BP1 RRM Domain Deletion Mutation; NcVVDY50W/I74V/I85V-LINKERB-G3BP1dRRM:
MHTLYAPGGYDIMGWLCQIMNRPNPQVELGPVDTSVALVLCDLKQKDTPVVYASEAFLYMTGYSNAEVLGRNC
RFLQSPDGMVKPKSTRKYVDSNTINTMRKAIDRNAEVQVEVVNFKKNGQRFVNFLTMIPVRDETGEYRYSMGFQ
CETEGSGGSGGSGGSGGTGGSGGSGMVMEKPSPLLVGREFVRQYYTLLNQAPDMLHRFYGKNSSYVHGGLDSN
GKPADAVYGQKEIHRKVMSQNFTNCHTKIRHVDAHATLNDGVVVQVMGLLSNNNQALRRFMQTFVLAPEGSV
ANKFYVHNDIFRYQDEVFGGFVTEPQEESEEEVEEPEERQQTPEVVPDDSGTFYDQAVVSNDMEEHLEEPVAEPE
PDPEPEPEQEPVSEIQEEKPEPVLEETAPEDAQKSSSPAPADIAQTVQEDLRTFSWASVTSKNLPPSGAVPVTGIPPH
VVKVPASQPRPESKPESQIPPQRPQRDQRVREQRINIPPQRGPRPIREAGEQGDIEPRRMVRHPDSHEKKTRAARE
GDRRDNRLRGPGGPRGGLGGGMRGPPRGGMVQKPGFGVGRGLAPRQKLIPMARRAWLEHGHSCFLCEIVIRS
QFHTTYEPEA (SEQ ID NO: 76)
NcVivid Y50W, I52C, C71V, I74V and I85V substitutions with a long 3′ GSG repeat linker sequence,
and G3BP1 RGG Domain Deletion Mutation; NcVVDY50W/I74V/I85V-LINKERB-G3BP1dRGG:
MHTLYAPGGYDIMGWLCQIMNRPNPQVELGPVDTSVALVLCDLKQKDTPVVYASEAFLYMTGYSNAEVLGRNC
RFLQSPDGMVKPKSTRKYVDSNTINTMRKAIDRNAEVQVEVVNFKKNGQRFVNFLTMIPVRDETGEYRYSMGFQ
CETEGSGGSGGSGGSGGTGGSGGSGMVMEKPSPLLVGREFVRQYYTLLNQAPDMLHRFYGKNSSYVHGGLDSN
GKPADAVYGQKEIHRKVMSQNFTNCHTKIRHVDAHATLNDGVVVQVMGLLSNNNQALRRFMQTFVLAPEGSV
ANKFYVHNDIFRYQDEVFGGFVTEPQEESEEEVEEPEERQQTPEVVPDDSGTFYDQAVVSNDMEEHLEEPVAEPE
PDPEPEPEQEPVSEIQEEKPEPVLEETAPEDAQKSSSPAPADIAQTVQEDLRTFSWASVTSKNLPPSGAVPVTGIPPH
VVKVPASQPRPESKPESQIPPQRPQRDQRVREQRINIPPQRGPRPIREAGEQGDIEPRRMVRHPDSHQLFIGNLPH
EVDKSELKDFFQSYGNVVELRINSGGKLPNFGFVVFDDSEPVQKVLSNRPIMFRGEVRLNVE (SEQ ID NO: 77)
NcVivid Y50W, I52C, C71V, I74V and I85V substitutions with a long 3′ GSG repeat linker sequence,
and G3BP1 RRMRGG Domain Deletion Mutation; NcVVDY50W/I74V/I85V-LINKERB-G3BP1dRRMRGG:
MHTLYAPGGYDIMGWLCQIMNRPNPQVELGPVDTSVALVLCDLKQKDTPVVYASEAFLYMTGYSNAEVLGRNC
RFLQSPDGMVKPKSTRKYVDSNTINTMRKAIDRNAEVQVEVVNFKKNGQRFVNFLTMIPVRDETGEYRYSMGFQ
CETEGSGGSGGSGGSGGTGGSGGSGMVMEKPSPLLVGREFVRQYYTLLNQAPDMLHRFYGKNSSYVHGGLDSN
GKPADAVYGQKEIHRKVMSQNFTNCHTKIRHVDAHATLNDGVVVQVMGLLSNNNQALRRFMQTFVLAPEGSV
ANKFYVHNDIFRYQDEVFGGFVTEPQEESEEEVEEPEERQQTPEVVPDDSGTFYDQAVVSNDMEEHLEEPVAEPE
PDPEPEPEQEPVSEIQEEKPEPVLEETAPEDAQKSSSPAPADIAQTVQEDLRTFSWASVTSKNLPPSGAVPVTGIPPH
VVKVPASQPRPESKPESQIPPQRPQRDQRVREQRINIPPQRGPRPIREAGEQGDIEPRRMVRHPDSH (SEQ ID
NO: 78)
Cry2Olig with G3BP1 Full Length Protein; Cry2Olig-G3BP1
MKMDKKTIVWFRRDLRIEDNPALAAAAHEGSVFPVFIWCPEEEGQFYPGRASRWWMKQSLAHLSQSLKALGSDL
TLIKTHNTISAILDCIRVTGATKVVFNHLYDPVSLVRDHTVKEKLVERGISVQSYNGDLLYEPWEIYCEKGKPFTSFNSY
WKKCLDMSIESVMLPPPWRLMPITAAAEAIWACSIEELGLENEAEKPSNALLTRAWSPGWSNADKLLNEFIEKQLI
DYAKNSKKVVGNSTSLLSPYLHFGEISVRHVFQCARMKQIIWARDKNSEGEESADLFLRGIGLREYSRYICFNFPFTH
EQSLLSHLRFFPWDADVDKFKAWRQGRTGYPLVDAGMRELWATGWMHNRIRVIVSSFAVKFLLLPWKWGMKY
FWDTLLDADLECDILGWQYISGSIPDGHELDRLDNPALQGAKYDPEGEYIRQWLPELARLPTEWIHHPWDAPLTVL
KASGVELGTNYAKPIVDIDTARELLAKAISRTRGAQIMIGAAMVMEKPSPLLVGREFVRQYYTLLNQAPDMLHRFY
GKNSSYVHGGLDSNGKPADAVYGQKEIHRKVMSQNFTNCHTKIRHVDAHATLNDGVVVQVMGLLSNNNQALR
RFMQTFVLAPEGSVANKFYVHNDIFRYQDEVFGGFVTEPQEESEEEVEEPEERQQTPEVVPDDSGTFYDQAVVSN
DMEEHLEEPVAEPEPDPEPEPEQEPVSEIQEEKPEPVLEETAPEDAQKSSSPAPADIAQTVQEDLRTFSWASVTSKN
LPPSGAVPVTGIPPHVVKVPASQPRPESKPESQIPPQRPQRDQRVREQRINIPPQRGPRPIREAGEQGDIEPRRMV
RHPDSHQLFIGNLPHEVDKSELKDFFQSYGNVVELRINSGGKLPNFGFVVFDDSEPVQKVLSNRPIMFRGEVRLNV
EEKKTRAAREGDRRDNRLRGPGGPRGGLGGGMRGPPRGGMVQKPGFGVGRGLAPRQKLIPMARRAWLEHGH
SCFLCEIVIRSQFHTTYEPEA (SEQ ID NO: 79)
Cry2Olig with G3BP1 NTF2 Domain Deletion Mutation; Cry2Olig-G3BP1dNTF2
MKMDKKTIVWFRRDLRIEDNPALAAAAHEGSVFPVFIWCPEEEGQFYPGRASRWWMKQSLAHLSQSLKALGSDL
TLIKTHNTISAILDCIRVTGATKVVFNHLYDPVSLVRDHTVKEKLVERGISVQSYNGDLLYEPWEIYCEKGKPFTSFNSY
WKKCLDMSIESVMLPPPWRLMPITAAAEAIWACSIEELGLENEAEKPSNALLTRAWSPGWSNADKLLNEFIEKQLI
DYAKNSKKVVGNSTSLLSPYLHFGEISVRHVFQCARMKQIIWARDKNSEGEESADLFLRGIGLREYSRYICFNFPFTH
EQSLLSHLRFFPWDADVDKFKAWRQGRTGYPLVDAGMRELWATGWMHNRIRVIVSSFAVKFLLLPWKWGMKY
FWDTLLDADLECDILGWQYISGSIPDGHELDRLDNPALQGAKYDPEGEYIRQWLPELARLPTEWIHHPWDAPLTVL
KASGVELGTNYAKPIVDIDTARELLAKAISRTRGAQIMIGAAMGFVTEPQEESEEEVEEPEERQQTPEVVPDDSGTF
YDQAVVSNDMEEHLEEPVAEPEPDPEPEPEQEPVSEIQEEKPEPVLEETAPEDAQKSSSPAPADIAQTVQEDLRTFS
WASVTSKNLPPSGAVPVTGIPPHVVKVPASQPRPESKPESQIPPQRPQRDQRVREQRINIPPQRGPRPIREAGEQG
DIEPRRMVRHPDSHQLFIGNLPHEVDKSELKDFFQSYGNVVELRINSGGKLPNFGFVVFDDSEPVQKVLSNRPIMF
RGEVRLNVEEKKTRAAREGDRRDNRLRGPGGPRGGLGGGMRGPPRGGMVQKPGFGVGRGLAPRQKLIPMARR
AWLEHGHSCFLCEIVIRSQFHTTYEPEA (SEQ ID NO: 80)
Cry2Olig with G3BP1 Acidic Domain Deletion Mutation; Cry2Olig-G3BP1dAcidic
MKMDKKTIVWFRRDLRIEDNPALAAAAHEGSVFPVFIWCPEEEGQFYPGRASRWWMKQSLAHLSQSLKALGSDL
TLIKTHNTISAILDCIRVTGATKVVFNHLYDPVSLVRDHTVKEKLVERGISVQSYNGDLLYEPWEIYCEKGKPFTSFNSY
WKKCLDMSIESVMLPPPWRLMPITAAAEAIWACSIEELGLENEAEKPSNALLTRAWSPGWSNADKLLNEFIEKQLI
DYAKNSKKVVGNSTSLLSPYLHFGEISVRHVFQCARMKQIIWARDKNSEGEESADLFLRGIGLREYSRYICFNFPFTH
EQSLLSHLRFFPWDADVDKFKAWRQGRTGYPLVDAGMRELWATGWMHNRIRVIVSSFAVKFLLLPWKWGMKY
FWDTLLDADLECDILGWQYISGSIPDGHELDRLDNPALQGAKYDPEGEYIRQWLPELARLPTEWIHHPWDAPLTVL
KASGVELGTNYAKPIVDIDTARELLAKAISRTRGAQIMIGAAMVMEKPSPLLVGREFVRQYYTLLNQAPDMLHRFY
GKNSSYVHGGLDSNGKPADAVYGQKEIHRKVMSQNFTNCHTKIRHVDAHATLNDGVVVQVMGLLSNNNQALR
RFMQTFVLAPEGSVANKFYVHNDIFRYQDEVFGTAPEDAQKSSSPAPADIAQTVQEDLRTFSWASVTSKNLPPSG
AVPVTGIPPHVVKVPASQPRPESKPESQIPPQRPQRDQRVREQRINIPPQRGPRPIREAGEQGDIEPRRMVRHPDS
HQLFIGNLPHEVDKSELKDFFQSYGNVVELRINSGGKLPNFGFVVFDDSEPVQKVLSNRPIMFRGEVRLNVEEKKTR
AAREGDRRDNRLRGPGGPRGGLGGGMRGPPRGGMVQKPGFGVGRGLAPRQKLIPMARRAWLEHGHSCFLCEI
VIRSQFHTTYEPEA (SEQ ID NO: 81)
Cry2Olig with G3BP1 PxxP Domain Deletion Mutation; Cry2Olig-G3BP1dPxxP
MKMDKKTIVWFRRDLRIEDNPALAAAAHEGSVFPVFIWCPEEEGQFYPGRASRWWMKQSLAHLSQSLKALGSDL
TLIKTHNTISAILDCIRVTGATKVVFNHLYDPVSLVRDHTVKEKLVERGISVQSYNGDLLYEPWEIYCEKGKPFTSFNSY
WKKCLDMSIESVMLPPPWRLMPITAAAEAIWACSIEELGLENEAEKPSNALLTRAWSPGWSNADKLLNEFIEKQLI
DYAKNSKKVVGNSTSLLSPYLHFGEISVRHVFQCARMKQIIWARDKNSEGEESADLFLRGIGLREYSRYICFNFPFTH
EQSLLSHLRFFPWDADVDKFKAWRQGRTGYPLVDAGMRELWATGWMHNRIRVIVSSFAVKFLLLPWKWGMKY
FWDTLLDADLECDILGWQYISGSIPDGHELDRLDNPALQGAKYDPEGEYIRQWLPELARLPTEWIHHPWDAPLTVL
KASGVELGTNYAKPIVDIDTARELLAKAISRTRGAQIMIGAAMVMEKPSPLLVGREFVRQYYTLLNQAPDMLHRFY
GKNSSYVHGGLDSNGKPADAVYGQKEIHRKVMSQNFTNCHTKIRHVDAHATLNDGVVVQVMGLLSNNNQALR
RFMQTFVLAPEGSVANKFYVHNDIFRYQDEVFGGFVTEPQEESEEEVEEPEERQQTPEVVPDDSGTFYDQAVVSN
DMEEHLEEPVAEPEPDPEPEPEQEPVSEIQEEKPEPVLEEQLFIGNLPHEVDKSELKDFFQSYGNVVELRINSGGKLP
NFGFVVFDDSEPVQKVLSNRPIMFRGEVRLNVEEKKTRAAREGDRRDNRLRGPGGPRGGLGGGMRGPPRGGM
VQKPGFGVGRGLAPRQKLIPMARRAWLEHGHSCFLCEIVIRSQFHTTYEPEA (SEQ ID NO: 82)
Cry2Olig with G3BP1 RRM Domain Deletion Mutation; Cry2Olig-G3BP1dRRM
MKMDKKTIVWFRRDLRIEDNPALAAAAHEGSVFPVFIWCPEEEGQFYPGRASRWWMKQSLAHLSQSLKALGSDL
TLIKTHNTISAILDCIRVTGATKVVFNHLYDPVSLVRDHTVKEKLVERGISVQSYNGDLLYEPWEIYCEKGKPFTSFNSY
WKKCLDMSIESVMLPPPWRLMPITAAAEAIWACSIEELGLENEAEKPSNALLTRAWSPGWSNADKLLNEFIEKQLI
DYAKNSKKVVGNSTSLLSPYLHFGEISVRHVFQCARMKQIIWARDKNSEGEESADLFLRGIGLREYSRYICFNFPFTH
EQSLLSHLRFFPWDADVDKFKAWRQGRTGYPLVDAGMRELWATGWMHNRIRVIVSSFAVKFLLLPWKWGMKY
FWDTLLDADLECDILGWQYISGSIPDGHELDRLDNPALQGAKYDPEGEYIRQWLPELARLPTEWIHHPWDAPLTVL
KASGVELGTNYAKPIVDIDTARELLAKAISRTRGAQIMIGAAMVMEKPSPLLVGREFVRQYYTLLNQAPDMLHRFY
GKNSSYVHGGLDSNGKPADAVYGQKEIHRKVMSQNFTNCHTKIRHVDAHATLNDGVVVQVMGLLSNNNQALR
RFMQTFVLAPEGSVANKFYVHNDIFRYQDEVFGGFVTEPQEESEEEVEEPEERQQTPEVVPDDSGTFYDQAVVSN
DMEEHLEEPVAEPEPDPEPEPEQEPVSEIQEEKPEPVLEETAPEDAQKSSSPAPADIAQTVQEDLRTFSWASVTSKN
LPPSGAVPVTGIPPHVVKVPASQPRPESKPESQIPPQRPQRDQRVREQRINIPPQRGPRPIREAGEQGDIEPRRMV
RHPDSHEKKTRAAREGDRRDNRLRGPGGPRGGLGGGMRGPPRGGMVQKPGFGVGRGLAPRQKLIPMARRAW
LEHGHSCFLCEIVIRSQFHTTYEPEA (SEQ ID NO: 83)
Cry2Olig with G3BP1 RGG Domain Deletion Mutation; Cry2Olig-G3BP1dRGG
MKMDKKTIVWFRRDLRIEDNPALAAAAHEGSVFPVFIWCPEEEGQFYPGRASRWWMKQSLAHLSQSLKALGSDL
TLIKTHNTISAILDCIRVTGATKVVFNHLYDPVSLVRDHTVKEKLVERGISVQSYNGDLLYEPWEIYCEKGKPFTSFNSY
WKKCLDMSIESVMLPPPWRLMPITAAAEAIWACSIEELGLENEAEKPSNALLTRAWSPGWSNADKLLNEFIEKQLI
DYAKNSKKVVGNSTSLLSPYLHFGEISVRHVFQCARMKQIIWARDKNSEGEESADLFLRGIGLREYSRYICFNFPFTH
EQSLLSHLRFFPWDADVDKFKAWRQGRTGYPLVDAGMRELWATGWMHNRIRVIVSSFAVKFLLLPWKWGMKY
FWDTLLDADLECDILGWQYISGSIPDGHELDRLDNPALQGAKYDPEGEYIRQWLPELARLPTEWIHHPWDAPLTVL
KASGVELGTNYAKPIVDIDTARELLAKAISRTRGAQIMIGAAMVMEKPSPLLVGREFVRQYYTLLNQAPDMLHRFY
GKNSSYVHGGLDSNGKPADAVYGQKEIHRKVMSQNFTNCHTKIRHVDAHATLNDGVVVQVMGLLSNNNQALR
RFMQTFVLAPEGSVANKFYVHNDIFRYQDEVFGGFVTEPQEESEEEVEEPEERQQTPEVVPDDSGTFYDQAVVSN
DMEEHLEEPVAEPEPDPEPEPEQEPVSEIQEEKPEPVLEETAPEDAQKSSSPAPADIAQTVQEDLRTFSWASVTSKN
LPPSGAVPVTGIPPHVVKVPASQPRPESKPESQIPPQRPQRDQRVREQRINIPPQRGPRPIREAGEQGDIEPRRMV
RHPDSHQLFIGNLPHEVDKSELKDFFQSYGNVVELRINSGGKLPNFGFVVFDDSEPVQKVLSNRPIMFRGEVRLNV
E (SEQ ID NO: 84)
Cry2Olig with G3BP1 RRMRGG Domain Deletion Mutation; Cry2Olig-G3BP1dRRMRGG
MKMDKKTIVWFRRDLRIEDNPALAAAAHEGSVFPVFIWCPEEEGQFYPGRASRWWMKQSLAHLSQSLKALGSDL
TLIKTHNTISAILDCIRVTGATKVVFNHLYDPVSLVRDHTVKEKLVERGISVQSYNGDLLYEPWEIYCEKGKPFTSFNSY
WKKCLDMSIESVMLPPPWRLMPITAAAEAIWACSIEELGLENEAEKPSNALLTRAWSPGWSNADKLLNEFIEKQLI
DYAKNSKKVVGNSTSLLSPYLHFGEISVRHVFQCARMKQIIWARDKNSEGEESADLFLRGIGLREYSRYICFNFPFTH
EQSLLSHLRFFPWDADVDKFKAWRQGRTGYPLVDAGMRELWATGWMHNRIRVIVSSFAVKFLLLPWKWGMKY
FWDTLLDADLECDILGWQYISGSIPDGHELDRLDNPALQGAKYDPEGEYIRQWLPELARLPTEWIHHPWDAPLTVL
KASGVELGTNYAKPIVDIDTARELLAKAISRTRGAQIMIGAAMVMEKPSPLLVGREFVRQYYTLLNQAPDMLHRFY
GKNSSYVHGGLDSNGKPADAVYGQKEIHRKVMSQNFTNCHTKIRHVDAHATLNDGVVVQVMGLLSNNNQALR
RFMQTFVLAPEGSVANKFYVHNDIFRYQDEVFGGFVTEPQEESEEEVEEPEERQQTPEVVPDDSGTFYDQAVVSN
DMEEHLEEPVAEPEPDPEPEPEQEPVSEIQEEKPEPVLEETAPEDAQKSSSPAPADIAQTVQEDLRTFSWASVTSKN
LPPSGAVPVTGIPPHVVKVPASQPRPESKPESQIPPQRPQRDQRVREQRINIPPQRGPRPIREAGEQGDIEPRRMV
RHPDSH (SEQ ID NO: 85)
G3BP1 Full Length Protein with Cry2Olig; G3BP1-Cry2Olig
MVMEKPSPLLVGREFVRQYYTLLNQAPDMLHRFYGKNSSYVHGGLDSNGKPADAVYGQKEIHRKVMSQNFTNC
HTKIRHVDAHATLNDGVVVQVMGLLSNNNQALRRFMQTFVLAPEGSVANKFYVHNDIFRYQDEVFGGFVTEPQE
ESEEEVEEPEERQQTPEVVPDDSGTFYDQAVVSNDMEEHLEEPVAEPEPDPEPEPEQEPVSEIQEEKPEPVLEETAP
EDAQKSSSPAPADIAQTVQEDLRTFSWASVTSKNLPPSGAVPVTGIPPHVVKVPASQPRPESKPESQIPPQRPQRD
QRVREQRINIPPQRGPRPIREAGEQGDIEPRRMVRHPDSHQLFIGNLPHEVDKSELKDFFQSYGNVVELRINSGGKL
PNFGFVVFDDSEPVQKVLSNRPIMFRGEVRLNVEEKKTRAAREGDRRDNRLRGPGGPRGGLGGGMRGPPRGGM
VQKPGFGVGRGLAPRQKLIPMARRAWLEHGHSCFLCEIVIRSQFHTTYEPEAMKMDKKTIVWFRRDLRIEDNPAL
AAAAHEGSVFPVFIWCPEEEGQFYPGRASRWWMKQSLAHLSQSLKALGSDLTLIKTHNTISAILDCIRVTGATKVVF
NHLYDPVSLVRDHTVKEKLVERGISVQSYNGDLLYEPWEIYCEKGKPFTSFNSYWKKCLDMSIESVMLPPPWRLMP
ITAAAEAIWACSIEELGLENEAEKPSNALLTRAWSPGWSNADKLLNEFIEKQLIDYAKNSKKVVGNSTSLLSPYLHFG
EISVRHVFQCARMKQIIWARDKNSEGEESADLFLRGIGLREYSRYICFNFPFTHEQSLLSHLRFFPWDADVDKFKAW
RQGRTGYPLVDAGMRELWATGWMHNRIRVIVSSFAVKFLLLPWKWGMKYFWDTLLDADLECDILGWQYISGSI
PDGHELDRLDNPALQGAKYDPEGEYIRQWLPELARLPTEWIHHPWDAPLTVLKASGVELGTNYAKPIVDIDTAREL
LAKAISRTRGAQIMIGAA (SEQ ID NO: 86)
G3BP1 NTF2 Domain Deletion Mutation with Cry2Olig; G3BP1dNTF2-Cry2Olig
MGFVTEPQEESEEEVEEPEERQQTPEVVPDDSGTFYDQAVVSNDMEEHLEEPVAEPEPDPEPEPEQEPVSEIQEEK
PEPVLEETAPEDAQKSSSPAPADIAQTVQEDLRTFSWASVTSKNLPPSGAVPVTGIPPHVVKVPASQPRPESKPESQ
IPPQRPQRDQRVREQRINIPPQRGPRPIREAGEQGDIEPRRMVRHPDSHQLFIGNLPHEVDKSELKDFFQSYGNVV
ELRINSGGKLPNFGFVVFDDSEPVQKVLSNRPIMFRGEVRLNVEEKKTRAAREGDRRDNRLRGPGGPRGGLGGG
MRGPPRGGMVQKPGFGVGRGLAPRQKLIPMARRAWLEHGHSCFLCEIVIRSQFHTTYEPEAMKMDKKTIVWFR
RDLRIEDNPALAAAAHEGSVFPVFIWCPEEEGQFYPGRASRWWMKQSLAHLSQSLKALGSDLTLIKTHNTISAILDC
IRVTGATKVVFNHLYDPVSLVRDHTVKEKLVERGISVQSYNGDLLYEPWEIYCEKGKPFTSFNSYWKKCLDMSIESV
MLPPPWRLMPITAAAEAIWACSIEELGLENEAEKPSNALLTRAWSPGWSNADKLLNEFIEKQLIDYAKNSKKVVGN
STSLLSPYLHFGEISVRHVFQCARMKQIIWARDKNSEGEESADLFLRGIGLREYSRYICFNFPFTHEQSLLSHLRFFPW
DADVDKFKAWRQGRTGYPLVDAGMRELWATGWMHNRIRVIVSSFAVKFLLLPWKWGMKYFWDTLLDADLEC
DILGWQYISGSIPDGHELDRLDNPALQGAKYDPEGEYIRQWLPELARLPTEWIHHPWDAPLTVLKASGVELGTNYA
KPIVDIDTARELLAKAISRTRGAQIMIGAA (SEQ ID NO: 87)
G3BP1 Acidic Domain Deletion Mutation with Cry2Olig; G3BP1dAcidic-Cry2Olig
MVMEKPSPLLVGREFVRQYYTLLNQAPDMLHRFYGKNSSYVHGGLDSNGKPADAVYGQKEIHRKVMSQNFTNC
HTKIRHVDAHATLNDGVVVQVMGLLSNNNQALRRFMQTFVLAPEGSVANKFYVHNDIFRYQDEVFGTAPEDAQ
KSSSPAPADIAQTVQEDLRTFSWASVTSKNLPPSGAVPVTGIPPHVVKVPASQPRPESKPESQIPPQRPQRDQRVR
EQRINIPPQRGPRPIREAGEQGDIEPRRMVRHPDSHQLFIGNLPHEVDKSELKDFFQSYGNVVELRINSGGKLPNFG
FVVFDDSEPVQKVLSNRPIMFRGEVRLNVEEKKTRAAREGDRRDNRLRGPGGPRGGLGGGMRGPPRGGMVQKP
GFGVGRGLAPRQKLIPMARRAWLEHGHSCFLCEIVIRSQFHTTYEPEAMKMDKKTIVWFRRDLRIEDNPALAAAA
HEGSVFPVFIWCPEEEGQFYPGRASRWWMKQSLAHLSQSLKALGSDLTLIKTHNTISAILDCIRVTGATKVVFNHLY
DPVSLVRDHTVKEKLVERGISVQSYNGDLLYEPWEIYCEKGKPFTSFNSYWKKCLDMSIESVMLPPPWRLMPITAA
AEAIWACSIEELGLENEAEKPSNALLTRAWSPGWSNADKLLNEFIEKQLIDYAKNSKKVVGNSTSLLSPYLHFGEISV
RHVFQCARMKQIIWARDKNSEGEESADLFLRGIGLREYSRYICFNFPFTHEQSLLSHLRFFPWDADVDKFKAWRQG
RTGYPLVDAGMRELWATGWMHNRIRVIVSSFAVKFLLLPWKWGMKYFWDTLLDADLECDILGWQYISGSIPDG
HELDRLDNPALQGAKYDPEGEYIRQWLPELARLPTEWIHHPWDAPLTVLKASGVELGTNYAKPIVDIDTARELLAK
AISRTRGAQIMIGAA (SEQ ID NO: 88)
G3BP1 PxxP Domain Deletion Mutation with Cry2Olig; G3BP1dPxxP-Cry2Olig
MVMEKPSPLLVGREFVRQYYTLLNQAPDMLHRFYGKNSSYVHGGLDSNGKPADAVYGQKEIHRKVMSQNFTNC
HTKIRHVDAHATLNDGVVVQVMGLLSNNNQALRRFMQTFVLAPEGSVANKFYVHNDIFRYQDEVFGGFVTEPQE
ESEEEVEEPEERQQTPEVVPDDSGTFYDQAVVSNDMEEHLEEPVAEPEPDPEPEPEQEPVSEIQEEKPEPVLEEQLFI
GNLPHEVDKSELKDFFQSYGNVVELRINSGGKLPNFGFVVFDDSEPVQKVLSNRPIMFRGEVRLNVEEKKTRAARE
GDRRDNRLRGPGGPRGGLGGGMRGPPRGGMVQKPGFGVGRGLAPRQKLIPMARRAWLEHGHSCFLCEIVIRS
QFHTTYEPEAMKMDKKTIVWFRRDLRIEDNPALAAAAHEGSVFPVFIWCPEEEGQFYPGRASRWWMKQSLAHL
SQSLKALGSDLTLIKTHNTISAILDCIRVTGATKVVFNHLYDPVSLVRDHTVKEKLVERGISVQSYNGDLLYEPWEIYCE
KGKPFTSFNSYWKKCLDMSIESVMLPPPWRLMPITAAAEAIWACSIEELGLENEAEKPSNALLTRAWSPGWSNAD
KLLNEFIEKQLIDYAKNSKKVVGNSTSLLSPYLHFGEISVRHVFQCARMKQIIWARDKNSEGEESADLFLRGIGLREYS
RYICFNFPFTHEQSLLSHLRFFPWDADVDKFKAWRQGRTGYPLVDAGMRELWATGWMHNRIRVIVSSFAVKFLLL
PWKWGMKYFWDTLLDADLECDILGWQYISGSIPDGHELDRLDNPALQGAKYDPEGEYIRQWLPELARLPTEWIH
HPWDAPLTVLKASGVELGTNYAKPIVDIDTARELLAKAISRTRGAQIMIGAA (SEQ ID NO: 89)
G3BP1 RRM Domain Deletion Mutation with Cry2Olig; G3BP1dRRM-Cry2Olig
MVMEKPSPLLVGREFVRQYYTLLNQAPDMLHRFYGKNSSYVHGGLDSNGKPADAVYGQKEIHRKVMSQNFTNC
HTKIRHVDAHATLNDGVVVQVMGLLSNNNQALRRFMQTFVLAPEGSVANKFYVHNDIFRYQDEVFGGFVTEPQE
ESEEEVEEPEERQQTPEVVPDDSGTFYDQAVVSNDMEEHLEEPVAEPEPDPEPEPEQEPVSEIQEEKPEPVLEETAP
EDAQKSSSPAPADIAQTVQEDLRTFSWASVTSKNLPPSGAVPVTGIPPHVVKVPASQPRPESKPESQIPPQRPQRD
QRVREQRINIPPQRGPRPIREAGEQGDIEPRRMVRHPDSHEKKTRAAREGDRRDNRLRGPGGPRGGLGGGMRG
PPRGGMVQKPGFGVGRGLAPRQKLIPMARRAWLEHGHSCFLCEIVIRSQFHTTYEPEAMKMDKKTIVWFRRDLRI
EDNPALAAAAHEGSVFPVFIWCPEEEGQFYPGRASRWWMKQSLAHLSQSLKALGSDLTLIKTHNTISAILDCIRVT
GATKVVFNHLYDPVSLVRDHTVKEKLVERGISVQSYNGDLLYEPWEIYCEKGKPFTSFNSYWKKCLDMSIESVMLPP
PWRLMPITAAAEAIWACSIEELGLENEAEKPSNALLTRAWSPGWSNADKLLNEFIEKQLIDYAKNSKKVVGNSTSLL
SPYLHFGEISVRHVFQCARMKQIIWARDKNSEGEESADLFLRGIGLREYSRYICFNFPFTHEQSLLSHLRFFPWDADV
DKFKAWRQGRTGYPLVDAGMRELWATGWMHNRIRVIVSSFAVKFLLLPWKWGMKYFWDTLLDADLECDILGW
QYISGSIPDGHELDRLDNPALQGAKYDPEGEYIRQWLPELARLPTEWIHHPWDAPLTVLKASGVELGTNYAKPIVDI
DTARELLAKAISRTRGAQIMIGAA (SEQ ID NO: 90)
G3BP1 RGG Domain Deletion Mutation with Cry2Olig; G3BP1dRGG-Cry2Olig
MVMEKPSPLLVGREFVRQYYTLLNQAPDMLHRFYGKNSSYVHGGLDSNGKPADAVYGQKEIHRKVMSQNFTNC
HTKIRHVDAHATLNDGVVVQVMGLLSNNNQALRRFMQTFVLAPEGSVANKFYVHNDIFRYQDEVFGGFVTEPQE
ESEEEVEEPEERQQTPEVVPDDSGTFYDQAVVSNDMEEHLEEPVAEPEPDPEPEPEQEPVSEIQEEKPEPVLEETAP
EDAQKSSSPAPADIAQTVQEDLRTFSWASVTSKNLPPSGAVPVTGIPPHVVKVPASQPRPESKPESQIPPQRPQRD
QRVREQRINIPPQRGPRPIREAGEQGDIEPRRMVRHPDSHQLFIGNLPHEVDKSELKDFFQSYGNVVELRINSGGKL
PNFGFVVFDDSEPVQKVLSNRPIMFRGEVRLNVEMKMDKKTIVWFRRDLRIEDNPALAAAAHEGSVFPVFIWCPE
EEGQFYPGRASRWWMKQSLAHLSQSLKALGSDLTLIKTHNTISAILDCIRVTGATKVVFNHLYDPVSLVRDHTVKEK
LVERGISVQSYNGDLLYEPWEIYCEKGKPFTSFNSYWKKCLDMSIESVMLPPPWRLMPITAAAEAIWACSIEELGLE
NEAEKPSNALLTRAWSPGWSNADKLLNEFIEKQLIDYAKNSKKVVGNSTSLLSPYLHFGEISVRHVFQCARMKQIIW
ARDKNSEGEESADLFLRGIGLREYSRYICFNFPFTHEQSLLSHLRFFPWDADVDKFKAWRQGRTGYPLVDAGMREL
WATGWMHNRIRVIVSSFAVKFLLLPWKWGMKYFWDTLLDADLECDILGWQYISGSIPDGHELDRLDNPALQGAK
YDPEGEYIRQWLPELARLPTEWIHHPWDAPLTVLKASGVELGTNYAKPIVDIDTARELLAKAISRTRGAQIMIGAA
(SEQ ID NO: 91)
G3BP1 RRMRGG Domain Deletion Mutation with Cry2Olig; G3BP1dRRMRGG-Cry2Olig
MVMEKPSPLLVGREFVRQYYTLLNQAPDMLHRFYGKNSSYVHGGLDSNGKPADAVYGQKEIHRKVMSQNFTNC
HTKIRHVDAHATLNDGVVVQVMGLLSNNNQALRRFMQTFVLAPEGSVANKFYVHNDIFRYQDEVFGGFVTEPQE
ESEEEVEEPEERQQTPEVVPDDSGTFYDQAVVSNDMEEHLEEPVAEPEPDPEPEPEQEPVSEIQEEKPEPVLEETAP
EDAQKSSSPAPADIAQTVQEDLRTFSWASVTSKNLPPSGAVPVTGIPPHVVKVPASQPRPESKPESQIPPQRPQRD
QRVREQRINIPPQRGPRPIREAGEQGDIEPRRMVRHPDSHMKMDKKTIVWFRRDLRIEDNPALAAAAHEGSVFP
VFIWCPEEEGQFYPGRASRWWMKQSLAHLSQSLKALGSDLTLIKTHNTISAILDCIRVTGATKVVFNHLYDPVSLVR
DHTVKEKLVERGISVQSYNGDLLYEPWEIYCEKGKPFTSFNSYWKKCLDMSIESVMLPPPWRLMPITAAAEAIWAC
SIEELGLENEAEKPSNALLTRAWSPGWSNADKLLNEFIEKQLIDYAKNSKKVVGNSTSLLSPYLHFGEISVRHVFQCA
RMKQIIWARDKNSEGEESADLFLRGIGLREYSRYICFNFPFTHEQSLLSHLRFFPWDADVDKFKAWRQGRTGYPLV
DAGMRELWATGWMHNRIRVIVSSFAVKFLLLPWKWGMKYFWDTLLDADLECDILGWQYISGSIPDGHELDRLD
NPALQGAKYDPEGEYIRQWLPELARLPTEWIHHPWDAPLTVLKASGVELGTNYAKPIVDIDTARELLAKAISRTRGA
QIMIGAA (SEQ ID NO: 92)
Cry2PHR with G3BP1 Full Length Protein; Cry2PHR-G3BP1
MKMDKKTIVWFRRDLRIEDNPALAAAAHEGSVFPVFIWCPEEEGQFYPGRASRWWMKQSLAHLSQSLKALGSDL
TLIKTHNTISAILDCIRVTGATKVVFNHLYDPVSLVRDHTVKEKLVERGISVQSYNGDLLYEPWEIYCEKGKPFTSFNSY
WKKCLDMSIESVMLPPPWRLMPITAAAEAIWACSIEELGLENEAEKPSNALLTRAWSPGWSNADKLLNEFIEKQLI
DYAKNSKKVVGNSTSLLSPYLHFGEISVRHVFQCARMKQIIWARDKNSEGEESADLFLRGIGLREYSRYICFNFPFTH
EQSLLSHLRFFPWDADVDKFKAWRQGRTGYPLVDAGMRELWATGWMHNRIRVIVSSFAVKFLLLPWKWGMKY
FWDTLLDADLECDILGWQYISGSIPDGHELDRLDNPALQGAKYDPEGEYIRQWLPELARLPTEWIHHPWDAPLTVL
KASGVELGTNYAKPIVDIDTARELLAKAISRTREAQIMIGAAMVMEKPSPLLVGREFVRQYYTLLNQAPDMLHRFY
GKNSSYVHGGLDSNGKPADAVYGQKEIHRKVMSQNFTNCHTKIRHVDAHATLNDGVVVQVMGLLSNNNQALR
RFMQTFVLAPEGSVANKFYVHNDIFRYQDEVFGGFVTEPQEESEEEVEEPEERQQTPEVVPDDSGTFYDQAVVSN
DMEEHLEEPVAEPEPDPEPEPEQEPVSEIQEEKPEPVLEETAPEDAQKSSSPAPADIAQTVQEDLRTFSWASVTSKN
LPPSGAVPVTGIPPHVVKVPASQPRPESKPESQIPPQRPQRDQRVREQRINIPPQRGPRPIREAGEQGDIEPRRMV
RHPDSHQLFIGNLPHEVDKSELKDFFQSYGNVVELRINSGGKLPNFGFVVFDDSEPVQKVLSNRPIMFRGEVRLNV
EEKKTRAAREGDRRDNRLRGPGGPRGGLGGGMRGPPRGGMVQKPGFGVGRGLAPRQKLIPMARRAWLEHGH
SCFLCEIVIRSQFHTTYEPEA (SEQ ID NO: 93)
Cry2PHR with G3BP1 NTF2 Domain Deletion Mutation; Cry2PHR-G3BP1dNTF2
MKMDKKTIVWFRRDLRIEDNPALAAAAHEGSVFPVFIWCPEEEGQFYPGRASRWWMKQSLAHLSQSLKALGSDL
TLIKTHNTISAILDCIRVTGATKVVFNHLYDPVSLVRDHTVKEKLVERGISVQSYNGDLLYEPWEIYCEKGKPFTSFNSY
WKKCLDMSIESVMLPPPWRLMPITAAAEAIWACSIEELGLENEAEKPSNALLTRAWSPGWSNADKLLNEFIEKQLI
DYAKNSKKVVGNSTSLLSPYLHFGEISVRHVFQCARMKQIIWARDKNSEGEESADLFLRGIGLREYSRYICFNFPFTH
EQSLLSHLRFFPWDADVDKFKAWRQGRTGYPLVDAGMRELWATGWMHNRIRVIVSSFAVKFLLLPWKWGMKY
FWDTLLDADLECDILGWQYISGSIPDGHELDRLDNPALQGAKYDPEGEYIRQWLPELARLPTEWIHHPWDAPLTVL
KASGVELGTNYAKPIVDIDTARELLAKAISRTREAQIMIGAAMGFVTEPQEESEEEVEEPEERQQTPEVVPDDSGTFY
DQAVVSNDMEEHLEEPVAEPEPDPEPEPEQEPVSEIQEEKPEPVLEETAPEDAQKSSSPAPADIAQTVQEDLRTFS
WASVTSKNLPPSGAVPVTGIPPHVVKVPASQPRPESKPESQIPPQRPQRDQRVREQRINIPPQRGPRPIREAGEQG
DIEPRRMVRHPDSHQLFIGNLPHEVDKSELKDFFQSYGNVVELRINSGGKLPNFGFVVFDDSEPVQKVLSNRPIMF
RGEVRLNVEEKKTRAAREGDRRDNRLRGPGGPRGGLGGGMRGPPRGGMVQKPGFGVGRGLAPRQKLIPMARR
AWLEHGHSCFLCEIVIRSQFHTTYEPEA (SEQ ID NO: 94)
Cry2PHR with G3BP1 Acidic Domain Deletion Mutation; Cry2PHR-G3BP1dAcidic
MKMDKKTIVWFRRDLRIEDNPALAAAAHEGSVFPVFIWCPEEEGQFYPGRASRWWMKQSLAHLSQSLKALGSDL
TLIKTHNTISAILDCIRVTGATKVVFNHLYDPVSLVRDHTVKEKLVERGISVQSYNGDLLYEPWEIYCEKGKPFTSFNSY
WKKCLDMSIESVMLPPPWRLMPITAAAEAIWACSIEELGLENEAEKPSNALLTRAWSPGWSNADKLLNEFIEKQLI
DYAKNSKKVVGNSTSLLSPYLHFGEISVRHVFQCARMKQIIWARDKNSEGEESADLFLRGIGLREYSRYICFNFPFTH
EQSLLSHLRFFPWDADVDKFKAWRQGRTGYPLVDAGMRELWATGWMHNRIRVIVSSFAVKFLLLPWKWGMKY
FWDTLLDADLECDILGWQYISGSIPDGHELDRLDNPALQGAKYDPEGEYIRQWLPELARLPTEWIHHPWDAPLTVL
KASGVELGTNYAKPIVDIDTARELLAKAISRTREAQIMIGAAMVMEKPSPLLVGREFVRQYYTLLNQAPDMLHRFY
GKNSSYVHGGLDSNGKPADAVYGQKEIHRKVMSQNFTNCHTKIRHVDAHATLNDGVVVQVMGLLSNNNQALR
RFMQTFVLAPEGSVANKFYVHNDIFRYQDEVFGTAPEDAQKSSSPAPADIAQTVQEDLRTFSWASVTSKNLPPSG
AVPVTGIPPHVVKVPASQPRPESKPESQIPPQRPQRDQRVREQRINIPPQRGPRPIREAGEQGDIEPRRMVRHPDS
HQLFIGNLPHEVDKSELKDFFQSYGNVVELRINSGGKLPNFGFVVFDDSEPVQKVLSNRPIMFRGEVRLNVEEKKTR
AAREGDRRDNRLRGPGGPRGGLGGGMRGPPRGGMVQKPGFGVGRGLAPRQKLIPMARRAWLEHGHSCFLCEI
VIRSQFHTTYEPEA (SEQ ID NO: 95)
Cry2PHR with G3BP1 PxxP Domain Deletion Mutation; Cry2PHR-G3BP1dPxxP
MKMDKKTIVWFRRDLRIEDNPALAAAAHEGSVFPVFIWCPEEEGQFYPGRASRWWMKQSLAHLSQSLKALGSDL
TLIKTHNTISAILDCIRVTGATKVVFNHLYDPVSLVRDHTVKEKLVERGISVQSYNGDLLYEPWEIYCEKGKPFTSFNSY
WKKCLDMSIESVMLPPPWRLMPITAAAEAIWACSIEELGLENEAEKPSNALLTRAWSPGWSNADKLLNEFIEKQLI
DYAKNSKKVVGNSTSLLSPYLHFGEISVRHVFQCARMKQIIWARDKNSEGEESADLFLRGIGLREYSRYICFNFPFTH
EQSLLSHLRFFPWDADVDKFKAWRQGRTGYPLVDAGMRELWATGWMHNRIRVIVSSFAVKFLLLPWKWGMKY
FWDTLLDADLECDILGWQYISGSIPDGHELDRLDNPALQGAKYDPEGEYIRQWLPELARLPTEWIHHPWDAPLTVL
KASGVELGTNYAKPIVDIDTARELLAKAISRTREAQIMIGAAMVMEKPSPLLVGREFVRQYYTLLNQAPDMLHRFY
GKNSSYVHGGLDSNGKPADAVYGQKEIHRKVMSQNFTNCHTKIRHVDAHATLNDGVVVQVMGLLSNNNQALR
RFMQTFVLAPEGSVANKFYVHNDIFRYQDEVFGGFVTEPQEESEEEVEEPEERQQTPEVVPDDSGTFYDQAVVSN
DMEEHLEEPVAEPEPDPEPEPEQEPVSEIQEEKPEPVLEEQLFIGNLPHEVDKSELKDFFQSYGNVVELRINSGGKLP
NFGFVVFDDSEPVQKVLSNRPIMFRGEVRLNVEEKKTRAAREGDRRDNRLRGPGGPRGGLGGGMRGPPRGGM
VQKPGFGVGRGLAPRQKLIPMARRAWLEHGHSCFLCEIVIRSQFHTTYEPEA (SEQ ID NO: 96)
Cry2PHR with G3BP1 RRM Domain Deletion Mutation; Cry2PHR-G3BP1dRRM
MKMDKKTIVWFRRDLRIEDNPALAAAAHEGSVFPVFIWCPEEEGQFYPGRASRWWMKQSLAHLSQSLKALGSDL
TLIKTHNTISAILDCIRVTGATKVVFNHLYDPVSLVRDHTVKEKLVERGISVQSYNGDLLYEPWEIYCEKGKPFTSFNSY
WKKCLDMSIESVMLPPPWRLMPITAAAEAIWACSIEELGLENEAEKPSNALLTRAWSPGWSNADKLLNEFIEKQLI
DYAKNSKKVVGNSTSLLSPYLHFGEISVRHVFQCARMKQIIWARDKNSEGEESADLFLRGIGLREYSRYICFNFPFTH
EQSLLSHLRFFPWDADVDKFKAWRQGRTGYPLVDAGMRELWATGWMHNRIRVIVSSFAVKFLLLPWKWGMKY
FWDTLLDADLECDILGWQYISGSIPDGHELDRLDNPALQGAKYDPEGEYIRQWLPELARLPTEWIHHPWDAPLTVL
KASGVELGTNYAKPIVDIDTARELLAKAISRTREAQIMIGAAMVMEKPSPLLVGREFVRQYYTLLNQAPDMLHRFY
GKNSSYVHGGLDSNGKPADAVYGQKEIHRKVMSQNFTNCHTKIRHVDAHATLNDGVVVQVMGLLSNNNQALR
RFMQTFVLAPEGSVANKFYVHNDIFRYQDEVFGGFVTEPQEESEEEVEEPEERQQTPEVVPDDSGTFYDQAVVSN
DMEEHLEEPVAEPEPDPEPEPEQEPVSEIQEEKPEPVLEETAPEDAQKSSSPAPADIAQTVQEDLRTFSWASVTSKN
LPPSGAVPVTGIPPHVVKVPASQPRPESKPESQIPPQRPQRDQRVREQRINIPPQRGPRPIREAGEQGDIEPRRMV
RHPDSHEKKTRAAREGDRRDNRLRGPGGPRGGLGGGMRGPPRGGMVQKPGFGVGRGLAPRQKLIPMARRAW
LEHGHSCFLCEIVIRSQFHTTYEPEA (SEQ ID NO: 97)
Cry2PHR with G3BP1 RGG Domain Deletion Mutation; Cry2PHR-G3BP1dRGG
MKMDKKTIVWFRRDLRIEDNPALAAAAHEGSVFPVFIWCPEEEGQFYPGRASRWWMKQSLAHLSQSLKALGSDL
TLIKTHNTISAILDCIRVTGATKVVFNHLYDPVSLVRDHTVKEKLVERGISVQSYNGDLLYEPWEIYCEKGKPFTSFNSY
WKKCLDMSIESVMLPPPWRLMPITAAAEAIWACSIEELGLENEAEKPSNALLTRAWSPGWSNADKLLNEFIEKQLI
DYAKNSKKVVGNSTSLLSPYLHFGEISVRHVFQCARMKQIIWARDKNSEGEESADLFLRGIGLREYSRYICFNFPFTH
EQSLLSHLRFFPWDADVDKFKAWRQGRTGYPLVDAGMRELWATGWMHNRIRVIVSSFAVKFLLLPWKWGMKY
FWDTLLDADLECDILGWQYISGSIPDGHELDRLDNPALQGAKYDPEGEYIRQWLPELARLPTEWIHHPWDAPLTVL
KASGVELGTNYAKPIVDIDTARELLAKAISRTREAQIMIGAAMVMEKPSPLLVGREFVRQYYTLLNQAPDMLHRFY
GKNSSYVHGGLDSNGKPADAVYGQKEIHRKVMSQNFTNCHTKIRHVDAHATLNDGVVVQVMGLLSNNNQALR
RFMQTFVLAPEGSVANKFYVHNDIFRYQDEVFGGFVTEPQEESEEEVEEPEERQQTPEVVPDDSGTFYDQAVVSN
DMEEHLEEPVAEPEPDPEPEPEQEPVSEIQEEKPEPVLEETAPEDAQKSSSPAPADIAQTVQEDLRTFSWASVTSKN
LPPSGAVPVTGIPPHVVKVPASQPRPESKPESQIPPQRPQRDQRVREQRINIPPQRGPRPIREAGEQGDIEPRRMV
RHPDSHQLFIGNLPHEVDKSELKDFFQSYGNVVELRINSGGKLPNFGFVVFDDSEPVQKVLSNRPIMFRGEVRLNV
E (SEQ ID NO: 98)
Cry2PHR with G3BP1 RRMRGG Domain Deletion Mutation; Cry2PHR-G3BP1dRRMRGG
MKMDKKTIVWFRRDLRIEDNPALAAAAHEGSVFPVFIWCPEEEGQFYPGRASRWWMKQSLAHLSQSLKALGSDL
TLIKTHNTISAILDCIRVTGATKVVFNHLYDPVSLVRDHTVKEKLVERGISVQSYNGDLLYEPWEIYCEKGKPFTSFNSY
WKKCLDMSIESVMLPPPWRLMPITAAAEAIWACSIEELGLENEAEKPSNALLTRAWSPGWSNADKLLNEFIEKQLI
DYAKNSKKVVGNSTSLLSPYLHFGEISVRHVFQCARMKQIIWARDKNSEGEESADLFLRGIGLREYSRYICFNFPFTH
EQSLLSHLRFFPWDADVDKFKAWRQGRTGYPLVDAGMRELWATGWMHNRIRVIVSSFAVKFLLLPWKWGMKY
FWDTLLDADLECDILGWQYISGSIPDGHELDRLDNPALQGAKYDPEGEYIRQWLPELARLPTEWIHHPWDAPLTVL
KASGVELGTNYAKPIVDIDTARELLAKAISRTREAQIMIGAAMVMEKPSPLLVGREFVRQYYTLLNQAPDMLHRFY
GKNSSYVHGGLDSNGKPADAVYGQKEIHRKVMSQNFTNCHTKIRHVDAHATLNDGVVVQVMGLLSNNNQALR
RFMQTFVLAPEGSVANKFYVHNDIFRYQDEVFGGFVTEPQEESEEEVEEPEERQQTPEVVPDDSGTFYDQAVVSN
DMEEHLEEPVAEPEPDPEPEPEQEPVSEIQEEKPEPVLEETAPEDAQKSSSPAPADIAQTVQEDLRTFSWASVTSKN
LPPSGAVPVTGIPPHVVKVPASQPRPESKPESQIPPQRPQRDQRVREQRINIPPQRGPRPIREAGEQGDIEPRRMV
RHPDSH (SEQ ID NO: 99)
G3BP1 Full Length Protein with Cry2PHR; G3BP1-Cry2PHR
MVMEKPSPLLVGREFVRQYYTLLNQAPDMLHRFYGKNSSYVHGGLDSNGKPADAVYGQKEIHRKVMSQNFTNC
HTKIRHVDAHATLNDGVVVQVMGLLSNNNQALRRFMQTFVLAPEGSVANKFYVHNDIFRYQDEVFGGFVTEPQE
ESEEEVEEPEERQQTPEVVPDDSGTFYDQAVVSNDMEEHLEEPVAEPEPDPEPEPEQEPVSEIQEEKPEPVLEETAP
EDAQKSSSPAPADIAQTVQEDLRTFSWASVTSKNLPPSGAVPVTGIPPHVVKVPASQPRPESKPESQIPPQRPQRD
QRVREQRINIPPQRGPRPIREAGEQGDIEPRRMVRHPDSHQLFIGNLPHEVDKSELKDFFQSYGNVVELRINSGGKL
PNFGFVVFDDSEPVQKVLSNRPIMFRGEVRLNVEEKKTRAAREGDRRDNRLRGPGGPRGGLGGGMRGPPRGGM
VQKPGFGVGRGLAPRQKLIPMARRAWLEHGHSCFLCEIVIRSQFHTTYEPEAMKMDKKTIVWFRRDLRIEDNPAL
AAAAHEGSVFPVFIWCPEEEGQFYPGRASRWWMKQSLAHLSQSLKALGSDLTLIKTHNTISAILDCIRVTGATKVVF
NHLYDPVSLVRDHTVKEKLVERGISVQSYNGDLLYEPWEIYCEKGKPFTSFNSYWKKCLDMSIESVMLPPPWRLMP
ITAAAEAIWACSIEELGLENEAEKPSNALLTRAWSPGWSNADKLLNEFIEKQLIDYAKNSKKVVGNSTSLLSPYLHFG
EISVRHVFQCARMKQIIWARDKNSEGEESADLFLRGIGLREYSRYICFNFPFTHEQSLLSHLRFFPWDADVDKFKAW
RQGRTGYPLVDAGMRELWATGWMHNRIRVIVSSFAVKFLLLPWKWGMKYFWDTLLDADLECDILGWQYISGSI
PDGHELDRLDNPALQGAKYDPEGEYIRQWLPELARLPTEWIHHPWDAPLTVLKASGVELGTNYAKPIVDIDTAREL
LAKAISRTREAQIMIGAA (SEQ ID NO: 100)
G3BP1 NTF2 Domain Deletion Mutation with Cry2PHR; G3BP1dNTF2-Cry2PHR
MGFVTEPQEESEEEVEEPEERQQTPEVVPDDSGTFYDQAVVSNDMEEHLEEPVAEPEPDPEPEPEQEPVSEIQEEK
PEPVLEETAPEDAQKSSSPAPADIAQTVQEDLRTFSWASVTSKNLPPSGAVPVTGIPPHVVKVPASQPRPESKPESQ
IPPQRPQRDQRVREQRINIPPQRGPRPIREAGEQGDIEPRRMVRHPDSHQLFIGNLPHEVDKSELKDFFQSYGNVV
ELRINSGGKLPNFGFVVFDDSEPVQKVLSNRPIMFRGEVRLNVEEKKTRAAREGDRRDNRLRGPGGPRGGLGGG
MRGPPRGGMVQKPGFGVGRGLAPRQKLIPMARRAWLEHGHSCFLCEIVIRSQFHTTYEPEAMKMDKKTIVWFR
RDLRIEDNPALAAAAHEGSVFPVFIWCPEEEGQFYPGRASRWWMKQSLAHLSQSLKALGSDLTLIKTHNTISAILDC
IRVTGATKVVFNHLYDPVSLVRDHTVKEKLVERGISVQSYNGDLLYEPWEIYCEKGKPFTSFNSYWKKCLDMSIESV
MLPPPWRLMPITAAAEAIWACSIEELGLENEAEKPSNALLTRAWSPGWSNADKLLNEFIEKQLIDYAKNSKKVVGN
STSLLSPYLHFGEISVRHVFQCARMKQIIWARDKNSEGEESADLFLRGIGLREYSRYICFNFPFTHEQSLLSHLRFFPW
DADVDKFKAWRQGRTGYPLVDAGMRELWATGWMHNRIRVIVSSFAVKFLLLPWKWGMKYFWDTLLDADLEC
DILGWQYISGSIPDGHELDRLDNPALQGAKYDPEGEYIRQWLPELARLPTEWIHHPWDAPLTVLKASGVELGTNYA
KPIVDIDTARELLAKAISRTREAQIMIGAA (SEQ ID NO: 101)
G3BP1 Acidic Domain Deletion Mutation with Cry2PHR; G3BP1dAcidic-Cry2PHR
MVMEKPSPLLVGREFVRQYYTLLNQAPDMLHRFYGKNSSYVHGGLDSNGKPADAVYGQKEIHRKVMSQNFTNC
HTKIRHVDAHATLNDGVVVQVMGLLSNNNQALRRFMQTFVLAPEGSVANKFYVHNDIFRYQDEVFGTAPEDAQ
KSSSPAPADIAQTVQEDLRTFSWASVTSKNLPPSGAVPVTGIPPHVVKVPASQPRPESKPESQIPPQRPQRDQRVR
EQRINIPPQRGPRPIREAGEQGDIEPRRMVRHPDSHQLFIGNLPHEVDKSELKDFFQSYGNVVELRINSGGKLPNFG
FVVFDDSEPVQKVLSNRPIMFRGEVRLNVEEKKTRAAREGDRRDNRLRGPGGPRGGLGGGMRGPPRGGMVQKP
GFGVGRGLAPRQKLIPMARRAWLEHGHSCFLCEIVIRSQFHTTYEPEAMKMDKKTIVWFRRDLRIEDNPALAAAA
HEGSVFPVFIWCPEEEGQFYPGRASRWWMKQSLAHLSQSLKALGSDLTLIKTHNTISAILDCIRVTGATKVVFNHLY
DPVSLVRDHTVKEKLVERGISVQSYNGDLLYEPWEIYCEKGKPFTSFNSYWKKCLDMSIESVMLPPPWRLMPITAA
AEAIWACSIEELGLENEAEKPSNALLTRAWSPGWSNADKLLNEFIEKQLIDYAKNSKKVVGNSTSLLSPYLHFGEISV
RHVFQCARMKQIIWARDKNSEGEESADLFLRGIGLREYSRYICFNFPFTHEQSLLSHLRFFPWDADVDKFKAWRQG
RTGYPLVDAGMRELWATGWMHNRIRVIVSSFAVKFLLLPWKWGMKYFWDTLLDADLECDILGWQYISGSIPDG
HELDRLDNPALQGAKYDPEGEYIRQWLPELARLPTEWIHHPWDAPLTVLKASGVELGTNYAKPIVDIDTARELLAK
AISRTREAQIMIGAA (SEQ ID NO: 102)
G3BP1 PxxP Domain Deletion Mutation with Cry2PHR; G3BP1dPxxP- Cry2PHR
MVMEKPSPLLVGREFVRQYYTLLNQAPDMLHRFYGKNSSYVHGGLDSNGKPADAVYGQKEIHRKVMSQNFTNC
HTKIRHVDAHATLNDGVVVQVMGLLSNNNQALRRFMQTFVLAPEGSVANKFYVHNDIFRYQDEVFGGFVTEPQE
ESEEEVEEPEERQQTPEVVPDDSGTFYDQAVVSNDMEEHLEEPVAEPEPDPEPEPEQEPVSEIQEEKPEPVLEEQLFI
GNLPHEVDKSELKDFFQSYGNVVELRINSGGKLPNFGFVVFDDSEPVQKVLSNRPIMFRGEVRLNVEEKKTRAARE
GDRRDNRLRGPGGPRGGLGGGMRGPPRGGMVQKPGFGVGRGLAPRQKLIPMARRAWLEHGHSCFLCEIVIRS
QFHTTYEPEAMKMDKKTIVWFRRDLRIEDNPALAAAAHEGSVFPVFIWCPEEEGQFYPGRASRWWMKQSLAHL
SQSLKALGSDLTLIKTHNTISAILDCIRVTGATKVVFNHLYDPVSLVRDHTVKEKLVERGISVQSYNGDLLYEPWEIYCE
KGKPFTSFNSYWKKCLDMSIESVMLPPPWRLMPITAAAEAIWACSIEELGLENEAEKPSNALLTRAWSPGWSNAD
KLLNEFIEKQLIDYAKNSKKVVGNSTSLLSPYLHFGEISVRHVFQCARMKQIIWARDKNSEGEESADLFLRGIGLREYS
RYICFNFPFTHEQSLLSHLRFFPWDADVDKFKAWRQGRTGYPLVDAGMRELWATGWMHNRIRVIVSSFAVKFLLL
PWKWGMKYFWDTLLDADLECDILGWQYISGSIPDGHELDRLDNPALQGAKYDPEGEYIRQWLPELARLPTEWIH
HPWDAPLTVLKASGVELGTNYAKPIVDIDTARELLAKAISRTREAQIMIGAA (SEQ ID NO: 103)
G3BP1 RRM Domain Deletion Mutation with Cry2PHR; G3BP1dRRM- Cry2PHR
MVMEKPSPLLVGREFVRQYYTLLNQAPDMLHRFYGKNSSYVHGGLDSNGKPADAVYGQKEIHRKVMSQNFTNC
HTKIRHVDAHATLNDGVVVQVMGLLSNNNQALRRFMQTFVLAPEGSVANKFYVHNDIFRYQDEVFGGFVTEPQE
ESEEEVEEPEERQQTPEVVPDDSGTFYDQAVVSNDMEEHLEEPVAEPEPDPEPEPEQEPVSEIQEEKPEPVLEETAP
EDAQKSSSPAPADIAQTVQEDLRTFSWASVTSKNLPPSGAVPVTGIPPHVVKVPASQPRPESKPESQIPPQRPQRD
QRVREQRINIPPQRGPRPIREAGEQGDIEPRRMVRHPDSHEKKTRAAREGDRRDNRLRGPGGPRGGLGGGMRG
PPRGGMVQKPGFGVGRGLAPRQKLIPMARRAWLEHGHSCFLCEIVIRSQFHTTYEPEAMKMDKKTIVWFRRDLRI
EDNPALAAAAHEGSVFPVFIWCPEEEGQFYPGRASRWWMKQSLAHLSQSLKALGSDLTLIKTHNTISAILDCIRVT
GATKVVFNHLYDPVSLVRDHTVKEKLVERGISVQSYNGDLLYEPWEIYCEKGKPFTSFNSYWKKCLDMSIESVMLPP
PWRLMPITAAAEAIWACSIEELGLENEAEKPSNALLTRAWSPGWSNADKLLNEFIEKQLIDYAKNSKKVVGNSTSLL
SPYLHFGEISVRHVFQCARMKQIIWARDKNSEGEESADLFLRGIGLREYSRYICFNFPFTHEQSLLSHLRFFPWDADV
DKFKAWRQGRTGYPLVDAGMRELWATGWMHNRIRVIVSSFAVKFLLLPWKWGMKYFWDTLLDADLECDILGW
QYISGSIPDGHELDRLDNPALQGAKYDPEGEYIRQWLPELARLPTEWIHHPWDAPLTVLKASGVELGTNYAKPIVDI
DTARELLAKAISRTREAQIMIGAA (SEQ ID NO: 104)
G3BP1 RGG Domain Deletion Mutation with Cry2PHR; G3BP1dRGG-Cry2PHR
MVMEKPSPLLVGREFVRQYYTLLNQAPDMLHRFYGKNSSYVHGGLDSNGKPADAVYGQKEIHRKVMSQNFTNC
HTKIRHVDAHATLNDGVVVQVMGLLSNNNQALRRFMQTFVLAPEGSVANKFYVHNDIFRYQDEVFGGFVTEPQE
ESEEEVEEPEERQQTPEVVPDDSGTFYDQAVVSNDMEEHLEEPVAEPEPDPEPEPEQEPVSEIQEEKPEPVLEETAP
EDAQKSSSPAPADIAQTVQEDLRTFSWASVTSKNLPPSGAVPVTGIPPHVVKVPASQPRPESKPESQIPPQRPQRD
QRVREQRINIPPQRGPRPIREAGEQGDIEPRRMVRHPDSHQLFIGNLPHEVDKSELKDFFQSYGNVVELRINSGGKL
PNFGFVVFDDSEPVQKVLSNRPIMFRGEVRLNVEMKMDKKTIVWFRRDLRIEDNPALAAAAHEGSVFPVFIWCPE
EEGQFYPGRASRWWMKQSLAHLSQSLKALGSDLTLIKTHNTISAILDCIRVTGATKVVFNHLYDPVSLVRDHTVKEK
LVERGISVQSYNGDLLYEPWEIYCEKGKPFTSFNSYWKKCLDMSIESVMLPPPWRLMPITAAAEAIWACSIEELGLE
NEAEKPSNALLTRAWSPGWSNADKLLNEFIEKQLIDYAKNSKKVVGNSTSLLSPYLHFGEISVRHVFQCARMKQIIW
ARDKNSEGEESADLFLRGIGLREYSRYICFNFPFTHEQSLLSHLRFFPWDADVDKFKAWRQGRTGYPLVDAGMREL
WATGWMHNRIRVIVSSFAVKFLLLPWKWGMKYFWDTLLDADLECDILGWQYISGSIPDGHELDRLDNPALQGAK
YDPEGEYIRQWLPELARLPTEWIHHPWDAPLTVLKASGVELGTNYAKPIVDIDTARELLAKAISRTREAQIMIGAA
(SEQ ID NO: 105)
G3BP1 RRMRGG Domain Deletion Mutation with Cry2PHR; G3BP1dRRMRGG-Cry2PHR
MVMEKPSPLLVGREFVRQYYTLLNQAPDMLHRFYGKNSSYVHGGLDSNGKPADAVYGQKEIHRKVMSQNFTNC
HTKIRHVDAHATLNDGVVVQVMGLLSNNNQALRRFMQTFVLAPEGSVANKFYVHNDIFRYQDEVFGGFVTEPQE
ESEEEVEEPEERQQTPEVVPDDSGTFYDQAVVSNDMEEHLEEPVAEPEPDPEPEPEQEPVSEIQEEKPEPVLEETAP
EDAQKSSSPAPADIAQTVQEDLRTFSWASVTSKNLPPSGAVPVTGIPPHVVKVPASQPRPESKPESQIPPQRPQRD
QRVREQRINIPPQRGPRPIREAGEQGDIEPRRMVRHPDSHMKMDKKTIVWFRRDLRIEDNPALAAAAHEGSVFP
VFIWCPEEEGQFYPGRASRWWMKQSLAHLSQSLKALGSDLTLIKTHNTISAILDCIRVTGATKVVFNHLYDPVSLVR
DHTVKEKLVERGISVQSYNGDLLYEPWEIYCEKGKPFTSFNSYWKKCLDMSIESVMLPPPWRLMPITAAAEAIWAC
SIEELGLENEAEKPSNALLTRAWSPGWSNADKLLNEFIEKQLIDYAKNSKKVVGNSTSLLSPYLHFGEISVRHVFQCA
RMKQIIWARDKNSEGEESADLFLRGIGLREYSRYICFNFPFTHEQSLLSHLRFFPWDADVDKFKAWRQGRTGYPLV
DAGMRELWATGWMHNRIRVIVSSFAVKFLLLPWKWGMKYFWDTLLDADLECDILGWQYISGSIPDGHELDRLD
NPALQGAKYDPEGEYIRQWLPELARLPTEWIHHPWDAPLTVLKASGVELGTNYAKPIVDIDTARELLAKAISRTREA
QIMIGAA (SEQ ID NO: 106)
AsLOV2
GEFLATTLERIEKNFVITDP RLPDNPIIFASDSFLQLTEY SREEILGRNCRFLQGPETDR
ATVRKIRDAIDNQTEVTVQL INYTKSGKKFWNLFHLQPMR DQKGDVQYFIGVQLDGTEHV
RDAAEREGVMLIKKTAENID EAAKELPDANLRPEDLWANH G (SEQ ID NO: 107)
EL222
MLDMGQDRPI DGSGAPGADD TRVEVQPPAQ WVLDLIEASP IASVVSDPRL ADNPLIAINQ AFTDLTGYSE
EECVGRNCRF LAGSGTEPWL TDKIRQGVRE HKPVLVEILN YKKDGTPFRN AVLVAPIYDD DDELLYFLGS
QVEVDDDQPN MGMARRERAA EMLKTLSPRQ LEVTTLVASG LRNKEVAARL GLSEKTVKMH RGLVMEKLNL
KTSADLVRIA VEAGI (SEQ ID NO: 108)
Ytva
MASFQSFGIP GQLEVIKKAL DHVRVGVVIT DPALEDNPIV YVNQGFVQMT GYETEEILGK NCRFLQGKHT
DPAEVDNIRT ALQNKEPVTV QIQNYKKDGT MFWNELNIDP MEIEDKTYFV GIQNDITKQK
EYEKLLEDSL TEITALSTPI VPIRNGISAL PLVGNLTEER FNSIVCTLTN ILSTSKDDYL IIDLSGLAQV NEQTADQIFK
LSHLLKLTGT ELIITGIKPE LAMKMNKLDA NFSSLKTYSN VKDAVKVLPI M (SEQ ID NO: 109)
RsLOV
MDQKQFEKIRAVFDRSGVALTLVDMSLPEQPVVLANPPFLRMTGYTEGQILGFNCRFLQRGDENAQARAD
IRDALKLGRELQVVLRNYRANDEPFDNLLFLHPVGGRPDAPDYFLGSQFELGRSGNSEEAAAAGHAGALT
GELARIGTVAARLEMDSRRHLAQAAAALVRAWERRG (SEQ ID NO: 110)

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will appreciate that numerous changes and modifications can be made to the preferred embodiments of the invention and that such changes and modifications can be made without departing from the spirit of the invention. It is, therefore, intended that the appended claims cover all such equivalent variations as fall within the true spirit and scope of the invention.

1. A nucleotide sequence encoding a chimeric polypeptide, comprising: a first nucleotide sequence encoding a light-induced oligomerization domain and a second nucleotide sequence encoding a low complexity domain from a membraneless organelle target protein.

2. The nucleotide sequence of claim 1, wherein the light-induced oligomerization domain is selected from the group consisting of CRY 2 PHR, CRY 20LIG, NcVVD, NcVVDY50W, NcVVDY50W/I74V/I85V/LINKERA, NcVVDY50W/I52C/I74V/I85V/LINKERA, NcVVDY50W/C71V/I74V/I85V/LINKERA, NcVVDY50W/I52C/C71V/I74V/I85V/LINKERA, NcVVDY50W/I74V/I85V/LINKERB, NcVVDY50W/I52C/I74V/I85V/LINKERB, NcVVDY50W/C71V/I74V/I85V/LINKERB, NcVVDY50W/I52C/C71V/I74V/I85V/LINKERB, NcLOV, and VfAU1LOV.

3. The nucleotide sequence of claim 2, wherein the light-induced oligomerization domain is NcVVD.

4. The nucleotide sequence of claim 2, wherein the light-induced oligomerization domain is NcVVDY50W.

5. The nucleotide sequence of claim 2, wherein the light-induced oligomerization domain is NcVVDY50W/I74V/I85V/LINKERA.

6. The nucleotide sequence of claim 2, wherein the light-induced oligomerization domain is NcVVDY50W/I52/I74V/I85V/LINKERA.

7. The nucleotide sequence of claim 2, wherein the light-induced oligomerization domain is NcVVDY50W/C71V/I74V/I85V/LINKERA.

8. The nucleotide sequence of claim 2, wherein the light-induced oligomerization domain is NcVVDY50W/I52C/C71V/I74V/I85V/LINKERA.

9. The nucleotide sequence of claim 2, wherein the light-induced oligomerization domain is NcVVDY50W/I74V/I85V/LINKERB.

10. The nucleotide sequence of claim 2, wherein the light-induced oligomerization domain is NcVVDY50W/I52/I74V/I85V/LINKERB.

11. The nucleotide sequence of claim 2, wherein the light-induced oligomerization domain is NcVVDY50W/C71V/I74V/I85V/LINKERB.

12. The nucleotide sequence of claim 2, wherein the light-induced oligomerization domain is NcVVDY50W/I52C/C71V/I74V/I85V/LINKERB.

13. The nucleotide sequence of claim 2, wherein the light-induced oligomerization domain is CRY 2 PHR.

14. The nucleotide sequence of claim 2, wherein the light-induced oligomerization domain is CRY 20LIG.

15. The nucleotide sequence of claim 1, wherein the light-induced oligomerization domain is a LOV domain.

16. The nucleotide sequence of claim 1, wherein the light-induced oligomerization domain is a PHR domain.

17. The nucleotide sequence of claim 1, wherein the low complexity domain from a membraneless organelle target protein is selected from the group consisting of PABC1, TIAR, G3BP1, G3BP2, DDX6, TDRD3, and ATXN2.

18. The nucleotide sequence of claim 17, wherein the low complexity domain from a membraneless organelle target protein is G 3B P1.

19.-31. (canceled)

32. An expression vector comprising the nucleotide sequence of claim 1.

33. (canceled)

34. A cell comprising the nucleotide sequence of claim 1.

35.-99. (canceled)


SEQUENCE LISTING (110 sequences)

SEQ ID NO: 1 - protein, 498 aa, Arabidopsis sp.
MKMDKKTIVWFRRDLRIEDNPALAAAAHEGSVFPVFIWCPEEEGQFYPGRASRWWMKQSLAHLSQSLKALGSDLTLIKTHNTISAILDCIRVTGATKVVFNHLYDPVSLVRDHTVKEKLVERGISVQSYNGDLLYEPWEIYCEKGKPFTSFNSYWKKCLDMSIESVMLPPPWRLMPITAAAEAIWACSIEELGLENEAEKPSNALLTRAWSPGWSNADKLLNEFIEKQLIDYAKNSKKVVGNSTSLLSPYLHFGEISVRHVFQCARMKQIIWARDKNSEGEESADLFLRGIGLREYSRYICFNFPFTHEQSLLSHLRFFPWDADVDKFKAWRQGRTGYPLVDAGMRELWATGWMHNRIRVIVSSFAVKFLLLPWKWGMKYFWDTLLDADLECDILGWQYISGSIPDGHELDRLDNPALQGAKYDPEGEYIRQWLPELARLPTEWIHHPWDAPLTVLKASGVELGTNYAKPIVDIDTARELLAKAISRTREAQIMIGAA

SEQ ID NO: 2 - protein, 498 aa, Arabidopsis sp.
MKMDKKTIVWFRRDLRIEDNPALAAAAHEGSVFPVFIWCPEEEGQFYPGRASRWWMKQSLAHLSQSLKALGSDLTLIKTHNTISAILDCIRVTGATKVVFNHLYDPVSLVRDHTVKEKLVERGISVQSYNGDLLYEPWEIYCEKGKPFTSFNSYWKKCLDMSIESVMLPPPWRLMPITAAAEAIWACSIEELGLENEAEKPSNALLTRAWSPGWSNADKLLNEFIEKQLIDYAKNSKKVVGNSTSLLSPYLHFGEISVRHVFQCARMKQIIWARDKNSEGEESADLFLRGIGLREYSRYICFNFPFTHEQSLLSHLRFFPWDADVDKFKAWRQGRTGYPLVDAGMRELWATGWMHNRIRVIVSSFAVKFLLLPWKWGMKYFWDTLLDADLECDILGWQYISGSIPDGHELDRLDNPALQGAKYDPEGEYIRQWLPELARLPTEWIHHPWDAPLTVLKASGVELGTNYAKPIVDIDTARELLAKAISRTRGAQIMIGAA

SEQ ID NO: 3 - protein, 119 aa, Arabidopsis sp.
MTSCALILCDLKQKDTPIVYASEAFLYMTGYSNAEVLGRNCRFLQSPDGMVKPKSTRKYVDSNTINTMRKAIDRNAEVQVEVVNFKKNGQRFVNFLTMIPVRDETGEYRYSMGFQCETE

SEQ ID NO: 4 - protein, 151 aa, Arabidopsis sp.
MHTLYAPGGYDIMGYLIQIMNRPNPQVELGPVDTSCALILCDLKQKDTPIVYASEAFLYMTGYSNAEVLGRNCRFLQSPDGMVKPKSTRKYVDSNTINTMRKAIDRNAEVQVEVVNFKKNGQRFVNFLTMIPVRDETGEYRYSMGFQCETE

SEQ ID NO: 5 - protein, 151 aa, Arabidopsis sp.
MHTLYAPGGYDIMGWLIQIMNRPNPQVELGPVDTSCALILCDLKQKDTPIVYASEAFLYMTGYSNAEVLGRNCRFLQSPDGMVKPKSTRKYVDSNTINTMRKAIDRNAEVQVEVVNFKKNGQRFVNFLTMIPVRDETGEYRYSMGFQCETE

SEQ ID NO: 6 - protein, 348 aa, Arabidopsis sp.
MNGLTPPLMFCSRSDDPSSTSNINLDDVFADVFFNSNGELLDIDEIDDFGDNTCPKSSMSVDDDASSQVFQGHLFGNALSSIALSDSGDLSTGIYESQGNASRGKSLRTKSSGSISSELTEAQKVERRERNREHAKRSRVRKKFLLESLQQSVNELNHENNCLKESIREHLGPRGDSLIAQCSPEADTLLTDNPSKANRILEDPDYSLVKALQMAQQNFVITDASLPDNPIVYASRGFLTLTGYSLDQILGRNCRFLQGPETDPRAVDKIRNAITKGVDTSVCLLNYRQDGTTFWNLFFVAGLRDSKGNIVNYVGVQSKVSEDYAKLLVNEQNIEYKGVRTSNMLRRK

SEQ ID NO: 7 - protein, 145 aa, Arabidopsis sp.
PDYSLVKALQMAQQNFVITDASLPDNPIVYASRGFLTLTGYSLDQILGRNCRFLQGPETDPRAVDKIRNAITKGVDTSVCLLNYRQDGTTFWNLFFVAGLRDSKGNIVNYVGVQSKVSEDYAKLLVNEQNIEYKGVRTSNMLRRK

SEQ ID NO: 8 - DNA, 435 bp, Arabidopsis sp.
  source 1..435: mol_type = genomic DNA
cctgactacagtctcgtgaaggctctgcaaatggcacaacagaattttgtcattacagacgcctccctcccagacaaccctatcgtctacgccagtagagggtttctgacactgacaggctattctctcgaccagatcctgggcaggaactgcaggtttctgcaagggccagaaacagacccaagagctgtggataagatcaggaatgccatcaccaaaggcgttgataccagtgtctgtctgctgaattatagacaggatggcacaaccttctggaatctcttcttcgtggctggactcagagattctaagggcaatattgtcaactacgtcggagtgcagtcaaaggtgagcgaagattatgccaagctgctggtcaacgagcagaacattgagtacaaaggtgtgcgcaccagtaacatgctgcgcagaaag

SEQ ID NO: 9 - protein, 653 aa, synthetic construct
  REGION 1..653: note = Synthetic construct
MHTLYAPGGYDIMGWLIQIMNRPNPQVELGPVDTSCALILCDLKQKDTPIVYASEAFLYMTGYSNAEVLGRNCRFLQSPDGMVKPKSTRKYVDSNTINTMRKAIDRNAEVQVEVVNFKKNGQRFVNFLTMIPVRDETGEYRYSMGFQCETEMVMEKPSPLLVGREFVRQYYTLLNQAPDMLHRFYGKNSSYVHGGLDSNGKPADAVYGQKEIHRKVMSQNFTNCHTKIRHVDAHATLNDGVVVQVMGLLSNNNQALRRFMQTFVLAPEGSVANKFYVHNDIFRYQDEVFGGFVTEPQEESEEEVEEPEERQQTPEVVPDDSGTFYDQAVVSNDMEEHLEEPVAEPEPDPEPEPEQEPVSEIQEEKPEPVLEETAPEDAQKSSSPAPADIAQTVQEDLRTFSWASVTSKNLPPSGAVPVTGIPPHVVKVPASQPRPESKPESQIPPQRPQRDQRVREQRINIPPQRGPRPIREAGEQGDIEPRRMVRHPDSHQLFIGNLPHEVDKSELKDFFQSYGNVVELRINSGGKLPNFGFVVFDDSEPVQKVLSNRPIMFRGEVRLNVEEKKTRAAREGDRRDNRLRGPGGPRGGLGGGMRGPPRGGMVQKPGFGVGRGLAPRQKLIPMARRAWLEHGHSCFLCEIVIRSQFHTTYEPEA

SEQ ID NO: 10 - protein, 515 aa, synthetic construct
  REGION 1..515: note = Synthetic construct
MHTLYAPGGYDIMGWLIQIMNRPNPQVELGPVDTSCALILCDLKQKDTPIVYASEAFLYMTGYSNAEVLGRNCRFLQSPDGMVKPKSTRKYVDSNTINTMRKAIDRNAEVQVEVVNFKKNGQRFVNFLTMIPVRDETGEYRYSMGFQCETEMGFVTEPQEESEEEVEEPEERQQTPEVVPDDSGTFYDQAVVSNDMEEHLEEPVAEPEPDPEPEPEQEPVSEIQEEKPEPVLEETAPEDAQKSSSPAPADIAQTVQEDLRTFSWASVTSKNLPPSGAVPVTGIPPHVVKVPASQPRPESKPESQIPPQRPQRDQRVREQRINIPPQRGPRPIREAGEQGDIEPRRMVRHPDSHQLFIGNLPHEVDKSELKDFFQSYGNVVELRINSGGKLPNFGFVVFDDSEPVQKVLSNRPIMFRGEVRLNVEEKKTRAAREGDRRDNRLRGPGGPRGGLGGGMRGPPRGGMVQKPGFGVGRGLAPRQKLIPMARRAWLEHGHSCFLCEIVIRSQFHTTYEPEA

SEQ ID NO: 11 - protein, 571 aa, synthetic construct
  REGION 1..571: note = Synthetic construct
MHTLYAPGGYDIMGWLIQIMNRPNPQVELGPVDTSCALILCDLKQKDTPIVYASEAFLYMTGYSNAEVLGRNCRFLQSPDGMVKPKSTRKYVDSNTINTMRKAIDRNAEVQVEVVNFKKNGQRFVNFLTMIPVRDETGEYRYSMGFQCETEMVMEKPSPLLVGREFVRQYYTLLNQAPDMLHRFYGKNSSYVHGGLDSNGKPADAVYGQKEIHRKVMSQNFTNCHTKIRHVDAHATLNDGVVVQVMGLLSNNNQALRRFMQTFVLAPEGSVANKFYVHNDIFRYQDEVFGTAPEDAQKSSSPAPADIAQTVQEDLRTFSWASVTSKNLPPSGAVPVTGIPPHVVKVPASQPRPESKPESQIPPQRPQRDQRVREQRINIPPQRGPRPIREAGEQGDIEPRRMVRHPDSHQLFIGNLPHEVDKSELKDFFQSYGNVVELRINSGGKLPNFGFVVFDDSEPVQKVLSNRPIMFRGEVRLNVEEKKTRAAREGDRRDNRLRGPGGPRGGLGGGMRGPPRGGMVQKPGFGVGRGLAPRQKLIPMARRAWLEHGHSCFLCEIVIRSQFHTTYEPEA

SEQ ID NO: 12 - protein, 534 aa, synthetic construct
  REGION 1..534: note = Synthetic construct
MHTLYAPGGYDIMGWLIQIMNRPNPQVELGPVDTSCALILCDLKQKDTPIVYASEAFLYMTGYSNAEVLGRNCRFLQSPDGMVKPKSTRKYVDSNTINTMRKAIDRNAEVQVEVVNFKKNGQRFVNFLTMIPVRDETGEYRYSMGFQCETEMVMEKPSPLLVGREFVRQYYTLLNQAPDMLHRFYGKNSSYVHGGLDSNGKPADAVYGQKEIHRKVMSQNFTNCHTKIRHVDAHATLNDGVVVQVMGLLSNNNQALRRFMQTFVLAPEGSVANKFYVHNDIFRYQDEVFGGFVTEPQEESEEEVEEPEERQQTPEVVPDDSGTFYDQAVVSNDMEEHLEEPVAEPEPDPEPEPEQEPVSEIQEEKPEPVLEEQLFIGNLPHEVDKSELKDFFQSYGNVVELRINSGGKLPNFGFVVFDDSEPVQKVLSNRPIMFRGEVRLNVEEKKTRAAREGDRRDNRLRGPGGPRGGLGGGMRGPPRGGMVQKPGFGVGRGLAPRQKLIPMARRAWLEHGHSCFLCEIVIRSQFHTTYEPEA

SEQ ID NO: 13 - protein, 582 aa, synthetic construct
  REGION 1..582: note = Synthetic construct
MHTLYAPGGYDIMGWLIQIMNRPNPQVELGPVDTSCALILCDLKQKDTPIVYASEAFLYMTGYSNAEVLGRNCRFLQSPDGMVKPKSTRKYVDSNTINTMRKAIDRNAEVQVEVVNFKKNGQRFVNFLTMIPVRDETGEYRYSMGFQCETEMVMEKPSPLLVGREFVRQYYTLLNQAPDMLHRFYGKNSSYVHGGLDSNGKPADAVYGQKEIHRKVMSQNFTNCHTKIRHVDAHATLNDGVVVQVMGLLSNNNQALRRFMQTFVLAPEGSVANKFYVHNDIFRYQDEVFGGFVTEPQEESEEEVEEPEERQQTPEVVPDDSGTFYDQAVVSNDMEEHLEEPVAEPEPDPEPEPEQEPVSEIQEEKPEPVLEETAPEDAQKSSSPAPADIAQTVQEDLRTFSWASVTSKNLPPSGAVPVTGIPPHVVKVPASQPRPESKPESQIPPQRPQRDQRVREQRINIPPQRGPRPIREAGEQGDIEPRRMVRHPDSHEKKTRAAREGDRRDNRLRGPGGPRGGLGGGMRGPPRGGMVQKPGFGVGRGLAPRQKLIPMARRAWLEHGHSCFLCEIVIRSQFHTTYEPEA

SEQ ID NO: 14 - protein, 562 aa, synthetic construct
  REGION 1..562: note = Synthetic construct
MHTLYAPGGYDIMGWLIQIMNRPNPQVELGPVDTSCALILCDLKQKDTPIVYASEAFLYMTGYSNAEVLGRNCRFLQSPDGMVKPKSTRKYVDSNTINTMRKAIDRNAEVQVEVVNFKKNGQRFVNFLTMIPVRDETGEYRYSMGFQCETEMVMEKPSPLLVGREFVRQYYTLLNQAPDMLHRFYGKNSSYVHGGLDSNGKPADAVYGQKEIHRKVMSQNFTNCHTKIRHVDAHATLNDGVVVQVMGLLSNNNQALRRFMQTFVLAPEGSVANKFYVHNDIFRYQDEVFGGFVTEPQEESEEEVEEPEERQQTPEVVPDDSGTFYDQAVVSNDMEEHLEEPVAEPEPDPEPEPEQEPVSEIQEEKPEPVLEETAPEDAQKSSSPAPADIAQTVQEDLRTFSWASVTSKNLPPSGAVPVTGIPPHVVKVPASQPRPESKPESQIPPQRPQRDQRVREQRINIPPQRGPRPIREAGEQGDIEPRRMVRHPDSHQLFIGNLPHEVDKSELKDFFQSYGNVVELRINSGGKLPNFGFVVFDDSEPVQKVLSNRPIMFRGEVRLNVE

SEQ ID NO: 15 - protein, 491 aa, synthetic construct
  REGION 1..491: note = Synthetic construct
MHTLYAPGGYDIMGWLIQIMNRPNPQVELGPVDTSCALILCDLKQKDTPIVYASEAFLYMTGYSNAEVLGRNCRFLQSPDGMVKPKSTRKYVDSNTINTMRKAIDRNAEVQVEVVNFKKNGQRFVNFLTMIPVRDETGEYRYSMGFQCETEMVMEKPSPLLVGREFVRQYYTLLNQAPDMLHRFYGKNSSYVHGGLDSNGKPADAVYGQKEIHRKVMSQNFTNCHTKIRHVDAHATLNDGVVVQVMGLLSNNNQALRRFMQTFVLAPEGSVANKFYVHNDIFRYQDEVFGGFVTEPQEESEEEVEEPEERQQTPEVVPDDSGTFYDQAVVSNDMEEHLEEPVAEPEPDPEPEPEQEPVSEIQEEKPEPVLEETAPEDAQKSSSPAPADIAQTVQEDLRTFSWASVTSKNLPPSGAVPVTGIPPHVVKVPASQPRPESKPESQIPPQRPQRDQRVREQRINIPPQRGPRPIREAGEQGDIEPRRMVRHPDSH

SEQ ID NO: 16 - protein, 653 aa, synthetic construct
  REGION 1..653: note = Synthetic construct
MVMEKPSPLLVGREFVRQYYTLLNQAPDMLHRFYGKNSSYVHGGLDSNGKPADAVYGQKEIHRKVMSQNFTNCHTKIRHVDAHATLNDGVVVQVMGLLSNNNQALRRFMQTFVLAPEGSVANKFYVHNDIFRYQDEVFGGFVTEPQEESEEEVEEPEERQQTPEVVPDDSGTFYDQAVVSNDMEEHLEEPVAEPEPDPEPEPEQEPVSEIQEEKPEPVLEETAPEDAQKSSSPAPADIAQTVQEDLRTFSWASVTSKNLPPSGAVPVTGIPPHVVKVPASQPRPESKPESQIPPQRPQRDQRVREQRINIPPQRGPRPIREAGEQGDIEPRRMVRHPDSHQLFIGNLPHEVDKSELKDFFQSYGNVVELRINSGGKLPNFGFVVFDDSEPVQKVLSNRPIMFRGEVRLNVEEKKTRAAREGDRRDNRLRGPGGPRGGLGGGMRGPPRGGMVQKPGFGVGRGLAPRQKLIPMARRAWLEHGHSCFLCEIVIRSQFHTTYEPEAMHTLYAPGGYDIMGWLIQIMNRPNPQVELGPVDTSCALILCDLKQKDTPIVYASEAFLYMTGYSNAEVLGRNCRFLQSPDGMVKPKSTRKYVDSNTINTMRKAIDRNAEVQVEVVNFKKNGQRFVNFLTMIPVRDETGEYRYSMGFQCETE

SEQ ID NO: 17 - protein, 515 aa, synthetic construct
  REGION 1..515: note = Synthetic construct
MGFVTEPQEESEEEVEEPEERQQTPEVVPDDSGTFYDQAVVSNDMEEHLEEPVAEPEPDPEPEPEQEPVSEIQEEKPEPVLEETAPEDAQKSSSPAPADIAQTVQEDLRTFSWASVTSKNLPPSGAVPVTGIPPHVVKVPASQPRPESKPESQIPPQRPQRDQRVREQRINIPPQRGPRPIREAGEQGDIEPRRMVRHPDSHQLFIGNLPHEVDKSELKDFFQSYGNVVELRINSGGKLPNFGFVVFDDSEPVQKVLSNRPIMFRGEVRLNVEEKKTRAAREGDRRDNRLRGPGGPRGGLGGGMRGPPRGGMVQKPGFGVGRGLAPRQKLIPMARRAWLEHGHSCFLCEIVIRSQFHTTYEPEAMHTLYAPGGYDIMGWLIQIMNRPNPQVELGPVDTSCALILCDLKQKDTPIVYASEAFLYMTGYSNAEVLGRNCRFLQSPDGMVKPKSTRKYVDSNTINTMRKAIDRNAEVQVEVVNFKKNGQRFVNFLTMIPVRDETGEYRYSMGFQCETE

SEQ ID NO: 18 - protein, 571 aa, synthetic construct
  REGION 1..571: note = Synthetic construct
MVMEKPSPLLVGREFVRQYYTLLNQAPDMLHRFYGKNSSYVHGGLDSNGKPADAVYGQKEIHRKVMSQNFTNCHTKIRHVDAHATLNDGVVVQVMGLLSNNNQALRRFMQTFVLAPEGSVANKFYVHNDIFRYQDEVFGTAPEDAQKSSSPAPADIAQTVQEDLRTFSWASVTSKNLPPSGAVPVTGIPPHVVKVPASQPRPESKPESQIPPQRPQRDQRVREQRINIPPQRGPRPIREAGEQGDIEPRRMVRHPDSHQLFIGNLPHEVDKSELKDFFQSYGNVVELRINSGGKLPNFGFVVFDDSEPVQKVLSNRPIMFRGEVRLNVEEKKTRAAREGDRRDNRLRGPGGPRGGLGGGMRGPPRGGMVQKPGFGVGRGLAPRQKLIPMARRAWLEHGHSCFLCEIVIRSQFHTTYEPEAMHTLYAPGGYDIMGWLIQIMNRPNPQVELGPVDTSCALILCDLKQKDTPIVYASEAFLYMTGYSNAEVLGRNCRFLQSPDGMVKPKSTRKYVDSNTINTMRKAIDRNAEVQVEVVNFKKNGQRFVNFLTMIPVRDETGEYRYSMGFQCETE

SEQ ID NO: 19 - protein, 534 aa, synthetic construct
  REGION 1..534: note = Synthetic construct
MVMEKPSPLLVGREFVRQYYTLLNQAPDMLHRFYGKNSSYVHGGLDSNGKPADAVYGQKEIHRKVMSQNFTNCHTKIRHVDAHATLNDGVVVQVMGLLSNNNQALRRFMQTFVLAPEGSVANKFYVHNDIFRYQDEVFGGFVTEPQEESEEEVEEPEERQQTPEVVPDDSGTFYDQAVVSNDMEEHLEEPVAEPEPDPEPEPEQEPVSEIQEEKPEPVLEEQLFIGNLPHEVDKSELKDFFQSYGNVVELRINSGGKLPNFGFVVFDDSEPVQKVLSNRPIMFRGEVRLNVEEKKTRAAREGDRRDNRLRGPGGPRGGLGGGMRGPPRGGMVQKPGFGVGRGLAPRQKLIPMARRAWLEHGHSCFLCEIVIRSQFHTTYEPEAMHTLYAPGGYDIMGWLIQIMNRPNPQVELGPVDTSCALILCDLKQKDTPIVYASEAFLYMTGYSNAEVLGRNCRFLQSPDGMVKPKSTRKYVDSNTINTMRKAIDRNAEVQVEVVNFKKNGQRFVNFLTMIPVRDETGEYRYSMGFQCETE

SEQ ID NO: 20 - protein, 582 aa, synthetic construct
  REGION 1..582: note = Synthetic construct
MVMEKPSPLLVGREFVRQYYTLLNQAPDMLHRFYGKNSSYVHGGLDSNGKPADAVYGQKEIHRKVMSQNFTNCHTKIRHVDAHATLNDGVVVQVMGLLSNNNQALRRFMQTFVLAPEGSVANKFYVHNDIFRYQDEVFGGFVTEPQEESEEEVEEPEERQQTPEVVPDDSGTFYDQAVVSNDMEEHLEEPVAEPEPDPEPEPEQEPVSEIQEEKPEPVLEETAPEDAQKSSSPAPADIAQTVQEDLRTFSWASVTSKNLPPSGAVPVTGIPPHVVKVPASQPRPESKPESQIPPQRPQRDQRVREQRINIPPQRGPRPIREAGEQGDIEPRRMVRHPDSHEKKTRAAREGDRRDNRLRGPGGPRGGLGGGMRGPPRGGMVQKPGFGVGRGLAPRQKLIPMARRAWLEHGHSCFLCEIVIRSQFHTTYEPEAMHTLYAPGGYDIMGWLIQIMNRPNPQVELGPVDTSCALILCDLKQKDTPIVYASEAFLYMTGYSNAEVLGRNCRFLQSPDGMVKPKSTRKYVDSNTINTMRKAIDRNAEVQVEVVNFKKNGQRFVNFLTMIPVRDETGEYRYSMGFQCETE

SEQ ID NO: 21 - protein, 562 aa, synthetic construct
  REGION 1..562: note = Synthetic construct
MVMEKPSPLLVGREFVRQYYTLLNQAPDMLHRFYGKNSSYVHGGLDSNGKPADAVYGQKEIHRKVMSQNFTNCHTKIRHVDAHATLNDGVVVQVMGLLSNNNQALRRFMQTFVLAPEGSVANKFYVHNDIFRYQDEVFGGFVTEPQEESEEEVEEPEERQQTPEVVPDDSGTFYDQAVVSNDMEEHLEEPVAEPEPDPEPEPEQEPVSEIQEEKPEPVLEETAPEDAQKSSSPAPADIAQTVQEDLRTFSWASVTSKNLPPSGAVPVTGIPPHVVKVPASQPRPESKPESQIPPQRPQRDQRVREQRINIPPQRGPRPIREAGEQGDIEPRRMVRHPDSHQLFIGNLPHEVDKSELKDFFQSYGNVVELRINSGGKLPNFGFVVFDDSEPVQKVLSNRPIMFRGEVRLNVEMHTLYAPGGYDIMGWLIQIMNRPNPQVELGPVDTSCALILCDLKQKDTPIVYASEAFLYMTGYSNAEVLGRNCRFLQSPDGMVKPKSTRKYVDSNTINTMRKAIDRNAEVQVEVVNFKKNGQRFVNFLTMIPVRDETGEYRYSMGFQCETE

SEQ ID NO: 22 - protein, 491 aa, synthetic construct
  REGION 1..491: note = Synthetic construct
MVMEKPSPLLVGREFVRQYYTLLNQAPDMLHRFYGKNSSYVHGGLDSNGKPADAVYGQKEIHRKVMSQNFTNCHTKIRHVDAHATLNDGVVVQVMGLLSNNNQALRRFMQTFVLAPEGSVANKFYVHNDIFRYQDEVFGGFVTEPQEESEEEVEEPEERQQTPEVVPDDSGTFYDQAVVSNDMEEHLEEPVAEPEPDPEPEPEQEPVSEIQEEKPEPVLEETAPEDAQKSSSPAPADIAQTVQEDLRTFSWASVTSKNLPPSGAVPVTGIPPHVVKVPASQPRPESKPESQIPPQRPQRDQRVREQRINIPPQRGPRPIREAGEQGDIEPRRMVRHPDSHMHTLYAPGGYDIMGWLIQIMNRPNPQVELGPVDTSCALILCDLKQKDTPIVYASEAFLYMTGYSNAEVLGRNCRFLQSPDGMVKPKSTRKYVDSNTINTMRKAIDRNAEVQVEVVNFKKNGQRFVNFLTMIPVRDETGEYRYSMGFQCETE

SEQ ID NO: 23 - protein, 665 aa, synthetic construct
  REGION 1..665: note = Synthetic construct
MHTLYAPGGYDIMGWLIQIMNRPNPQVELGPVDTSCALVLCDLKQKDTPVVYASEAFLYMTGYSNAEVLGRNCRFLQSPDGMVKPKSTRKYVDSNTINTMRKAIDRNAEVQVEVVNFKKNGQRFVNFLTMIPVRDETGEYRYSMGFQCETEGSGGSGGSGGSGMVMEKPSPLLVGREFVRQYYTLLNQAPDMLHRFYGKNSSYVHGGLDSNGKPADAVYGQKEIHRKVMSQNFTNCHTKIRHVDAHATLNDGVVVQVMGLLSNNNQALRRFMQTFVLAPEGSVANKFYVHNDIFRYQDEVFGGFVTEPQEESEEEVEEPEERQQTPEVVPDDSGTFYDQAVVSNDMEEHLEEPVAEPEPDPEPEPEQEPVSEIQEEKPEPVLEETAPEDAQKSSSPAPADIAQTVQEDLRTFSWASVTSKNLPPSGAVPVTGIPPHVVKVPASQPRPESKPESQIPPQRPQRDQRVREQRINIPPQRGPRPIREAGEQGDIEPRRMVRHPDSHQLFIGNLPHEVDKSELKDFFQSYGNVVELRINSGGKLPNFGFVVFDDSEPVQKVLSNRPIMFRGEVRLNVEEKKTRAAREGDRRDNRLRGPGGPRGGLGGGMRGPPRGGMVQKPGFGVGRGLAPRQKLIPMARRAWLEHGHSCFLCEIVIRSQFHTTYEPEA

SEQ ID NO: 24 - protein, 527 aa, synthetic construct
  REGION 1..527: note = Synthetic construct
MHTLYAPGGYDIMGWLIQIMNRPNPQVELGPVDTSCALVLCDLKQKDTPVVYASEAFLYMTGYSNAEVLGRNCRFLQSPDGMVKPKSTRKYVDSNTINTMRKAIDRNAEVQVEVVNFKKNGQRFVNFLTMIPVRDETGEYRYSMGFQCETEGSGGSGGSGGSGMGFVTEPQEESEEEVEEPEERQQTPEVVPDDSGTFYDQAVVSNDMEEHLEEPVAEPEPDPEPEPEQEPVSEIQEEKPEPVLEETAPEDAQKSSSPAPADIAQTVQEDLRTFSWASVTSKNLPPSGAVPVTGIPPHVVKVPASQPRPESKPESQIPPQRPQRDQRVREQRINIPPQRGPRPIREAGEQGDIEPRRMVRHPDSHQLFIGNLPHEVDKSELKDFFQSYGNVVELRINSGGKLPNFGFVVFDDSEPVQKVLSNRPIMFRGEVRLNVEEKKTRAAREGDRRDNRLRGPGGPRGGLGGGMRGPPRGGMVQKPGFGVGRGLAPRQKLIPMARRAWLEHGHSCFLCEIVIRSQFHTTYEPEA

SEQ ID NO: 25 - protein, 583 aa, synthetic construct
  REGION 1..583: note = Synthetic construct
MHTLYAPGGYDIMGWLIQIMNRPNPQVELGPVDTSCALVLCDLKQKDTPVVYASEAFLYMTGYSNAEVLGRNCRFLQSPDGMVKPKSTRKYVDSNTINTMRKAIDRNAEVQVEVVNFKKNGQRFVNFLTMIPVRDETGEYRYSMGFQCETEGSGGSGGSGGSGMVMEKPSPLLVGREFVRQYYTLLNQAPDMLHRFYGKNSSYVHGGLDSNGKPADAVYGQKEIHRKVMSQNFTNCHTKIRHVDAHATLNDGVVVQVMGLLSNNNQALRRFMQTFVLAPEGSVANKFYVHNDIFRYQDEVFGTAPEDAQKSSSPAPADIAQTVQEDLRTFSWASVTSKNLPPSGAVPVTGIPPHVVKVPASQPRPESKPESQIPPQRPQRDQRVREQRINIPPQRGPRPIREAGEQGDIEPRRMVRHPDSHQLFIGNLPHEVDKSELKDFFQSYGNVVELRINSGGKLPNFGFVVFDDSEPVQKVLSNRPIMFRGEVRLNVEEKKTRAAREGDRRDNRLRGPGGPRGGLGGGMRGPPRGGMVQKPGFGVGRGLAPRQKLIPMARRAWLEHGHSCFLCEIVIRSQFHTTYEPEA

SEQ ID NO: 26 - protein, 546 aa, synthetic construct
  REGION 1..546: note = Synthetic construct
MHTLYAPGGYDIMGWLIQIMNRPNPQVELGPVDTSCALVLCDLKQKDTPVVYASEAFLYMTGYSNAEVLGRNCRFLQSPDGMVKPKSTRKYVDSNTINTMRKAIDRNAEVQVEVVNFKKNGQRFVNFLTMIPVRDETGEYRYSMGFQCETEGSGGSGGSGGSGMVMEKPSPLLVGREFVRQYYTLLNQAPDMLHRFYGKNSSYVHGGLDSNGKPADAVYGQKEIHRKVMSQNFTNCHTKIRHVDAHATLNDGVVVQVMGLLSNNNQALRRFMQTFVLAPEGSVANKFYVHNDIFRYQDEVFGGFVTEPQEESEEEVEEPEERQQTPEVVPDDSGTFYDQAVVSNDMEEHLEEPVAEPEPDPEPEPEQEPVSEIQEEKPEPVLEEQLFIGNLPHEVDKSELKDFFQSYGNVVELRINSGGKLPNFGFVVFDDSEPVQKVLSNRPIMFRGEVRLNVEEKKTRAAREGDRRDNRLRGPGGPRGGLGGGMRGPPRGGMVQKPGFGVGRGLAPRQKLIPMARRAWLEHGHSCFLCEIVIRSQFHTTYEPEA

SEQ ID NO: 27 - protein, 594 aa, synthetic construct
  REGION 1..594: note = Synthetic construct
MHTLYAPGGYDIMGWLIQIMNRPNPQVELGPVDTSCALVLCDLKQKDTPVVYASEAFLYMTGYSNAEVLGRNCRFLQSPDGMVKPKSTRKYVDSNTINTMRKAIDRNAEVQVEVVNFKKNGQRFVNFLTMIPVRDETGEYRYSMGFQCETEGSGGSGGSGGSGMVMEKPSPLLVGREFVRQYYTLLNQAPDMLHRFYGKNSSYVHGGLDSNGKPADAVYGQKEIHRKVMSQNFTNCHTKIRHVDAHATLNDGVVVQVMGLLSNNNQALRRFMQTFVLAPEGSVANKFYVHNDIFRYQDEVFGGFVTEPQEESEEEVEEPEERQQTPEVVPDDSGTFYDQAVVSNDMEEHLEEPVAEPEPDPEPEPEQEPVSEIQEEKPEPVLEETAPEDAQKSSSPAPADIAQTVQEDLRTFSWASVTSKNLPPSGAVPVTGIPPHVVKVPASQPRPESKPESQIPPQRPQRDQRVREQRINIPPQRGPRPIREAGEQGDIEPRRMVRHPDSHEKKTRAAREGDRRDNRLRGPGGPRGGLGGGMRGPPRGGMVQKPGFGVGRGLAPRQKLIPMARRAWLEHGHSCFLCEIVIRSQFHTTYEPEA

SEQ ID NO: 28 - protein, 574 aa, synthetic construct
  REGION 1..574: note = Synthetic construct
MHTLYAPGGYDIMGWLIQIMNRPNPQVELGPVDTSCALVLCDLKQKDTPVVYASEAFLYMTGYSNAEVLGRNCRFLQSPDGMVKPKSTRKYVDSNTINTMRKAIDRNAEVQVEVVNFKKNGQRFVNFLTMIPVRDETGEYRYSMGFQCETEGSGGSGGSGGSGMVMEKPSPLLVGREFVRQYYTLLNQAPDMLHRFYGKNSSYVHGGLDSNGKPADAVYGQKEIHRKVMSQNFTNCHTKIRHVDAHATLNDGVVVQVMGLLSNNNQALRRFMQTFVLAPEGSVANKFYVHNDIFRYQDEVFGGFVTEPQEESEEEVEEPEERQQTPEVVPDDSGTFYDQAVVSNDMEEHLEEPVAEPEPDPEPEPEQEPVSEIQEEKPEPVLEETAPEDAQKSSSPAPADIAQTVQEDLRTFSWASVTSKNLPPSGAVPVTGIPPHVVKVPASQPRPESKPESQIPPQRPQRDQRVREQRINIPPQRGPRPIREAGEQGDIEPRRMVRHPDSHQLFIGNLPHEVDKSELKDFFQSYGNVVELRINSGGKLPNFGFVVFDDSEPVQKVLSNRPIMFRGEVRLNVE

SEQ ID NO: 29 - protein, 503 aa, synthetic construct
  REGION 1..503: note = Synthetic construct
MHTLYAPGGYDIMGWLIQIMNRPNPQVELGPVDTSCALVLCDLKQKDTPVVYASEAFLYMTGYSNAEVLGRNCRFLQSPDGMVKPKSTRKYVDSNTINTMRKAIDRNAEVQVEVVNFKKNGQRFVNFLTMIPVRDETGEYRYSMGFQCETEGSGGSGGSGGSGMVMEKPSPLLVGREFVRQYYTLLNQAPDMLHRFYGKNSSYVHGGLDSNGKPADAVYGQKEIHRKVMSQNFTNCHTKIRHVDAHATLNDGVVVQVMGLLSNNNQALRRFMQTFVLAPEGSVANKFYVHNDIFRYQDEVFGGFVTEPQEESEEEVEEPEERQQTPEVVPDDSGTFYDQAVVSNDMEEHLEEPVAEPEPDPEPEPEQEPVSEIQEEKPEPVLEETAPEDAQKSSSPAPADIAQTVQEDLRTFSWASVTSKNLPPSGAVPVTGIPPHVVKVPASQPRPESKPESQIPPQRPQRDQRVREQRINIPPQRGPRPIREAGEQGDIEPRRMVRHPDSH

SEQ ID NO: 30 - protein, 665 aa, synthetic construct
  REGION 1..665: note = Synthetic construct
MHTLYAPGGYDIMGWLCQIMNRPNPQVELGPVDTSCALVLCDLKQKDTPVVYASEAFLYMTGYSNAEVLGRNCRFLQSPDGMVKPKSTRKYVDSNTINTMRKAIDRNAEVQVEVVNFKKNGQRFVNFLTMIPVRDETGEYRYSMGFQCETEGSGGSGGSGGSGMVMEKPSPLLVGREFVRQYYTLLNQAPDMLHRFYGKNSSYVHGGLDSNGKPADAVYGQKEIHRKVMSQNFTNCHTKIRHVDAHATLNDGVVVQVMGLLSNNNQALRRFMQTFVLAPEGSVANKFYVHNDIFRYQDEVFGGFVTEPQEESEEEVEEPEERQQTPEVVPDDSGTFYDQAVVSNDMEEHLEEPVAEPEPDPEPEPEQEPVSEIQEEKPEPVLEETAPEDAQKSSSPAPADIAQTVQEDLRTFSWASVTSKNLPPSGAVPVTGIPPHVVKVPASQPRPESKPESQIPPQRPQRDQRVREQRINIPPQRGPRPIREAGEQGDIEPRRMVRHPDSHQLFIGNLPHEVDKSELKDFFQSYGNVVELRINSGGKLPNFGFVVFDDSEPVQKVLSNRPIMFRGEVRLNVEEKKTRAAREGDRRDNRLRGPGGPRGGLGGGMRGPPRGGMVQKPGFGVGRGLAPRQKLIPMARRAWLEHGHSCFLCEIVIRSQFHTTYEPEA

SEQ ID NO: 31 - protein, 527 aa, synthetic construct
  REGION 1..527: note = Synthetic construct
MHTLYAPGGYDIMGWLCQIMNRPNPQVELGPVDTSCALVLCDLKQKDTPVVYASEAFLYMTGYSNAEVLGRNCRFLQSPDGMVKPKSTRKYVDSNTINTMRKAIDRNAEVQVEVVNFKKNGQRFVNFLTMIPVRDETGEYRYSMGFQCETEGSGGSGGSGGSGMGFVTEPQEESEEEVEEPEERQQTPEVVPDDSGTFYDQAVVSNDMEEHLEEPVAEPEPDPEPEPEQEPVSEIQEEKPEPVLEETAPEDAQKSSSPAPADIAQTVQEDLRTFSWASVTSKNLPPSGAVPVTGIPPHVVKVPASQPRPESKPESQIPPQRPQRDQRVREQRINIPPQRGPRPIREAGEQGDIEPRRMVRHPDSHQLFIGNLPHEVDKSELKDFFQSYGNVVELRINSGGKLPNFGFVVFDDSEPVQKVLSNRPIMFRGEVRLNVEEKKTRAAREGDRRDNRLRGPGGPRGGLGGGMRGPPRGGMVQKPGFGVGRGLAPRQKLIPMARRAWLEHGHSCFLCEIVIRSQFHTTYEPEA

SEQ ID NO: 32 - protein, 583 aa, synthetic construct
  REGION 1..583: note = Synthetic construct
MHTLYAPGGYDIMGWLCQIMNRPNPQVELGPVDTSCALVLCDLKQKDTPVVYASEAFLYMTGYSNAEVLGRNCRFLQSPDGMVKPKSTRKYVDSNTINTMRKAIDRNAEVQVEVVNFKKNGQRFVNFLTMIPVRDETGEYRYSMGFQCETEGSGGSGGSGGSGMVMEKPSPLLVGREFVRQYYTLLNQAPDMLHRFYGKNSSYVHGGLDSNGKPADAVYGQKEIHRKVMSQNFTNCHTKIRHVDAHATLNDGVVVQVMGLLSNNNQALRRFMQTFVLAPEGSVANKFYVHNDIFRYQDEVFGTAPEDAQKSSSPAPADIAQTVQEDLRTFSWASVTSKNLPPSGAVPVTGIPPHVVKVPASQPRPESKPESQIPPQRPQRDQRVREQRINIPPQRGPRPIREAGEQGDIEPRRMVRHPDSHQLFIGNLPHEVDKSELKDFFQSYGNVVELRINSGGKLPNFGFVVFDDSEPVQKVLSNRPIMFRGEVRLNVEEKKTRAAREGDRRDNRLRGPGGPRGGLGGGMRGPPRGGMVQKPGFGVGRGLAPRQKLIPMARRAWLEHGHSCFLCEIVIRSQFHTTYEPEA

SEQ ID NO: 33 - protein, 546 aa, synthetic construct
  REGION 1..546: note = Synthetic construct
MHTLYAPGGYDIMGWLCQIMNRPNPQVELGPVDTSCALVLCDLKQKDTPVVYASEAFLYMTGYSNAEVLGRNCRFLQSPDGMVKPKSTRKYVDSNTINTMRKAIDRNAEVQVEVVNFKKNGQRFVNFLTMIPVRDETGEYRYSMGFQCETEGSGGSGGSGGSGMVMEKPSPLLVGREFVRQYYTLLNQAPDMLHRFYGKNSSYVHGGLDSNGKPADAVYGQKEIHRKVMSQNFTNCHTKIRHVDAHATLNDGVVVQVMGLLSNNNQALRRFMQTFVLAPEGSVANKFYVHNDIFRYQDEVFGGFVTEPQEESEEEVEEPEERQQTPEVVPDDSGTFYDQAVVSNDMEEHLEEPVAEPEPDPEPEPEQEPVSEIQEEKPEPVLEEQLFIGNLPHEVDKSELKDFFQSYGNVVELRINSGGKLPNFGFVVFDDSEPVQKVLSNRPIMFRGEVRLNVEEKKTRAAREGDRRDNRLRGPGGPRGGLGGGMRGPPRGGMVQKPGFGVGRGLAPRQKLIPMARRAWLEHGHSCFLCEIVIRSQFHTTYEPEA

SEQ ID NO: 34 - protein, 594 aa, synthetic construct
  REGION 1..594: note = Synthetic construct
MHTLYAPGGYDIMGWLCQIMNRPNPQVELGPVDTSCALVLCDLKQKDTPVVYASEAFLYMTGYSNAEVLGRNCRFLQSPDGMVKPKSTRKYVDSNTINTMRKAIDRNAEVQVEVVNFKKNGQRFVNFLTMIPVRDETGEYRYSMGFQCETEGSGGSGGSGGSGMVMEKPSPLLVGREFVRQYYTLLNQAPDMLHRFYGKNSSYVHGGLDSNGKPADAVYGQKEIHRKVMSQNFTNCHTKIRHVDAHATLNDGVVVQVMGLLSNNNQALRRFMQTFVLAPEGSVANKFYVHNDIFRYQDEVFGGFVTEPQEESEEEVEEPEERQQTPEVVPDDSGTFYDQAVVSNDMEEHLEEPVAEPEPDPEPEPEQEPVSEIQEEKPEPVLEETAPEDAQKSSSPAPADIAQTVQEDLRTFSWASVTSKNLPPSGAVPVTGIPPHVVKVPASQPRPESKPESQIPPQRPQRDQRVREQRINIPPQRGPRPIREAGEQGDIEPRRMVRHPDSHEKKTRAAREGDRRDNRLRGPGGPRGGLGGGMRGPPRGGMVQKPGFGVGRGLAPRQKLIPMARRAWLEHGHSCFLCEIVIRSQFHTTYEPEA

SEQ ID NO: 35 - protein, 574 aa, synthetic construct
  REGION 1..574: note = Synthetic construct
MHTLYAPGGYDIMGWLCQIMNRPNPQVELGPVDTSCALVLCDLKQKDTPVVYASEAFLYMTGYSNAEVLGRNCRFLQSPDGMVKPKSTRKYVDSNTINTMRKAIDRNAEVQVEVVNFKKNGQRFVNFLTMIPVRDETGEYRYSMGFQCETEGSGGSGGSGGSGMVMEKPSPLLVGREFVRQYYTLLNQAPDMLHRFYGKNSSYVHGGLDSNGKPADAVYGQKEIHRKVMSQNFTNCHTKIRHVDAHATLNDGVVVQVMGLLSNNNQALRRFMQTFVLAPEGSVANKFYVHNDIFRYQDEVFGGFVTEPQEESEEEVEEPEERQQTPEVVPDDSGTFYDQAVVSNDMEEHLEEPVAEPEPDPEPEPEQEPVSEIQEEKPEPVLEETAPEDAQKSSSPAPADIAQTVQEDLRTFSWASVTSKNLPPSGAVPVTGIPPHVVKVPASQPRPESKPESQIPPQRPQRDQRVREQRINIPPQRGPRPIREAGEQGDIEPRRMVRHPDSHQLFIGNLPHEVDKSELKDFFQSYGNVVELRINSGGKLPNFGFVVFDDSEPVQKVLSNRPIMFRGEVRLNVE

SEQ ID NO: 36 - protein, 503 aa, synthetic construct
  REGION 1..503: note = Synthetic construct
MHTLYAPGGYDIMGWLCQIMNRPNPQVELGPVDTSCALVLCDLKQKDTPVVYASEAFLYMTGYSNAEVLGRNCRFLQSPDGMVKPKSTRKYVDSNTINTMRKAIDRNAEVQVEVVNFKKNGQRFVNFLTMIPVRDETGEYRYSMGFQCETEGSGGSGGSGGSGMVMEKPSPLLVGREFVRQYYTLLNQAPDMLHRFYGKNSSYVHGGLDSNGKPADAVYGQKEIHRKVMSQNFTNCHTKIRHVDAHATLNDGVVVQVMGLLSNNNQALRRFMQTFVLAPEGSVANKFYVHNDIFRYQDEVFGGFVTEPQEESEEEVEEPEERQQTPEVVPDDSGTFYDQAVVSNDMEEHLEEPVAEPEPDPEPEPEQEPVSEIQEEKPEPVLEETAPEDAQKSSSPAPADIAQTVQEDLRTFSWASVTSKNLPPSGAVPVTGIPPHVVKVPASQPRPESKPESQIPPQRPQRDQRVREQRINIPPQRGPRPIREAGEQGDIEPRRMVRHPDSH

SEQ ID NO: 37 - protein, 665 aa, synthetic construct
  REGION 1..665: note = Synthetic construct
MHTLYAPGGYDIMGWLIQIMNRPNPQVELGPVDTSVALVLCDLKQKDTPVVYASEAFLYMTGYSNAEVLGRNCRFLQSPDGMVKPKSTRKYVDSNTINTMRKAIDRNAEVQVEVVNFKKNGQRFVNFLTMIPVRDETGEYRYSMGFQCETEGSGGSGGSGGSGMVMEKPSPLLVGREFVRQYYTLLNQAPDMLHRFYGKNSSYVHGGLDSNGKPADAVYGQKEIHRKVMSQNFTNCHTKIRHVDAHATLNDGVVVQVMGLLSNNNQALRRFMQTFVLAPEGSVANKFYVHNDIFRYQDEVFGGFVTEPQEESEEEVEEPEERQQTPEVVPDDSGTFYDQAVVSNDMEEHLEEPVAEPEPDPEPEPEQEPVSEIQEEKPEPVLEETAPEDAQKSSSPAPADIAQTVQEDLRTFSWASVTSKNLPPSGAVPVTGIPPHVVKVPASQPRPESKPESQIPPQRPQRDQRVREQRINIPPQRGPRPIREAGEQGDIEPRRMVRHPDSHQLFIGNLPHEVDKSELKDFFQSYGNVVELRINSGGKLPNFGFVVFDDSEPVQKVLSNRPIMFRGEVRLNVEEKKTRAAREGDRRDNRLRGPGGPRGGLGGGMRGPPRGGMVQKPGFGVGRGLAPRQKLIPMARRAWLEHGHSCFLCEIVIRSQFHTTYEPEA

SEQ ID NO: 38 - protein, 527 aa, synthetic construct
  REGION 1..527: note = Synthetic construct
MHTLYAPGGYDIMGWLIQIMNRPNPQVELGPVDTSVALVLCDLKQKDTPVVYASEAFLYMTGYSNAEVLGRNCRFLQSPDGMVKPKSTRKYVDSNTINTMRKAIDRNAEVQVEVVNFKKNGQRFVNFLTMIPVRDETGEYRYSMGFQCETEGSGGSGGSGGSGMGFVTEPQEESEEEVEEPEERQQTPEVVPDDSGTFYDQAVVSNDMEEHLEEPVAEPEPDPEPEPEQEPVSEIQEEKPEPVLEETAPEDAQKSSSPAPADIAQTVQEDLRTFSWASVTSKNLPPSGAVPVTGIPPHVVKVPASQPRPESKPESQIPPQRPQRDQRVREQRINIPPQRGPRPIREAGEQGDIEPRRMVRHPDSHQLFIGNLPHEVDKSELKDFFQSYGNVVELRINSGGKLPNFGFVVFDDSEPVQKVLSNRPIMFRGEVRLNVEEKKTRAAREGDRRDNRLRGPGGPRGGLGGGMRGPPRGGMVQKPGFGVGRGLAPRQKLIPMARRAWLEHGHSCFLCEIVIRSQFHTTYEPEA

SEQ ID NO: 39 - protein, 583 aa, synthetic construct
  REGION 1..583: note = Synthetic construct
MHTLYAPGGYDIMGWLIQIMNRPNPQVELGPVDTSVALVLCDLKQKDTPVVYASEAFLYMTGYSNAEVLGRNCRFLQSPDGMVKPKSTRKYVDSNTINTMRKAIDRNAEVQVEVVNFKKNGQRFVNFLTMIPVRDETGEYRYSMGFQCETEGSGGSGGSGGSGMVMEKPSPLLVGREFVRQYYTLLNQAPDMLHRFYGKNSSYVHGGLDSNGKPADAVYGQKEIHRKVMSQNFTNCHTKIRHVDAHATLNDGVVVQVMGLLSNNNQALRRFMQTFVLAPEGSVANKFYVHNDIFRYQDEVFGTAPEDAQKSSSPAPADIAQTVQEDLRTFSWASVTSKNLPPSGAVPVTGIPPHVVKVPASQPRPESKPESQIPPQRPQRDQRVREQRINIPPQRGPRPIREAGEQGDIEPRRMVRHPDSHQLFIGNLPHEVDKSELKDFFQSYGNVVELRINSGGKLPNFGFVVFDDSEPVQKVLSNRPIMFRGEVRLNVEEKKTRAAREGDRRDNRLRGPGGPRGGLGGGMRGPPRGGMVQKPGFGVGRGLAPRQKLIPMARRAWLEHGHSCFLCEIVIRSQFHTTYEPEA

SEQ ID NO: 40 - protein, 546 aa, synthetic construct
  REGION 1..546: note = Synthetic construct
MHTLYAPGGYDIMGWLIQIMNRPNPQVELGPVDTSVALVLCDLKQKDTPVVYASEAFLYMTGYSNAEVLGRNCRFLQSPDGMVKPKSTRKYVDSNTINTMRKAIDRNAEVQVEVVNFKKNGQRFVNFLTMIPVRDETGEYRYSMGFQCETEGSGGSGGSGGSGMVMEKPSPLLVGREFVRQYYTLLNQAPDMLHRFYGKNSSYVHGGLDSNGKPADAVYGQKEIHRKVMSQNFTNCHTKIRHVDAHATLNDGVVVQVMGLLSNNNQALRRFMQTFVLAPEGSVANKFYVHNDIFRYQDEVFGGFVTEPQEESEEEVEEPEERQQTPEVVPDDSGTFYDQAVVSNDMEEHLEEPVAEPEPDPEPEPEQEPVSEIQEEKPEPVLEEQLFIGNLPHEVDKSELKDFFQSYGNVVELRINSGGKLPNFGFVVFDDSEPVQKVLSNRPIMFRGEVRLNVEEKKTRAAREGDRRDNRLRGPGGPRGGLGGGMRGPPRGGMVQKPGFGVGRGLAPRQKLIPMARRAWLEHGHSCFLCEIVIRSQFHTTYEPEA

SEQ ID NO: 41 - protein, 594 aa, synthetic construct
  REGION 1..594: note = Synthetic construct
MHTLYAPGGYDIMGWLIQIMNRPNPQVELGPVDTSVALVLCDLKQKDTPVVYASEAFLYMTGYSNAEVLGRNCRFLQSPDGMVKPKSTRKYVDSNTINTMRKAIDRNAEVQVEVVNFKKNGQRFVNFLTMIPVRDETGEYRYSMGFQCETEGSGGSGGSGGSGMVMEKPSPLLVGREFVRQYYTLLNQAPDMLHRFYGKNSSYVHGGLDSNGKPADAVYGQKEIHRKVMSQNFTNCHTKIRHVDAHATLNDGVVVQVMGLLSNNNQALRRFMQTFVLAPEGSVANKFYVHNDIFRYQDEVFGGFVTEPQEESEEEVEEPEERQQTPEVVPDDSGTFYDQAVVSNDMEEHLEEPVAEPEPDPEPEPEQEPVSEIQEEKPEPVLEETAPEDAQKSSSPAPADIAQTVQEDLRTFSWASVTSKNLPPSGAVPVTGIPPHVVKVPASQPRPESKPESQIPPQRPQRDQRVREQRINIPPQRGPRPIREAGEQGDIEPRRMVRHPDSHEKKTRAAREGDRRDNRLRGPGGPRGGLGGGMRGPPRGGMVQKPGFGVGRGLAPRQKLIPMARRAWLEHGHSCFLCEIVIRSQFHTTYEPEA

SEQ ID NO: 42 - protein, 574 aa, synthetic construct
  REGION 1..574: note = Synthetic construct
MHTLYAPGGYDIMGWLIQIMNRPNPQVELGPVDTSVALVLCDLKQKDTPVVYASEAFLYMTGYSNAEVLGRNCRFLQSPDGMVKPKSTRKYVDSNTINTMRKAIDRNAEVQVEVVNFKKNGQRFVNFLTMIPVRDETGEYRYSMGFQCETEGSGGSGGSGGSGMVMEKPSPLLVGREFVRQYYTLLNQAPDMLHRFYGKNSSYVHGGLDSNGKPADAVYGQKEIHRKVMSQNFTNCHTKIRHVDAHATLNDGVVVQVMGLLSNNNQALRRFMQTFVLAPEGSVANKFYVHNDIFRYQDEVFGGFVTEPQEESEEEVEEPEERQQTPEVVPDDSGTFYDQAVVSNDMEEHLEEPVAEPEPDPEPEPEQEPVSEIQEEKPEPVLEETAPEDAQKSSSPAPADIAQTVQEDLRTFSWASVTSKNLPPSGAVPVTGIPPHVVKVPASQPRPESKPESQIPPQRPQRDQRVREQRINIPPQRGPRPIREAGEQGDIEPRRMVRHPDSHQLFIGNLPHEVDKSELKDFFQSYGNVVELRINSGGKLPNFGFVVFDDSEPVQKVLSNRPIMFRGEVRLNVE

SEQ ID NO: 43 - protein, 503 aa, synthetic construct
  REGION 1..503: note = Synthetic construct
MHTLYAPGGYDIMGWLIQIMNRPNPQVELGPVDTSVALVLCDLKQKDTPVVYASEAFLYMTGYSNAEVLGRNCRFLQSPDGMVKPKSTRKYVDSNTINTMRKAIDRNAEVQVEVVNFKKNGQRFVNFLTMIPVRDETGEYRYSMGFQCETEGSGGSGGSGGSGMVMEKPSPLLVGREFVRQYYTLLNQAPDMLHRFYGKNSSYVHGGLDSNGKPADAVYGQKEIHRKVMSQNFTNCHTKIRHVDAHATLNDGVVVQVMGLLSNNNQALRRFMQTFVLAPEGSVANKFYVHNDIFRYQDEVFGGFVTEPQEESEEEVEEPEERQQTPEVVPDDSGTFYDQAVVSNDMEEHLEEPVAEPEPDPEPEPEQEPVSEIQEEKPEPVLEETAPEDAQKSSSPAPADIAQTVQEDLRTFSWASVTSKNLPPSGAVPVTGIPPHVVKVPASQPRPESKPESQIPPQRPQRDQRVREQRINIPPQRGPRPIREAGEQGDIEPRRMVRHPDSH

SEQ ID NO: 44 - protein, 665 aa, synthetic construct
  REGION 1..665: note = Synthetic construct
MHTLYAPGGYDIMGWLCQIMNRPNPQVELGPVDTSVALVLCDLKQKDTPVVYASEAFLYMTGYSNAEVLGRNCRFLQSPDGMVKPKSTRKYVDSNTINTMRKAIDRNAEVQVEVVNFKKNGQRFVNFLTMIPVRDETGEYRYSMGFQCETEGSGGSGGSGGSGMVMEKPSPLLVGREFVRQYYTLLNQAPDMLHRFYGKNSSYVHGGLDSNGKPADAVYGQKEIHRKVMSQNFTNCHTKIRHVDAHATLNDGVVVQVMGLLSNNNQALRRFMQTFVLAPEGSVANKFYVHNDIFRYQDEVFGGFVTEPQEESEEEVEEPEERQQTPEVVPDDSGTFYDQAVVSNDMEEHLEEPVAEPEPDPEPEPEQEPVSEIQEEKPEPVLEETAPEDAQKSSSPAPADIAQTVQEDLRTFSWASVTSKNLPPSGAVPVTGIPPHVVKVPASQPRPESKPESQIPPQRPQRDQRVREQRINIPPQRGPRPIREAGEQGDIEPRRMVRHPDSHQLFIGNLPHEVDKSELKDFFQSYGNVVELRINSGGKLPNFGFVVFDDSEPVQKVLSNRPIMFRGEVRLNVEEKKTRAAREGDRRDNRLRGPGGPRGGLGGGMRGPPRGGMVQKPGFGVGRGLAPRQKLIPMARRAWLEHGHSCFLCEIVIRSQFHTTYEPEA

SEQ ID NO: 45 - protein, 527 aa, synthetic construct
  REGION 1..527: note = Synthetic construct
MHTLYAPGGYDIMGWLCQIMNRPNPQVELGPVDTSVALVLCDLKQKDTPVVYASEAFLYMTGYSNAEVLGRNCRFLQSPDGMVKPKSTRKYVDSNTINTMRKAIDRNAEVQVEVVNFKKNGQRFVNFLTMIPVRDETGEYRYSMGFQCETEGSGGSGGSGGSGMGFVTEPQEESEEEVEEPEERQQTPEVVPDDSGTFYDQAVVSNDMEEHLEEPVAEPEPDPEPEPEQEPVSEIQEEKPEPVLEETAPEDAQKSSSPAPADIAQTVQEDLRTFSWASVTSKNLPPSGAVPVTGIPPHVVKVPASQPRPESKPESQIPPQRPQRDQRVREQRINIPPQRGPRPIREAGEQGDIEPRRMVRHPDSHQLFIGNLPHEVDKSELKDFFQSYGNVVELRINSGGKLPNFGFVVFDDSEPVQKVLSNRPIMFRGEVRLNVEEKKTRAAREGDRRDNRLRGPGGPRGGLGGGMRGPPRGGMVQKPGFGVGRGLAPRQKLIPMARRAWLEHGHSCFLCEIVIRSQFHTTYEPEA

SEQ ID NO: 46 - protein, 583 aa, synthetic construct
  REGION 1..583: note = Synthetic construct
MHTLYAPGGYDIMGWLCQIMNRPNPQVELGPVDTSVALVLCDLKQKDTPVVYASEAFLYMTGYSNAEVLGRNCRFLQSPDGMVKPKSTRKYVDSNTINTMRKAIDRNAEVQVEVVNFKKNGQRFVNFLTMIPVRDETGEYRYSMGFQCETEGSGGSGGSGGSGMVMEKPSPLLVGREFVRQYYTLLNQAPDMLHRFYGKNSSYVHGGLDSNGKPADAVYGQKEIHRKVMSQNFTNCHTKIRHVDAHATLNDGVVVQVMGLLSNNNQALRRFMQTFVLAPEGSVANKFYVHNDIFRYQDEVFGTAPEDAQKSSSPAPADIAQTVQEDLRTFSWASVTSKNLPPSGAVPVTGIPPHVVKVPASQPRPESKPESQIPPQRPQRDQRVREQRINIPPQRGPRPIREAGEQGDIEPRRMVRHPDSHQLFIGNLPHEVDKSELKDFFQSYGNVVELRINSGGKLPNFGFVVFDDSEPVQKVLSNRPIMFRGEVRLNVEEKKTRAAREGDRRDNRLRGPGGPRGGLGGGMRGPPRGGMVQKPGFGVGRGLAPRQKLIPMARRAWLEHGHSCFLCEIVIRSQFHTTYEPEA

SEQ ID NO: 47 - protein, 546 aa, synthetic construct
  REGION 1..546: note = Synthetic construct
MHTLYAPGGYDIMGWLCQIMNRPNPQVELGPVDTSVALVLCDLKQKDTPVVYASEAFLYMTGYSNAEVLGRNCRFLQSPDGMVKPKSTRKYVDSNTINTMRKAIDRNAEVQVEVVNFKKNGQRFVNFLTMIPVRDETGEYRYSMGFQCETEGSGGSGGSGGSGMVMEKPSPLLVGREFVRQYYTLLNQAPDMLHRFYGKNSSYVHGGLDSNGKPADAVYGQKEIHRKVMSQNFTNCHTKIRHVDAHATLNDGVVVQVMGLLSNNNQALRRFMQTFVLAPEGSVANKFYVHNDIFRYQDEVFGGFVTEPQEESEEEVEEPEERQQTPEVVPDDSGTFYDQAVVSNDMEEHLEEPVAEPEPDPEPEPEQEPVSEIQEEKPEPVLEEQLFIGNLPHEVDKSELKDFFQSYGNVVELRINSGGKLPNFGFVVFDDSEPVQKVLSNRPIMFRGEVRLNVEEKKTRAAREGDRRDNRLRGPGGPRGGLGGGMRGPPRGGMVQKPGFGVGRGLAPRQKLIPMARRAWLEHGHSCFLCEIVIRSQFHTTYEPEA

SEQ ID NO: 48 - protein, 594 aa, synthetic construct
  REGION 1..594: note = Synthetic construct
MHTLYAPGGYDIMGWLCQIMNRPNPQVELGPVDTSVALVLCDLKQKDTPVVYASEAFLYMTGYSNAEVLGRNCRFLQSPDGMVKPKSTRKYVDSNTINTMRKAIDRNAEVQVEVVNFKKNGQRFVNFLTMIPVRDETGEYRYSMGFQCETEGSGGSGGSGGSGMVMEKPSPLLVGREFVRQYYTLLNQAPDMLHRFYGKNSSYVHGGLDSNGKPADAVYGQKEIHRKVMSQNFTNCHTKIRHVDAHATLNDGVVVQVMGLLSNNNQALRRFMQTFVLAPEGSVANKFYVHNDIFRYQDEVFGGFVTEPQEESEEEVEEPEERQQTPEVVPDDSGTFYDQAVVSNDMEEHLEEPVAEPEPDPEPEPEQEPVSEIQEEKPEPVLEETAPEDAQKSSSPAPADIAQTVQEDLRTFSWASVTSKNLPPSGAVPVTGIPPHVVKVPASQPRPESKPESQIPPQRPQRDQRVREQRINIPPQRGPRPIREAGEQGDIEPRRMVRHPDSHEKKTRAAREGDRRDNRLRGPGGPRGGLGGGMRGPPRGGMVQKPGFGVGRGLAPRQKLIPMARRAWLEHGHSCFLCEIVIRSQFHTTYEPEA

SEQ ID NO: 49 - protein, 574 aa, synthetic construct
  REGION 1..574: note = Synthetic construct
MHTLYAPGGYDIMGWLCQIMNRPNPQVELGPVDTSVALVLCDLKQKDTPVVYASEAFLYMTGYSNAEVLGRNCRFLQSPDGMVKPKSTRKYVDSNTINTMRKAIDRNAEVQVEVVNFKKNGQRFVNFLTMIPVRDETGEYRYSMGFQCETEGSGGSGGSGGSGMVMEKPSPLLVGREFVRQYYTLLNQAPDMLHRFYGKNSSYVHGGLDSNGKPADAVYGQKEIHRKVMSQNFTNCHTKIRHVDAHATLNDGVVVQVMGLLSNNNQALRRFMQTFVLAPEGSVANKFYVHNDIFRYQDEVFGGFVTEPQEESEEEVEEPEERQQTPEVVPDDSGTFYDQAVVSNDMEEHLEEPVAEPEPDPEPEPEQEPVSEIQEEKPEPVLEETAPEDAQKSSSPAPADIAQTVQEDLRTFSWASVTSKNLPPSGAVPVTGIPPHVVKVPASQPRPESKPESQIPPQRPQRDQRVREQRINIPPQRGPRPIREAGEQGDIEPRRMVRHPDSHQLFIGNLPHEVDKSELKDFFQSYGNVVELRINSGGKLPNFGFVVFDDSEPVQKVLSNRPIMFRGEVRLNVE

SEQ ID NO: 50 - protein, 503 aa, synthetic construct
  REGION 1..503: note = Synthetic construct
MHTLYAPGGYDIMGWLCQIMNRPNPQVELGPVDTSVALVLCDLKQKDTPVVYASEAFLYMTGYSNAEVLGRNCRFLQSPDGMVKPKSTRKYVDSNTINTMRKAIDRNAEVQVEVVNFKKNGQRFVNFLTMIPVRDETGEYRYSMGFQCETEGSGGSGGSGGSGMVMEKPSPLLVGREFVRQYYTLLNQAPDMLHRFYGKNSSYVHGGLDSNGKPADAVYGQKEIHRKVMSQNFTNCHTKIRHVDAHATLNDGVVVQVMGLLSNNNQALRRFMQTFVLAPEGSVANKFYVHNDIFRYQDEVFGGFVTEPQEESEEEVEEPEERQQTPEVVPDDSGTFYDQAVVSNDMEEHLEEPVAEPEPDPEPEPEQEPVSEIQEEKPEPVLEETAPEDAQKSSSPAPADIAQTVQEDLRTFSWASVTSKNLPPSGAVPVTGIPPHVVKVPASQPRPESKPESQIPPQRPQRDQRVREQRINIPPQRGPRPIREAGEQGDIEPRRMVRHPDSH

SEQ ID NO: 51 - protein, 674 aa, synthetic construct
  REGION 1..674: note = Synthetic construct
MHTLYAPGGYDIMGWLIQIMNRPNPQVELGPVDTSCALVLCDLKQKDTPVVYASEAFLYMTGYSNAEVLGRNCRFLQSPDGMVKPKSTRKYVDSNTINTMRKAIDRNAEVQVEVVNFKKNGQRFVNFLTMIPVRDETGEYRYSMGFQCETEGSGGSGGSGGSGGTGGSGGSGMVMEKPSPLLVGREFVRQYYTLLNQAPDMLHRFYGKNSSYVHGGLDSNGKPADAVYGQKEIHRKVMSQNFTNCHTKIRHVDAHATLNDGVVVQVMGLLSNNNQALRRFMQTFVLAPEGSVANKFYVHNDIFRYQDEVFGGFVTEPQEESEEEVEEPEERQQTPEVVPDDSGTFYDQAVVSNDMEEHLEEPVAEPEPDPEPEPEQEPVSEIQEEKPEPVLEETAPEDAQKSSSPAPADIAQTVQEDLRTFSWASVTSKNLPPSGAVPVTGIPPHVVKVPASQPRPESKPESQIPPQRPQRDQRVREQRINIPPQRGPRPIREAGEQGDIEPRRMVRHPDSHQLFIGNLPHEVDKSELKDFFQSYGNVVELRINSGGKLPNFGFVVFDDSEPVQKVLSNRPIMFRGEVRLNVEEKKTRAAREGDRRDNRLRGPGGPRGGLGGGMRGPPRGGMVQKPGFGVGRGLAPRQKLIPMARRAWLEHGHSCFLCEIVIRSQFHTTYEPEA

SEQ ID NO: 52 - protein, 536 aa, synthetic construct
  REGION 1..536: note = Synthetic construct
MHTLYAPGGYDIMGWLIQIMNRPNPQVELGPVDTSCALVLCDLKQKDTPVVYASEAFLYMTGYSNAEVLGRNCRFLQSPDGMVKPKSTRKYVDSNTINTMRKAIDRNAEVQVEVVNFKKNGQRFVNFLTMIPVRDETGEYRYSMGFQCETEGSGGSGGSGGSGGTGGSGGSGMGFVTEPQEESEEEVEEPEERQQTPEVVPDDSGTFYDQAVVSNDMEEHLEEPVAEPEPDPEPEPEQEPVSEIQEEKPEPVLEETAPEDAQKSSSPAPADIAQTVQEDLRTFSWASVTSKNLPPSGAVPVTGIPPHVVKVPASQPRPESKPESQIPPQRPQRDQRVREQRINIPPQRGPRPIREAGEQGDIEPRRMVRHPDSHQLFIGNLPHEVDKSELKDFFQSYGNVVELRINSGGKLPNFGFVVFDDSEPVQKVLSNRPIMFRGEVRLNVEEKKTRAAREGDRRDNRLRGPGGPRGGLGGGMRGPPRGGMVQKPGFGVGRGLAPRQKLIPMARRAWLEHGHSCFLCEIVIRSQFHTTYEPEA

SEQ ID NO: 53 - protein, 592 aa, synthetic construct
  REGION 1..592: note = Synthetic construct
MHTLYAPGGYDIMGWLIQIMNRPNPQVELGPVDTSCALVLCDLKQKDTPVVYASEAFLYMTGYSNAEVLGRNCRFLQSPDGMVKPKSTRKYVDSNTINTMRKAIDRNAEVQVEVVNFKKNGQRFVNFLTMIPVRDETGEYRYSMGFQCETEGSGGSGGSGGSGGTGGSGGSGMVMEKPSPLLVGREFVRQYYTLLNQAPDMLHRFYGKNSSYVHGGLDSNGKPADAVYGQKEIHRKVMSQNFTNCHTKIRHVDAHATLNDGVVVQVMGLLSNNNQALRRFMQTFVLAPEGSVANKFYVHNDIFRYQDEVFGTAPEDAQKSSSPAPADIAQTVQEDLRTFSWASVTSKNLPPSGAVPVTGIPPHVVKVPASQPRPESKPESQIPPQRPQRDQRVREQRINIPPQRGPRPIREAGEQGDIEPRRMVRHPDSHQLFIGNLPHEVDKSELKDFFQSYGNVVELRINSGGKLPNFGFVVFDDSEPVQKVLSNRPIMFRGEVRLNVEEKKTRAAREGDRRDNRLRGPGGPRGGLGGGMRGPPRGGMVQKPGFGVGRGLAPRQKLIPMARRAWLEHGHSCFLCEIVIRSQFHTTYEPEA

SEQ ID NO: 54 - protein, 555 aa, synthetic construct
  REGION 1..555: note = Synthetic construct
MHTLYAPGGYDIMGWLIQIMNRPNPQVELGPVDTSCALVLCDLKQKDTPVVYASEAFLYMTGYSNAEVLGRNCRFLQSPDGMVKPKSTRKYVDSNTINTMRKAIDRNAEVQVEVVNFKKNGQRFVNFLTMIPVRDETGEYRYSMGFQCETEGSGGSGGSGGSGGTGGSGGSGMVMEKPSPLLVGREFVRQYYTLLNQAPDMLHRFYGKNSSYVHGGLDSNGKPADAVYGQKEIHRKVMSQNFTNCHTKIRHVDAHATLNDGVVVQVMGLLSNNNQALRRFMQTFVLAPEGSVANKFYVHNDIFRYQDEVFGGFVTEPQEESEEEVEEPEERQQTPEVVPDDSGTFYDQAVVSNDMEEHLEEPVAEPEPDPEPEPEQEPVSEIQEEKPEPVLEEQLFIGNLPHEVDKSELKDFFQSYGNVVELRINSGGKLPNFGFVVFDDSEPVQKVLSNRPIMFRGEVRLNVEEKKTRAAREGDRRDNRLRGPGGPRGGLGGGMRGPPRGGMVQKPGFGVGRGLAPRQKLIPMARRAWLEHGHSCFLCEIVIRSQFHTTYEPEA

SEQ ID NO: 55 - protein, 603 aa, synthetic construct
  REGION 1..603: note = Synthetic construct
MHTLYAPGGYDIMGWLIQIMNRPNPQVELGPVDTSCALVLCDLKQKDTPVVYASEAFLYMTGYSNAEVLGRNCRFLQSPDGMVKPKSTRKYVDSNTINTMRKAIDRNAEVQVEVVNFKKNGQRFVNFLTMIPVRDETGEYRYSMGFQCETEGSGGSGGSGGSGGTGGSGGSGMVMEKPSPLLVGREFVRQYYTLLNQAPDMLHRFYGKNSSYVHGGLDSNGKPADAVYGQKEIHRKVMSQNFTNCHTKIRHVDAHATLNDGVVVQVMGLLSNNNQALRRFMQTFVLAPEGSVANKFYVHNDIFRYQDEVFGGFVTEPQEESEEEVEEPEERQQTPEVVPDDSGTFYDQAVVSNDMEEHLEEPVAEPEPDPEPEPEQEPVSEIQEEKPEPVLEETAPEDAQKSSSPAPADIAQTVQEDLRTFSWASVTSKNLPPSGAVPVTGIPPHVVKVPASQPRPESKPESQIPPQRPQRDQRVREQRINIPPQRGPRPIREAGEQGDIEPRRMVRHPDSHEKKTRAAREGDRRDNRLRGPGGPRGGLGGGMRGPPRGGMVQKPGFGVGRGLAPRQKLIPMARRAWLEHGHSCFLCEIVIRSQFHTTYEPEA

SEQ ID NO: 56 - protein, 583 aa, synthetic construct
  REGION 1..583: note = Synthetic construct
MHTLYAPGGYDIMGWLIQIMNRPNPQVELGPVDTSCALVLCDLKQKDTPVVYASEAFLYMTGYSNAEVLGRNCRFLQSPDGMVKPKSTRKYVDSNTINTMRKAIDRNAEVQVEVVNFKKNGQRFVNFLTMIPVRDETGEYRYSMGFQCETEGSGGSGGSGGSGGTGGSGGSGMVMEKPSPLLVGREFVRQYYTLLNQAPDMLHRFYGKNSSYVHGGLDSNGKPADAVYGQKEIHRKVMSQNFTNCHTKIRHVDAHATLNDGVVVQVMGLLSNNNQALRRFMQTFVLAPEGSVANKFYVHNDIFRYQDEVFGGFVTEPQEESEEEVEEPEERQQTPEVVPDDSGTFYDQAVVSNDMEEHLEEPVAEPEPDPEPEPEQEPVSEIQEEKPEPVLEETAPEDAQKSSSPAPADIAQTVQEDLRTFSWASVTSKNLPPSGAVPVTGIPPHVVKVPASQPRPESKPESQIPPQRPQRDQRVREQRINIPPQRGPRPIREAGEQGDIEPRRMVRHPDSHQLFIGNLPHEVDKSELKDFFQSYGNVVELRINSGGKLPNFGFVVFDDSEPVQKVLSNRPIMFRGEVRLNVE

SEQ ID NO: 57 - protein, 512 aa, synthetic construct
  REGION 1..512: note = Synthetic construct
MHTLYAPGGYDIMGWLIQIMNRPNPQVELGPVDTSCALVLCDLKQKDTPVVYASEAFLYMTGYSNAEVLGRNCRFLQSPDGMVKPKSTRKYVDSNTINTMRKAIDRNAEVQVEVVNFKKNGQRFVNFLTMIPVRDETGEYRYSMGFQCETEGSGGSGGSGGSGGTGGSGGSGMVMEKPSPLLVGREFVRQYYTLLNQAPDMLHRFYGKNSSYVHGGLDSNGKPADAVYGQKEIHRKVMSQNFTNCHTKIRHVDAHATLNDGVVVQVMGLLSNNNQALRRFMQTFVLAPEGSVANKFYVHNDIFRYQDEVFGGFVTEPQEESEEEVEEPEERQQTPEVVPDDSGTFYDQAVVSNDMEEHLEEPVAEPEPDPEPEPEQEPVSEIQEEKPEPVLEETAPEDAQKSSSPAPADIAQTVQEDLRTFSWASVTSKNLPPSGAVPVTGIPPHVVKVPASQPRPESKPESQIPPQRPQRDQRVREQRINIPPQRGPRPIREAGEQGDIEPRRMVRHPDSH

SEQ ID NO: 58 - protein, 674 aa, synthetic construct
  REGION 1..674: note = Synthetic construct
MHTLYAPGGYDIMGWLCQIMNRPNPQVELGPVDTSCALVLCDLKQKDTPVVYASEAFLYMTGYSNAEVLGRNCRFLQSPDGMVKPKSTRKYVDSNTINTMRKAIDRNAEVQVEVVNFKKNGQRFVNFLTMIPVRDETGEYRYSMGFQCETEGSGGSGGSGGSGGTGGSGGSGMVMEKPSPLLVGREFVRQYYTLLNQAPDMLHRFYGKNSSYVHGGLDSNGKPADAVYGQKEIHRKVMSQNFTNCHTKIRHVDAHATLNDGVVVQVMGLLSNNNQALRRFMQTFVLAPEGSVANKFYVHNDIFRYQDEVFGGFVTEPQEESEEEVEEPEERQQTPEVVPDDSGTFYDQAVVSNDMEEHLEEPVAEPEPDPEPEPEQEPVSEIQEEKPEPVLEETAPEDAQKSSSPAPADIAQTVQEDLRTFSWASVTSKNLPPSGAVPVTGIPPHVVKVPASQPRPESKPESQIPPQRPQRDQRVREQRINIPPQRGPRPIREAGEQGDIEPRRMVRHPDSHQLFIGNLPHEVDKSELKDFFQSYGNVVELRINSGGKLPNFGFVVFDDSEPVQKVLSNRPIMFRGEVRLNVEEKKTRAAREGDRRDNRLRGPGGPRGGLGGGMRGPPRGGMVQKPGFGVGRGLAPRQKLIPMARRAWLEHGHSCFLCEIVIRSQFHTTYEPEA

SEQ ID NO: 59 - protein, 536 aa, synthetic construct
  REGION 1..536: note = Synthetic construct
MHTLYAPGGYDIMGWLCQIMNRPNPQVELGPVDTSCALVLCDLKQKDTPVVYASEAFLYMTGYSNAEVLGRNCRFLQSPDGMVKPKSTRKYVDSNTINTMRKAIDRNAEVQVEVVNFKKNGQRFVNFLTMIPVRDETGEYRYSMGFQCETEGSGGSGGSGGSGGTGGSGGSGMGFVTEPQEESEEEVEEPEERQQTPEVVPDDSGTFYDQAVVSNDMEEHLEEPVAEPEPDPEPEPEQEPVSEIQEEKPEPVLEETAPEDAQKSSSPAPADIAQTVQEDLRTFSWASVTSKNLPPSGAVPVTGIPPHVVKVPASQPRPESKPESQIPPQRPQRDQRVREQRINIPPQRGPRPIREAGEQGDIEPRRMVRHPDSHQLFIGNLPHEVDKSELKDFFQSYGNVVELRINSGGKLPNFGFVVFDDSEPVQKVLSNRPIMFRGEVRLNVEEKKTRAAREGDRRDNRLRGPGGPRGGLGGGMRGPPRGGMVQKPGFGVGRGLAPRQKLIPMARRAWLEHGHSCFLCEIVIRSQFHTTYEPEA

SEQ ID NO: 60 - protein, 592 aa, synthetic construct
  REGION 1..592: note = Synthetic construct
MHTLYAPGGYDIMGWLCQIMNRPNPQVELGPVDTSCALVLCDLKQKDTPVVYASEAFLYMTGYSNAEVLGRNCRFLQSPDGMVKPKSTRKYVDSNTINTMRKAIDRNAEVQVEVVNFKKNGQRFVNFLTMIPVRDETGEYRYSMGFQCETEGSGGSGGSGGSGGTGGSGGSGMVMEKPSPLLVGREFVRQYYTLLNQAPDMLHRFYGKNSSYVHGGLDSNGKPADAVYGQKEIHRKVMSQNFTNCHTKIRHVDAHATLNDGVVVQVMGLLSNNNQALRRFMQTFVLAPEGSVANKFYVHNDIFRYQDEVFGTAPEDAQKSSSPAPADIAQTVQEDLRTFSWASVTSKNLPPSGAVPVTGIPPHVVKVPASQPRPESKPESQIPPQRPQRDQRVREQRINIPPQRGPRPIREAGEQGDIEPRRMVRHPDSHQLFIGNLPHEVDKSELKDFFQSYGNVVELRINSGGKLPNFGFVVFDDSEPVQKVLSNRPIMFRGEVRLNVEEKKTRAAREGDRRDNRLRGPGGPRGGLGGGMRGPPRGGMVQKPGFGVGRGLAPRQKLIPMARRAWLEHGHSCFLCEIVIRSQFHTTYEPEA

SEQ ID NO: 61 - protein, 555 aa, synthetic construct
  REGION 1..555: note = Synthetic construct
MHTLYAPGGYDIMGWLCQIMNRPNPQVELGPVDTSCALVLCDLKQKDTPVVYASEAFLYMTGYSNAEVLGRNCRFLQSPDGMVKPKSTRKYVDSNTINTMRKAIDRNAEVQVEVVNFKKNGQRFVNFLTMIPVRDETGEYRYSMGFQCETEGSGGSGGSGGSGGTGGSGGSGMVMEKPSPLLVGREFVRQYYTLLNQAPDMLHRFYGKNSSYVHGGLDSNGKPADAVYGQKEIHRKVMSQNFTNCHTKIRHVDAHATLNDGVVVQVMGLLSNNNQALRRFMQTFVLAPEGSVANKFYVHNDIFRYQDEVFGGFVTEPQEESEEEVEEPEERQQTPEVVPDDSGTFYDQAVVSNDMEEHLEEPVAEPEPDPEPEPEQEPVSEIQEEKPEPVLEEQLFIGNLPHEVDKSELKDFFQSYGNVVELRINSGGKLPNFGFVVFDDSEPVQKVLSNRPIMFRGEVRLNVEEKKTRAAREGDRRDNRLRGPGGPRGGLGGGMRGPPRGGMVQKPGFGVGRGLAPRQKLIPMARRAWLEHGHSCFLCEIVIRSQFHTTYEPEA

SEQ ID NO: 62 - protein, 603 aa, synthetic construct
  REGION 1..603: note = Synthetic construct
MHTLYAPGGYDIMGWLCQIMNRPNPQVELGPVDTSCALVLCDLKQKDTPVVYASEAFLYMTGYSNAEVLGRNCRFLQSPDGMVKPKSTRKYVDSNTINTMRKAIDRNAEVQVEVVNFKKNGQRFVNFLTMIPVRDETGEYRYSMGFQCETEGSGGSGGSGGSGGTGGSGGSGMVMEKPSPLLVGREFVRQYYTLLNQAPDMLHRFYGKNSSYVHGGLDSNGKPADAVYGQKEIHRKVMSQNFTNCHTKIRHVDAHATLNDGVVVQVMGLLSNNNQALRRFMQTFVLAPEGSVANKFYVHNDIFRYQDEVFGGFVTEPQEESEEEVEEPEERQQTPEVVPDDSGTFYDQAVVSNDMEEHLEEPVAEPEPDPEPEPEQEPVSEIQEEKPEPVLEETAPEDAQKSSSPAPADIAQTVQEDLRTFSWASVTSKNLPPSGAVPVTGIPPHVVKVPASQPRPESKPESQIPPQRPQRDQRVREQRINIPPQRGPRPIREAGEQGDIEPRRMVRHPDSHEKKTRAAREGDRRDNRLRGPGGPRGGLGGGMRGPPRGGMVQKPGFGVGRGLAPRQKLIPMARRAWLEHGHSCFLCEIVIRSQFHTTYEPEA

SEQ ID NO: 63 - protein, 583 aa, synthetic construct
  REGION 1..583: note = Synthetic construct
MHTLYAPGGYDIMGWLCQIMNRPNPQVELGPVDTSCALVLCDLKQKDTPVVYASEAFLYMTGYSNAEVLGRNCRFLQSPDGMVKPKSTRKYVDSNTINTMRKAIDRNAEVQVEVVNFKKNGQRFVNFLTMIPVRDETGEYRYSMGFQCETEGSGGSGGSGGSGGTGGSGGSGMVMEKPSPLLVGREFVRQYYTLLNQAPDMLHRFYGKNSSYVHGGLDSNGKPADAVYGQKEIHRKVMSQNFTNCHTKIRHVDAHATLNDGVVVQVMGLLSNNNQALRRFMQTFVLAPEGSVANKFYVHNDIFRYQDEVFGGFVTEPQEESEEEVEEPEERQQTPEVVPDDSGTFYDQAVVSNDMEEHLEEPVAEPEPDPEPEPEQEPVSEIQEEKPEPVLEETAPEDAQKSSSPAPADIAQTVQEDLRTFSWASVTSKNLPPSGAVPVTGIPPHVVKVPASQPRPESKPESQIPPQRPQRDQRVREQRINIPPQRGPRPIREAGEQGDIEPRRMVRHPDSHQLFIGNLPHEVDKSELKDFFQSYGNVVELRINSGGKLPNFGFVVFDDSEPVQKVLSNRPIMFRGEVRLNVE

SEQ ID NO: 64 - protein, 512 aa, synthetic construct
  REGION 1..512: note = Synthetic construct
MHTLYAPGGYDIMGWLCQIMNRPNPQVELGPVDTSCALVLCDLKQKDTPVVYASEAFLYMTGYSNAEVLGRNCRFLQSPDGMVKPKSTRKYVDSNTINTMRKAIDRNAEVQVEVVNFKKNGQRFVNFLTMIPVRDETGEYRYSMGFQCETEGSGGSGGSGGSGGTGGSGGSGMVMEKPSPLLVGREFVRQYYTLLNQAPDMLHRFYGKNSSYVHGGLDSNGKPADAVYGQKEIHRKVMSQNFTNCHTKIRHVDAHATLNDGVVVQVMGLLSNNNQALRRFMQTFVLAPEGSVANKFYVHNDIFRYQDEVFGGFVTEPQEESEEEVEEPEERQQTPEVVPDDSGTFYDQAVVSNDMEEHLEEPVAEPEPDPEPEPEQEPVSEIQEEKPEPVLEETAPEDAQKSSSPAPADIAQTVQEDLRTFSWASVTSKNLPPSGAVPVTGIPPHVVKVPASQPRPESKPESQIPPQRPQRDQRVREQRINIPPQRGPRPIREAGEQGDIEPRRMVRHPDSH

SEQ ID NO: 65 - protein, 674 aa, synthetic construct
  REGION 1..674: note = Synthetic construct
MHTLYAPGGYDIMGWLIQIMNRPNPQVELGPVDTSVALVLCDLKQKDTPVVYASEAFLYMTGYSNAEVLGRNCRFLQSPDGMVKPKSTRKYVDSNTINTMRKAIDRNAEVQVEVVNFKKNGQRFVNFLTMIPVRDETGEYRYSMGFQCETEGSGGSGGSGGSGGTGGSGGSGMVMEKPSPLLVGREFVRQYYTLLNQAPDMLHRFYGKNSSYVHGGLDSNGKPADAVYGQKEIHRKVMSQNFTNCHTKIRHVDAHATLNDGVVVQVMGLLSNNNQALRRFMQTFVLAPEGSVANKFYVHNDIFRYQDEVFGGFVTEPQEESEEEVEEPEERQQTPEVVPDDSGTFYDQAVVSNDMEEHLEEPVAEPEPDPEPEPEQEPVSEIQEEKPEPVLEETAPEDAQKSSSPAPADIAQTVQEDLRTFSWASVTSKNLPPSGAVPVTGIPPHVVKVPASQPRPESKPESQIPPQRPQRDQRVREQRINIPPQRGPRPIREAGEQGDIEPRRMVRHPDSHQLFIGNLPHEVDKSELKDFFQSYGNVVELRINSGGKLPNFGFVVFDDSEPVQKVLSNRPIMFRGEVRLNVEEKKTRAAREGDRRDNRLRGPGGPRGGLGGGMRGPPRGGMVQKPGFGVGRGLAPRQKLIPMARRAWLEHGHSCFLCEIVIRSQFHTTYEPEA

SEQ ID NO: 66 - protein, 536 aa, synthetic construct
  REGION 1..536: note = Synthetic construct
MHTLYAPGGYDIMGWLIQIMNRPNPQVELGPVDTSVALVLCDLKQKDTPVVYASEAFLYMTGYSNAEVLGRNCRFLQSPDGMVKPKSTRKYVDSNTINTMRKAIDRNAEVQVEVVNFKKNGQRFVNFLTMIPVRDETGEYRYSMGFQCETEGSGGSGGSGGSGGTGGSGGSGMGFVTEPQEESEEEVEEPEERQQTPEVVPDDSGTFYDQAVVSNDMEEHLEEPVAEPEPDPEPEPEQEPVSEIQEEKPEPVLEETAPEDAQKSSSPAPADIAQTVQEDLRTFSWASVTSKNLPPSGAVPVTGIPPHVVKVPASQPRPESKPESQIPPQRPQRDQRVREQRINIPPQRGPRPIREAGEQGDIEPRRMVRHPDSHQLFIGNLPHEVDKSELKDFFQSYGNVVELRINSGGKLPNFGFVVFDDSEPVQKVLSNRPIMFRGEVRLNVEEKKTRAAREGDRRDNRLRGPGGPRGGLGGGMRGPPRGGMVQKPGFGVGRGLAPRQKLIPMARRAWLEHGHSCFLCEIVIRSQFHTTYEPEA

SEQ ID NO: 67 - protein, 592 aa, synthetic construct
  REGION 1..592: note = Synthetic construct
MHTLYAPGGYDIMGWLIQIMNRPNPQVELGPVDTSVALVLCDLKQKDTPVVYASEAFLYMTGYSNAEVLGRNCRFLQSPDGMVKPKSTRKYVDSNTINTMRKAIDRNAEVQVEVVNFKKNGQRFVNFLTMIPVRDETGEYRYSMGFQCETEGSGGSGGSGGSGGTGGSGGSGMVMEKPSPLLVGREFVRQYYTLLNQAPDMLHRFYGKNSSYVHGGLDSNGKPADAVYGQKEIHRKVMSQNFTNCHTKIRHVDAHATLNDGVVVQVMGLLSNNNQALRRFMQTFVLAPEGSVANKFYVHNDIFRYQDEVFGTAPEDAQKSSSPAPADIAQTVQEDLRTFSWASVTSKNLPPSGAVPVTGIPPHVVKVPASQPRPESKPESQIPPQRPQRDQRVREQRINIPPQRGPRPIREAGEQGDIEPRRMVRHPDSHQLFIGNLPHEVDKSELKDFFQSYGNVVELRINSGGKLPNFGFVVFDDSEPVQKVLSNRPIMFRGEVRLNVEEKKTRAAREGDRRDNRLRGPGGPRGGLGGGMRGPPRGGMVQKPGFGVGRGLAPRQKLIPMARRAWLEHGHSCFLCEIVIRSQFHTTYEPEA

SEQ ID NO: 68 - protein, 555 aa, synthetic construct
  REGION 1..555: note = Synthetic construct
MHTLYAPGGYDIMGWLIQIMNRPNPQVELGPVDTSVALVLCDLKQKDTPVVYASEAFLYMTGYSNAEVLGRNCRFLQSPDGMVKPKSTRKYVDSNTINTMRKAIDRNAEVQVEVVNFKKNGQRFVNFLTMIPVRDETGEYRYSMGFQCETEGSGGSGGSGGSGGTGGSGGSGMVMEKPSPLLVGREFVRQYYTLLNQAPDMLHRFYGKNSSYVHGGLDSNGKPADAVYGQKEIHRKVMSQNFTNCHTKIRHVDAHATLNDGVVVQVMGLLSNNNQALRRFMQTFVLAPEGSVANKFYVHNDIFRYQDEVFGGFVTEPQEESEEEVEEPEERQQTPEVVPDDSGTFYDQAVVSNDMEEHLEEPVAEPEPDPEPEPEQEPVSEIQEEKPEPVLEEQLFIGNLPHEVDKSELKDFFQSYGNVVELRINSGGKLPNFGFVVFDDSEPVQKVLSNRPIMFRGEVRLNVEEKKTRAAREGDRRDNRLRGPGGPRGGLGGGMRGPPRGGMVQKPGFGVGRGLAPRQKLIPMARRAWLEHGHSCFLCEIVIRSQFHTTYEPEA

SEQ ID NO: 69 - protein, 603 aa, synthetic construct
  REGION 1..603: note = Synthetic construct
MHTLYAPGGYDIMGWLIQIMNRPNPQVELGPVDTSVALVLCDLKQKDTPVVYASEAFLYMTGYSNAEVLGRNCRFLQSPDGMVKPKSTRKYVDSNTINTMRKAIDRNAEVQVEVVNFKKNGQRFVNFLTMIPVRDETGEYRYSMGFQCETEGSGGSGGSGGSGGTGGSGGSGMVMEKPSPLLVGREFVRQYYTLLNQAPDMLHRFYGKNSSYVHGGLDSNGKPADAVYGQKEIHRKVMSQNFTNCHTKIRHVDAHATLNDGVVVQVMGLLSNNNQALRRFMQTFVLAPEGSVANKFYVHNDIFRYQDEVFGGFVTEPQEESEEEVEEPEERQQTPEVVPDDSGTFYDQAVVSNDMEEHLEEPVAEPEPDPEPEPEQEPVSEIQEEKPEPVLEETAPEDAQKSSSPAPADIAQTVQEDLRTFSWASVTSKNLPPSGAVPVTGIPPHVVKVPASQPRPESKPESQIPPQRPQRDQRVREQRINIPPQRGPRPIREAGEQGDIEPRRMVRHPDSHEKKTRAAREGDRRDNRLRGPGGPRGGLGGGMRGPPRGGMVQKPGFGVGRGLAPRQKLIPMARRAWLEHGHSCFLCEIVIRSQFHTTYEPEA

SEQ ID NO: 70 - protein, 583 aa, synthetic construct
  REGION 1..583: note = Synthetic construct
MHTLYAPGGYDIMGWLIQIMNRPNPQVELGPVDTSVALVLCDLKQKDTPVVYASEAFLYMTGYSNAEVLGRNCRFLQSPDGMVKPKSTRKYVDSNTINTMRKAIDRNAEVQVEVVNFKKNGQRFVNFLTMIPVRDETGEYRYSMGFQCETEGSGGSGGSGGSGGTGGSGGSGMVMEKPSPLLVGREFVRQYYTLLNQAPDMLHRFYGKNSSYVHGGLDSNGKPADAVYGQKEIHRKVMSQNFTNCHTKIRHVDAHATLNDGVVVQVMGLLSNNNQALRRFMQTFVLAPEGSVANKFYVHNDIFRYQDEVFGGFVTEPQEESEEEVEEPEERQQTPEVVPDDSGTFYDQAVVSNDMEEHLEEPVAEPEPDPEPEPEQEPVSEIQEEKPEPVLEETAPEDAQKSSSPAPADIAQTVQEDLRTFSWASVTSKNLPPSGAVPVTGIPPHVVKVPASQPRPESKPESQIPPQRPQRDQRVREQRINIPPQRGPRPIREAGEQGDIEPRRMVRHPDSHQLFIGNLPHEVDKSELKDFFQSYGNVVELRINSGGKLPNFGFVVFDDSEPVQKVLSNRPIMFRGEVRLNVE

SEQ ID NO: 71 - protein, 512 aa, synthetic construct
  REGION 1..512: note = Synthetic construct
MHTLYAPGGYDIMGWLIQIMNRPNPQVELGPVDTSVALVLCDLKQKDTPVVYASEAFLYMTGYSNAEVLGRNCRFLQSPDGMVKPKSTRKYVDSNTINTMRKAIDRNAEVQVEVVNFKKNGQRFVNFLTMIPVRDETGEYRYSMGFQCETEGSGGSGGSGGSGGTGGSGGSGMVMEKPSPLLVGREFVRQYYTLLNQAPDMLHRFYGKNSSYVHGGLDSNGKPADAVYGQKEIHRKVMSQNFTNCHTKIRHVDAHATLNDGVVVQVMGLLSNNNQALRRFMQTFVLAPEGSVANKFYVHNDIFRYQDEVFGGFVTEPQEESEEEVEEPEERQQTPEVVPDDSGTFYDQAVVSNDMEEHLEEPVAEPEPDPEPEPEQEPVSEIQEEKPEPVLEETAPEDAQKSSSPAPADIAQTVQEDLRTFSWASVTSKNLPPSGAVPVTGIPPHVVKVPASQPRPESKPESQIPPQRPQRDQRVREQRINIPPQRGPRPIREAGEQGDIEPRRMVRHPDSH

SEQ ID NO: 72 - protein, 674 aa, synthetic construct
  REGION 1..674: note = Synthetic construct
MHTLYAPGGYDIMGWLCQIMNRPNPQVELGPVDTSVALVLCDLKQKDTPVVYASEAFLYMTGYSNAEVLGRNCRFLQSPDGMVKPKSTRKYVDSNTINTMRKAIDRNAEVQVEVVNFKKNGQRFVNFLTMIPVRDETGEYRYSMGFQCETEGSGGSGGSGGSGGTGGSGGSGMVMEKPSPLLVGREFVRQYYTLLNQAPDMLHRFYGKNSSYVHGGLDSNGKPADAVYGQKEIHRKVMSQNFTNCHTKIRHVDAHATLNDGVVVQVMGLLSNNNQALRRFMQTFVLAPEGSVANKFYVHNDIFRYQDEVFGGFVTEPQEESEEEVEEPEERQQTPEVVPDDSGTFYDQAVVSNDMEEHLEEPVAEPEPDPEPEPEQEPVSEIQEEKPEPVLEETAPEDAQKSSSPAPADIAQTVQEDLRTFSWASVTSKNLPPSGAVPVTGIPPHVVKVPASQPRPESKPESQIPPQRPQRDQRVREQRINIPPQRGPRPIREAGEQGDIEPRRMVRHPDSHQLFIGNLPHEVDKSELKDFFQSYGNVVELRINSGGKLPNFGFVVFDDSEPVQKVLSNRPIMFRGEVRLNVEEKKTRAAREGDRRDNRLRGPGGPRGGLGGGMRGPPRGGMVQKPGFGVGRGLAPRQKLIPMARRAWLEHGHSCFLCEIVIRSQFHTTYEPEA

SEQ ID NO: 73 - protein, 536 aa, synthetic construct
  REGION 1..536: note = Synthetic construct
MHTLYAPGGYDIMGWLCQIMNRPNPQVELGPVDTSVALVLCDLKQKDTPVVYASEAFLYMTGYSNAEVLGRNCRFLQSPDGMVKPKSTRKYVDSNTINTMRKAIDRNAEVQVEVVNFKKNGQRFVNFLTMIPVRDETGEYRYSMGFQCETEGSGGSGGSGGSGGTGGSGGSGMGFVTEPQEESEEEVEEPEERQQTPEVVPDDSGTFYDQAVVSNDMEEHLEEPVAEPEPDPEPEPEQEPVSEIQEEKPEPVLEETAPEDAQKSSSPAPADIAQTVQEDLRTFSWASVTSKNLPPSGAVPVTGIPPHVVKVPASQPRPESKPESQIPPQRPQRDQRVREQRINIPPQRGPRPIREAGEQGDIEPRRMVRHPDSHQLFIGNLPHEVDKSELKDFFQSYGNVVELRINSGGKLPNFGFVVFDDSEPVQKVLSNRPIMFRGEVRLNVEEKKTRAAREGDRRDNRLRGPGGPRGGLGGGMRGPPRGGMVQKPGFGVGRGLAPRQKLIPMARRAWLEHGHSCFLCEIVIRSQFHTTYEPEA

SEQ ID NO: 74 - protein, 592 aa, synthetic construct
  REGION 1..592: note = Synthetic construct
MHTLYAPGGYDIMGWLCQIMNRPNPQVELGPVDTSVALVLCDLKQKDTPVVYASEAFLYMTGYSNAEVLGRNCRFLQSPDGMVKPKSTRKYVDSNTINTMRKAIDRNAEVQVEVVNFKKNGQRFVNFLTMIPVRDETGEYRYSMGFQCETEGSGGSGGSGGSGGTGGSGGSGMVMEKPSPLLVGREFVRQYYTLLNQAPDMLHRFYGKNSSYVHGGLDSNGKPADAVYGQKEIHRKVMSQNFTNCHTKIRHVDAHATLNDGVVVQVMGLLSNNNQALRRFMQTFVLAPEGSVANKFYVHNDIFRYQDEVFGTAPEDAQKSSSPAPADIAQTVQEDLRTFSWASVTSKNLPPSGAVPVTGIPPHVVKVPASQPRPESKPESQIPPQRPQRDQRVREQRINIPPQRGPRPIREAGEQGDIEPRRMVRHPDSHQLFIGNLPHEVDKSELKDFFQSYGNVVELRINSGGKLPNFGFVVFDDSEPVQKVLSNRPIMFRGEVRLNVEEKKTRAAREGDRRDNRLRGPGGPRGGLGGGMRGPPRGGMVQKPGFGVGRGLAPRQKLIPMARRAWLEHGHSCFLCEIVIRSQFHTTYEPEA

SEQ ID NO: 75 - protein, 555 aa, synthetic construct
  REGION 1..555: note = Synthetic construct
MHTLYAPGGYDIMGWLCQIMNRPNPQVELGPVDTSVALVLCDLKQKDTPVVYASEAFLYMTGYSNAEVLGRNCRFLQSPDGMVKPKSTRKYVDSNTINTMRKAIDRNAEVQVEVVNFKKNGQRFVNFLTMIPVRDETGEYRYSMGFQCETEGSGGSGGSGGSGGTGGSGGSGMVMEKPSPLLVGREFVRQYYTLLNQAPDMLHRFYGKNSSYVHGGLDSNGKPADAVYGQKEIHRKVMSQNFTNCHTKIRHVDAHATLNDGVVVQVMGLLSNNNQALRRFMQTFVLAPEGSVANKFYVHNDIFRYQDEVFGGFVTEPQEESEEEVEEPEERQQTPEVVPDDSGTFYDQAVVSNDMEEHLEEPVAEPEPDPEPEPEQEPVSEIQEEKPEPVLEEQLFIGNLPHEVDKSELKDFFQSYGNVVELRINSGGKLPNFGFVVFDDSEPVQKVLSNRPIMFRGEVRLNVEEKKTRAAREGDRRDNRLRGPGGPRGGLGGGMRGPPRGGMVQKPGFGVGRGLAPRQKLIPMARRAWLEHGHSCFLCEIVIRSQFHTTYEPEA

SEQ ID NO: 76 - protein, 603 aa, synthetic construct
  REGION 1..603: note = Synthetic construct
MHTLYAPGGYDIMGWLCQIMNRPNPQVELGPVDTSVALVLCDLKQKDTPVVYASEAFLYMTGYSNAEVLGRNCRFLQSPDGMVKPKSTRKYVDSNTINTMRKAIDRNAEVQVEVVNFKKNGQRFVNFLTMIPVRDETGEYRYSMGFQCETEGSGGSGGSGGSGGTGGSGGSGMVMEKPSPLLVGREFVRQYYTLLNQAPDMLHRFYGKNSSYVHGGLDSNGKPADAVYGQKEIHRKVMSQNFTNCHTKIRHVDAHATLNDGVVVQVMGLLSNNNQALRRFMQTFVLAPEGSVANKFYVHNDIFRYQDEVFGGFVTEPQEESEEEVEEPEERQQTPEVVPDDSGTFYDQAVVSNDMEEHLEEPVAEPEPDPEPEPEQEPVSEIQEEKPEPVLEETAPEDAQKSSSPAPADIAQTVQEDLRTFSWASVTSKNLPPSGAVPVTGIPPHVVKVPASQPRPESKPESQIPPQRPQRDQRVREQRINIPPQRGPRPIREAGEQGDIEPRRMVRHPDSHEKKTRAAREGDRRDNRLRGPGGPRGGLGGGMRGPPRGGMVQKPGFGVGRGLAPRQKLIPMARRAWLEHGHSCFLCEIVIRSQFHTTYEPEA

SEQ ID NO: 77 - protein, 583 aa, synthetic construct
  REGION 1..583: note = Synthetic construct
MHTLYAPGGYDIMGWLCQIMNRPNPQVELGPVDTSVALVLCDLKQKDTPVVYASEAFLYMTGYSNAEVLGRNCRFLQSPDGMVKPKSTRKYVDSNTINTMRKAIDRNAEVQVEVVNFKKNGQRFVNFLTMIPVRDETGEYRYSMGFQCETEGSGGSGGSGGSGGTGGSGGSGMVMEKPSPLLVGREFVRQYYTLLNQAPDMLHRFYGKNSSYVHGGLDSNGKPADAVYGQKEIHRKVMSQNFTNCHTKIRHVDAHATLNDGVVVQVMGLLSNNNQALRRFMQTFVLAPEGSVANKFYVHNDIFRYQDEVFGGFVTEPQEESEEEVEEPEERQQTPEVVPDDSGTFYDQAVVSNDMEEHLEEPVAEPEPDPEPEPEQEPVSEIQEEKPEPVLEETAPEDAQKSSSPAPADIAQTVQEDLRTFSWASVTSKNLPPSGAVPVTGIPPHVVKVPASQPRPESKPESQIPPQRPQRDQRVREQRINIPPQRGPRPIREAGEQGDIEPRRMVRHPDSHQLFIGNLPHEVDKSELKDFFQSYGNVVELRINSGGKLPNFGFVVFDDSEPVQKVLSNRPIMFRGEVRLNVE

SEQ ID NO: 78 - protein, 512 aa, synthetic construct
  REGION 1..512: note = Synthetic construct
MHTLYAPGGYDIMGWLCQIMNRPNPQVELGPVDTSVALVLCDLKQKDTPVVYASEAFLYMTGYSNAEVLGRNCRFLQSPDGMVKPKSTRKYVDSNTINTMRKAIDRNAEVQVEVVNFKKNGQRFVNFLTMIPVRDETGEYRYSMGFQCETEGSGGSGGSGGSGGTGGSGGSGMVMEKPSPLLVGREFVRQYYTLLNQAPDMLHRFYGKNSSYVHGGLDSNGKPADAVYGQKEIHRKVMSQNFTNCHTKIRHVDAHATLNDGVVVQVMGLLSNNNQALRRFMQTFVLAPEGSVANKFYVHNDIFRYQDEVFGGFVTEPQEESEEEVEEPEERQQTPEVVPDDSGTFYDQAVVSNDMEEHLEEPVAEPEPDPEPEPEQEPVSEIQEEKPEPVLEETAPEDAQKSSSPAPADIAQTVQEDLRTFSWASVTSKNLPPSGAVPVTGIPPHVVKVPASQPRPESKPESQIPPQRPQRDQRVREQRINIPPQRGPRPIREAGEQGDIEPRRMVRHPDSH

SEQ ID NO: 79 - protein, 1000 aa, synthetic construct
  REGION 1..1000: note = Synthetic construct
MKMDKKTIVWFRRDLRIEDNPALAAAAHEGSVFPVFIWCPEEEGQFYPGRASRWWMKQSLAHLSQSLKALGSDLTLIKTHNTISAILDCIRVTGATKVVFNHLYDPVSLVRDHTVKEKLVERGISVQSYNGDLLYEPWEIYCEKGKPFTSFNSYWKKCLDMSIESVMLPPPWRLMPITAAAEAIWACSIEELGLENEAEKPSNALLTRAWSPGWSNADKLLNEFIEKQLIDYAKNSKKVVGNSTSLLSPYLHFGEISVRHVFQCARMKQIIWARDKNSEGEESADLFLRGIGLREYSRYICFNFPFTHEQSLLSHLRFFPWDADVDKFKAWRQGRTGYPLVDAGMRELWATGWMHNRIRVIVSSFAVKFLLLPWKWGMKYFWDTLLDADLECDILGWQYISGSIPDGHELDRLDNPALQGAKYDPEGEYIRQWLPELARLPTEWIHHPWDAPLTVLKASGVELGTNYAKPIVDIDTARELLAKAISRTRGAQIMIGAAMVMEKPSPLLVGREFVRQYYTLLNQAPDMLHRFYGKNSSYVHGGLDSNGKPADAVYGQKEIHRKVMSQNFTNCHTKIRHVDAHATLNDGVVVQVMGLLSNNNQALRRFMQTFVLAPEGSVANKFYVHNDIFRYQDEVFGGFVTEPQEESEEEVEEPEERQQTPEVVPDDSGTFYDQAVVSNDMEEHLEEPVAEPEPDPEPEPEQEPVSEIQEEKPEPVLEETAPEDAQKSSSPAPADIAQTVQEDLRTFSWASVTSKNLPPSGAVPVTGIPPHVVKVPASQPRPESKPESQIPPQRPQRDQRVREQRINIPPQRGPRPIREAGEQGDIEPRRMVRHPDSHQLFIGNLPHEVDKSELKDFFQSYGNVVELRINSGGKLPNFGFVVFDDSEPVQKVLSNRPIMFRGEVRLNVEEKKTRAAREGDRRDNRLRGPGGPRGGLGGGMRGPPRGGMVQKPGFGVGRGLAPRQKLIPMARRAWLEHGHSCFLCEIVIRSQFHTTYEPEA

SEQ ID NO: 80 - protein, 862 aa, synthetic construct
  REGION 1..862: note = Synthetic construct
MKMDKKTIVWFRRDLRIEDNPALAAAAHEGSVFPVFIWCPEEEGQFYPGRASRWWMKQSLAHLSQSLKALGSDLTLIKTHNTISAILDCIRVTGATKVVFNHLYDPVSLVRDHTVKEKLVERGISVQSYNGDLLYEPWEIYCEKGKPFTSFNSYWKKCLDMSIESVMLPPPWRLMPITAAAEAIWACSIEELGLENEAEKPSNALLTRAWSPGWSNADKLLNEFIEKQLIDYAKNSKKVVGNSTSLLSPYLHFGEISVRHVFQCARMKQIIWARDKNSEGEESADLFLRGIGLREYSRYICFNFPFTHEQSLLSHLRFFPWDADVDKFKAWRQGRTGYPLVDAGMRELWATGWMHNRIRVIVSSFAVKFLLLPWKWGMKYFWDTLLDADLECDILGWQYISGSIPDGHELDRLDNPALQGAKYDPEGEYIRQWLPELARLPTEWIHHPWDAPLTVLKASGVELGTNYAKPIVDIDTARELLAKAISRTRGAQIMIGAAMGFVTEPQEESEEEVEEPEERQQTPEVVPDDSGTFYDQAVVSNDMEEHLEEPVAEPEPDPEPEPEQEPVSEIQEEKPEPVLEETAPEDAQKSSSPAPADIAQTVQEDLRTFSWASVTSKNLPPSGAVPVTGIPPHVVKVPASQPRPESKPESQIPPQRPQRDQRVREQRINIPPQRGPRPIREAGEQGDIEPRRMVRHPDSHQLFIGNLPHEVDKSELKDFFQSYGNVVELRINSGGKLPNFGFVVFDDSEPVQKVLSNRPIMFRGEVRLNVEEKKTRAAREGDRRDNRLRGPGGPRGGLGGGMRGPPRGGMVQKPGFGVGRGLAPRQKLIPMARRAWLEHGHSCFLCEIVIRSQFHTTYEPEA

SEQ ID NO: 81 - protein, 918 aa, synthetic construct
  REGION 1..918: note = Synthetic construct
MKMDKKTIVWFRRDLRIEDNPALAAAAHEGSVFPVFIWCPEEEGQFYPGRASRWWMKQSLAHLSQSLKALGSDLTLIKTHNTISAILDCIRVTGATKVVFNHLYDPVSLVRDHTVKEKLVERGISVQSYNGDLLYEPWEIYCEKGKPFTSFNSYWKKCLDMSIESVMLPPPWRLMPITAAAEAIWACSIEELGLENEAEKPSNALLTRAWSPGWSNADKLLNEFIEKQLIDYAKNSKKVVGNSTSLLSPYLHFGEISVRHVFQCARMKQIIWARDKNSEGEESADLFLRGIGLREYSRYICFNFPFTHEQSLLSHLRFFPWDADVDKFKAWRQGRTGYPLVDAGMRELWATGWMHNRIRVIVSSFAVKFLLLPWKWGMKYFWDTLLDADLECDILGWQYISGSIPDGHELDRLDNPALQGAKYDPEGEYIRQWLPELARLPTEWIHHPWDAPLTVLKASGVELGTNYAKPIVDIDTARELLAKAISRTRGAQIMIGAAMVMEKPSPLLVGREFVRQYYTLLNQAPDMLHRFYGKNSSYVHGGLDSNGKPADAVYGQKEIHRKVMSQNFTNCHTKIRHVDAHATLNDGVVVQVMGLLSNNNQALRRFMQTFVLAPEGSVANKFYVHNDIFRYQDEVFGTAPEDAQKSSSPAPADIAQTVQEDLRTFSWASVTSKNLPPSGAVPVTGIPPHVVKVPASQPRPESKPESQIPPQRPQRDQRVREQRINIPPQRGPRPIREAGEQGDIEPRRMVRHPDSHQLFIGNLPHEVDKSELKDFFQSYGNVVELRINSGGKLPNFGFVVFDDSEPVQKVLSNRPIMFRGEVRLNVEEKKTRAAREGDRRDNRLRGPGGPRGGLGGGMRGPPRGGMVQKPGFGVGRGLAPRQKLIPMARRAWLEHGHSCFLCEIVIRSQFHTTYEPEA

SEQ ID NO: 82 - protein, 881 aa, synthetic construct
  REGION 1..881: note = Synthetic construct
MKMDKKTIVWFRRDLRIEDNPALAAAAHEGSVFPVFIWCPEEEGQFYPGRASRWWMKQSLAHLSQSLKALGSDLTLIKTHNTISAILDCIRVTGATKVVFNHLYDPVSLVRDHTVKEKLVERGISVQSYNGDLLYEPWEIYCEKGKPFTSFNSYWKKCLDMSIESVMLPPPWRLMPITAAAEAIWACSIEELGLENEAEKPSNALLTRAWSPGWSNADKLLNEFIEKQLIDYAKNSKKVVGNSTSLLSPYLHFGEISVRHVFQCARMKQIIWARDKNSEGEESADLFLRGIGLREYSRYICFNFPFTHEQSLLSHLRFFPWDADVDKFKAWRQGRTGYPLVDAGMRELWATGWMHNRIRVIVSSFAVKFLLLPWKWGMKYFWDTLLDADLECDILGWQYISGSIPDGHELDRLDNPALQGAKYDPEGEYIRQWLPELARLPTEWIHHPWDAPLTVLKASGVELGTNYAKPIVDIDTARELLAKAISRTRGAQIMIGAAMVMEKPSPLLVGREFVRQYYTLLNQAPDMLHRFYGKNSSYVHGGLDSNGKPADAVYGQKEIHRKVMSQNFTNCHTKIRHVDAHATLNDGVVVQVMGLLSNNNQALRRFMQTFVLAPEGSVANKFYVHNDIFRYQDEVFGGFVTEPQEESEEEVEEPEERQQTPEVVPDDSGTFYDQAVVSNDMEEHLEEPVAEPEPDPEPEPEQEPVSEIQEEKPEPVLEEQLFIGNLPHEVDKSELKDFFQSYGNVVELRINSGGKLPNFGFVVFDDSEPVQKVLSNRPIMFRGEVRLNVEEKKTRAAREGDRRDNRLRGPGGPRGGLGGGMRGPPRGGMVQKPGFGVGRGLAPRQKLIPMARRAWLEHGHSCFLCEIVIRSQFHTTYEPEA

SEQ ID NO: 83 - protein, 929 aa, synthetic construct
  REGION 1..929: note = Synthetic construct
MKMDKKTIVWFRRDLRIEDNPALAAAAHEGSVFPVFIWCPEEEGQFYPGRASRWWMKQSLAHLSQSLKALGSDLTLIKTHNTISAILDCIRVTGATKVVFNHLYDPVSLVRDHTVKEKLVERGISVQSYNGDLLYEPWEIYCEKGKPFTSFNSYWKKCLDMSIESVMLPPPWRLMPITAAAEAIWACSIEELGLENEAEKPSNALLTRAWSPGWSNADKLLNEFIEKQLIDYAKNSKKVVGNSTSLLSPYLHFGEISVRHVFQCARMKQIIWARDKNSEGEESADLFLRGIGLREYSRYICFNFPFTHEQSLLSHLRFFPWDADVDKFKAWRQGRTGYPLVDAGMRELWATGWMHNRIRVIVSSFAVKFLLLPWKWGMKYFWDTLLDADLECDILGWQYISGSIPDGHELDRLDNPALQGAKYDPEGEYIRQWLPELARLPTEWIHHPWDAPLTVLKASGVELGTNYAKPIVDIDTARELLAKAISRTRGAQIMIGAAMVMEKPSPLLVGREFVRQYYTLLNQAPDMLHRFYGKNSSYVHGGLDSNGKPADAVYGQKEIHRKVMSQNFTNCHTKIRHVDAHATLNDGVVVQVMGLLSNNNQALRRFMQTFVLAPEGSVANKFYVHNDIFRYQDEVFGGFVTEPQEESEEEVEEPEERQQTPEVVPDDSGTFYDQAVVSNDMEEHLEEPVAEPEPDPEPEPEQEPVSEIQEEKPEPVLEETAPEDAQKSSSPAPADIAQTVQEDLRTFSWASVTSKNLPPSGAVPVTGIPPHVVKVPASQPRPESKPESQIPPQRPQRDQRVREQRINIPPQRGPRPIREAGEQGDIEPRRMVRHPDSHEKKTRAAREGDRRDNRLRGPGGPRGGLGGGMRGPPRGGMVQKPGFGVGRGLAPRQKLIPMARRAWLEHGHSCFLCEIVIRSQFHTTYEPEA

SEQ ID NO: 84 - protein, 909 aa, synthetic construct
  REGION 1..909: note = Synthetic construct
MKMDKKTIVWFRRDLRIEDNPALAAAAHEGSVFPVFIWCPEEEGQFYPGRASRWWMKQSLAHLSQSLKALGSDLTLIKTHNTISAILDCIRVTGATKVVFNHLYDPVSLVRDHTVKEKLVERGISVQSYNGDLLYEPWEIYCEKGKPFTSFNSYWKKCLDMSIESVMLPPPWRLMPITAAAEAIWACSIEELGLENEAEKPSNALLTRAWSPGWSNADKLLNEFIEKQLIDYAKNSKKVVGNSTSLLSPYLHFGEISVRHVFQCARMKQIIWARDKNSEGEESADLFLRGIGLREYSRYICFNFPFTHEQSLLSHLRFFPWDADVDKFKAWRQGRTGYPLVDAGMRELWATGWMHNRIRVIVSSFAVKFLLLPWKWGMKYFWDTLLDADLECDILGWQYISGSIPDGHELDRLDNPALQGAKYDPEGEYIRQWLPELARLPTEWIHHPWDAPLTVLKASGVELGTNYAKPIVDIDTARELLAKAISRTRGAQIMIGAAMVMEKPSPLLVGREFVRQYYTLLNQAPDMLHRFYGKNSSYVHGGLDSNGKPADAVYGQKEIHRKVMSQNFTNCHTKIRHVDAHATLNDGVVVQVMGLLSNNNQALRRFMQTFVLAPEGSVANKFYVHNDIFRYQDEVFGGFVTEPQEESEEEVEEPEERQQTPEVVPDDSGTFYDQAVVSNDMEEHLEEPVAEPEPDPEPEPEQEPVSEIQEEKPEPVLEETAPEDAQKSSSPAPADIAQTVQEDLRTFSWASVTSKNLPPSGAVPVTGIPPHVVKVPASQPRPESKPESQIPPQRPQRDQRVREQRINIPPQRGPRPIREAGEQGDIEPRRMVRHPDSHQLFIGNLPHEVDKSELKDFFQSYGNVVELRINSGGKLPNFGFVVFDDSEPVQKVLSNRPIMFRGEVRLNVE

SEQ ID NO: 85 - protein, 838 aa, synthetic construct
  REGION 1..838: note = Synthetic construct
MKMDKKTIVWFRRDLRIEDNPALAAAAHEGSVFPVFIWCPEEEGQFYPGRASRWWMKQSLAHLSQSLKALGSDLTLIKTHNTISAILDCIRVTGATKVVFNHLYDPVSLVRDHTVKEKLVERGISVQSYNGDLLYEPWEIYCEKGKPFTSFNSYWKKCLDMSIESVMLPPPWRLMPITAAAEAIWACSIEELGLENEAEKPSNALLTRAWSPGWSNADKLLNEFIEKQLIDYAKNSKKVVGNSTSLLSPYLHFGEISVRHVFQCARMKQIIWARDKNSEGEESADLFLRGIGLREYSRYICFNFPFTHEQSLLSHLRFFPWDADVDKFKAWRQGRTGYPLVDAGMRELWATGWMHNRIRVIVSSFAVKFLLLPWKWGMKYFWDTLLDADLECDILGWQYISGSIPDGHELDRLDNPALQGAKYDPEGEYIRQWLPELARLPTEWIHHPWDAPLTVLKASGVELGTNYAKPIVDIDTARELLAKAISRTRGAQIMIGAAMVMEKPSPLLVGREFVRQYYTLLNQAPDMLHRFYGKNSSYVHGGLDSNGKPADAVYGQKEIHRKVMSQNFTNCHTKIRHVDAHATLNDGVVVQVMGLLSNNNQALRRFMQTFVLAPEGSVANKFYVHNDIFRYQDEVFGGFVTEPQEESEEEVEEPEERQQTPEVVPDDSGTFYDQAVVSNDMEEHLEEPVAEPEPDPEPEPEQEPVSEIQEEKPEPVLEETAPEDAQKSSSPAPADIAQTVQEDLRTFSWASVTSKNLPPSGAVPVTGIPPHVVKVPASQPRPESKPESQIPPQRPQRDQRVREQRINIPPQRGPRPIREAGEQGDIEPRRMVRHPDSH

SEQ ID NO: 86 - protein, 1000 aa, synthetic construct
  REGION 1..1000: note = Synthetic construct
MVMEKPSPLLVGREFVRQYYTLLNQAPDMLHRFYGKNSSYVHGGLDSNGKPADAVYGQKEIHRKVMSQNFTNCHTKIRHVDAHATLNDGVVVQVMGLLSNNNQALRRFMQTFVLAPEGSVANKFYVHNDIFRYQDEVFGGFVTEPQEESEEEVEEPEERQQTPEVVPDDSGTFYDQAVVSNDMEEHLEEPVAEPEPDPEPEPEQEPVSEIQEEKPEPVLEETAPEDAQKSSSPAPADIAQTVQEDLRTFSWASVTSKNLPPSGAVPVTGIPPHVVKVPASQPRPESKPESQIPPQRPQRDQRVREQRINIPPQRGPRPIREAGEQGDIEPRRMVRHPDSHQLFIGNLPHEVDKSELKDFFQSYGNVVELRINSGGKLPNFGFVVFDDSEPVQKVLSNRPIMFRGEVRLNVEEKKTRAAREGDRRDNRLRGPGGPRGGLGGGMRGPPRGGMVQKPGFGVGRGLAPRQKLIPMARRAWLEHGHSCFLCEIVIRSQFHTTYEPEAMKMDKKTIVWFRRDLRIEDNPALAAAAHEGSVFPVFIWCPEEEGQFYPGRASRWWMKQSLAHLSQSLKALGSDLTLIKTHNTISAILDCIRVTGATKVVFNHLYDPVSLVRDHTVKEKLVERGISVQSYNGDLLYEPWEIYCEKGKPFTSFNSYWKKCLDMSIESVMLPPPWRLMPITAAAEAIWACSIEELGLENEAEKPSNALLTRAWSPGWSNADKLLNEFIEKQLIDYAKNSKKVVGNSTSLLSPYLHFGEISVRHVFQCARMKQIIWARDKNSEGEESADLFLRGIGLREYSRYICFNFPFTHEQSLLSHLRFFPWDADVDKFKAWRQGRTGYPLVDAGMRELWATGWMHNRIRVIVSSFAVKFLLLPWKWGMKYFWDTLLDADLECDILGWQYISGSIPDGHELDRLDNPALQGAKYDPEGEYIRQWLPELARLPTEWIHHPWDAPLTVLKASGVELGTNYAKPIVDIDTARELLAKAISRTRGAQIMIGAA

SEQ ID NO: 87 - protein, 862 aa, synthetic construct
  REGION 1..862: note = Synthetic construct
MGFVTEPQEESEEEVEEPEERQQTPEVVPDDSGTFYDQAVVSNDMEEHLEEPVAEPEPDPEPEPEQEPVSEIQEEKPEPVLEETAPEDAQKSSSPAPADIAQTVQEDLRTFSWASVTSKNLPPSGAVPVTGIPPHVVKVPASQPRPESKPESQIPPQRPQRDQRVREQRINIPPQRGPRPIREAGEQGDIEPRRMVRHPDSHQLFIGNLPHEVDKSELKDFFQSYGNVVELRINSGGKLPNFGFVVFDDSEPVQKVLSNRPIMFRGEVRLNVEEKKTRAAREGDRRDNRLRGPGGPRGGLGGGMRGPPRGGMVQKPGFGVGRGLAPRQKLIPMARRAWLEHGHSCFLCEIVIRSQFHTTYEPEAMKMDKKTIVWFRRDLRIEDNPALAAAAHEGSVFPVFIWCPEEEGQFYPGRASRWWMKQSLAHLSQSLKALGSDLTLIKTHNTISAILDCIRVTGATKVVFNHLYDPVSLVRDHTVKEKLVERGISVQSYNGDLLYEPWEIYCEKGKPFTSFNSYWKKCLDMSIESVMLPPPWRLMPITAAAEAIWACSIEELGLENEAEKPSNALLTRAWSPGWSNADKLLNEFIEKQLIDYAKNSKKVVGNSTSLLSPYLHFGEISVRHVFQCARMKQIIWARDKNSEGEESADLFLRGIGLREYSRYICFNFPFTHEQSLLSHLRFFPWDADVDKFKAWRQGRTGYPLVDAGMRELWATGWMHNRIRVIVSSFAVKFLLLPWKWGMKYFWDTLLDADLECDILGWQYISGSIPDGHELDRLDNPALQGAKYDPEGEYIRQWLPELARLPTEWIHHPWDAPLTVLKASGVELGTNYAKPIVDIDTARELLAKAISRTRGAQIMIGAA

SEQ ID NO: 88 - protein, 918 aa, synthetic construct
  REGION 1..918: note = Synthetic construct
MVMEKPSPLLVGREFVRQYYTLLNQAPDMLHRFYGKNSSYVHGGLDSNGKPADAVYGQKEIHRKVMSQNFTNCHTKIRHVDAHATLNDGVVVQVMGLLSNNNQALRRFMQTFVLAPEGSVANKFYVHNDIFRYQDEVFGTAPEDAQKSSSPAPADIAQTVQEDLRTFSWASVTSKNLPPSGAVPVTGIPPHVVKVPASQPRPESKPESQIPPQRPQRDQRVREQRINIPPQRGPRPIREAGEQGDIEPRRMVRHPDSHQLFIGNLPHEVDKSELKDFFQSYGNVVELRINSGGKLPNFGFVVFDDSEPVQKVLSNRPIMFRGEVRLNVEEKKTRAAREGDRRDNRLRGPGGPRGGLGGGMRGPPRGGMVQKPGFGVGRGLAPRQKLIPMARRAWLEHGHSCFLCEIVIRSQFHTTYEPEAMKMDKKTIVWFRRDLRIEDNPALAAAAHEGSVFPVFIWCPEEEGQFYPGRASRWWMKQSLAHLSQSLKALGSDLTLIKTHNTISAILDCIRVTGATKVVFNHLYDPVSLVRDHTVKEKLVERGISVQSYNGDLLYEPWEIYCEKGKPFTSFNSYWKKCLDMSIESVMLPPPWRLMPITAAAEAIWACSIEELGLENEAEKPSNALLTRAWSPGWSNADKLLNEFIEKQLIDYAKNSKKVVGNSTSLLSPYLHFGEISVRHVFQCARMKQIIWARDKNSEGEESADLFLRGIGLREYSRYICFNFPFTHEQSLLSHLRFFPWDADVDKFKAWRQGRTGYPLVDAGMRELWATGWMHNRIRVIVSSFAVKFLLLPWKWGMKYFWDTLLDADLECDILGWQYISGSIPDGHELDRLDNPALQGAKYDPEGEYIRQWLPELARLPTEWIHHPWDAPLTVLKASGVELGTNYAKPIVDIDTARELLAKAISRTRGAQIMIGAA

SEQ ID NO: 89 - protein, 881 aa, synthetic construct
  REGION 1..881: note = Synthetic construct
MVMEKPSPLLVGREFVRQYYTLLNQAPDMLHRFYGKNSSYVHGGLDSNGKPADAVYGQKEIHRKVMSQNFTNCHTKIRHVDAHATLNDGVVVQVMGLLSNNNQALRRFMQTFVLAPEGSVANKFYVHNDIFRYQDEVFGGFVTEPQEESEEEVEEPEERQQTPEVVPDDSGTFYDQAVVSNDMEEHLEEPVAEPEPDPEPEPEQEPVSEIQEEKPEPVLEEQLFIGNLPHEVDKSELKDFFQSYGNVVELRINSGGKLPNFGFVVFDDSEPVQKVLSNRPIMFRGEVRLNVEEKKTRAAREGDRRDNRLRGPGGPRGGLGGGMRGPPRGGMVQKPGFGVGRGLAPRQKLIPMARRAWLEHGHSCFLCEIVIRSQFHTTYEPEAMKMDKKTIVWFRRDLRIEDNPALAAAAHEGSVFPVFIWCPEEEGQFYPGRASRWWMKQSLAHLSQSLKALGSDLTLIKTHNTISAILDCIRVTGATKVVFNHLYDPVSLVRDHTVKEKLVERGISVQSYNGDLLYEPWEIYCEKGKPFTSFNSYWKKCLDMSIESVMLPPPWRLMPITAAAEAIWACSIEELGLENEAEKPSNALLTRAWSPGWSNADKLLNEFIEKQLIDYAKNSKKVVGNSTSLLSPYLHFGEISVRHVFQCARMKQIIWARDKNSEGEESADLFLRGIGLREYSRYICFNFPFTHEQSLLSHLRFFPWDADVDKFKAWRQGRTGYPLVDAGMRELWATGWMHNRIRVIVSSFAVKFLLLPWKWGMKYFWDTLLDADLECDILGWQYISGSIPDGHELDRLDNPALQGAKYDPEGEYIRQWLPELARLPTEWIHHPWDAPLTVLKASGVELGTNYAKPIVDIDTARELLAKAISRTRGAQIMIGAA

SEQ ID NO: 90 - protein, 929 aa, synthetic construct
  REGION 1..929: note = Synthetic construct
MVMEKPSPLLVGREFVRQYYTLLNQAPDMLHRFYGKNSSYVHGGLDSNGKPADAVYGQKEIHRKVMSQNFTNCHTKIRHVDAHATLNDGVVVQVMGLLSNNNQALRRFMQTFVLAPEGSVANKFYVHNDIFRYQDEVFGGFVTEPQEESEEEVEEPEERQQTPEVVPDDSGTFYDQAVVSNDMEEHLEEPVAEPEPDPEPEPEQEPVSEIQEEKPEPVLEETAPEDAQKSSSPAPADIAQTVQEDLRTFSWASVTSKNLPPSGAVPVTGIPPHVVKVPASQPRPESKPESQIPPQRPQRDQRVREQRINIPPQRGPRPIREAGEQGDIEPRRMVRHPDSHEKKTRAAREGDRRDNRLRGPGGPRGGLGGGMRGPPRGGMVQKPGFGVGRGLAPRQKLIPMARRAWLEHGHSCFLCEIVIRSQFHTTYEPEAMKMDKKTIVWFRRDLRIEDNPALAAAAHEGSVFPVFIWCPEEEGQFYPGRASRWWMKQSLAHLSQSLKALGSDLTLIKTHNTISAILDCIRVTGATKVVFNHLYDPVSLVRDHTVKEKLVERGISVQSYNGDLLYEPWEIYCEKGKPFTSFNSYWKKCLDMSIESVMLPPPWRLMPITAAAEAIWACSIEELGLENEAEKPSNALLTRAWSPGWSNADKLLNEFIEKQLIDYAKNSKKVVGNSTSLLSPYLHFGEISVRHVFQCARMKQIIWARDKNSEGEESADLFLRGIGLREYSRYICFNFPFTHEQSLLSHLRFFPWDADVDKFKAWRQGRTGYPLVDAGMRELWATGWMHNRIRVIVSSFAVKFLLLPWKWGMKYFWDTLLDADLECDILGWQYISGSIPDGHELDRLDNPALQGAKYDPEGEYIRQWLPELARLPTEWIHHPWDAPLTVLKASGVELGTNYAKPIVDIDTARELLAKAISRTRGAQIMIGAA

SEQ ID NO: 91 - protein, 909 aa, synthetic construct
  REGION 1..909: note = Synthetic construct
MVMEKPSPLLVGREFVRQYYTLLNQAPDMLHRFYGKNSSYVHGGLDSNGKPADAVYGQKEIHRKVMSQNFTNCHTKIRHVDAHATLNDGVVVQVMGLLSNNNQALRRFMQTFVLAPEGSVANKFYVHNDIFRYQDEVFGGFVTEPQEESEEEVEEPEERQQTPEVVPDDSGTFYDQAVVSNDMEEHLEEPVAEPEPDPEPEPEQEPVSEIQEEKPEPVLEETAPEDAQKSSSPAPADIAQTVQEDLRTFSWASVTSKNLPPSGAVPVTGIPPHVVKVPASQPRPESKPESQIPPQRPQRDQRVREQRINIPPQRGPRPIREAGEQGDIEPRRMVRHPDSHQLFIGNLPHEVDKSELKDFFQSYGNVVELRINSGGKLPNFGFVVFDDSEPVQKVLSNRPIMFRGEVRLNVEMKMDKKTIVWFRRDLRIEDNPALAAAAHEGSVFPVFIWCPEEEGQFYPGRASRWWMKQSLAHLSQSLKALGSDLTLIKTHNTISAILDCIRVTGATKVVFNHLYDPVSLVRDHTVKEKLVERGISVQSYNGDLLYEPWEIYCEKGKPFTSFNSYWKKCLDMSIESVMLPPPWRLMPITAAAEAIWACSIEELGLENEAEKPSNALLTRAWSPGWSNADKLLNEFIEKQLIDYAKNSKKVVGNSTSLLSPYLHFGEISVRHVFQCARMKQIIWARDKNSEGEESADLFLRGIGLREYSRYICFNFPFTHEQSLLSHLRFFPWDADVDKFKAWRQGRTGYPLVDAGMRELWATGWMHNRIRVIVSSFAVKFLLLPWKWGMKYFWDTLLDADLECDILGWQYISGSIPDGHELDRLDNPALQGAKYDPEGEYIRQWLPELARLPTEWIHHPWDAPLTVLKASGVELGTNYAKPIVDIDTARELLAKAISRTRGAQIMIGAA

SEQ ID NO: 92 - protein, 838 aa, synthetic construct
  REGION 1..838: note = Synthetic construct
MVMEKPSPLLVGREFVRQYYTLLNQAPDMLHRFYGKNSSYVHGGLDSNGKPADAVYGQKEIHRKVMSQNFTNCHTKIRHVDAHATLNDGVVVQVMGLLSNNNQALRRFMQTFVLAPEGSVANKFYVHNDIFRYQDEVFGGFVTEPQEESEEEVEEPEERQQTPEVVPDDSGTFYDQAVVSNDMEEHLEEPVAEPEPDPEPEPEQEPVSEIQEEKPEPVLEETAPEDAQKSSSPAPADIAQTVQEDLRTFSWASVTSKNLPPSGAVPVTGIPPHVVKVPASQPRPESKPESQIPPQRPQRDQRVREQRINIPPQRGPRPIREAGEQGDIEPRRMVRHPDSHMKMDKKTIVWFRRDLRIEDNPALAAAAHEGSVFPVFIWCPEEEGQFYPGRASRWWMKQSLAHLSQSLKALGSDLTLIKTHNTISAILDCIRVTGATKVVFNHLYDPVSLVRDHTVKEKLVERGISVQSYNGDLLYEPWEIYCEKGKPFTSFNSYWKKCLDMSIESVMLPPPWRLMPITAAAEAIWACSIEELGLENEAEKPSNALLTRAWSPGWSNADKLLNEFIEKQLIDYAKNSKKVVGNSTSLLSPYLHFGEISVRHVFQCARMKQIIWARDKNSEGEESADLFLRGIGLREYSRYICFNFPFTHEQSLLSHLRFFPWDADVDKFKAWRQGRTGYPLVDAGMRELWATGWMHNRIRVIVSSFAVKFLLLPWKWGMKYFWDTLLDADLECDILGWQYISGSIPDGHELDRLDNPALQGAKYDPEGEYIRQWLPELARLPTEWIHHPWDAPLTVLKASGVELGTNYAKPIVDIDTARELLAKAISRTRGAQIMIGAA

SEQ ID NO: 93 - protein, 1000 aa, synthetic construct
  REGION 1..1000: note = Synthetic construct
MKMDKKTIVWFRRDLRIEDNPALAAAAHEGSVFPVFIWCPEEEGQFYPGRASRWWMKQSLAHLSQSLKALGSDLTLIKTHNTISAILDCIRVTGATKVVFNHLYDPVSLVRDHTVKEKLVERGISVQSYNGDLLYEPWEIYCEKGKPFTSFNSYWKKCLDMSIESVMLPPPWRLMPITAAAEAIWACSIEELGLENEAEKPSNALLTRAWSPGWSNADKLLNEFIEKQLIDYAKNSKKVVGNSTSLLSPYLHFGEISVRHVFQCARMKQIIWARDKNSEGEESADLFLRGIGLREYSRYICFNFPFTHEQSLLSHLRFFPWDADVDKFKAWRQGRTGYPLVDAGMRELWATGWMHNRIRVIVSSFAVKFLLLPWKWGMKYFWDTLLDADLECDILGWQYISGSIPDGHELDRLDNPALQGAKYDPEGEYIRQWLPELARLPTEWIHHPWDAPLTVLKASGVELGTNYAKPIVDIDTARELLAKAISRTREAQIMIGAAMVMEKPSPLLVGREFVRQYYTLLNQAPDMLHRFYGKNSSYVHGGLDSNGKPADAVYGQKEIHRKVMSQNFTNCHTKIRHVDAHATLNDGVVVQVMGLLSNNNQALRRFMQTFVLAPEGSVANKFYVHNDIFRYQDEVFGGFVTEPQEESEEEVEEPEERQQTPEVVPDDSGTFYDQAVVSNDMEEHLEEPVAEPEPDPEPEPEQEPVSEIQEEKPEPVLEETAPEDAQKSSSPAPADIAQTVQEDLRTFSWASVTSKNLPPSGAVPVTGIPPHVVKVPASQPRPESKPESQIPPQRPQRDQRVREQRINIPPQRGPRPIREAGEQGDIEPRRMVRHPDSHQLFIGNLPHEVDKSELKDFFQSYGNVVELRINSGGKLPNFGFVVFDDSEPVQKVLSNRPIMFRGEVRLNVEEKKTRAAREGDRRDNRLRGPGGPRGGLGGGMRGPPRGGMVQKPGFGVGRGLAPRQKLIPMARRAWLEHGHSCFLCEIVIRSQFHTTYEPEA

SEQ ID NO: 94 - protein, 862 aa, synthetic construct
  REGION 1..862: note = Synthetic construct
MKMDKKTIVWFRRDLRIEDNPALAAAAHEGSVFPVFIWCPEEEGQFYPGRASRWWMKQSLAHLSQSLKALGSDLTLIKTHNTISAILDCIRVTGATKVVFNHLYDPVSLVRDHTVKEKLVERGISVQSYNGDLLYEPWEIYCEKGKPFTSFNSYWKKCLDMSIESVMLPPPWRLMPITAAAEAIWACSIEELGLENEAEKPSNALLTRAWSPGWSNADKLLNEFIEKQLIDYAKNSKKVVGNSTSLLSPYLHFGEISVRHVFQCARMKQIIWARDKNSEGEESADLFLRGIGLREYSRYICFNFPFTHEQSLLSHLRFFPWDADVDKFKAWRQGRTGYPLVDAGMRELWATGWMHNRIRVIVSSFAVKFLLLPWKWGMKYFWDTLLDADLECDILGWQYISGSIPDGHELDRLDNPALQGAKYDPEGEYIRQWLPELARLPTEWIHHPWDAPLTVLKASGVELGTNYAKPIVDIDTARELLAKAISRTREAQIMIGAAMGFVTEPQEESEEEVEEPEERQQTPEVVPDDSGTFYDQAVVSNDMEEHLEEPVAEPEPDPEPEPEQEPVSEIQEEKPEPVLEETAPEDAQKSSSPAPADIAQTVQEDLRTFSWASVTSKNLPPSGAVPVTGIPPHVVKVPASQPRPESKPESQIPPQRPQRDQRVREQRINIPPQRGPRPIREAGEQGDIEPRRMVRHPDSHQLFIGNLPHEVDKSELKDFFQSYGNVVELRINSGGKLPNFGFVVFDDSEPVQKVLSNRPIMFRGEVRLNVEEKKTRAAREGDRRDNRLRGPGGPRGGLGGGMRGPPRGGMVQKPGFGVGRGLAPRQKLIPMARRAWLEHGHSCFLCEIVIRSQFHTTYEPEA

SEQ ID NO: 95 - protein, 918 aa, synthetic construct
  REGION 1..918: note = Synthetic construct
MKMDKKTIVWFRRDLRIEDNPALAAAAHEGSVFPVFIWCPEEEGQFYPGRASRWWMKQSLAHLSQSLKALGSDLTLIKTHNTISAILDCIRVTGATKVVFNHLYDPVSLVRDHTVKEKLVERGISVQSYNGDLLYEPWEIYCEKGKPFTSFNSYWKKCLDMSIESVMLPPPWRLMPITAAAEAIWACSIEELGLENEAEKPSNALLTRAWSPGWSNADKLLNEFIEKQLIDYAKNSKKVVGNSTSLLSPYLHFGEISVRHVFQCARMKQIIWARDKNSEGEESADLFLRGIGLREYSRYICFNFPFTHEQSLLSHLRFFPWDADVDKFKAWRQGRTGYPLVDAGMRELWATGWMHNRIRVIVSSFAVKFLLLPWKWGMKYFWDTLLDADLECDILGWQYISGSIPDGHELDRLDNPALQGAKYDPEGEYIRQWLPELARLPTEWIHHPWDAPLTVLKASGVELGTNYAKPIVDIDTARELLAKAISRTREAQIMIGAAMVMEKPSPLLVGREFVRQYYTLLNQAPDMLHRFYGKNSSYVHGGLDSNGKPADAVYGQKEIHRKVMSQNFTNCHTKIRHVDAHATLNDGVVVQVMGLLSNNNQALRRFMQTFVLAPEGSVANKFYVHNDIFRYQDEVFGTAPEDAQKSSSPAPADIAQTVQEDLRTFSWASVTSKNLPPSGAVPVTGIPPHVVKVPASQPRPESKPESQIPPQRPQRDQRVREQRINIPPQRGPRPIREAGEQGDIEPRRMVRHPDSHQLFIGNLPHEVDKSELKDFFQSYGNVVELRINSGGKLPNFGFVVFDDSEPVQKVLSNRPIMFRGEVRLNVEEKKTRAAREGDRRDNRLRGPGGPRGGLGGGMRGPPRGGMVQKPGFGVGRGLAPRQKLIPMARRAWLEHGHSCFLCEIVIRSQFHTTYEPEA

SEQ ID NO: 96 - protein, 881 aa, synthetic construct
  REGION 1..881: note = Synthetic construct
MKMDKKTIVWFRRDLRIEDNPALAAAAHEGSVFPVFIWCPEEEGQFYPGRASRWWMKQSLAHLSQSLKALGSDLTLIKTHNTISAILDCIRVTGATKVVFNHLYDPVSLVRDHTVKEKLVERGISVQSYNGDLLYEPWEIYCEKGKPFTSFNSYWKKCLDMSIESVMLPPPWRLMPITAAAEAIWACSIEELGLENEAEKPSNALLTRAWSPGWSNADKLLNEFIEKQLIDYAKNSKKVVGNSTSLLSPYLHFGEISVRHVFQCARMKQIIWARDKNSEGEESADLFLRGIGLREYSRYICFNFPFTHEQSLLSHLRFFPWDADVDKFKAWRQGRTGYPLVDAGMRELWATGWMHNRIRVIVSSFAVKFLLLPWKWGMKYFWDTLLDADLECDILGWQYISGSIPDGHELDRLDNPALQGAKYDPEGEYIRQWLPELARLPTEWIHHPWDAPLTVLKASGVELGTNYAKPIVDIDTARELLAKAISRTREAQIMIGAAMVMEKPSPLLVGREFVRQYYTLLNQAPDMLHRFYGKNSSYVHGGLDSNGKPADAVYGQKEIHRKVMSQNFTNCHTKIRHVDAHATLNDGVVVQVMGLLSNNNQALRRFMQTFVLAPEGSVANKFYVHNDIFRYQDEVFGGFVTEPQEESEEEVEEPEERQQTPEVVPDDSGTFYDQAVVSNDMEEHLEEPVAEPEPDPEPEPEQEPVSEIQEEKPEPVLEEQLFIGNLPHEVDKSELKDFFQSYGNVVELRINSGGKLPNFGFVVFDDSEPVQKVLSNRPIMFRGEVRLNVEEKKTRAAREGDRRDNRLRGPGGPRGGLGGGMRGPPRGGMVQKPGFGVGRGLAPRQKLIPMARRAWLEHGHSCFLCEIVIRSQFHTTYEPEA

SEQ ID NO: 97 - protein, 929 aa, synthetic construct
  REGION 1..929: note = Synthetic construct
MKMDKKTIVWFRRDLRIEDNPALAAAAHEGSVFPVFIWCPEEEGQFYPGRASRWWMKQSLAHLSQSLKALGSDLTLIKTHNTISAILDCIRVTGATKVVFNHLYDPVSLVRDHTVKEKLVERGISVQSYNGDLLYEPWEIYCEKGKPFTSFNSYWKKCLDMSIESVMLPPPWRLMPITAAAEAIWACSIEELGLENEAEKPSNALLTRAWSPGWSNADKLLNEFIEKQLIDYAKNSKKVVGNSTSLLSPYLHFGEISVRHVFQCARMKQIIWARDKNSEGEESADLFLRGIGLREYSRYICFNFPFTHEQSLLSHLRFFPWDADVDKFKAWRQGRTGYPLVDAGMRELWATGWMHNRIRVIVSSFAVKFLLLPWKWGMKYFWDTLLDADLECDILGWQYISGSIPDGHELDRLDNPALQGAKYDPEGEYIRQWLPELARLPTEWIHHPWDAPLTVLKASGVELGTNYAKPIVDIDTARELLAKAISRTREAQIMIGAAMVMEKPSPLLVGREFVRQYYTLLNQAPDMLHRFYGKNSSYVHGGLDSNGKPADAVYGQKEIHRKVMSQNFTNCHTKIRHVDAHATLNDGVVVQVMGLLSNNNQALRRFMQTFVLAPEGSVANKFYVHNDIFRYQDEVFGGFVTEPQEESEEEVEEPEERQQTPEVVPDDSGTFYDQAVVSNDMEEHLEEPVAEPEPDPEPEPEQEPVSEIQEEKPEPVLEETAPEDAQKSSSPAPADIAQTVQEDLRTFSWASVTSKNLPPSGAVPVTGIPPHVVKVPASQPRPESKPESQIPPQRPQRDQRVREQRINIPPQRGPRPIREAGEQGDIEPRRMVRHPDSHEKKTRAAREGDRRDNRLRGPGGPRGGLGGGMRGPPRGGMVQKPGFGVGRGLAPRQKLIPMARRAWLEHGHSCFLCEIVIRSQFHTTYEPEA

SEQ ID NO: 98 - protein, 909 aa, synthetic construct
  REGION 1..909: note = Synthetic construct
MKMDKKTIVWFRRDLRIEDNPALAAAAHEGSVFPVFIWCPEEEGQFYPGRASRWWMKQSLAHLSQSLKALGSDLTLIKTHNTISAILDCIRVTGATKVVFNHLYDPVSLVRDHTVKEKLVERGISVQSYNGDLLYEPWEIYCEKGKPFTSFNSYWKKCLDMSIESVMLPPPWRLMPITAAAEAIWACSIEELGLENEAEKPSNALLTRAWSPGWSNADKLLNEFIEKQLIDYAKNSKKVVGNSTSLLSPYLHFGEISVRHVFQCARMKQIIWARDKNSEGEESADLFLRGIGLREYSRYICFNFPFTHEQSLLSHLRFFPWDADVDKFKAWRQGRTGYPLVDAGMRELWATGWMHNRIRVIVSSFAVKFLLLPWKWGMKYFWDTLLDADLECDILGWQYISGSIPDGHELDRLDNPALQGAKYDPEGEYIRQWLPELARLPTEWIHHPWDAPLTVLKASGVELGTNYAKPIVDIDTARELLAKAISRTREAQIMIGAAMVMEKPSPLLVGREFVRQYYTLLNQAPDMLHRFYGKNSSYVHGGLDSNGKPADAVYGQKEIHRKVMSQNFTNCHTKIRHVDAHATLNDGVVVQVMGLLSNNNQALRRFMQTFVLAPEGSVANKFYVHNDIFRYQDEVFGGFVTEPQEESEEEVEEPEERQQTPEVVPDDSGTFYDQAVVSNDMEEHLEEPVAEPEPDPEPEPEQEPVSEIQEEKPEPVLEETAPEDAQKSSSPAPADIAQTVQEDLRTFSWASVTSKNLPPSGAVPVTGIPPHVVKVPASQPRPESKPESQIPPQRPQRDQRVREQRINIPPQRGPRPIREAGEQGDIEPRRMVRHPDSHQLFIGNLPHEVDKSELKDFFQSYGNVVELRINSGGKLPNFGFVVFDDSEPVQKVLSNRPIMFRGEVRLNVE

SEQ ID NO: 99 - protein, 838 aa, synthetic construct
  REGION 1..838: note = Synthetic construct
MKMDKKTIVWFRRDLRIEDNPALAAAAHEGSVFPVFIWCPEEEGQFYPGRASRWWMKQSLAHLSQSLKALGSDLTLIKTHNTISAILDCIRVTGATKVVFNHLYDPVSLVRDHTVKEKLVERGISVQSYNGDLLYEPWEIYCEKGKPFTSFNSYWKKCLDMSIESVMLPPPWRLMPITAAAEAIWACSIEELGLENEAEKPSNALLTRAWSPGWSNADKLLNEFIEKQLIDYAKNSKKVVGNSTSLLSPYLHFGEISVRHVFQCARMKQIIWARDKNSEGEESADLFLRGIGLREYSRYICFNFPFTHEQSLLSHLRFFPWDADVDKFKAWRQGRTGYPLVDAGMRELWATGWMHNRIRVIVSSFAVKFLLLPWKWGMKYFWDTLLDADLECDILGWQYISGSIPDGHELDRLDNPALQGAKYDPEGEYIRQWLPELARLPTEWIHHPWDAPLTVLKASGVELGTNYAKPIVDIDTARELLAKAISRTREAQIMIGAAMVMEKPSPLLVGREFVRQYYTLLNQAPDMLHRFYGKNSSYVHGGLDSNGKPADAVYGQKEIHRKVMSQNFTNCHTKIRHVDAHATLNDGVVVQVMGLLSNNNQALRRFMQTFVLAPEGSVANKFYVHNDIFRYQDEVFGGFVTEPQEESEEEVEEPEERQQTPEVVPDDSGTFYDQAVVSNDMEEHLEEPVAEPEPDPEPEPEQEPVSEIQEEKPEPVLEETAPEDAQKSSSPAPADIAQTVQEDLRTFSWASVTSKNLPPSGAVPVTGIPPHVVKVPASQPRPESKPESQIPPQRPQRDQRVREQRINIPPQRGPRPIREAGEQGDIEPRRMVRHPDSH

SEQ ID NO: 100 - protein, 1000 aa, synthetic construct
  REGION 1..1000: note = Synthetic construct
MVMEKPSPLLVGREFVRQYYTLLNQAPDMLHRFYGKNSSYVHGGLDSNGKPADAVYGQKEIHRKVMSQNFTNCHTKIRHVDAHATLNDGVVVQVMGLLSNNNQALRRFMQTFVLAPEGSVANKFYVHNDIFRYQDEVFGGFVTEPQEESEEEVEEPEERQQTPEVVPDDSGTFYDQAVVSNDMEEHLEEPVAEPEPDPEPEPEQEPVSEIQEEKPEPVLEETAPEDAQKSSSPAPADIAQTVQEDLRTFSWASVTSKNLPPSGAVPVTGIPPHVVKVPASQPRPESKPESQIPPQRPQRDQRVREQRINIPPQRGPRPIREAGEQGDIEPRRMVRHPDSHQLFIGNLPHEVDKSELKDFFQSYGNVVELRINSGGKLPNFGFVVFDDSEPVQKVLSNRPIMFRGEVRLNVEEKKTRAAREGDRRDNRLRGPGGPRGGLGGGMRGPPRGGMVQKPGFGVGRGLAPRQKLIPMARRAWLEHGHSCFLCEIVIRSQFHTTYEPEAMKMDKKTIVWFRRDLRIEDNPALAAAAHEGSVFPVFIWCPEEEGQFYPGRASRWWMKQSLAHLSQSLKALGSDLTLIKTHNTISAILDCIRVTGATKVVFNHLYDPVSLVRDHTVKEKLVERGISVQSYNGDLLYEPWEIYCEKGKPFTSFNSYWKKCLDMSIESVMLPPPWRLMPITAAAEAIWACSIEELGLENEAEKPSNALLTRAWSPGWSNADKLLNEFIEKQLIDYAKNSKKVVGNSTSLLSPYLHFGEISVRHVFQCARMKQIIWARDKNSEGEESADLFLRGIGLREYSRYICFNFPFTHEQSLLSHLRFFPWDADVDKFKAWRQGRTGYPLVDAGMRELWATGWMHNRIRVIVSSFAVKFLLLPWKWGMKYFWDTLLDADLECDILGWQYISGSIPDGHELDRLDNPALQGAKYDPEGEYIRQWLPELARLPTEWIHHPWDAPLTVLKASGVELGTNYAKPIVDIDTARELLAKAISRTREAQIMIGAA

SEQ ID NO: 101 - protein, 862 aa, synthetic construct
  REGION 1..862: note = Synthetic construct
MGFVTEPQEESEEEVEEPEERQQTPEVVPDDSGTFYDQAVVSNDMEEHLEEPVAEPEPDPEPEPEQEPVSEIQEEKPEPVLEETAPEDAQKSSSPAPADIAQTVQEDLRTFSWASVTSKNLPPSGAVPVTGIPPHVVKVPASQPRPESKPESQIPPQRPQRDQRVREQRINIPPQRGPRPIREAGEQGDIEPRRMVRHPDSHQLFIGNLPHEVDKSELKDFFQSYGNVVELRINSGGKLPNFGFVVFDDSEPVQKVLSNRPIMFRGEVRLNVEEKKTRAAREGDRRDNRLRGPGGPRGGLGGGMRGPPRGGMVQKPGFGVGRGLAPRQKLIPMARRAWLEHGHSCFLCEIVIRSQFHTTYEPEAMKMDKKTIVWFRRDLRIEDNPALAAAAHEGSVFPVFIWCPEEEGQFYPGRASRWWMKQSLAHLSQSLKALGSDLTLIKTHNTISAILDCIRVTGATKVVFNHLYDPVSLVRDHTVKEKLVERGISVQSYNGDLLYEPWEIYCEKGKPFTSFNSYWKKCLDMSIESVMLPPPWRLMPITAAAEAIWACSIEELGLENEAEKPSNALLTRAWSPGWSNADKLLNEFIEKQLIDYAKNSKKVVGNSTSLLSPYLHFGEISVRHVFQCARMKQIIWARDKNSEGEESADLFLRGIGLREYSRYICFNFPFTHEQSLLSHLRFFPWDADVDKFKAWRQGRTGYPLVDAGMRELWATGWMHNRIRVIVSSFAVKFLLLPWKWGMKYFWDTLLDADLECDILGWQYISGSIPDGHELDRLDNPALQGAKYDPEGEYIRQWLPELARLPTEWIHHPWDAPLTVLKASGVELGTNYAKPIVDIDTARELLAKAISRTREAQIMIGAA

SEQ ID NO: 102 - protein, 918 aa, synthetic construct
  REGION 1..918: note = Synthetic construct
MVMEKPSPLLVGREFVRQYYTLLNQAPDMLHRFYGKNSSYVHGGLDSNGKPADAVYGQKEIHRKVMSQNFTNCHTKIRHVDAHATLNDGVVVQVMGLLSNNNQALRRFMQTFVLAPEGSVANKFYVHNDIFRYQDEVFGTAPEDAQKSSSPAPADIAQTVQEDLRTFSWASVTSKNLPPSGAVPVTGIPPHVVKVPASQPRPESKPESQIPPQRPQRDQRVREQRINIPPQRGPRPIREAGEQGDIEPRRMVRHPDSHQLFIGNLPHEVDKSELKDFFQSYGNVVELRINSGGKLPNFGFVVFDDSEPVQKVLSNRPIMFRGEVRLNVEEKKTRAAREGDRRDNRLRGPGGPRGGLGGGMRGPPRGGMVQKPGFGVGRGLAPRQKLIPMARRAWLEHGHSCFLCEIVIRSQFHTTYEPEAMKMDKKTIVWFRRDLRIEDNPALAAAAHEGSVFPVFIWCPEEEGQFYPGRASRWWMKQSLAHLSQSLKALGSDLTLIKTHNTISAILDCIRVTGATKVVFNHLYDPVSLVRDHTVKEKLVERGISVQSYNGDLLYEPWEIYCEKGKPFTSFNSYWKKCLDMSIESVMLPPPWRLMPITAAAEAIWACSIEELGLENEAEKPSNALLTRAWSPGWSNADKLLNEFIEKQLIDYAKNSKKVVGNSTSLLSPYLHFGEISVRHVFQCARMKQIIWARDKNSEGEESADLFLRGIGLREYSRYICFNFPFTHEQSLLSHLRFFPWDADVDKFKAWRQGRTGYPLVDAGMRELWATGWMHNRIRVIVSSFAVKFLLLPWKWGMKYFWDTLLDADLECDILGWQYISGSIPDGHELDRLDNPALQGAKYDPEGEYIRQWLPELARLPTEWIHHPWDAPLTVLKASGVELGTNYAKPIVDIDTARELLAKAISRTREAQIMIGAA

SEQ ID NO: 103 - protein, 881 aa, synthetic construct
  REGION 1..881: note = Synthetic construct
MVMEKPSPLLVGREFVRQYYTLLNQAPDMLHRFYGKNSSYVHGGLDSNGKPADAVYGQKEIHRKVMSQNFTNCHTKIRHVDAHATLNDGVVVQVMGLLSNNNQALRRFMQTFVLAPEGSVANKFYVHNDIFRYQDEVFGGFVTEPQEESEEEVEEPEERQQTPEVVPDDSGTFYDQAVVSNDMEEHLEEPVAEPEPDPEPEPEQEPVSEIQEEKPEPVLEEQLFIGNLPHEVDKSELKDFFQSYGNVVELRINSGGKLPNFGFVVFDDSEPVQKVLSNRPIMFRGEVRLNVEEKKTRAAREGDRRDNRLRGPGGPRGGLGGGMRGPPRGGMVQKPGFGVGRGLAPRQKLIPMARRAWLEHGHSCFLCEIVIRSQFHTTYEPEAMKMDKKTIVWFRRDLRIEDNPALAAAAHEGSVFPVFIWCPEEEGQFYPGRASRWWMKQSLAHLSQSLKALGSDLTLIKTHNTISAILDCIRVTGATKVVFNHLYDPVSLVRDHTVKEKLVERGISVQSYNGDLLYEPWEIYCEKGKPFTSFNSYWKKCLDMSIESVMLPPPWRLMPITAAAEAIWACSIEELGLENEAEKPSNALLTRAWSPGWSNADKLLNEFIEKQLIDYAKNSKKVVGNSTSLLSPYLHFGEISVRHVFQCARMKQIIWARDKNSEGEESADLFLRGIGLREYSRYICFNFPFTHEQSLLSHLRFFPWDADVDKFKAWRQGRTGYPLVDAGMRELWATGWMHNRIRVIVSSFAVKFLLLPWKWGMKYFWDTLLDADLECDILGWQYISGSIPDGHELDRLDNPALQGAKYDPEGEYIRQWLPELARLPTEWIHHPWDAPLTVLKASGVELGTNYAKPIVDIDTARELLAKAISRTREAQIMIGAA

SEQ ID NO: 104 - protein, 929 aa, synthetic construct
  REGION 1..929: note = Synthetic construct
MVMEKPSPLLVGREFVRQYYTLLNQAPDMLHRFYGKNSSYVHGGLDSNGKPADAVYGQKEIHRKVMSQNFTNCHTKIRHVDAHATLNDGVVVQVMGLLSNNNQALRRFMQTFVLAPEGSVANKFYVHNDIFRYQDEVFGGFVTEPQEESEEEVEEPEERQQTPEVVPDDSGTFYDQAVVSNDMEEHLEEPVAEPEPDPEPEPEQEPVSEIQEEKPEPVLEETAPEDAQKSSSPAPADIAQTVQEDLRTFSWASVTSKNLPPSGAVPVTGIPPHVVKVPASQPRPESKPESQIPPQRPQRDQRVREQRINIPPQRGPRPIREAGEQGDIEPRRMVRHPDSHEKKTRAAREGDRRDNRLRGPGGPRGGLGGGMRGPPRGGMVQKPGFGVGRGLAPRQKLIPMARRAWLEHGHSCFLCEIVIRSQFHTTYEPEAMKMDKKTIVWFRRDLRIEDNPALAAAAHEGSVFPVFIWCPEEEGQFYPGRASRWWMKQSLAHLSQSLKALGSDLTLIKTHNTISAILDCIRVTGATKVVFNHLYDPVSLVRDHTVKEKLVERGISVQSYNGDLLYEPWEIYCEKGKPFTSFNSYWKKCLDMSIESVMLPPPWRLMPITAAAEAIWACSIEELGLENEAEKPSNALLTRAWSPGWSNADKLLNEFIEKQLIDYAKNSKKVVGNSTSLLSPYLHFGEISVRHVFQCARMKQIIWARDKNSEGEESADLFLRGIGLREYSRYICFNFPFTHEQSLLSHLRFFPWDADVDKFKAWRQGRTGYPLVDAGMRELWATGWMHNRIRVIVSSFAVKFLLLPWKWGMKYFWDTLLDADLECDILGWQYISGSIPDGHELDRLDNPALQGAKYDPEGEYIRQWLPELARLPTEWIHHPWDAPLTVLKASGVELGTNYAKPIVDIDTARELLAKAISRTREAQIMIGAA

SEQ ID NO: 105 - protein, 909 aa, synthetic construct
  REGION 1..909: note = Synthetic construct
MVMEKPSPLLVGREFVRQYYTLLNQAPDMLHRFYGKNSSYVHGGLDSNGKPADAVYGQKEIHRKVMSQNFTNCHTKIRHVDAHATLNDGVVVQVMGLLSNNNQALRRFMQTFVLAPEGSVANKFYVHNDIFRYQDEVFGGFVTEPQEESEEEVEEPEERQQTPEVVPDDSGTFYDQAVVSNDMEEHLEEPVAEPEPDPEPEPEQEPVSEIQEEKPEPVLEETAPEDAQKSSSPAPADIAQTVQEDLRTFSWASVTSKNLPPSGAVPVTGIPPHVVKVPASQPRPESKPESQIPPQRPQRDQRVREQRINIPPQRGPRPIREAGEQGDIEPRRMVRHPDSHQLFIGNLPHEVDKSELKDFFQSYGNVVELRINSGGKLPNFGFVVFDDSEPVQKVLSNRPIMFRGEVRLNVEMKMDKKTIVWFRRDLRIEDNPALAAAAHEGSVFPVFIWCPEEEGQFYPGRASRWWMKQSLAHLSQSLKALGSDLTLIKTHNTISAILDCIRVTGATKVVFNHLYDPVSLVRDHTVKEKLVERGISVQSYNGDLLYEPWEIYCEKGKPFTSFNSYWKKCLDMSIESVMLPPPWRLMPITAAAEAIWACSIEELGLENEAEKPSNALLTRAWSPGWSNADKLLNEFIEKQLIDYAKNSKKVVGNSTSLLSPYLHFGEISVRHVFQCARMKQIIWARDKNSEGEESADLFLRGIGLREYSRYICFNFPFTHEQSLLSHLRFFPWDADVDKFKAWRQGRTGYPLVDAGMRELWATGWMHNRIRVIVSSFAVKFLLLPWKWGMKYFWDTLLDADLECDILGWQYISGSIPDGHELDRLDNPALQGAKYDPEGEYIRQWLPELARLPTEWIHHPWDAPLTVLKASGVELGTNYAKPIVDIDTARELLAKAISRTREAQIMIGAA

SEQ ID NO: 106 - protein, 838 aa, synthetic construct
  REGION 1..838: note = Synthetic construct
MVMEKPSPLLVGREFVRQYYTLLNQAPDMLHRFYGKNSSYVHGGLDSNGKPADAVYGQKEIHRKVMSQNFTNCHTKIRHVDAHATLNDGVVVQVMGLLSNNNQALRRFMQTFVLAPEGSVANKFYVHNDIFRYQDEVFGGFVTEPQEESEEEVEEPEERQQTPEVVPDDSGTFYDQAVVSNDMEEHLEEPVAEPEPDPEPEPEQEPVSEIQEEKPEPVLEETAPEDAQKSSSPAPADIAQTVQEDLRTFSWASVTSKNLPPSGAVPVTGIPPHVVKVPASQPRPESKPESQIPPQRPQRDQRVREQRINIPPQRGPRPIREAGEQGDIEPRRMVRHPDSHMKMDKKTIVWFRRDLRIEDNPALAAAAHEGSVFPVFIWCPEEEGQFYPGRASRWWMKQSLAHLSQSLKALGSDLTLIKTHNTISAILDCIRVTGATKVVFNHLYDPVSLVRDHTVKEKLVERGISVQSYNGDLLYEPWEIYCEKGKPFTSFNSYWKKCLDMSIESVMLPPPWRLMPITAAAEAIWACSIEELGLENEAEKPSNALLTRAWSPGWSNADKLLNEFIEKQLIDYAKNSKKVVGNSTSLLSPYLHFGEISVRHVFQCARMKQIIWARDKNSEGEESADLFLRGIGLREYSRYICFNFPFTHEQSLLSHLRFFPWDADVDKFKAWRQGRTGYPLVDAGMRELWATGWMHNRIRVIVSSFAVKFLLLPWKWGMKYFWDTLLDADLECDILGWQYISGSIPDGHELDRLDNPALQGAKYDPEGEYIRQWLPELARLPTEWIHHPWDAPLTVLKASGVELGTNYAKPIVDIDTARELLAKAISRTREAQIMIGAA

SEQ ID NO: 107 - protein, 161 aa, Avena sativa
GEFLATTLERIEKNFVITDPRLPDNPIIFASDSFLQLTEYSREEILGRNCRFLQGPETDRATVRKIRDAIDNQTEVTVQLINYTKSGKKFWNLFHLQPMRDQKGDVQYFIGVQLDGTEHVRDAAEREGVMLIKKTAENIDEAAKELPDANLRPEDLWANHG

SEQ ID NO: 108 - protein, 225 aa, Erythrobacter litoralis
MLDMGQDRPIDGSGAPGADDTRVEVQPPAQWVLDLIEASPIASVVSDPRLADNPLIAINQAFTDLTGYSEEECVGRNCRFLAGSGTEPWLTDKIRQGVREHKPVLVEILNYKKDGTPFRNAVLVAPIYDDDDELLYFLGSQVEVDDDQPNMGMARRERAAEMLKTLSPRQLEVTTLVASGLRNKEVAARLGLSEKTVKMHRGLVMEKLNLKTSADLVRIAVEAGI

SEQ ID NO: 109 - protein, 261 aa, Arabidopsis sp.
MASFQSFGIPGQLEVIKKALDHVRVGVVITDPALEDNPIVYVNQGFVQMTGYETEEILGKNCRFLQGKHTDPAEVDNIRTALQNKEPVTVQIQNYKKDGTMFWNELNIDPMEIEDKTYFVGIQNDITKQKEYEKLLEDSLTEITALSTPIVPIRNGISALPLVGNLTEERFNSIVCTLTNILSTSKDDYLIIDLSGLAQVNEQTADQIFKLSHLLKLTGTELIITGIKPELAMKMNKLDANFSSLKTYSNVKDAVKVLPIM

SEQ ID NO: 110 - protein, 176 aa, Rhodobacter sphaeroides
MDQKQFEKIRAVFDRSGVALTLVDMSLPEQPVVLANPPFLRMTGYTEGQILGFNCRFLQRGDENAQARADIRDALKLGRELQVVLRNYRANDEPFDNLLFLHPVGGRPDAPDYFLGSQFELGRSGNSEEAAAAGHAGALTGELARIGTVAARLEMDSRRHLAQAAAALVRAWERRG